(19) United States
(12) Patent Application Publication
(10) Pub. No.: US 2006/0009633 A9
(43) Pub. Date: Jan. 12, 2006

(54) Complementary DNAs encoding proteins with signal peptides

(75) Inventors: Jean-Baptiste Dumas Milne Edwards, Paris (FR); Lydie Bougueleret, Petit Lancy (CH); Severin Jobert, Paris (FR); Catherine Clusel, Vincennes (FR); Aymeric Duclert, Saint-Maur (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: GENSET, S.A., Paris (FR)

(21) Appl. No.: 09/978,360

(22) Filed: Oct. 15, 2001

Related U.S. Application Data
(63) Continuation-in-part of application No. 09/663,600, filed on Sep. 15, 2000, now Pat. No. 6,573,068.
Application No. 09/663,600 is a continuation-in-part of application No. 09/191,997, filed on Nov. 13, 1998, now abandoned.
(63) Continuation-in-part of application No. 09/215,435, filed on Dec. 17, 1998, now abandoned.
(63) Continuation-in-part of application No. PCT/IB98/02122, filed on Dec. 17, 1998.
(63) Continuation-in-part of application No. 09/247,155, filed on Feb. 9, 1999, now Pat. No. 6,312,922.
(63) Continuation-in-part of application No. PCT/IB99/00282, filed on Feb. 9, 1999.
(63) Continuation-in-part of application No. 09/599,360, filed on Jun. 21, 2000, now Pat. No. 6,548,633.
Application No. 09/599,360 is a continuation-in-part of application No. 09/469,099, filed on Dec. 21, 1999, now abandoned.
(63) Continuation-in-part of application No. PCT/IB00/00951, filed on Jun. 21, 2000.
(60) Provisional application No. 60/066,677, filed on Nov. 13, 1997.
(60) Provisional application No. 60/069,957, filed on Dec. 17, 1997.
(60) Provisional application No. 60/074,121, filed on Feb. 9, 1998.
(60) Provisional application No. 60/099,273, filed on Sep. 4, 1998.
(60) Provisional application No. 60/096,116, filed on Aug. 10, 1998.
(60) Provisional application No. 60/081,563, filed on Apr. 13, 1998.
(60) Provisional application No. 60/069,957, filed on Dec. 17, 1997.
(60) Provisional application No. 60/074,121, filed on Feb. 9, 1998.
(60) Provisional application No. 60/081,563, filed on Apr. 13, 1998.
(60) Provisional application No. 60/096,116, filed on Aug. 10, 1998.
(60) Provisional application No. 60/099,273, filed on Sep. 4, 1998.
(60) Provisional application No. 60/096,116, filed on Aug. 10, 1998.
(60) Provisional application No. 60/081,563, filed on Apr. 13, 1998.
(60) Provisional application No. 60/074,121, filed on Feb. 9, 1998.
(60) Provisional application No. 60/069,957, filed on Dec. 17, 1997.
(60) Provisional application No. 60/074,121, filed on Feb. 9, 1998.
(60) Provisional application No. 60/081,563, filed on Apr. 13, 1998.
(60) Provisional application No. 60/096,116, filed on Aug. 10, 1998.
(60) Provisional application No. 60/099,273, filed on Sep. 4, 1998.
(60) Provisional application No. 60/099,273, filed on Sep. 4, 1998.
(60) Provisional application No. 60/096,116, filed on Aug. 10, 1998.
(60) Provisional application No. 60/081,563, filed on Apr. 13, 1998.
(60) Provisional application No. 60/074,121, filed on Feb. 9, 1998.
(60) Provisional application No. 60/113,686, filed on Dec. 22, 1998.
(60) Provisional application No. 60/141,032, filed on Jun. 25, 1999.
(60) Provisional application No. 60/141,032, filed on Jun. 25, 1999.

(51) Int. Cl. C12Q 1/68; C07H 21/04; C07K 14/47

(52) U.S. Cl. 536/23.5; 435/6; 435/320.1; 435/325; 530/350; 530/388.1

(57) ABSTRACT

The sequences of cDNAs encoding secreted proteins are disclosed. The cDNAs can be used to express secreted proteins or fragments thereof or to obtain antibodies capable of specifically binding to the secreted proteins. The cDNAs may also be used in diagnostic, forensic, gene therapy, and chromosome mapping procedures. The cDNAs may also be used to design expression vectors and secretion vectors.

### RELATED U.S. APPLICATION DATA

The present application is a continuation-in-part of:

U.S. CIP application Ser. No. 09/663,600, filed Sep. 15, 2000, and claims priority from U.S. application Ser. No. 09/191,997, filed Nov. 13, 1998; U.S. Provisional Application Serial No. 60/066,677, filed Nov. 13, 1997; U.S. Provisional Application Serial No. 60/069,957, filed Dec. 17, 1997; U.S. Provisional Application Serial No. 60/074,121, filed Feb. 9, 1998; U.S. Provisional Application Serial No. 60/081,563, filed Apr. 13, 1998; U.S. Provisional Application Serial No. 60/096,116, filed Aug. 10, 1998, and U.S. Provisional Application Serial No. 60/099,273, filed Sep. 4, 1998, the entireties of which are hereby incorporated by reference;

U.S. patent application Ser. No. 09/215,435 and PCT Application PCT/IB98/02122, filed Dec. 17, 1998, and claims priority from U.S. Provisional Patent Application Serial No. 60/069,957, filed Dec. 17, 1997; U.S. Provisional Patent Application Serial No. 60/074,121, filed Feb. 9, 1998; U.S. Provisional Patent Application Serial No. 60/081,563, filed Apr. 13, 1998; U.S. Provisional Patent Application Serial No. 60/096,116, filed Aug. 10, 1998; and U.S. Provisional Patent Application Serial No. 60/099,273, filed Sep. 4, 1998, the disclosures of which are incorporated herein by reference in their entirety;

U.S. patent application Ser. No. 09/247,155 and PCT Application PCT/IB99/00282 filed Feb. 9, 1999, and claims priority from U.S. Provisional Patent Application Serial No. 60/074,121, filed Feb. 9, 1998; U.S. Provisional Patent Application Serial No. 60/081,563, filed Apr. 13, 1998; U.S. Provisional Patent Application Serial No. 60/096,116, filed Aug. 10, 1998; and U.S. Provisional Patent Application Serial No. 60/099,273, filed Sep. 4, 1998, the disclosures of which are incorporated herein by reference in their entirety; and

U.S. CIP application Ser. No. 09/599,360 and PCT Application PCT/1B00/00951 filed Jun. 21, 2000, and claims priority from U.S. application Ser. No. 09/469,099, filed Dec. 21, 1999; U.S. Provisional Patent Application Serial No. 60/113,686, filed Dec. 22, 1998; and U.S. Provisional Patent Application Serial No. 60/141,032, filed Jun. 25, 1999, the disclosures of which are incorporated herein by reference in their entireties.

### BACKGROUND OF THE INVENTION

The estimated 50,000-100,000 genes scattered along the human chromosomes offer tremendous promise for the understanding, diagnosis, and treatment of human diseases. In addition, probes capable of specifically hybridizing to loci distributed throughout the human genome find applications in the construction of high resolution chromosome maps and in the identification of individuals.

In the past, the characterization of even a single human gene was a painstaking process, requiring years of effort. Recent developments in the areas of cloning vectors, DNA sequencing, and computer technology have merged to greatly accelerate the rate at which human genes can be isolated, sequenced, mapped, and characterized.

Currently, two different approaches are being pursued for identifying and characterizing the genes distributed along the human genome. In one approach, large fragments of genomic DNA are isolated, cloned, and sequenced. Potential open reading frames in these genomic sequences are identified using bio-informatics software. However, this approach entails sequencing large stretches of human DNA which do not encode proteins in order to find the protein encoding sequences scattered throughout the genome. In addition to requiring extensive sequencing, the bio-informatics software may mischaracterize the genomic sequences obtained, i.e., labeling non-coding DNA as coding DNA and vice versa.

An alternative approach takes a more direct route to identifying and characterizing human genes. In this approach, complementary DNAs (cDNAs) are synthesized from isolated messenger RNAs (mRNAs) which encode human proteins. Using this approach, sequencing is only performed on DNA which is derived from protein coding fragments of the genome. Often, only short stretches of the cDNAs are sequenced to obtain sequences called expressed sequence tags (ESTs). The ESTs may then be used to isolate or purify cDNAs which include sequences adjacent to the EST sequences. The cDNAs may contain all of the sequence of the EST which was used to obtain them or only a fragment of the sequence of the EST which was used to obtain them. In addition, the cDNAs may contain the full coding sequence of the gene from which the EST was derived or, alternatively, the cDNAs may include fragments of the coding sequence of the gene from which the EST was derived. It will be appreciated that there may be several cDNAs which include the EST sequence as a result of alternate splicing or the activity of alternative promoters.

In the past, these short EST sequences were often obtained from oligo-dT primed cDNA libraries. Accordingly, they mainly corresponded to the 3′ untranslated region of the mRNA. In part, the prevalence of EST sequences derived from the 3′ end of the mRNA is a result of the fact that typical techniques for obtaining cDNAs, are not well suited for isolating cDNA sequences derived from the 5′ ends of mRNAs (Adams et al., Nature 377:3-174, 1996, Hillier et al., Genome Res. 6:807-828, 1996). In addition, in those reported instances where longer cDNA sequences have been obtained, the reported sequences typically correspond to coding sequences and do not include the full 5′ untranslated region (5′UTR) of the mRNA from which the cDNA is derived. Indeed, 5′UTRs have been shown to affect either the stability or translation of mRNAs. Thus, regulation of gene expression may be achieved through the use of alternative 5′UTRs as shown, for instance, for the translation of the tissue inhibitor of metalloprotease mRNA in mitogenically activated cells (Waterhouse et al., J Biol. Chem. 265:5585-9. 1990). Furthermore, modification of 5′UTR through mutation, insertion or translocation events may even be implied in pathogenesis. For instance, the fragile X syndrome, the most common cause of inherited mental retardation, is partly due to an insertion of multiple CGG trinucleotides in the 5′UTR of the fragile X mRNA resulting in the inhibition of protein synthesis via ribosome stalling (Feng et al., Science 268:731-4, 1995). An aberrant mutation in regions of the 5′UTR known to inhibit translation of the proto-oncogene c-myc was shown to result in upregulation of c-myc protein levels in cells derived from patients with multiple myelomas (Willis et al., Curr Top Microbiol Immunol 224:269-76, 1997). In addition, the use of oligo-dT primed cDNA libraries does not allow the isolation of complete 5′UTRs since such incomplete sequences obtained by this process may not include the first exon of the mRNA, particularly in situations where the first exon is short. Furthermore, they may not include some exons, often short ones, which are located upstream of splicing sites. Thus, there is a need to obtain sequences derived from the 5′ ends of mRNAs.

Moreover, despite the great amount of EST data that large-scale sequencing projects have yielded (Adams et al., Nature 377:174, 1996, Hillier et al., Genome Res. 6:807-828, 1996), information concerning the biological function of the mRNAs corresponding to such obtained cDNAs has revealed to be limited. Indeed, whereas the knowledge of the complete coding sequence is absolutely necessary to investigate the biological function of mRNAs, ESTs yield only partial coding sequences. So far, large-scale full-length cDNA cloning has been achieved only with limited success because of the poor efficiency of methods for constructing full-length cDNA libraries. Indeed, such methods require either a large amount of mRNA (Ederly et al., 1995), thus resulting in non representative full-length libraries when small amounts of tissue are available or require PCR amplification (Maruyama et al., 1994; CLONTECHniques, 1996) to obtain a reasonable number of clones, thus yielding strongly biased cDNA libraries where rare and long cDNAs are lost. Thus, there is a need to obtain full-length cDNAs, i.e. cDNAs containing the full coding sequence of their corresponding mRNAs.

While many sequences derived from human chromosomes have practical applications, approaches based on the identification and characterization of those chromosomal sequences which encode a protein product are particularly relevant to diagnostic and therapeutic uses. Of the 50,000-100,000 protein coding genes, those genes encoding proteins which are secreted from the cell in which they are synthesized, as well as the secreted proteins themselves, are particularly valuable as potential therapeutic agents. Such proteins are often involved in cell to cell communication and may be responsible for producing a clinically relevant response in their target cells. In fact, several secretory proteins, including tissue plasminogen activator, G-CSF, GM-CSF, erythropoietin, human growth hormone, insulin, interferon-α, interferon-β, interferon-γ, and interleukin-2, are currently in clinical use. These proteins are used to treat a wide range of conditions, including acute myocardial infarction, acute ischemic stroke, anemia, diabetes, growth hormone deficiency, hepatitis, kidney carcinoma, chemotherapy induced neutropenia and multiple sclerosis. For these reasons, cDNAs encoding secreted proteins or fragments thereof represent a particularly valuable source of therapeutic agents. Thus, there is a need for the identification and characterization of secreted proteins and the nucleic acids encoding them.

In addition to being therapeutically useful themselves, secretory proteins include short peptides, called signal peptides, at their amino termini which direct their secretion. These signal peptides are encoded by the signal sequences located at the 5′ ends of the coding sequences of genes encoding secreted proteins. Because these signal peptides will direct the extracellular secretion of any protein to which they are operably linked, the signal sequences may be exploited to direct the efficient secretion of any protein by operably linking the signal sequences to a gene encoding the protein for which secretion is desired. In addition, fragments of the signal peptides called membrane-translocating sequences, may also be used to direct the intracellular import of a peptide or protein of interest. This may prove beneficial in gene therapy strategies in which it is desired to deliver a particular gene product to cells other than the cells in which it is produced. Signal sequences encoding signal peptides also find application in simplifying protein purification techniques. In such applications, the extracellular secretion of the desired protein greatly facilitates purification by reducing the number of undesired proteins from which the desired protein must be selected. Thus, there exists a need to identify and characterize the 5′ fragments of the genes for secretory proteins which encode signal peptides.

Sequences coding for secreted proteins may also find application as therapeutics or diagnostics. In particular, such sequences may be used to determine whether an individual is likely to express a detectable phenotype, such as a disease, as a consequence of a mutation in the coding sequence for a secreted protein. In instances where the individual is at risk of suffering from a disease or other undesirable phenotype as a result of a mutation in such a coding sequence, the undesirable phenotype may be corrected by introducing a normal coding sequence using gene therapy. Alternatively, if the undesirable phenotype results from overexpression of the protein encoded by the coding sequence, expression of the protein may be reduced using antisense or triple helix based strategies.

The secreted human polypeptides encoded by the coding sequences may also be used as therapeutics by administering them directly to an individual having a condition, such as a disease, resulting from a mutation in the sequence encoding the polypeptide. In such an instance, the condition can be cured or ameliorated by administering the polypeptide to the individual.

In addition, the secreted human polypeptides or fragments thereof may be used to generate antibodies useful in determining the tissue type or species of origin of a biological sample. The antibodies may also be used to determine the cellular localization of the secreted human polypeptides or the cellular localization of polypeptides which have been fused to the human polypeptides. In addition, the antibodies may also be used in immunoaffinity chromatography techniques to isolate, purify, or enrich the human polypeptide or a target polypeptide which has been fused to the human polypeptide.

Public information on the number of human genes for which the promoters and upstream regulatory regions have been identified and characterized is quite limited. In part, this may be due to the difficulty of isolating such regulatory sequences. Upstream regulatory sequences such as transcription factor binding sites are typically too short to be utilized as probes for isolating promoters from human genomic libraries. Recently, some approaches have been developed to isolate human promoters. One of them consists of making a CpG island library (Cross et al., Nature Genetics 6: 236-244, 1994). The second consists of isolating human genomic DNA sequences containing SpeI binding sites by the use of SpeI binding protein. (Mortlock et al., Genome Res. 6:327-335, 1996). Both of these approaches have their limits due to a lack of specificity and of comprehensiveness. Thus, there exists a need to identify and systematically characterize the 5′ fragments of the genes.

cDNAs including the 5′ ends of their corresponding mRNA may be used to efficiently identify and isolate 5′UTRs and upstream regulatory regions which control the location, developmental stage, rate, and quantity of protein synthesis, as well as the stability of the mRNA (Theil et al., BioFactors 4:87-93, (1993). Once identified and characterized, these regulatory regions may be utilized in gene therapy or protein purification schemes to obtain the desired amount and locations of protein synthesis or to inhibit, reduce, or prevent the synthesis of undesirable gene products.

In addition, cDNAs containing the 5′ ends of secretory protein genes may include sequences useful as probes for chromosome mapping and the identification of individuals. Thus, there is a need to identify and characterize the sequences upstream of the 5′ coding sequences of genes encoding secretory proteins.

### SUMMARY OF THE INVENTION

The present invention relates to purified, isolated, or recombinant cDNAs which encode secreted proteins or fragments thereof. Preferably, the purified, isolated or recombinant cDNAs contain the entire open reading frame of their corresponding mRNAs, including a start codon and a stop codon. For example, the cDNAs may include nucleic acids encoding the signal peptide as well as the mature protein. Such cDNAs will be referred herein as “full-length” cDNAs. Alternatively, the cDNAs may contain a fragment of the open reading frame. Such cDNAs will be referred herein as “ESTs” or “5′ESTs”. In some embodiments, the fragment may encode only the sequence of the mature protein. Alternatively, the fragment may encode only a fragment of the mature protein. A further aspect of the present invention is a nucleic acid which encodes the signal peptide of a secreted protein.

The term “corresponding mRNA” refers to the mRNA which was the template for the cDNA synthesis which produced the cDNA of the present invention.

As used herein, the term “purified” does not require absolute purity; rather, it is intended as a relative definition. Purification of starting material or natural material is at least one order of magnitude, preferably two or three orders, and more preferably four or five orders of magnitude is expressly contemplated. As an example, purification from 0.1% concentration to 10% concentration is two orders of magnitude.

To illustrate, individual cDNA clones isolated from a cDNA library have been conventionally purified to electrophoretic homogeneity. The sequences obtained from these clones could not be obtained directly either from the library or from total human DNA. The cDNA clones are not naturally occurring as such, but rather are obtained via manipulation of a partially purified naturally occurring substance (messenger RNA). The conversion of mRNA into a cDNA library involves the creation of a synthetic substance (cDNA) and pure individual cDNA clones can be isolated from the synthetic library by clonal selection. Thus, creating a cDNA library from messenger RNA and subsequently isolating individual clones from that library results in an approximately 10⁴-10⁶ fold purification of the native message.

The term “purified” is further used herein to describe a polypeptide or polynucleotide of the invention which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero-dimers, trimers, etc. The term “purified” may also be used to specify the separation of covalently closed polynucleotides from linear polynucleotides. A polynucleotide is substantially pure when at least about 50%, preferably 60 to 75% of a sample exhibits a single polynucleotide sequence and conformation (linear versus covalently close). A substantially pure polypeptide or polynucleotide typically comprises about 50%, preferably 60 to 90% weight/weight of a polypeptide or polynucleotide sample, respectively, more usually about 95%, and preferably is over about 99% pure. Polypeptide and polynucleotide purity, or homogeneity, is indicated by a number of means well known in the art, such as agarose or polyacrylamide gel electrophoresis of a sample, followed by visualizing a single band upon staining the gel. For certain purposes higher resolution can be provided by using HPLC or other means well known in the art. As an alternative embodiment, purification of the polypeptides and polynucleotides of the present invention may be expressed as “at least” a percent purity relative to heterologous polypeptides and polynucleotides (DNA, RNA or both). As a preferred embodiment, the polypeptides and polynucleotides of the present invention are at least; 10%, 20%, 30%, 40%, 50%, 60%, 70%, 80%, 90%, 95%, 96%, 96%, 98%, 99%, or 100% pure relative to heterologous polypeptides and polynucleotides, respectively. As a further preferred embodiment the polypeptides and polynucleotides have a purity ranging from any number, to the thousandth position, between 90% and 100% (e.g., a polypeptide or polynucleotide at least 99.995% pure) relative to either heterologous polypeptides or polynucleotides, respectively, or as a weight/weight ratio relative to all compounds and molecules other than those existing in the carrier. Each number representing a percent purity, to the thousandth position, may be claimed as individual species of purity.

As used herein, the term “recombinant polynucleotide” means that the cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

Unless otherwise specified, nucleotides and amino acids of polynucleotide and polypeptide fragments (respectively) of the present invention are contiguous and not interrupted by heterologous sequences.

The term “isolated” requires that the material be removed from its original environment (e.g., the natural environment if it is naturally occurring). For example, a naturally occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment. Specifically excluded from the definition of “isolated” are: naturally occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies, and/or further wherein the polynucleotide of the present invention makes up less than 5% (or alternatively 1%, 2%, 3%, 4%, 10%, 25%, 50%, 75%, or 90%, 95%, or 99%) of the number of nucleic acid inserts in the vector molecules. Further specifically excluded are whole cell genomic DNA or whole cell RNA preparations (including said whole cell preparations which are mechanically sheared or enzymaticly digested). Further specifically excluded are the above whole cell preparations as either an in vitro preparation or as a heterogeneous mixture separated by electrophoresis (including blot transfers of the same) wherein the polynucleotide of the invention have not been further separated from the heterologous polynucleotides in the electrophoresis medium (e.g., further separating by excising a single band from a heterogeneous band population in an agarose gel or nylon blot).

Thus, cDNAs encoding secreted polypeptides or fragments thereof which are present in cDNA libraries in which one or more cDNAs encoding secreted polypeptides or fragments thereof make up 5% or more of the number of nucleic acid inserts in the backbone molecules are “enriched recombinant cDNAs” as defined herein. Likewise, cDNAs encoding secreted polypeptides or fragments thereof which are in a population of plasmids in which one or more cDNAs of the present invention have been inserted such that they represent 5% or more of the number of inserts in the plasmid backbone are “enriched recombinant cDNAs” as defined herein. However, cDNAs encoding secreted polypeptides or fragments thereof which are in cDNA libraries in which the cDNAs encoding secreted polypeptides or fragments thereof constitute less than 5% of the number of nucleic acid inserts in the population of backbone molecules, such as libraries in which backbone molecules having a cDNA insert encoding a secreted polypeptide are extremely rare, are not “enriched recombinant cDNAs.”

The term “polypetide” refers to a polymer of amino acids without regard to the length of the polymer; thus, “peptides,” “oligopeptides”, and “proteins” are included within the definition of polypeptide and used interchangeably herein. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12, 1983; Seifter et al., Meth Enzymol 182:626-646, 1990; Rattan et al., Ann NY Acad Sci 663:48-62, 1992). Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring. The term “polypeptide” may also be used interchangeably with the term “protein”.

As used interchangeably herein, the terms “nucleic acid molecule”, “oligonucleotides”, and “polynucleotides” include RNA or, DNA (either single or double stranded, coding, non-coding, complementary or antisense), or RNA/DNA hybrid sequences of more than one nucleotide in either single chain or duplex form (although each of the above species may be particularly specified). The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications (a) an alternative linking group, (b) an analogous form of purine, (c) an analogous form of pyrimidine, or (d) an analogous sugar; for examples of analogous linking groups, purine, pyrimidines, and sugars see for example PCT publication No. WO 95/04064. Preferred modifications of the present invention include, but are not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v) ybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine. Methylenemethylimino linked oligonucleosides as well as mixed backbone compounds having, may be prepared as described in U.S. Pat. Nos. 5,378,825; 5,386,023; 5,489,677; 5,602,240; and 5,610,289. Formacetal and thioformacetal linked oligonucleosides may be prepared as described in U.S. Pat. Nos. 5,264,562 and 5,264,564. Ethylene oxide linked oligonucleosides may be prepared as described in U.S. Pat. No. 5,223,618. Phosphinate oligonucleotides may be prepared as described in U.S. Pat. No. 5,508,270. Alkyl phosphonate oligonucleotides may be prepared as described in U.S. Pat. No. 4,469,863. 3′-Deoxy-3′-methylene phosphonate oligonucleotides may be prepared as described in U.S. Pat. Nos. 5,610,289 or 5,625,050. Phosphoramidite oligonucleotides may be prepared as described in U.S. Pat. No. 5,256,775 or U.S. Pat. No. 5,366,878. Alkylphosphonothioate oligonucleotides may be prepared as described in published PCT applications WO 94/17093 and WO 94/02499. 3′-Deoxy-3′-amino phosphoramidate oligonucleotides may be prepared as described in U.S. Pat. No. 5,476,925. Phosphotriester oligonucleotides may be prepared as described in U.S. Pat. No. 5,023,243. Borano phosphate oligonucleotides may be prepared as described in U.S. Pat. Nos. 5,130,302 and 5,177,198.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 10 kb, 7.5 kb, 5 kb, 2.5 kb, 2 kb, 1.5 kb, or 1 kb in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 75, 50, 25, 20, 15, 10, 5, 4, 3, 2, or 1 genomic flanking gene(s).

The polynucleotide sequences of the invention may be prepared by any known method, including synthetic, recombinant, ex vivo generation, or a combination thereof, as well as utilizing any purification methods known in the art.

The terms “comprising”, “consisting of” and “consisting essentially of” may be interchanged for one another throughout the instant application”. The term “having” has the same meaning as “comprising” and may be replaced with either the term “consisting of” or “consisting essentially of”.

“Stringent”, “moderate,” and “low” hybridization conditions are as defined below.

A sequence which is “operably linked” to a regulatory sequence such as a promoter means that said regulatory element is in the correct location and orientation in relation to the nucleic acid to control RNA polymerase initiation and expression of the nucleic acid of interest. As used herein, the term “operably linked” refers to a linkage of polynucleotide elements in a functional relationship. For instance, a promoter or enhancer is operably linked to a coding sequence if it affects the transcription of the coding sequence.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which are capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide,” “complementary nucleic acid” and “complementary nucleotide sequence”. These terms are applied to pairs of polynucleotides based solely upon their sequences and not any particular set of conditions under which the two polynucleotides would actually bind. Preferably, a “complementary” sequence is a sequence which an A at each position where there is a T on the opposite strand, a T at each position where there is an A on the opposite strand, a G at each position where there is a C on the opposite strand and a C at each position where there is a G on the opposite strand.

The term “allele” is used herein to refer to variants of a nucleotide sequence. A biallelic polymorphism has two forms. Diploid organisms may be homozygous or heterozygous for an allelic form. Unless otherwise specified, the polynucleotides of the present invention encompass all allelic variants of the disclosed polynucleotides.

The term “upstream” is used herein to refer to a location that is toward the 5′ end of the polynucleotide from a specific reference point.

As used herein, the term “non-human animal” refers to any non-human vertebrate animal, including insects, birds, rodents and more usually mammals. Preferred non-human animals include: primates; farm animals such as swine, goats, sheep, donkeys, cattle, horses, chickens, rabbits; and rodents, more preferably rats or mice. As used herein, the term “animal” is used to refer to any species in the animal kingdom, preferably vertebrates, including birds and fish, and more preferable a mammal. Both the terms “animal” and “mammal” expressly embrace human subjects unless preceded with the term “non-human”.

The terms “vertebrate nucleic acid” and “vertebrate polpeptide” are used herein to refer to any nucleic acid or polypeptide respectively which are derived from a vertebrate species including birds and more usually mammals, preferably primates such as humans, farm animals such as swine, goats, sheep, donkeys, and horses, rabbits or rodents, more preferably rats or mice. As used herein, the term “vertebrate” is used to refer to any vertebrate, preferably a mammal. The term “vertebrate” expressly embraces human subjects unless preceded with the term “non-human”

“Stringent”, “moderate,” and “low” hybridization conditions are as defined below.

The term “capable of hybridizing to the polyA tail of said mRNA” refers to and embraces all primers containing stretches of thymidine residues, so-called oligo(dT) primers, that hybridize to the 3′ end of eukaryotic poly(A)+mRNAs to prime the synthesis of a first cDNA strand. Techniques for generating said oligo(dT) primers and hybridizing them to mRNA to subsequently prime the reverse transcription of said hybridized mRNA to generate a first cDNA strand are well known to those skilled in the art and are described in Current Protocols in Molecular Biology, John Wiley and Sons, Inc. 1997 and Sambrook et al., Molecular Cloning: A Laboratory Manual, Second Edition, Cold Spring Harbor Laboratory Press, 1989, the entire disclosures of which are incorporated herein by reference. Preferably, said oligo(dT) primers are present in a large excess in order to allow the hybridization of all mRNA 3′ends to at least one oligo(dT) molecule. The priming and reverse transcription step are preferably performed between 37° C. and 55° C. depending on the type of reverse transcriptase used.

Preferred oligo(dT) primers for priming reverse transcription of mRNAs are oligonucleotides containing a stretch of thymidine residues of sufficient length to hybridize specifically to the polyA tail of mRNAs, preferably of 12 to 18 thymidine residues in length. More preferably, such oligo(T) primers comprise an additional sequence upstream of the poly(dT) stretch in order to allow the addition of a given sequence to the 5′end of all first cDNA strands which may then be used to facilitate subsequent manipulation of the cDNA. Preferably, this added sequence is 8 to 60 residues in length. For instance, the addition of a restriction site in 5′ of cDNAs facilitates subcloning of the obtained cDNA. Alternatively, such an added 5′end may also be used to design primers of PCR to specifically amplify cDNA clones of interest.

In particular, the present invention relates to cDNAs which were derived from genes encoding secreted proteins. As used herein, a “secreted” protein is one which, when expressed in a suitable host cell, is transported across or through a membrane, including transport as a result of signal peptides in its amino acid sequence. “Secreted” proteins include without limitation proteins secreted wholly (e.g. soluble proteins), or partially (e.g. receptors) from the cell in which they are expressed. “Secreted” proteins also include without limitation proteins which are transported across the membrane of the endoplasmic reticulum.

cDNAs encoding secreted proteins may include nucleic acid sequences, called signal sequences, which encode signal peptides which direct the extracellular secretion of the proteins encoded by the cDNAs. Generally, the signal peptides are located at the amino termini of secreted proteins. Polypeptides comprising these signal peptides (as delineated in the sequence

Secreted proteins are translated by ribosomes associated with the “rough” endoplasmic reticulum. Generally, secreted proteins are co-translationally transferred to the membrane of the endoplasmic reticulum. Association of the ribosome with the endoplasmic reticulum during translation of secreted proteins is mediated by the signal peptide. The signal peptide is typically cleaved following its co-translational entry into the endoplasmic reticulum. After delivery to the endoplasmic reticulum, secreted proteins may proceed through the Golgi apparatus. In the Golgi apparatus, the proteins may undergo post-translational modification before entering secretory vesicles which transport them across the cell membrane.

The cDNAs of the present invention have several important applications. For example, they may be used to express the entire secreted protein which they encode. Alternatively, they may be used to express fragments of the secreted protein. The fragments may comprise the signal peptides encoded by the cDNAs or the mature proteins encoded by the cDNAs (i.e. the proteins generated when the signal peptide is cleaved off). The cDNAs and fragments thereof also have important applications as polynucleotides. For example, the cDNAs of the sequence listing and fragments thereof, may be used to distinguish human tissues/cells from non-human tissues/cells and to distinguish between human tissues/cells that do and do not express the polynucleotides comprising the cDNAs. By knowing the tissue expression pattern of the cDNAs, either through routine experimentation or by using the instant disclosure, the polynucleotides of the present invention may be used in methods of determining the identity of an unknown tissue/cell sample. As part of determining the identity of an unknown tissue/cell sample, the polynucleotides of the present invention may be used to determine what the unknown tissue/cell sample is and what the unknown sample is not. For example, if a cDNA is expressed in a particular tissue/cell type, and the unknown tissue/cell sample does not express the cDNA, it may be inferred that the unknown tissue/cells are either not human or not the same human tissue/cell type as that which expresses the cDNA. These methods of determining tissue/cell identity are based on methods which detect the presence or absence of the mRNA (or corresponding cDNA) in a tissue/cell sample using methods well know in the art (e.g., hybridization or PCR based methods).

In other useful applications, fragments of the cDNAs encoding signal peptides as well as degenerate polynucleotides encoding the same, may be ligated to sequences encoding either the polypeptide from the same gene or to sequences encoding a heterologous polypeptide to facilitate secretion.

Antibodies which specifically recognize the entire secreted proteins encoded by the cDNAs or fragments thereof having at least 6 consecutive amino acids, 8 consecutive amino acids, 10 consecutive amino acids, at least 15 consecutive amino acids, at least 25 consecutive amino acids, or at least 40 consecutive amino acids may also be obtained as described below. Antibodies which specifically recognize the mature protein generated when the signal peptide is cleaved may also be obtained as described below. Similarly, antibodies which specifically recognize the signal peptides encoded by the cDNAs may also be obtained.

In some embodiments, the cDNAs include the signal sequence. In other embodiments, the cDNAs may include the full coding sequence for the mature protein (i.e. the protein generated when the signal polypeptide is cleaved off). In addition, the cDNAs may include regulatory regions upstream of the translation start site or downstream of the stop codon which control the amount, location, or developmental stage of gene expression. As discussed above, secreted proteins are therapeutically important. Thus, the proteins expressed from the cDNAs may be useful in treating or controlling a variety of human conditions. The cDNAs may also be used to obtain the corresponding genomic DNA. The term “corresponding genomic DNA” refers to the genomic DNA which encodes mRNA which includes the sequence of one of the strands of the cDNA in which thymidine residues in the sequence of the cDNA are replaced by uracil residues in the RNA.

The cDNAs or genomic DNAs obtained therefrom may be used in forensic procedures to identify individuals or in diagnostic procedures to identify individuals having genetic diseases resulting from abnormal expression of the genes corresponding to the cDNAs. In addition, the present invention is useful for constructing a high resolution map of the human chromosomes.

The present invention also relates to secretion vectors capable of directing the secretion of a protein of interest. Such vectors may be used in gene therapy strategies in which it is desired to produce a gene product in one cell which is to be delivered to another location in the body. Secretion vectors may also facilitate the purification of desired proteins.

The present invention also relates to expression vectors capable of directing the expression of an inserted gene in a desired spatial or temporal manner or at a desired level. Such vectors may include sequences upstream of the cDNAs such as promoters or upstream regulatory sequences.

In addition, the present invention may also be used for gene therapy to control or treat genetic diseases. Signal peptides may also be fused to heterologous proteins to direct their extracellular secretion.

One embodiment of the present invention is a purified or isolated nucleic acid comprising the sequence of one of SEQ ID NOs: 1-405 or a sequence complementary thereto, allelic variants thereof, and degenerate variants thereof. In one aspect of this embodiment, the nucleic acid is recombinant.

Another embodiment of the present invention is a purified or isolated nucleic acid comprising at least 8 consecutive bases of the sequence of one of SEQ ID NOs: 1-405 or one of the sequences complementary thereto, allelic variants thereof, and degenerate variants thereof. In one aspect of this embodiment, the nucleic acid comprises at least 10, 12, 15, 18, 20, 25, 28, 30, 35, 40, 50, 75, 100, 150, 200, 300, 400, 500, 1000 or 2000 consecutive bases of one of the sequences of SEQ ID NOs: 1-405 or one of the sequences complementary thereto, allelic variants thereof, and degenerate variants thereof. The nucleic acid may be a recombinant nucleic acid.

In addition to the above preferred nucleic acid sizes, further preferred sub-genuses of nucleic acids comprise at least 8 nucleotides, wherein “at least 8” is defined as any integer between 8 and the integer representing the 3′ most nucleotide position as set forth in the sequence listing or elsewhere herein. Further included as preferred polynucleotides of the present invention are nucleic acid fragments at least 8 nucleotides in length, as described above, that are further specified in terms of their 5′ and 3′ position. The 5′ and 3′ positions are represented by the position numbers set forth in the sequence listing below. For allelic and degenerate variants, position 1 is defined as the 5′ most nucleotide of the ORF, i.e., the nucleotide “A” of the start codon with the remaining nucleotides numbered consecutively. Therefore, every combination of a 5′ and 3′ nucleotide position that a polynucleotide fragment of the present invention, at least 8 contiguous nucleotides in length, could occupy is included in the invention as an individual species. The polynucleotide fragments specified by 5′ and 3′ positions can be immediately envisaged and are therefore not individually listed solely for the purpose of not unnecessarily lengthening the specifications.

It is noted that the above species of polynucleotide fragments of the present invention may alternatively be described by the formula “a to b”; where “x” equals the 5″ most nucleotide position and “y” equals the 3″ most nucleotide position of the polynucleotide; and further where “x” equals an integer between 1 and the number of nucleotides of the polynucleotide sequence of the present invention minus 8, and where “y” equals an integer between 9 and the number of nucleotides of the polynucleotide sequence of the present invention; and where “x” is an integer smaller then “y” by at least 8.

The present invention also provides for the exclusion of any species of polynucleotide fragments of the present invention specified by 5′ and 3′ positions or sub-genuses of polynucleotides specified by size in nucleotides as described above. Any number of fragments specified by 5′ and 3′ positions or by size in nucleotides, as described above, may be excluded.

Another embodiment of the present invention is a vertebrate purified or isolated nucleic acid of at least 15, 18, 20, 23, 25, 28, 30, 35, 40, 50, 75, 100, 200, 300, 500 or 1000 nucleotides in length which hybridizes under stringent conditions to the sequence of one of SEQ ID NOs: 1-405 or a sequence complementary to one of the sequences of SEQ ID NOs: 1-405. In one aspect of this embodiment, the nucleic acid is recombinant.

Another embodiment of the present invention is a purified or isolated nucleic acid comprising the full coding sequences of one of SEQ ID NOs: 1-405, or an allelic variant thereof, wherein the full coding sequence optionally comprises the sequence encoding signal peptide as well as the sequence encoding mature protein. In one aspect of this embodiment, the nucleic acid is recombinant.

A further embodiment of the present invention is a purified or isolated nucleic acid comprising the nucleotides of one of SEQ ID NOs: 1-405, or an allelic variant thereof which encode a mature protein. In one aspect of this embodiment, the nucleic acid is recombinant. In another aspect of this embodiment, the nucleic acid is an expression vector wherein said nucleotides of one of SEQ ID NOs: 1-405, or an allelic variant thereof which encode a mature protein, are operably linked to a promoter.

Yet another embodiment of the present invention is a purified or isolated nucleic acid comprising the nucleotides of one of SEQ ID NOs: 1-405, or an allelic variant thereof, which encode the signal peptide. In one aspect of this embodiment, the nucleic acid is recombinant. In another aspect of this embodiment, the nucleic acid is an fusion vector wherein said nucleotides of one of SEQ ID NOs: 1-405, or an allelic variant thereof which encode the signal peptide, are operably linked to a second nucleic acid encoding an heterologous polypeptide.

Another embodiment of the present invention is a purified or isolated nucleic acid encoding a polypeptide comprising the sequence of one of the sequences of SEQ ID NOs: 406-810, or allelic variant thereof. In one aspect of this embodiment, the nucleic acid is recombinant.

Another embodiment of the present invention is a purified or isolated nucleic acid encoding a polypeptide comprising the sequence of a mature protein included in one of the sequences of SEQ ID NOs: 406-810, or allelic variant thereof. In one aspect of this embodiment, the nucleic acid is recombinant.

Another embodiment of the present invention is a purified or isolated nucleic acid encoding a polypeptide comprising the sequence of a signal peptide included in one of the sequences of SEQ ID NOs: 406-810, or allelic variant thereof. In one aspect of this embodiment, the nucleic acid is recombinant. In another aspect it is present in a vector of the invention.

Further embodiments of the invention include isolated polynucleotides that comprise, a nucleotide sequence at least 70% identical, more preferably at least 75% identical, and still more preferably at least 80%, 85%, 90%, 95%, 96%, 97%, 98%, or 99% identical to any of the polynucleotides of the present invention. Methods of determining identity include those well known in the art and described herein.

Yet another embodiment of the present invention is a purified or isolated protein comprising the sequence of one of SEQ ID NOs: 406-810, or allelic variant thereof.

Another embodiment of the present invention is a purified or isolated polypeptide comprising at least 5 or 8 consecutive amino acids of one of the sequences of SEQ ID NOs: 406-810. In one aspect of this embodiment, the purified or isolated polypeptide comprises at least 10, 12, 15, 20, 25, 30, 35, 40, 50, 60, 75, 100, 150 or 200 consecutive amino acids of one of the sequences of SEQ ID NOs: 406-810.

In addition to the above polypeptide fragments, further preferred sub-genuses of polypeptides comprise at least 8 amino acids, wherein “at least 8” is defined as any integer between 8 and the integer representing the C-terminal amino acid of the polypeptide of the present invention including the polypeptide sequences of the sequence listing below. Further included are species of polypeptide fragments at least 8 amino acids in length, as described above, that are further specified in terms of their N-terminal and C-terminal positions. Preferred species of polypeptide fragments specified by their N-terminal and C-terminal positions include the signal peptides delineated in the sequence listing below. However, included in the present invention as individual species are all polypeptide fragments, at least 8 amino acids in length, as described above, and may be particularly specified by a N-terminal and C-terminal position. That is, every combination of a N-terminal and C-terminal position that a fragment at least 8 contiguous amino acid residues in length could occupy, on any given amino acid sequence of the sequence listing or of the present invention is included in the present invention

The present invention also provides for the exclusion of any fragment species specified by N-terminal and C-terminal positions or of any fragment sub-genus specified by size in amino acid residues as described above. Any number of fragments specified by N-terminal and C-terminal positions or by size in amino acid residues as described above may be excluded as individual species.

The above polypeptide fragments of the present invention can be immediately envisaged using the above description and are therefore not individually listed solely for the purpose of not unnecessarily lengthening the specification. Moreover, the above fragments need not be active since they would be useful, for example, in immunoassays, in epitope mapping, epitope tagging, as vaccines, and as molecular weight markers. The above fragments may also be used to generate antibodies to a particular portion of the polypeptide. These antibodies can then be used in immunoassays well known in the art to distinguish between human and non-human cells and tissues or to determine whether cells or tissues in a biological sample are or are not of the same type which express the polypeptide of the present invention. Preferred polypeptide fragments of the present invention comprising a signal peptide may be used to facilitate secretion of either the polypeptide of the same gene or a heterologous polypeptide using methods well known in the art.

Another embodiment of the present invention is an isolated or purified polypeptide comprising a signal peptide of one of the polypeptides of SEQ ID NOs: 406-810.

Yet another embodiment of the present invention is an isolated or purified polypeptide comprising a mature protein of one of the polypeptides of SEQ ID NOs: 406-810.

Yet another embodiment of the present invention is an isolated or purified polypeptide comprising a fall length polypeptide, mature protein, or signal peptide encoded by an allelic variant of the polynucleotides of the present invention.

A further embodiment of the present invention are polypeptides having an amino acid sequence with at least 70% similarity, and more preferably at least 75%, 80%, 85%, 90%, 95%, 96%, 97%, 98%, or 99% similarity to a polypeptide of the present invention, as well as polypeptides having an amino acid sequence at least 70% identical, more preferably at least 75% identical, and still more preferably 80%, 85%, 90%, 95%, 96%, 97%, 98%, or 99% identical to a polypeptide of the present invention. Further included in the invention are isolated nucleic acid molecules encoding such polypeptides. Methods for determining identity include those well known in the art and described herein.

A further embodiment of the present invention is a method of making a protein comprising one of the sequences of SEQ ID NO: 406-810, comprising the steps of obtaining a cDNA comprising one of the sequences of sequence of SEQ ID NO: 1-405, inserting the cDNA in an expression vector such that the cDNA is operably linked to a promoter, and introducing the expression vector into a host cell whereby the host cell produces the protein encoded by said cDNA. In one aspect of this embodiment, the method further comprises the step of isolating the protein.

Another embodiment of the present invention is a protein obtainable by the method described in the preceding paragraph.

Another embodiment of the present invention is a method of making a protein comprising the amino acid sequence of the mature protein contained in one of the sequences of SEQ ID NO: 406-810, comprising the steps of obtaining a cDNA comprising one of the nucleotides sequence of sequence of SEQ ID NO: 1-405 which encode for the mature protein, inserting the cDNA in an expression vector such that the cDNA is operably linked to a promoter, and introducing the expression vector into a host cell whereby the host cell produces the mature protein encoded by the cDNA. In one aspect of this embodiment, the method further comprises the step of isolating the protein.

Another embodiment of the present invention is a mature protein obtainable by the method described in the preceding paragraph.

Another embodiment of the present invention is a host cell containing the purified or isolated nucleic acids comprising the sequence of one of SEQ ID NOs: 1-405 or a sequence complementary thereto described herein.

Another embodiment of the present invention is a host cell containing the purified or isolated nucleic acids comprising the full coding sequences of one of SEQ ID NOs: 1-405, wherein the full coding sequence comprises the sequence encoding the signal peptide and the sequence encoding the mature protein described herein.

Another embodiment of the present invention is a host cell containing the purified or isolated nucleic acids comprising the nucleotides of one of SEQ ID NOs: 1-405 which encode a mature protein which are described herein.

Another embodiment of the present invention is a host cell containing the purified or isolated nucleic acids comprising the nucleotides of one of SEQ ID NOs: 1-405 which encode the signal peptide which are described herein.

Another embodiment of the present invention is a purified or isolated antibody capable of specifically binding to a protein comprising the sequence of one of SEQ ID NOs: 406-810. In one aspect of this embodiment, the antibody is capable of binding to a polypeptide comprising at least 6 consecutive amino acids, at least 8 consecutive amino acids, or at least 10 consecutive amino acids of the sequence of one of SEQ ID NOs: 406-810.

Another embodiment of the present invention is an array of cDNAs or fragments thereof of at least 15 nucleotides in length which includes at least one of the sequences of SEQ ID NOs: 1-405, or one of the sequences complementary to the sequences of SEQ ID NOs: 1-405, or a fragment thereof of at least 15 consecutive nucleotides. In one aspect of this embodiment, the array includes at least two of the sequences of SEQ ID NOs: 1-405, the sequences complementary to the sequences of SEQ ID NOs: 1-405, or fragments thereof of at least 15 consecutive nucleotides. In another aspect of this embodiment, the array includes at least five of the sequences of SEQ ID NOs: 1-405, the sequences complementary to the sequences of SEQ ID NOs: 1-405, or fragments thereof of at least 15 consecutive nucleotides.

A further embodiment of the invention encompasses purified polynucleotides comprising an insert from a clone deposited in an ECACC deposit, which contains the sequences of SEQ ID NOs. 2-17 and 19-23, having an accession No. 99061735 and named SignalTag 15061999 or deposited in an ECACC deposit having an accession No. 98121805 and named SignalTag 166-191, which contains SEQ ID NOs.: 24-50, or a fragment of these nucleic acids comprising a contiguous span of at least 8, 10, 12, 15, 18, 20, 25, 28, 30, 35, 40, 50, 75, 100, 150, 200, 300, 400, 500, 1000 or 2000 nucleotides of said insert. In one aspect of this embodiment, the purified polynucleotide is recombinant.

An additional embodiment of the invention encompasses purified polypeptides which comprise, consist of, or consist essentially of an amino acid sequence encoded by the insert from a clone deposited in an ECACC deposit, which contains the sequences of SEQ ID NOs. 2-17 and 19-23, having an accession No. 99061735 and named SignalTag 15061999 or deposited in an ECACC deposit having an accession No. 98121805 and named SignalTag 166-191, which contains SEQ ID NOs.: 24-50, as well as polypeptides which comprise a fragment of said amino acid sequence consisting of a signal peptide, a mature protein, or a contiguous span of at least 5, 8, 10, 12, 15, 20, 25, 30, 35, 40, 50, 60, 75, 100, 150 or 200 amino acids encoded by said insert.

An additional embodiment of the invention encompasses purified polypeptides which comprise a contiguous span of at least 5, 8, 10, 12, 15, 20, 25, 30, 35, 40, 50, 60, 75, 100, 150 or 200 amino acids of SEQ ID NOs: 406-810, wherein said contiguous span comprises at least one of the amino acid positions which was not shown to be identical to a public sequence in the instant application. Also encompassed by the invention are purified polynucleotides encoding said polypeptides.

Another embodiment of the present invention is a computer readable medium having stored thereon a sequence selected from the group consisting of a cDNA code of SEQ ID NOs. 1-405 and a polypeptide code of SEQ ID NOs. 406-810.

Another embodiment of the present invention is a computer system comprising a processor and a data storage device wherein the data storage device has stored thereon a sequence selected from the group consisting of a cDNA code of SEQ ID NOs. 1-405 and a polypeptide code of SEQ ID NOs. 406-810. In some embodiments the computer system further comprises a sequence comparer and a data storage device having reference sequences stored thereon. For example, the sequence comparer may comprise a computer program which indicates polymorphisms. In other aspects of the computer system, the system further comprises an identifier which identifies features in said sequence.

Another embodiment of the present invention is a method for comparing a first sequence to a reference sequence wherein the first sequence is selected from the group consisting of a cDNA code of SEQ ID NOs. 1-405 and a polypeptide code of SEQ ID NOs. 406-810 comprising the steps of reading the first sequence and the reference sequence through use of a computer program which compares sequences and determining differences between the first sequence and the reference sequence with the computer program. In some aspects of this embodiment, said step of determining differences between the first sequence and the reference sequence comprises identifying polymorphisms.

Another aspect of the present invention is a method for determining the level of identity between a first sequence and a reference sequence, wherein the first sequence is selected from the group consisting of a cDNA code of SEQ ID NOs. 1-405 and a polypeptide code of SEQ ID NOs. 406-810, comprising the steps of reading the first sequence and the reference sequence through the use of a computer program which determines identity levels and determining identity between the first sequence and the reference sequence with the computer program.

Another embodiment of the present invention is a method for identifying a feature in a sequence selected from the group consisting of a cDNA code of SEQ ID NOs. 1-405 and a polypeptide code of SEQ ID NOs. 406-810 comprising the steps of reading the sequence through the use of a computer program which identifies features in sequences and identifying features in the sequence with said computer program. In one aspect of this embodiment, the computer program comprises a computer program which identifies open reading frames. In a further embodiment, the computer program comprises a program that identifies linear or structural motifs in a polypeptide sequence.

### BRIEF DESCRIPTION OF THE DRAWINGS

FIG. 1 is a table with all of the parameters that can be used for each step of cDNA analysis.

FIG. 2 is an analysis of the 43 amino terminal amino acids of all human SwissProt proteins to determine the frequency of false positives and false negatives using the techniques for signal peptide identification described herein.

FIG. 3 provides a diagram of a RT-PCR-based method to isolate cDNAs containing sequences adjacent to 5′ESTs used to obtain them

FIG. 4 is a block diagram of an exemplary computer system.

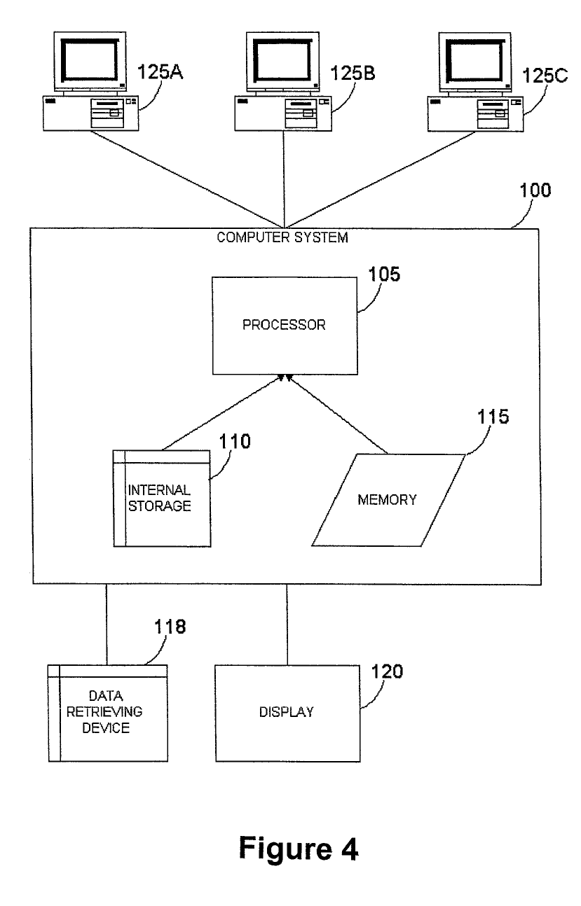

FIG. 5 is a flow diagram illustrating one embodiment of a process 200 for comparing a new nucleotide or protein sequence with a database of sequences in order to determine the identity levels between the new sequence and the sequences in the database.

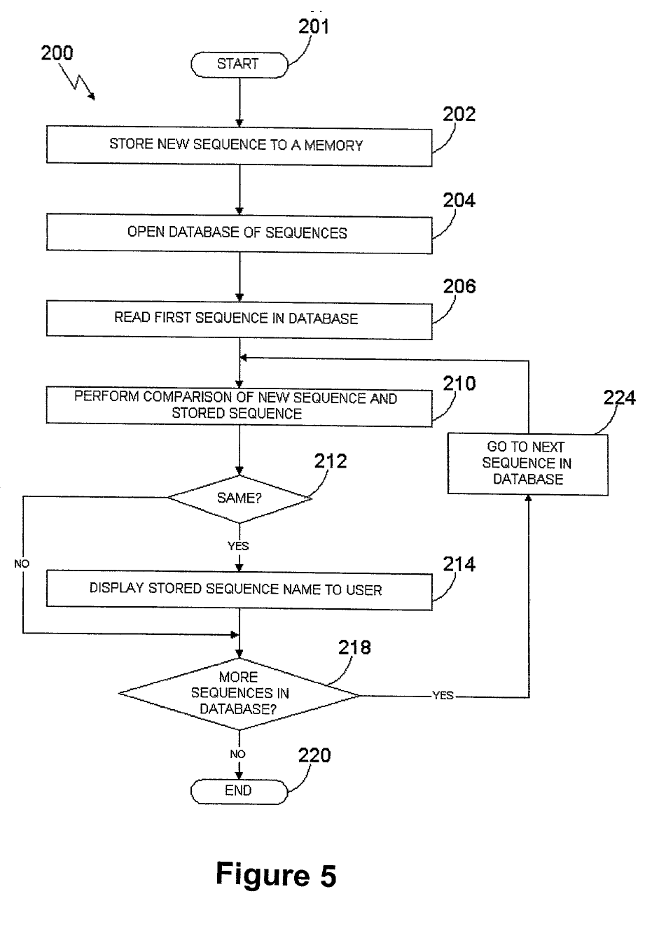

FIG. 6 is a flow diagram illustrating one embodiment of a process 250 in a computer for determining whether two sequences are homologous.

FIG. 7 is a flow diagram illustrating one embodiment of an identifier process 300 for detecting the presence of a feature in a sequence.

### BRIEF DESCRIPTION OF THE TABLES

Table I provides structural features of each cDNAs of SEQ ID NOs: 1-405, i.e., the locations of the full coding sequences, the locations of the nucleotides which encode the signal peptides, the locations of nucleotides which encode the mature proteins generated by cleavage of the signal peptides, the locations of stop codons, the locations of the polyA signals and the locations of polyA sites.

Table II provides structural features for each polypeptide of SEQ ID NOs: 406-810, i.e; the locations of the full length polypeptide, the locations of the signal peptides, and the locations of the mature polypeptide created by cleaving the signal peptide from the full length polypeptide.

Table III lists the positions of preferred fragments, defined as fragments not sharing more than 90% identity with any public sequence over at least 30 nucleotides in length, for some cDNAs of SEQ ID NOs: 1-405.

Table IVa provides the positions of fragments which are preferably included in the present invention while Table IVb provides the positions of fragments which are preferably excluded from the present invention. Tables IVa and IVb provides for the inclusion and exclusion of polynucleotides in addition to those described elsewhere in the specification and is therefore, not meant as limiting description.

Table V provides the applicant's internal designation number assigned to each sequence identification number and indicates whether the sequence is a nucleic acid sequence or a polypeptide sequence.

Table VI lists the Genset's libraries of tissues and cell types examined that express the polynucleotides of the present invention.

Table VII relates to the bias in spatial distribution of the polynucleotide sequences of the present invention.

Table VIII relates to the spatial distribution of the polynucleotide sequences of the sequence listing using information from public databases.

Table IX lists known biologically structural and functional domains for the cDNA of the present invention.

Table X lists antigenic peaks of predicted antigenic epitopes for cDNAs or the present invention.

Table XI lists the putative chromosomal location of the polynucleotides of the present invention.

### DETAILED DESCRIPTION OF THE PREFERRED EMBODIMENT

I. Obtaining cDNA Libraries Including the 5′Ends of their Corresponding mRNAS

The cDNAs of the present invention may include the entire coding sequence of the protein encoded by the corresponding mRNA, including the authentic translation start site, the signal sequence, and the sequence encoding the mature protein remaining after cleavage of the signal peptide. Such cDNAs are referred to herein as “full length cDNAs.” Alternatively, the cDNAs may include only the sequence encoding the mature protein remaining after cleavage of the signal peptide, or only the sequence encoding the signal peptide.

The methods explained therein can also be used to obtain cDNAs which encode less than the entire coding sequence of the secreted proteins encoded by the genes corresponding to the cDNAs. In some embodiments, the cDNAs isolated using these methods encode at least 5 amino acids of one of the proteins encoded by the sequences of SEQ ID NOs: 1-405. In further embodiments, the cDNAs encode at least 10, 12, 15, 20, 25, 30, 35, 40, 50, 60, 75, 100, 150 or 200 consecutive amino acids of the proteins encoded by the sequences of SEQ ID NOs: 1-405. In a preferred embodiment, the cDNAs encode a full length protein sequence, which includes the protein coding sequences of SEQ ID NOs: 1-405.

The cDNAs of the present invention were obtained from cDNA libraries derived from mRNAs having intact 5′ ends as described in Examples 1 to 5 using either a chemical or enzymatic approach.

### EXAMPLE 1

Preparation of mRNA

Total human RNAs or polyA+ RNAs derived from different tissues were respectively purchased from LABIMO and CLONTECH and used to generate cDNA libraries as described below. The purchased RNA had been isolated from cells or tissues using acid guanidium thiocyanate-phenol-chloroform extraction (Chomczyniski and Sacchi, Analytical Biochemistry 162:156-159, 1987). PolyA+ RNA was isolated from total RNA (LABIMO) by two passes of oligo dT chromatography, as described by Aviv and Leder, Proc. Natl. Acad. Sci. USA 69:1408-1412, 1972) in order to eliminate ribosomal RNA.

The quality and the integrity of the polyA+ RNAs were checked. Northern blots hybridized with a probe corresponding to an ubiquitous mRNA, such as elongation factor 1 or elongation factor 2, were used to confirm that the mRNAs were not degraded. Contamination of the polyA+ mRNAs by ribosomal sequences was checked using Northern blots and a probe derived from the sequence of the 28S rRNA. Preparations of mRNAs with less than 5% of rRNAs were used in library construction. To avoid constructing libraries with RNAs contaminated by exogenous sequences (prokaryotic or fungal), the presence of bacterial 16S ribosomal sequences or of two highly expressed fungal mRNAs was examined using PCR.

### EXAMPLE 2

Methods for Obtaining mRNAs having Intact 5′ Ends

Following preparation of the mRNAs from various tissues as described above, selection of mRNA with intact 5′ ends and specific attachment of an oligonucleotide tag to the 5′ end of such mRNA is performed using either a chemical or enzymatic approach. Both techniques take advantage of the presence of the “cap” structure, which characterizes the 5′end of intact mRNAs and which comprises a guanosine generally methylated once, at the 7 position.

The chemical modification approach involves the optional elimination of the 2′,3′-cis diol of the 3′ terminal ribose, the oxidation of the 2′, 3′, -cis diol of the ribose linked to the cap of the 5′ ends of the mRNAs into a dialdehyde, and the coupling of the dialdehyde to a derivatized oligonucleotide tag. Further detail regarding the chemical approaches for obtaining mRNAs having intact 5′ ends are disclosed in International Application No. WO96/34981, published Nov. 7, 1996, the disclosure of which is incorporated herein by reference in its entirety.

The enzymatic approach for ligating the oligonucleotide tag to the 5′ ends of mRNAs with intact 5′ ends involves the removal of the phosphate groups present on the 5′ ends of uncapped incomplete mRNAs, the subsequent decapping of mRNAs with intact 5′ ends and the ligation of the phosphate present at the 5′ end of the decapped mRNA to an oligonucleotide tag. Further detail regarding the enzymatic approaches for obtaining mRNAs having intact 5′ ends are disclosed in Dumas Milne Edwards J. B. (Doctoral Thesis of Paris VI University, Le clonage des ADNc complets: difficultes et perspectives nouvelles. Apports pour l'etude de la regulation de l'expression de la tryptophane hydroxylase de rat, 20 Dec. 1993), EP0 625572 and Kato et al., Gene 150:243-250 (1994), the disclosures of which are incorporated herein by reference in their entireties.

In either the chemical or the enzymatic approach, the oligonucleotide tag has a restriction enzyme site (e.g. EcoRI sites) therein to facilitate later cloning procedures. Following attachment of the oligonucleotide tag to the mRNA, the integrity of the mRNA was then examined by performing a Northern blot using a probe complementary to the oligonucleotide tag.

### EXAMPLE 3

cDNA Synthesis Using mRNA Templates having Intact 5′ Ends

For the mRNAs joined to oligonucleotide tags using either the chemical or the enzymatic method, first strand cDNA synthesis was performed using reverse transcriptase with an oligo-dT primer or random nonamer. In some instances, this oligo-dT primer contained an internal tag of at least 4 nucleotides which is different from one tissue to the other. In order to protect internal EcoRI sites in the cDNA from digestion at later steps in the procedure, methylated dCTP was used for first strand synthesis. After removal of RNA by an alkaline hydrolysis, the first strand of cDNA was precipitated using isopropanol in order to eliminate residual primers.

The second strand of the cDNA was then synthesized with a Klenow fragment using a primer corresponding to the 5′end of the ligated oligonucleotide. Preferably, the primer is 20-25 bases in length. Methylated dCTP was also used for second strand synthesis in order to protect internal EcoRI sites in the cDNA from digestion during the cloning process.

### EXAMPLE 4

Cloning of cDNAs Derived from mRNA with Intact 5′ Ends into BlueScript

Following second strand synthesis, the cDNAs were cloned into the phagemid pBlueScript II SK− vector (Stratagene). The ends of the cDNAs were blunted with T4 DNA polymerase (Biolabs) and the cDNA was digested with EcoRI. Since methylated dCTP was used during cDNA synthesis, the EcoRI site present in the tag was the only hemi-methylated site, hence the only site susceptible to EcoRI digestion. In some instances, to facilitate subcloning, an Hind III adaptor was added to the 3′ end of cDNAs.

The cDNAs were then size fractionated using either exclusion chromatography (AcA, Biosepra) or electrophoretic separation which yields 3 or 6 different fractions. The cDNAs were then directionally cloned either into pBlueScript using either the EcoRI and SmaI restriction sites or the EcoRI and Hind III restriction sites when the Hind III adaptator was present in the cDNAs. The ligation mixture was electroporated into bacteria and propagated under appropriate antibiotic selection.

### EXAMPLE 5

Selection of Clones having the Oligonucleotide Tag Attached Thereto

Clones containing the oligonucleotide tag attached to cDNAs were then selected as follows.

The plasmid DNAs containing cDNA libraries made as described above were purified (Qiagen). A positive selection of the tagged clones was performed as follows. Briefly, in this selection procedure, the plasmid DNA was converted to single stranded DNA using gene II endonuclease of the phage Fl in combination with an exonuclease (Chang et al., Gene 127:95-8, 1993) such as exonuclease III or T7 gene 6 exonuclease. The resulting single stranded DNA was then purified using paramagnetic beads as described by Fry et al., Biotechniques, 13: 124-131, 1992. In this procedure, the single stranded DNA was hybridized with a biotinylated oligonucleotide having a sequence corresponding to the 3′ end of the oligonucleotide tag described in example 2. Preferably, the primer has a length of 20-25 bases. Clones including a sequence complementary to the biotinylated oligonucleotide were captured by incubation with streptavidin coated magnetic beads followed by magnetic selection. After capture of the positive clones, the plasmid DNA was released from the magnetic beads and converted into double stranded DNA using a DNA polymerase such as the ThermoSequenase obtained from Amersham Pharmacia Biotech. Alternatively, protocols such as the Gene Trapper kit (Gibco BRL) may be used. The double stranded DNA was then electroporated into bacteria. The percentage of positive clones having the 5′ tag oligonucleotide was estimated to typically rank between 90 and 98% using dot blot analysis.

Following electroporation, the libraries were ordered in 384-microtiter plates (MTP). A copy of the MTP was stored for future needs. Then the libraries were transferred into 96 MTP.

II. Characterization of the 5′ Ends of Clones

In order to sequence only cDNAs which contain the 5′ ends of their corresponding MrRNA, a first round of sequencing was performed on the 5′ end of clones as described in example 6. In some instances, only a partial sequence of the clone, therein referred to as “5′EST” was obtained. In other instances, the complete sequence of the clone, herein referred to as a “cDNA” is obtained. A computer analysis was then performed on the 5′ ESTs or cDNAs as described in Examples 7 and 8 in order to evaluate the quality of the cDNA libraries and in order to select clones containing sequences of interest among cDNAs which contain the 5′ ends of their corresponding mRNA.

### EXAMPLE 6

Sequencing of The 5′End of cDNA Clones

The 5′ ends of cloned cDNAs were then sequenced as follows. Plasmid inserts were first amplified by PCR on PE 9600 thermocyclers (Perkin-Elmer, Applied Biosystems Division, Foster City, Calif.) using standard SETA-A and SETA-B primers (Genset SA), AmpliTaqGold (Perkin-Elmer), dNTPs (Boehringer), buffer and cycling conditions as recommended by the Perkin-Elmer Corporation.

PCR products were then sequenced using automatic ABI Prism 377 sequencers (Perkin Elmer). Sequencing reactions were performed using PE 9600 thermocyclers with standard dye-primer chemistry and ThermoSequenase (Amersham Pharmacia Biotech). The primers used were either T7 or 21M13 (available from Genset SA) as appropriate. The primers were labeled with the JOE, FAM, ROX and TAMRA dyes. The dNTPs and ddNTPs used in the sequencing reactions were purchased from Boehringer. Sequencing buffer, reagent concentrations and cycling conditions were as recommended by Amersham.

Following the sequencing reaction, the samples were precipitated with ethanol, resuspended in formamide loading buffer, and loaded on a standard 4% acrylamide gel. Electrophoresis was performed for 2.5 hours at 3000V on an ABI 377 sequencer, and the sequence data were collected and analyzed using the ABI Prism DNA Sequencing Analysis Software, version 2.1.2.

The sequence data obtained from the sequencing of 5′ ends of all cDNA libraries made as described above were transferred to a proprietary database, where quality control and validation steps were performed. A proprietary base-caller, working using a Unix system automatically flagged suspect peaks, taking into account the shape of the peaks, the inter-peak resolution, and the noise level. The proprietary base-caller also performed an automatic trimming. Any stretch of 25 or fewer bases having more than 4 suspect peaks was considered unreliable and was discarded. Sequences corresponding to cloning vector or ligation oligonucleotides were automatically removed from the sequences. However, the resulting sequences may contain 1 to 5 nucleotides belonging to the above mentioned sequences at their 5′ end. If needed, these can easily be removed on a case by case basis.

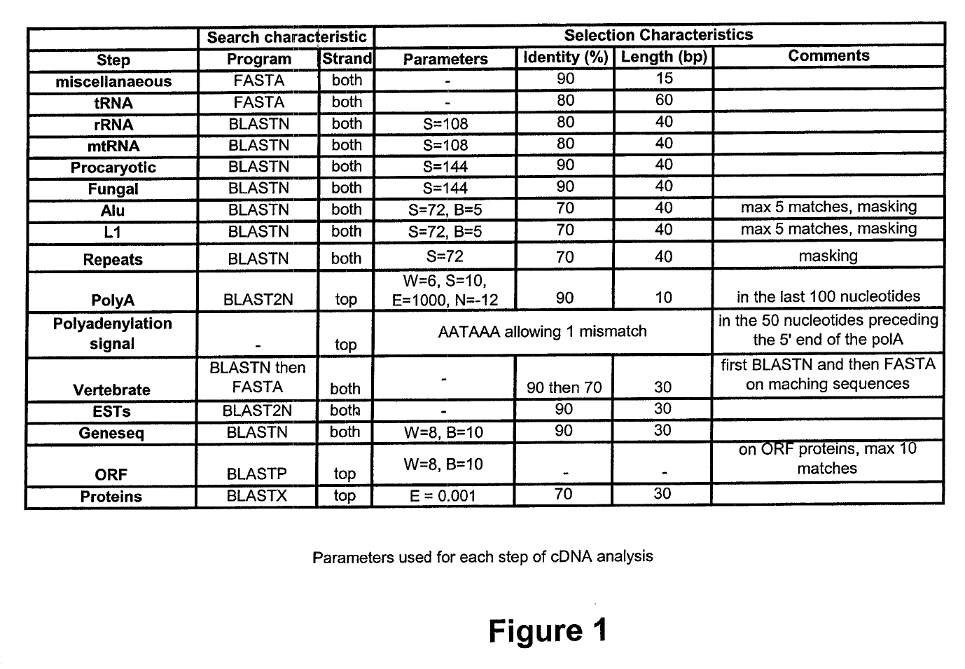

Following sequencing as described above, the sequences of the cDNA clones were entered in a database for storage and manipulation as described below. Before searching the cDNA clones in the database for sequences of interest, cDNAs derived from mRNAs which were not of interest were identified and eliminated, namely, endogenous contaminants (ribosomal RNAs, transfert RNAs, mitochondrial RNAs) and exogenous contaminants (prokaryotic RNAs and fungal RNAs) using software and parameters described in FIG. 1. In addition, cDNA sequences showing showing identity to repeated sequences (Alu, L1, THE and MER repeats, SSTR sequences or satellite, micro-satellite, or telomeric repeats) were identified and masked in further processing.

### EXAMPLE 7

Determination of Efficiency of 5′ End Selection

To determine the efficiency at which the above selection procedures isolated cDNAs which include the 5′ ends of their corresponding mRNAs, the sequences of 5′ESTs or cDNAs were aligned with a reference pool of complete mRNA/cDNA extracted from the EMBL release 57 using the FASTA algorithm. The reference mRNA/cDNA starting at the most 5′ transcription start site was obtained, and then compared to the 5′ transcription start site position of the 5′EST or cDNA. More than 75% of 5′ESTs or cDNAs had their 5′ ends close to the 5′ ends of the known sequence. As some of the mRNA sequences available in the EMBL database are deduced from genomic sequences, a 5′ end matching with these sequences will be counted as an internal match. Thus, the method used here underestimates the yield of 5′ESTs or cDNAs including the authentic 5′ ends of their corresponding mRNAs.

### EXAMPLE 8

Identification of Open Reading Frames Coding for Potential Signal Peptides

The obtained nucleic acid sequences were then screened to identify those having uninterrupted open reading frames (ORF) with a good coding probability using proprietary software. When the full-length cDNA was obtained, only complete ORFs, namely nucleic acid sequences beginning with a start codon and ending with a stop codon, longer than 150 nucleotides were considered. When only 5′EST sequences were obtained, both complete ORFS longer than 150 nucleotides and incomplete ORFs, namely nucleic acid sequences beginning with a start codon and extending up to the end of the 5′EST, longer than 60 nucleotides were considered.

The retrieved ORFs were then searched to identify potential signal motifs using slight modifications of the procedures disclosed in Von Heijne, Nucleic Acids Res. 14:46834690, 1986, the disclosure of which is incorporated herein by reference. Those 5′ESTs or cDNA sequences encoding a polypeptide with a score of at least 3.5 in the Von Heijne signal peptide identification matrix were considered to possess a signal sequence. Those 5′ESTs or cDNAs which matched a known human mRNA or EST sequence and had a 5′ end more than 30 nucleotides downstream of the known 5′ end were excluded from further analysis.

### EXAMPLE 9

Confirmation of Accuracy of Identification of Potential Signal Sequences in 5′ ESTs

The accuracy of the above procedure for identifying signal sequences encoding signal peptides was evaluated by applying the method to the 43 amino acids located at the N terminus of all human SwissProt proteins. The computed Von Heijne score for each protein was compared with the known characterization of the protein as being a secreted protein or a non-secreted protein. In this manner, the number of non-secreted proteins having a score higher than 3.5 (false positives) and the number of secreted proteins having a score lower than 3.5 (false negatives) could be calculated.

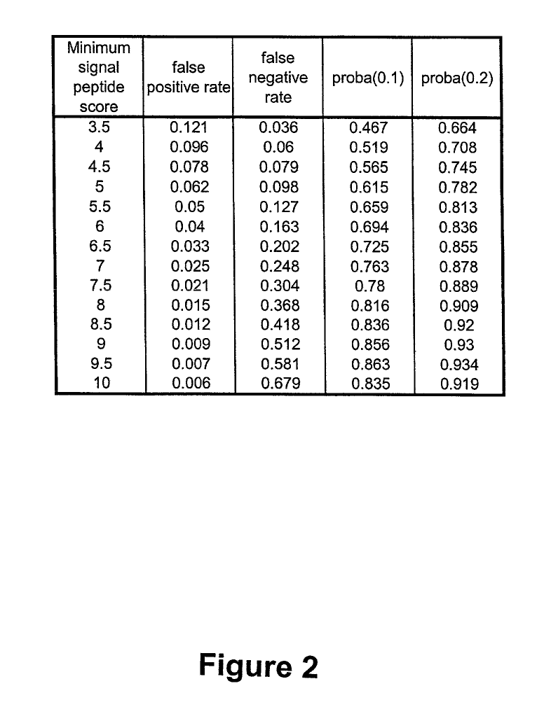

Using the results of the above analysis, the probability that a peptide encoded by the 5′ region of the mRNA is in fact a genuine signal peptide based on its Von Heijne's score was calculated based on either the assumption that 10% of human proteins are secreted or the assumption that 20% of human proteins are secreted. The results of this analysis are shown in FIG. 2.

Using the above method of identification of secretory proteins, 5′ ESTs of the following polypeptides known to be secreted were obtained: human glucagon, gamma interferon induced monokine precursor, secreted cyclophilin-like protein, human pleiotropin, and human biotinidase precursor. Thus, the above method successfully identified those 5′ ESTs which encode a signal peptide.

To confirm that the signal peptide encoded by the 5′ ESTs or cDNAs actually functions as a signal peptide, the signal sequences from the 5′ ESTs or cDNAs may be cloned into a vector designed for the identification of signal peptides. Such vectors are designed to confer the ability to grow in selective medium only to host cells containing a vector with an operably linked signal sequence. For example, to confirm that a 5′ EST or cDNA encodes a genuine signal peptide, the signal sequence of the 5′ EST or cDNA may be inserted upstream and in frame with a non-secreted form of the yeast invertase gene in signal peptide selection vectors such as those described in U.S. Pat. No. 5,536,637, the disclosure of which is incorporated herein by reference. Growth of host cells containing signal sequence selection vectors with the correctly inserted 5′ EST or cDNA signal sequence confirms that the 5′ EST or cDNA encodes a genuine signal peptide.

Alternatively, the presence of a signal peptide may be confirmed by cloning the 5′ESTs or cDNAs into expression vectors such as pXT1 as described below, or by constructing promoter-signal sequence-reporter gene vectors which encode fusion proteins between the signal peptide and an assayable reporter protein. After introduction of these vectors into a suitable host cell, such as COS cells or NIH 3T3 cells, the growth medium may be harvested and analyzed for the presence of the secreted protein. The medium from these cells is compared to the medium 10 from control cells containing vectors lacking the signal sequence or cDNA insert to identify vectors which encode a functional signal peptide or an authentic secreted protein.

### EXAMPLE 10

Evaluation of Expression Levels and Patterns of mRNAs Corresponding to 5′ ESTs or cDNAs

The spatial and temporal expression patterns of the mRNAs corresponding to the 5′ ESTs or cDNAs, as well as their expression levels, may be determined. Characterization of the spatial and temporal expression patterns and expression levels of these mRNAs is useful for constructing expression vectors capable of producing a desired level of gene product in a desired spatial or temporal manner, as will be discussed in more detail below.

In addition, cDNAs or 5′ ESTs whose corresponding mRNAs are associated with disease states may also be identified. For example, a particular disease may result from lack of expression, over expression, or under expression of an mRNA corresponding to a cDNA or 5′ EST. By comparing mRNA expression patterns and quantities in samples taken from healthy individuals with those from individuals suffering from a particular disease, cDNAs and 5′ ESTs responsible for the disease may be identified.

Expression levels and patterns of mRNAs corresponding to 5′ ESTs or cDNAs may be analyzed by solution hybridization with long probes as described in International Patent Application No. WO 97/05277, the entire contents of which are hereby incorporated by reference. Briefly, a 5′ EST, cDNA, or fragment thereof corresponding to the gene encoding the mRNA to be characterized is inserted at a cloning site immediately downstream of a bacteriophage (T3, T7 or SP6) RNA polymerase promoter to produce antisense RNA. Preferably, the 5′ EST or cDNA is 100 or more nucleotides in length. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridizations are performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, Ti, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

The 5′ ESTs, cDNAs, or fragments thereof may also be tagged with nucleotide sequences for the serial analysis of gene expression (SAGE) as disclosed in UK Patent Application No. 2 305 241 A, the entire contents of which are incorporated by reference. In this method, cDNAs are prepared from a cell, tissue, organism or other source of nucleic acid for which it is desired to determine gene expression patterns. The resulting cDNAs are separated into two pools. The cDNAs in each pool are cleaved with a first restriction endonuclease, called an “anchoring enzyme,” having a recognition site which is likely to be present at least once in most cDNAs. The fragments which contain the 5′ or 3′ most region of the cleaved cDNA are isolated by binding to a capture medium such as streptavidin coated beads. A first oligonucleotide linker having a first sequence for hybridization of an amplification primer and an internal restriction site for a “tagging endonuclease” is ligated to the digested cDNAs in the first pool. Digestion with the second endonuclease produces short “tag” fragments from the cDNAs.

A second oligonucleotide having a second sequence for hybridization of an amplification primer and an internal restriction site is ligated to the digested cDNAs in the second pool. The cDNA fragments in the second pool are also digested with the “tagging endonuclease” to generate short “tag” fragments derived from the cDNAs in the second pool. The “tags” resulting from digestion of the first and second pools with the anchoring enzyme and the tagging endonuclease are ligated to one another to produce “ditags.” In some embodiments, the ditags are concatamerized to produce ligation products containing from 2 to 200 ditags. The tag sequences are then determined and compared to the sequences of the 5′ ESTs or cDNAs to determine which 5′ ESTs or cDNAs are expressed in the cell, tissue, organism, or other source of nucleic acids from which the tags were derived. In this way, the expression pattern of the 5′ ESTs or cDNAs in the cell, tissue, organism, or other source of nucleic acids is obtained.

Quantitative analysis of gene expression may also be performed using arrays. As used herein, the term array means a one dimensional, two dimensional, or multidimensional arrangement of full length cDNAs (i.e. cDNAs which include the coding sequence for the signal peptide, the coding sequence for the mature protein, and a stop codon), cDNAs, 5′ ESTs or fragments of the full length cDNAs, cDNAs, or 5′ ESTs of sufficient length to permit specific detection of gene expression. Preferably, the fragments are at least 15 nucleotides in length. More preferably, the fragments are at least 100 nucleotides in length. More preferably, the fragments are more than 100 nucleotides in length. In some embodiments the fragments may be more than 500 nucleotides in length.

For example, quantitative analysis of gene expression may be performed with full length cDNAs, cDNAs, 5′ ESTs, or fragments thereof in a complementary DNA microarray as described by Schena et al. (Science 270:467-470, 1995; Proc. Natl. Acad. Sci. U.S.A. 93:10614-10619, 1996). Full length cDNAs, cDNAs, 5′ ESTs or fragments thereof are amplified by PCR and arrayed from 96-well microtiter plates onto silylated microscope slides using high-speed robotics. Printed arrays are incubated in a humid chamber to allow rehydration of the array elements and rinsed, once in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of the expression of genes may also be performed with full length cDNAs, cDNAs, 5′ ESTs, or fragments thereof in complementary DNA arrays as described by Pietu et al. (Genome Research 6:492-503, 1996). The full length cDNAs, cDNAs, 5′ ESTs or fragments thereof are PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis of the 5′ ESTs or cDNAs can be done through high density nucleotide arrays as described by Lockhart et al. (Nature Biotechnology 14: 1675-1680, 1996) and Sosnowsky et al. (Proc. Natl. Acad. Sci. 94:1119-1123, 1997). Oligonucleotides of 15-50 nucleotides corresponding to sequences of the 5′ ESTs or cDNAs are synthesized directly on the chip (Lockhart et al., supra) or synthesized and then addressed to the chip (Sosnowski et al., supra). Preferably, the oligonucleotides are about 20 nucleotides in length.

cDNA probes labeled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., supra and application of different electric fields (Sosnowsky et al., Proc. Natl. Acad. Sci. 94:1119-1123)., the dyes or labeling compounds are detected and quantified. Duplicate hybridizations are performed. Comparative analysis of the intensity of the signal originating from cDNA probes on the same target oligonucleotide in different cDNA samples indicates a differential expression of the mRNA corresponding to the 5′ EST or cDNA from which the oligonucleotide sequence has been designed.

III. Characterization of cDNAs including the 5′End of their Corresponding mRNA

### EXAMPLE 11

Characterization of the Complete Sequence of cDNA Clones

Clones which include the 5′end of their corresponding mRNA and which encode a new protein with a signal peptide as determined in the aforementioned procedure were then fully sequenced as follows.

First, both 5′ and 3′ ends of cloned cDNAs were sequenced twice in order to confirm the identity of the clone using a Die Terminator approach with the AmpliTaq DNA polymerase FS kit available from Perkin Elmer. Second, primer walking was performed if the full coding region had not been obtained yet using software such as OSP to choose primers and automated computer software such as ASMG (Sutton et al., Genome Science Technol. 1: 9-19, 1995) to construct contigs of walking sequences including the initial 5′ tag. Contigation was then performed using 5′ and 3′ sequences and eventually primer walking sequences. The sequence was considered complete when the resulting contigs included the full coding region as well as overlapping sequences with vector DNA on both ends. In addition, clones were entirely sequenced in order to obtain at least two sequences per clone. Preferably, the sequences were obtained from both sense and antisense strands. All the contigated sequences for each clone were then used to obtain a consensus sequence which was then submitted to the computer analysis described below.

Alternatively, clones which include the 5′end of their corresponding mRNA and which encode a new protein with a signal peptide, as determined in the aforementioned procedure, may be subcloned into an appropriate vector such as pED6dpc2 (DiscoverEase, Genetics Institute, Cambridge, Mass.) before full sequencing.

### EXAMPLE 12

Determination of Structural and Functional Features

Following identification of contaminants and masking of repeats, structural features, e.g. polyA tail and polyadenylation signal, of the sequences of cDNAs were subsequently determined using the algorithm, parameters and criteria defined in FIG. 1. Briefly, a polyA tail was defined as a homopolymeric stretch of at least 11 A with at most one alternative base within it. The polyA tail search was restricted to the last 100 nt of the sequence and limited to stretches of 11 consecutive A's because sequencing reactions are often not readable after such a polyA stretch. To search for a polyadenylation signal, the polyA tail was clipped from the full-length sequence. The 50 bp preceding the polyA tail were searched for the canonic polyadenylation AAUAAA signal allowing one mismatch to account for possible sequencing errors as well as known variation in the canonical sequence of the polyadenylation signal.

Functional features, e.g. ORFs and signal sequences, of the sequences of cDNAs were subsequently determined as follows. The 3 upper strand frames of cDNAs were searched for ORFs defined as the maximum length fragments beginning with a translation initiation codon and ending with a stop codon. ORFs encoding at least 80 amino acids were preferred. Each found ORF was then scanned for the presence of a signal peptide using the matrix method described in example 10.

Sequences of cDNAs were then compared, on a nucleotidic or proteic basis, to public sequences available at the time of filing.

### EXAMPLE 13

Selection of Full Length Sequences

cDNAs that had already been characterized by the aforementioned computer analysis were then submitted to an automatic procedure in order to preselect cDNAs containing sequences of interest.

a) Automatic Sequence Preselection

All cDNAs clipped for vector on both ends were considered. First, a negative selection was performed in order to eliminate sequences which resulted from either contaminants or artifacts as follows. Sequences matching contaminant sequences were discarded as well as those encoding ORF sequences exhibiting identity to repeats. Sequences lacking polyA tail were also discarded. Those cDNAs which matched a known human mRNA or EST sequence and had a 5′ end more than 30 nucleotides downstream of the known 5′ end were also excluded from further analysis. Only ORFs ending before the polyA tail were kept.

Then, for each remaining cDNA containing several ORFs, a preselection of ORFs was performed using the following criteria. The longest ORF was preferred. If the ORF sizes were similar, the chosen ORF was the one which signal peptide had the highest score according to Von Heijne method as defined in Example 10.

Sequences of cDNA clones were then compared pairwise with BLAST after masking of the repeat sequences. Sequences containing at least 90% identity over 30 nucleotides were clustered in the same class. Each cluster was then subjected to a clustal analysis that detects sequences resulting from internal priming or from alternative splicing, identical sequences or sequences with several frameshifts. This automatic analysis served as a basis for manual selection of the sequences.

b) Manual Sequence Selection

Manual selection was carried out using automatically generated reports for each sequenced cDNA clone. During the manual selection procedure, a selection was performed between clones belonging to the same class as follows. ORF sequences encoded by clones belonging to the same class were aligned and compared. If the identity between nucleotidic sequences of clones belonging to the same class was more than 90% over 30 nucleotide stretches or if the identity between amino acid sequences of clones belonging to the same class was more than 80% over 20 amino acid stretches, then the clones were considered as being identical. The chosen ORF was either the one exhibiting matches with known amino acid sequences or the best one according to the criteria mentioned in the automatic sequence preselection section. If the nucleotide and amino acid homologies were less than 90% and 80% respectively, the clones were said to encode distinct proteins which can be both selected if they contain sequences of interest.

Selection of full length cDNA clones encoding sequences of interest was performed using the following criteria. Structural parameters (initial tag, polyadenylation site and signal, eventually matches with public ESTs in 5′ or 3′ of the sequence) were first checked in order to confirm that the cDNA was complete in 5′ and in 3′. Then, homologies with known nucleic acids and proteins were examined in order to determine whether the clone sequence matched a known nucleic acid or protein sequence and, in the latter case, its covering rate and the date at which the sequence became public. If there was no extensive match with sequences other than ESTs or genomic DNA, or if the clone sequence included substantial new information, such as encoding a protein resulting from alternative splicing of an mRNA coding for an already known protein, the sequence was kept. Examples of such cloned full length cDNAs containing sequences of interest are described in Example 14. Sequences resulting from chimera or double inserts as assessed by identity to other sequences were discarded during this procedure.

### EXAMPLE 14

Characterization of Full-Length cDNAs

The procedure described above was used to obtain full-length cDNAs of the invention comprising the sequences of SEQ ID NOs: 1-405 derived from a variety of tissues. The polypeptides encoded by the extended or full-length cDNAs may be screened for the presence of known structural or functional motifs or for the presence of signatures or small amino acid sequences which are well conserved amongst the members of a protein family. Some of the results obtained for the polypeptides encoded by full-length cDNAs that were screened for the presence of known protein signatures and motifs using the Proscan software from the GCG package and the Prosite database are provided below.

Bacterial clones containing plasmids containing the full-length cDNAs are presently stored in the inventor's laboratories under the internal identification numbers provided. The inserts may be recovered from the deposited materials by growing an aliquot of the appropriate bacterial clone in the appropriate medium. The plasmid DNA can then be isolated using plasmid isolation procedures familiar to those skilled in the art such as alkaline lysis minipreps or large scale alkaline lysis plasmid isolation procedures. If desired the plasmid DNA may be further enriched by centrifugation on a cesium chloride gradient, size exclusion chromatography, or anion exchange chromatography. The plasmid DNA obtained using these procedures may then be manipulated using standard cloning techniques familiar to those skilled in the art. Alternatively, a PCR can be done with primers designed at both ends of the cDNA insertion. The PCR product which corresponds to the cDNA can then be manipulated using standard cloning techniques familiar to those skilled in the art.

Table I provides the sequence identification numbers of the cDNAs of the present invention, the locations of the first and last nucleotides of the full coding sequences in SEQ ID NOs: 1-405 (i.e. the nucleotides encoding both the signal peptide and the mature protein, listed under the heading FCS location in Table I), the locations of the first and last nucleotides in SEQ ID NOs: 1-405 which encode the signal peptides (listed under the heading SigPep Location in Table 1), the locations of the first and last nucleotides in SEQ ID NOs: 1-405 which encode the mature proteins generated by cleavage of the signal peptides (listed under the heading Mature Polypeptide Location in Table I), the locations in SEQ ID NOs: 1-405 of stop codons (listed under the heading Stop Codon Location in Table I), the locations of the first and last nucleotides in SEQ ID NOs: 1-405 of the polyA signals (listed under the heading Poly A Signal Location in Table I) and the locations of the first and last nucleotides of the polyA sites (listed under the heading Poly A Site Location in Table I).

Table II lists the sequence identification numbers of the polypeptides of SEQ ID NOs: 406-810, the locations of the first and last amino acid residues of SEQ ID NOs: 406-810 in the full length polypeptide (second column), the locations of the first and last amino acid residues of SEQ ID NOs: 406-810 in the signal peptides (third column), and the locations of the first and last amino acid residues of SEQ ID NOs: 406-810 in the mature polypeptide created by cleaving the signal peptide from the full length polypeptide (fourth column).

The nucleotide sequences of the sequences of SEQ ID NOs: 1-405 and the amino acid sequences encoded by SEQ ID NOs: 1-405 (i.e. amino acid sequences of SEQ ID NOs: 406-810) are provided in the appended sequence listing. In some instances, the sequences are preliminary and may include some incorrect or ambiguous sequences or amino acids. All instances of the symbol “n” in the nucleic acid sequences mean that the nucleotide can be adenine, guanine, cytosine or thymine. For each amino acid sequence, Applicants have identified what they have determined to be the reading frame best identifiable with sequence information available at the time of filing. In some instances the polypeptide sequences in the Sequence Listing contain the symbol “Xaa.” These “Xaa” symbols indicate either (1) a residue which cannot be identified because of nucleotide sequence ambiguity or (2) a stop codon in the determined sequence where applicants believe one should not exist (if the sequence were determined more accurately). Thus, “Xaa” indicates that a residue may be any of the twenty amino acids. In some instances, several possible identities of the unknown amino acids may be suggested by the genetic code.

The sequences of SEQ ID NOs: 1-405 can readily be screened for any errors therein and any sequence ambiguities can be resolved by resequencing a fragment containing such errors or ambiguities on both strands. Nucleic acid fragments for resolving sequencing errors or ambiguities may be obtained from the deposited clones or can be isolated using the techniques described herein. Resolution of any such ambiguities or errors may be facilitated by using primers which hybridize to sequences located close to the ambiguous or erroneous sequences. For example, the primers may hybridize to sequences within 50-75 bases of the ambiguity or error. Upon resolution of an error or ambiguity, the corresponding corrections can be made in the protein sequences encoded by the DNA containing the error or ambiguity. The amino acid sequence of the protein encoded by a particular clone can also be determined by expression of the clone in a suitable host cell, collecting the protein, and determining its sequence.

### EXAMPLE 15A

Categorization of cDNAs of the Present Invention

The nucleic acid sequences of the present invention (SEQ ID NOs. 1-405) were grouped based on their identity to known sequences as follows. All sequences were compared to public sequences available at the time of filing the priority applications.

In some instances, the cDNAs did not match any known vertebrate sequence nor any publicly available EST sequence, thus being completely new.

All sequences exhibiting more than 90% of identity to known sequences over at least 30 nucleotides were retrieved and further analyzed. For cDNAs referred to by their sequence identification numbers (first column), Table III gives the positions of preferred fragments within these sequences (second column entitled “Positions of preferred fragments”). Each fragment is represented by x-y where x and y are the start and end positions respectively of a given preferred fragment. Preferred fragments are separated from each other by a coma. As used herein the term “polynucleotide described in Table III” refers to the all of the preferred polynucleotide fragments defined in Table III in this manner.

For polynucleotides referred to by sequence identification numbers (first column), the second column of Table IVa provides the positions of fragments which are preferably included in the present invention (column 2) while the second column of IVb provides the positions of fragments which are preferably excluded from the present invention. Each fragment is represented by x-y where x and y are the start and end positions respectively of a given fragment. Fragments are separated from each other by a semi-column. Tables IVa and IVb provides for the inclusion and exclusion of polynucleotides in addition to those described elsewhere in the specification and is therefore, not meant as limiting description. As used herein the terms “polynucleotide described in Table IVa” and “polynucleotide described in Table UVb” refers to the all of the polynucleotide fragments defined in the second column of Tables IVa or IVb respectively in this manner.

The present invention encompasses isolated, purified, or recombinant nucleic acids which consist of, consist essentially of, or comprise a contiguous span of one of the sequences of SEQ ID Nos. 1-405 or a sequence complementary thereto, said contiguous span comprising at least 8, 10, 12, 15, 18, 20, 25, 28, 30, 35, 40, 50, 75, 100, 150, 200, 300, 400, 500, 1000 or 2000 nucleotides of the sequence of SEQ ID Nos. 1-405 or a sequence complementary thereto, to the extent that a contiguous span of these lengths is consistent with the lengths of the particular sequence, wherein the contiguous span comprises at least 1, 2, 3, 5, 10, 15, 18, 20, 25, 28, 30, 35, 40, 50, 75, 100, 150, 200, 300, 400 or 500 of a polynucleotide described in Table III or of a polynucleotide described in Table IVa, or a sequence complementary thereto. The present invention also encompasses isolated, purified, or recombinant nucleic acids comprising, consisting essentially of, or consisting of a contiguous span of at least 8, 10, 12, 15, 18, 20, 25, 28, 30, 35, 40, 50, 75, 100, 150, 200, 300, 400, 500, 1000 or 2000 nucleotides of a polynucleotide described in Table III or of a polynucleotide described in Table IVa or a sequence complementary thereto, to the extent that a contiguous span of these lengths is consistent with the length of the particular sequence described in Table III. The present invention also encompasses isolated, purified, or recombinant nucleic acids which comprise, consist of or consist essentially of a polynucleotide described in Table III or of a polynucleotide described in Table IVa, or a sequence complementary thereto. The present invention further encompasses any combination of the nucleic acids listed in this paragraph.

Cells containing the cDNAs (SEQ ID NOs: 1-405) of the present invention in the vector pBluescriptII SK− (Stratagene) are maintained in permanent deposit by the inventors at Genset, S. A., 24 Rue Royale, 75008 Paris, France.

Pool of cells containing the cDNAs of SEQ ID NOs: 1-405, from which the cells containing a particular polynucleotide is obtainable, were deposited with the European Collection of Cell Cultures (ECACC), Vaccine Research and Production Laboratory, Public Health Laboratory Service, Centre for Applied Microbiology and Reasearch, Porton Down, Salisbury, Wiltshire SP4 OJG, United Kingdom. Each cDNA clone has been transfected into separate bacterial cells (E-coli) for these composite deposits. In particular, cells containing the sequences of SEQ ID NOs: 2-17 and 19-23 were deposited on Jun. 17, 1999 in the pool having ECACC Accession No. 99061735 and designated SignalTag 15061999. In addition, cells containing the sequences of SEQ ID Nos: 24-50 were deposited on Dec. 18, 1998, in the pool having ECACC Accession No. 98121805 and designated SignalTag 166-191. Table IV provides the internal designation number assigned to each SEQ ID NO. and indicates whether the sequence is a nucleic acid sequence or a protein sequence.

Each cDNA can be removed from the Bluescript vector in which it was deposited by performing a BsH II double digestion to produce the appropriate fragment for each clone provided the cDNA clone sequence does not contain this restriction site. Alternatively, other restriction enzymes of the multicloning site of the vector may be used to recover the desired insert as indicated by the manufacturer.

Bacterial cells containing a particular clone can be obtained from the composite deposit as follows:

An oligonucleotide probe or probes should be designed to the sequence that is known for that particular clone. This sequence can be derived from the sequences provided herein, or from a combination of those sequences. The design of the oligonucleotide probe should preferably follow these parameters:
(a) It should be designed to an area of the sequence which has the fewest ambiguous bases (“N's”), if any;
(b) Preferably, the probe is designed to have a Tₘ of approx. 80° C. (assuming 2 degrees for each A or T and 4 degrees for each G or C). However, probes having melting temperatures between 40° C. and 80° C. may also be used provided that specificity is not lost.

The oligonucleotide should preferably be labeled with (−[³²P]ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantified by measurement in a scintillation counter. Preferably, specific activity of the resulting probe should be approximately 4×10⁶ dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 pg/ml of yeast RNA, and 10 mM EDTA (approximately 10 ml per 150 mm filter). Preferably, the probe is then added to the hybridization mix at a concentration greater than or equal to 1×10⁶ dpm/ml. The filter is then preferably incubated at 65° C. with gentle agitation overnight. The filter is then preferably washed in 500 ml of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

The plasmid DNA obtained using these procedures may then be manipulated using standard cloning techniques familiar to those skilled in the art. Alternatively, a PCR can be done with primers designed at both ends of the cDNA insertion. The PCR product which corresponds to the cDNA can then be manipulated using standard cloning techniques familiar to those skilled in the art.

Tissue expression of the cDNAs of the present invention was also examined. Table VI list the Genset's libraries of tissues and cell types examined that express the polynucleotides of the present invention. The tissues and cell types examined for polynucleotide expression were: brain, fetal brain, fetal kidney, fetal liver, pituitary gland, liver, placenta, prostate, salivary gland, stomach/intestine, and testis. For cDNAs referred to by sequence identification number (first column), the number of proprietary 5′ESTs expressed in a particular tissue referred to by its name is indicated in parentheses (second column). In addition, the bias in the spatial distribution of the polynucleotide sequences of the present invention is indicated in Table VII. The expression of these sequences were examined by comparing the relative proportions of the biological polynucleotides of a given tissue using the following statistical analysis. The under- or over-representation of a polynucleotide of a given cluster in a given tissue was performed using the normal approximation of the binomial distribution. When the observed proportion of a polynucleotide of a given tissue in a given consensus had less than 1% chance to occur randomly according to the chi2 test, the frequency bias was reported as “preferred”. The results are given in Table VII as follows. For some polynucleotides showing a bias in tissue distribution as referred to by sequence identification number in the first column, the list of tissues where the polynucleotides are over-represented is given in the second column entitled “preferential expression”.

In addition, the spatial distribution of the polynucleotide sequences of the present invention was investigated using information from public databases. The expression of the sequences of SEQ ID NOs:1-405 was examined by comparing them to the polynucleotide sequences in public databases. Table VIII lists tissues and cell types which express the polynucleotides of the sequence listing. Column one lists the sequence identification number and column two lists the corresponding tissues and cell types that were found to express the polynucleotide sequences using information from public databases. The number to the right of the tissue or cell type in column two represents the number of entries in the databases listing that tissue or cell type as expressing the sequence of column 1.

In one embodiment, polynucleotides of the invention selectively expressed in tissues may be used as markers to identify these tissues using any technique known to those skilled in the art those skilled in the art such as in situ PCR. Such tissue-specific markers may then be used to identify tissues of unknown origin, for example, forensic samples, differentiated tumor tissue that has metastasized to foreign bodily sites, or to differentiate different tissue types in a tissue cross-section using immunochemistry. For example, polynucleotides of the invention preferentially expressed in given tissues as indicated in Table VII may be used for this purpose. In addition, the polynucleotide of SEQ ID NO: 16 may be used to selectively identify liver tissue. The polynucleotide of SEQ ID NO:29 may be used to selectively identify prostate tissue. The polynucleotides of SEQ ID NO:21, 23 and 49 may be used to selectively identify normal or diseased brain tissue.

### EXAMPLE 15B

Functional Analysis of Predicted Protein Sequences

Following double-sequencing, contigated sequences were assembled for each of the cDNAs of the present invention and further reanalyzed. The following databases were used in sequence analyses: Genbank (release 117), EMBL (release 62), TrEmbl (release 13.4) Genseq (release 0011) Swissprot (release 38), PIR (release 64). In some cases, more preferred open reading frames differing from the ones previously selected in priority applications are indicated.

The polypeptides (SEQ ID NOs: 406-810) encoded by the cDNAs were screened for the presence of known structural or functional motifs or for the presence of signatures, small amino acid sequences that are well conserved amongst the members of a protein family. The search was conducted on the Pfam 5.2 database using HMMER-2.1.1 (for info see Sonnhammer et Durbin, http:/www.sanger.ac.uk/Pfam/), on the BLOCKSPLUS v 11.0 database using emotif (for info see Nevill-Manning et al., PNAS, 95, 5865-5871, (1998), http://motif.stanford/edu/EMOTIF) and on the Prosite 15.0 database using bla (Tatusov, R. L. & Koonin, E. V. CABIOS 10, No. 4) and pfscan (http://www.isrec.isb-sib.ch/cgi-bin/man.cgi?section=1 &topic=pfscan).

It should be noted that, in the numbering of amino acids in the protein sequences discussed below, and in Table IX, the first methionine encountered is designated as amino acid number 1, i.e.; the leader sequence is not numbered negatively. In the appended sequence listing, the first amino acid of the mature protein resulting from cleavage of the signal peptide is designated as amino acid number 1 and the first amino acid of the signal peptide is designated with the appropriate negative number, in accordance with the regulations governing sequence listings. Each of the references cited in this example are hereby incorporated by reference in their entireties.

Table IX lists known biologically structural and functional domains for the cDNA of the present invention corresponding to the sequence identification number indicated in the first column. Column 2 lists the positions of the domains where each domain is represented by x-y where x and y are the start and end positions respectively of a given domain. Column 3 lists the domain designation. Column 4 lists the database from which the domain was identified.

Protein of SEQ ID NO: 425 (Internal Designation 117-007-2-0-C4-FLC)

The protein of SEQ ID NO: 425 encoded by the cDNA of SEQ ID NO:20 found in liver is homologous to a human protein thought to be transmembraneous (Genseq accession number W88491). In addition, this protein displays homology to alpha-2-HS glycoprotein precursors (fetuins) of human and pigs. The 382-amino-acid-long protein of SEQ ID NO: 425, which is similar in size to fetuins, displays pfam cystatin domains 1 and 2 from positions 37 to 104 and from positions 157 to 254. It also displays the 12 conserved cysteines of this family (positions 36, 93, 104, 117, 137, 151, 154, 216, 224, 237, 254 and 368) and a conserved region around the second cysteine (positions 89 to 96). In addition, the potential active site QxVxG is also present in the protein of the invention (positions 198 to 202).

Mammalian fetuins are secreted glycoproteins synthesized in liver and selectively concentrated in bone matrix. Their functions include control of endocytosis, cell proliferation and differentiation, immune response, bone formation and resorption, and apoptosis. More specifically, fetuin levels in human plasma are regulated in the manner of a negative acute phase reactant (Lebreton et al., J. Clin. Invest. 64:1118-29 (1979)) and serum levels decline in some cancer patients correlating with impaired cellular immune function (Baskies et al., Cancer 45:3050-58 (1980)). During mouse embryogenesis, fetuin mRNA is expressed in a number of developing organs and tissues including the heart, kidney, lung, nervous system and liver (Yang et al., Biochem. Biophysic. Acta 1130:149-56 (1992)). Mammalian fetuin present in sub-populations of neurons in the developing central and peripheral nervous system is associated to cell survival (Saunders et al., Anat. Embryol 186:477-86 (1992)); Kitchener et al., Int J. Dev. Neurosci. 15:717-27 (1997)). Fetuin is able to promote growth in tissue culture (Puck et al. Proc. Natl. Acad. Sci. U. S. A., 59:192-99 (1968)), to enhance bone resorption (Coclasure et al., J. Clin. Endocrinol. Metab. 66:187-192 (1988)) and to stimulate adipogenesis in cell culture models (Cayatte et al., J. Biol. Chem. 265:5883-8 (1990)). Abnormal serum levels of fetuin are associated with alteration in cellular and biochemical properties of bone, Paget's disease, reduced bone quality and osteogenesis imperfecta (for a review see Binkert et al, J. Biol. Chem. 274:28514-20 (1999)). Part of the fetuin activities has been shown to depend upon their ability to inhibit the activity of TGF-beta cytokines and bone morphogenetic proteins (BMPs) through direct binding (Demetriou et al., J. Biol. Chem. 271:12755-61 (1996); Binkert et al., J. Biol. Chem. 274:28514-20 (1999)). These ligands are members of the TGF-beta superfamily comprising proteins belonging to the TGF-beta, activin/inhibin, DPP/VG1, and Mullerian Inhibiting Substance Family families mediating a wide range of biological processes in vertebrates and invertebrates, including regulation of cell proliferation, differentiation, recognition, and death, and thus play a major role in developmental processes, tissue recycling, and repair (J. Wrana and L. Attisano, “Mad-related Proteins in TGF-beta Signaling,” TIG 12:493-496, 1996; U.S. Pat. No. 5,981,483). In addition, fetuins are members of the cystatin superfamily which contains evolutionarily related proteins with diverse functions such as cysteine protease inhibitors, stefins, fetuins and kininogens (see review by Brown and Dziegielewska, Prot. Science, 6:5-12 (1997)).

It is believed that the protein of SEQ ID NO: 425 or part thereof is a member of the cystatin superfamily and, as such, plays a role in cellular proteolysis, endocytosis, cell proliferation and differentiation, immune response, bone formation and resorption, and/or apoptosis. Preferred polypeptides of the invention are polypeptides comprising the amino acids of SEQ ID NO:425 from positions 37 to 104, 89 to 96, 157 to 254, 198 to 202, and 36 to 368. Other preferred polypeptides of the invention are fragments of SEQ ID NO:425 having any of the biological activity described herein.

An embodiment of the present invention relates to methods of using the protein of the invention or part thereof to identify and/or quantify cytokines of the TGF-beta superfamily, more preferably TGF-1beta, TGF-2beta and BMP-2, BMP-4 and BMP-6 in a biological sample, and thus used in assays and diagnostic kits for the quantification of such cytokines in bodily fluids, in tissue samples, and in mammalian cell cultures. The binding activity of the protein of the invention or part thereof may be assessed using the assay described in Demetriou et al., J. Biol. Chem. 271:12755-61 (1996) or any other method familiar to those skilled in the art. Preferably, a defined quantity of the protein of the invention or part thereof is added to the sample under conditions allowing the formation of a complex between the protein of the invention or part thereof and the cytokine to be identified and/or quantified. Then, the presence of the complex and/or or the free protein of the invention or part thereof is assayed and eventually compared to a control using any of the techniques known by those skilled in the art.

Another embodiment of the invention relates to compositions and methods using the protein of the invention or part thereof to modulate the activity of members of the TGF beta superfamily, preferably members of TGF beta family, members of actin/inhibin family, members of DPP/VG1 family, and members of Mullerian inhibiting substance family, more preferably TGF-1beta, TGF-2beta, BMP-2, BMP-4 and BMP-6, in contexts where the production of such proteins is undesirable.

In a preferred embodiment, the protein of the invention or part thereof is used to inhibit and/or attenuate the effects of cytokines belonging to the TGF beta family, such as TGF-1beta, TGF-2beta and BMP-2, BMP-4 and BMP-6, by blocking the binding of endogenous cytokines to its natural receptor, thereby blocking cell proliferative or inhibitory signals generated by the ligand-receptor binding event. The protein of the invention or part thereof would thereby stimulate immune responses and reduce the deposition of extracellular matrix. Accordingly, the protein of the invention or part thereof, would be particularly suitable for the treatment of conditions such as fibrosis including pulmonary fibrosis, fibrosis associated with chronic liver disease, hepatic veno-occlusive and idiopathic interstitial pneumonitis, kidney disease, and radiotherapy or radiation accidents; proliferative vitreoretinopathy; systemic sclerosis; autoimmune disorders such as rheumatoid arthritis, Graves disease, systemic lupus erythematosus, Wegener's granulomatosis, sarcoidosis, polyarthritis, pemphigus, pemphigoid, erythema multiform, Sjogren's syndrome, inflammatory bowel disease, multiple sclerosis, myasthenia gravis keratitis, scleritis, Type I diabetes, insulin-dependent diabetes mellitus, Lupus Nephritis, and allergic encephalomyelitis; proliferative disorders including various forms of cancer such as leukemias, lymphomas (Hodgkins and non-Hodgkins), sarcomas, melanomas, adenomas, carcinomas of solid tissue, hypoxic tumors, squamous cell carcinomas of the mouth, throat, larynx, and lung, genitourinary cancers such as cervical and bladder cancer, hematopoietic cancers, head and neck cancers, and nervous system cancers, benign lesions such as papillomas, atherosclerosis, angiogenesis, and viral infections, in particular HIV infections. The protein of the invention or part thereof may also be used, as an antagonist of cytokines of the TGF-beta family, to elevate blood pressure through the inhibition of hypotension induced by TGF-beta. Methods which lower and/or maintain the level of circulating TGF-beta in a subject may result in a similar pressor effect and may prevent excessive hypotensive signal generation and resulting hypotension.

In another preferred embodiment, the protein of the invention or part thereof is used to block the normal interaction between activin and its receptor. The protein of the invention or part thereof would thereby stimulate the release of FSH. Accordingly, the protein of the invention or part thereof can be applied to the control of fertility in humans, domesticated animals, and animals of commercial interest. The action of activin on erythropoiesis can also be modulated by administering a modulating effective amount of the protein of the invention or part thereof. Thus, the protein of the invention or part thereof may be used in the diagnosis and/or treatment of activin-dependent tumors or for enhancing the survival of brain neurons.

In still another preferred embodiment, the protein of the invention or part thereof is used to modulate bone formation and bone cell differentiation through binding to bone morphogenetic proteins and/or to TGF-beta proteins. Therefore, the protein of the invention or part thereof may be used to repair or heal fractures, treat osteoporosis, address dental problems, and with implants to encourage bone growth. In addition, the protein of the invention or part thereof may be used in disorders where there is too much bone formation (for example, achondroplasia, Paget's disease, and osteoporosis). The utility of the protein of the invention or part thereof may be further confirmed using binding assays and animal models described in Demetriou et al., J. Biol. Chem. 271:12755-61 (1996) and in U.S. Pat. No. 5,981,483.

In still another embodiment, the invention relates to methods and compositions containing the protein of the invention or part thereof to treat and/or prevent the ill-effects of bacterial infection during pregnancy in mammals, such as spontaneous abortion and maternal death. In a preferred embodiment, the protein of the invention may be used to counteract the effects of the bacterial endotoxin lipopolysaccharide (LPS). The method to use such compositions is described in Dziegielewska and Andersen, Biol. Neonate, 74:372-5 (1998).

In another series of embodiments, the protein of the invention, or part thereof may be used to inhibit proteases, preferably cysteine proteases. Examples of cysteine proteases that may be inhibited by the protein of the invention or part thereof include, but are not limited to, the plant cysteine proteases such as papain, ficin, aleurain, oryzain and actinidin; mammalian cysteine proteases such as cathepsins B, H, J, L, N, S, T, 0, 02 and C, (cathepsin C is also known as dipeptidyl peptidase I), interleukin converting enzyme (ICE), calcium-activated neutral proteases, calpain I and II; bleomycin hydrolase, viral cysteine proteases such as picomian 2A and 3C, aphthovirus endopeptidase, cardiovirus endopeptidase, comovirus endopeptidase, potyvirus endopeptidases I and II, adenovirus endopeptidase, the two endopeptidases from chestnut blight virus, togavirus cysteine endopeptidase, as well as cysteine proteases of the polio and rhinoviruses; and cysteine proteases known to be essential for parasite lifecycles, such as the proteases from species of Plasmodia, Entamoeba, Onchocera, Trypanosoma, Leishmania, Haemonchus, Dictyostelium, Therileria, and Schistosoma, such as those associated with malaria (P. falciparum), trypanosomes (T. cruzi, the enzyme is also known as cruzain or cruzipain), murine P. vinckei, and the C. elegans cysteine protease. For an extensive listing of cysteine proteases that may be inhibited by the protein or part thereof of the present invention, see Rawlings et al., Biochem. J. 290:205-218 (1993). Assays for testing the inhibitory activities of cysteine protease inhibitors are presented in the U.S. Pat. No. 5,973,110, using methods for determining inhibition constants well known to those skilled in the art (see Fersht, ENZYME STRUCTURE AND MECHANISM, 2nd ed., W.H. Freeman and Co., New York, (1985)).

Since proteases play an important role in the regulation of many biological processes in virtually all living organisms as well as a major role in diseases, the protein of the invention or part thereof are useful in a wide variety of applications, such as those described in U.S. Pat. No. 6,004,933.

An embodiment of the present invention further relates to methods of using the protein of the invention or part thereof to quantify the amount of a given protease in a biological sample, and thus used in assays and diagnostic kits for the quantification of proteases in bodily fluids or other tissue samples, in addition to bacterial, fungal, plant, yeast, viral or mammalian cell cultures. In a preferred embodiment, the sample is assayed using a standard protease substrate. A known concentration of protease inhibitor is added, and allowed to bind to a particular protease present. The protease assay is then rerun, and the loss of activity is correlated to the protease inhibitor activity using techniques well known to those skilled in the art.

In addition, the protein of the invention or part thereof may be useful to remove, identify or inhibit contaminating proteases in a sample. Compositions comprising the polypeptides of the present invention may be added to biological samples as a “cocktail” with other protease inhibitors to prevent degradation of protein samples. The advantage of using a cocktail of protease inhibitors is that one is able to inhibit a wide range of proteases without knowing the specificity of any of the proteases. Using a cocktail of protease inhibitors also protects a protein sample from a wide range of future unknown proteases which may contaminate a protein sample from a vast number of sources. Such protease inhibitor cocktails (see for example the ready to use cocktails sold by Sigma) are widely used in research laboratory assays to inhibit proteases susceptible of degrading a protein of interest for which the assay is to be performed. For example, the protein of the invention or part thereof is added to samples where proteolytic degradation by contaminating proteases is undesirable. Alternatively, the protein of the invention or part thereof may be bound to a chromatographic support, either alone or in combination with other protease inhibitors, using techniques well known in the art, to form an affinity chromatography column. A sample containing the undesirable protease is run through the column to remove the protease. Alternatively, the same methods may be used to identify new proteases.

In a preferred embodiment, the protein of the invention or part thereof may be used to inhibit proteases implicated in a number of diseases where cellular proteolysis occur. In particular, the protein of the invention or part thereof may be useful to inhibit lysosomal cysteine proteases, both in vivo or in vitro, implicated in a wide spectrum of diseases characterized by tissue degradation including but not limited to arthritis, muscular dystrophy, inflammation, tumor invasion, glomerulonephritis, parasite-borne infections, Alzheimer's disease, periodontal disease, and cancer metastasis.

In another preferred embodiment, the protein of the invention or part thereof may be used to inhibit exogenous proteases, both in vivo or in vitro, implicated in a number of infectious diseases including but not limited to gingivitis, malaria, leishmaniasis, filariasis, osteoporosis and osteoarthritis, and other bacterial, and parasite-borne or viral infections. In particular, the protein of the invention or part thereof may offer applications in viral diseases where the proteolysis of primary polypeptide precursors is essential to the replication of the virus, as for HIV and HCV.

In another preferred embodiment, the protein of the invention or part thereof is used to prevent cells to undergo apoptosis. In a preferred embodiment, the apoptosis active polypeptide is added to an in vitro culture of mammalian cells in an amount effective to reduce apoptosis. For example, inhibiting the activity of apopain, a cysteine protease member of the ICE/CED-3 subfamily involved in apoptosis, attenuates apoptosis in vitro (U.S. Pat. No. 5,798,442). Furthermore, the protein of the invention or part thereof may be useful in the diagnosis, the treatment and/or the prevention of disorders in which apoptosis is deleterious, including but not limited to immune deficiency syndromes (including AIDS), type I diabetes, pathogenic infections, cardiovascular and neurological injury, alopecia, aging, Parkinson's disease and Alzheimer's disease.

Additionally, the protein of the invention or part thereof offer application in the treatment of inflammation and immune based disorders of the lung, airways, central nervous system and surrounding membranes, eyes, ears, joints, bones, connective tissues, cardiovascular system including the pericardium, gastrointestinal and urogenital systems, the skin and the mucosal membranes. These conditions include infectious diseases where active infection exists at any body site, such as meningitis and salpingitis; complications of infections including septic shock, disseminated intravascular coagulation, and/or adult respiratory distress syndrome; acute or chronic inflammation due to antigen, antibody and/or complement deposition; inflammatory conditions including arthritis, chalangitis, colitis, encephalitis, endocarditis, glomerulonephritis, hepatitis, myocarditis, pancreatitis, pericarditis, reperfusion injury and vasculitis. Immune-based diseases include but are not limited to conditions involving T-cells and/or macrophages such as acute and delayed hypersensitivity, graft rejection, and graft-versus-host disease; auto-immune diseases including Type I diabetes mellitus and multiple sclerosis. Bone and cartilage reabsorption as well as diseases resulting in excessive deposition of extracellular matrix such as interstitial pulmonary fibrosis, cirrhosis, systemic sclerosis, and keloid formation may also be treated with the protein of the invention or part thereof.

Furthermore, the protein of the present invention or part thereof find use in drug potentiation applications. For example, therapeutic agents such as antibiotics or antitumor drugs can be inactivated through proteolysis by endogenous proteases, thus rendering the administered drug less effective or inactive. Accordingly, the protein of the invention or part thereof may be administered to a patient in conjunction with a therapeutic agent in order to potentiate or increase the activity of the drug. This co-administration may be by simultaneous administration, such as a mixture of the protease inhibitor and the drug, or by separate simultaneous or sequential administration.

In addition, protease inhibitors have been shown to inhibit the growth of microorganisms including human pathogenic bacteria. For example, protease inhibitors are able to inhibit growth of all strains of group A streptococci, including antibiotic-resistant strains (Merigan, T. et al (1996) Ann Intern Med 124:1039-1050; Stoka, V. (1995) FEBS. Lett 370:101-104; Vonderfecht, S. et al (1988) J Clin Invest 82:2011-2016; Collins, A. et al (1991) Antimicrob Agents Chemother 35:2444-2446). Accordingly, the protein of the invention may or part thereof be used as antibacterial agents to retard or inhibit the growth of certain bacteria either in vitro or in vivo. Particularly, the polypeptides of the present invention may be used to inhibit the growth of group A streptococci on non-living matter such as instruments not conducive to other methods of preventing or removing contamination by group A streptococci, and in culture of living plant, fungi, and animal cells.

Protein of SEQ ID NO: 418 (Internal Designation 116-054-3-0-G12-FLC)

The protein of SEQ ID NO: 418 encoded by the cDNA of SEQ ID NO: 13 found in liver is homologous to the subunit 2 of NADH dehydrogenase (Genseq accession number Y14556) 35 and to the MLRQ subunit of NADH dehydrogenase (NADH-ubiquinone oxidoreductase, NADH-D or complex I) of bovine, murine and human species (Genbank accession numbers X64897, U59509 and EMBL accession number U94586 respectively). In addition, the 83-amino-acid-long protein of SEQ ID NO: 418 has a size similar to those of known MLRQ subunits as well as an hydrophobic N-terminal region of 25-30 amino acids.

Complex I is the first of 3 multienzyme complexes located in the mitochondrial membrane that make up the mitochondrial electron transport chain. Complex I accomplishes the first step in this process by accepting electrons from NADH and passing them through a flavin molecule to ubiquinone which then transfers electrons to the second enzyme complex in the chain.

Complex I contains approximately 40 polypeptide subunits of widely varying size and composition and is highly conserved in a variety of mammalian species including rat, rabbit, cow, and human (Cleeter, M. W. J. and Ragan, C. I. (1985) Biochem. J. 230: 739-46). The best characterized complex I is from bovine heart mitochondria and is composed of 41 polypeptides (Walker, J. E. et al. (1992) J. Mol. Biol. 226: 1051-72). Seven of these polypeptides are encoded by mitochondrial DNA, while the remaining 34 are nuclear gene products that are imported into the mitochondria. Six of these imported polypeptides are characterized by N-terminal signal peptide sequences which target these polypeptides to the mitochondria and are then cleaved from the mature proteins. A second group of polypeptides lack N-terminal targeting sequences and appear to contain import signals which lie within the mature protein (Walker et al., supra). The functions of many of the individual subunits in NADH-D are largely unknown. The 24-, 51-, and 75-kDa subunits have been identified as being catalytically important in electron transport, with the 51-kDa subunit forming part of the NADH binding site and containing the flavin moiety that is the initial electron acceptor (Ali, S. T. et al. (1993) Genomics 18:435-39). The location of other functionally important groups, such as the electron-carrying iron-sulfate centers, remains to be determined. Many of the smaller subunits (<30 kDa) contain hydrophobic sequences that may be folded into membrane spanning alpha-helices. These subunits presumably are anchored into the inner membrane of the mitochondria and interact via more hydrophilic parts of their sequence with globular proteins in the large extrinsic domain of NADH-D. The remaining proteins are likely to be globular and form part of a domain outside the lipid bilayer. The MLRQ subunit is one of the small (9 kDa) subunits that is nuclear encoded and contains no N-terminal extension to direct the protein into the mitochondrion, thus implying that the import signal should lie into the mature protein (Walker et al. supra). A potential membrane-spanning alpha-helix presumably anchors the MLRQ subunit to the inner membrane of the mitochondria, but the precise function of the subunit is unknown.

Mitochondriocytopathies due to complex I deficiency are frequently encountered and affect tissues with a high-energy demand such as brain (mental retardation, convulsions, movement disorders), heart (cardiomyopathy, conduction disorders), kidney (Fanconi syndrome), skeletal muscle (exercise intolerance, muscle weakness, hypotonia) and/or eye (opthmaloplegia, ptosis, cataract and retinopathy). Complex I is also thought to play a role in the regulation of apoptosis and necrosis. For a review on complex I, see Smeitink et al., Hum. Mol. Gent., 7: 1573-1579 (1998); Lenaz et al., Acta Biochem Pol 46:1-21 (1999); Lee and Wei, J Biomed Sci 7:2-15 (2000). In addition, defects and altered expression of complex I are associated with a variety of disease conditions in man, including neurodegenerative diseases, myopathies, and cancer (Singer, T. P. et al. (1995) Biochim. Biophys. Acta 1271:211-19; Selvanayagam, P. and Rajaraman, S. (1996) Lab. Invest. 74:592-99). Moreover, NADH-D reduction of the quinone moiety in chemotherapeutic agents such as doxorubicin is believed to contribute to the antitumor activity and/or mutagenicity of these drugs (Akman, S. A. et al. (1992) Biochemistry 31:3500-6).

It is believed that the protein of SEQ ID NO: 418 is a NADH-ubiquinone oxidoreductase MLRQ-like protein and/or plays a role in mitochondria electron transport. Preferred polypeptides of the invention are fragments of SEQ ID NO: 443 having any of the biological activities described herein

An object of the present invention are compositions and methods of targeting heterologous compounds, either polypeptides or polynucleotides to mitochondria by recombinantly or chemically fusing a fragment of the protein of the invention to an heterologous polypeptide or polynucleotide. Preferred fragments are signal peptide, amphiphilic alpha helices and/or any other fragments of the protein of the invention, or part thereof, that may contain targeting signals for mitochondria including but not limited to matrix targeting signals as defined in Herrman and Neupert, Curr. Opinion Microbiol. 3:210-4 (2000); Bhagwat et al. J. Biol. Chem. 274:24014-22 (1999), Murphy Trends Biotechnol. 15:326-30 (1997); Glaser et al. Plant Mol Biol 38:311-38 (1998); Ciminale et al. Oncogene 18:4505-14 (1999). Such heterologous compounds may be used to modulate mitochondria's activities. For example, they may be used to induce and/or prevent mitochondrial-induced apoptosis or necrosis. In addition, heterologous polynucleotides may be used for mitochondrial gene therapy to replace a defective mitochondrial gene and/or to inhibit the deleterious expression of a mitochondrial gene.

In another embodiment, the protein of the invention or part thereof is used to prevent cells to undergo apoptosis. In a preferred embodiment, the apoptosis active polypeptide is added to an in vitro culture of mammalian cells in an amount effective to reduce apoptosis. Furthermore, the protein of the invention or part thereof may be useful in the diagnosis, the treatment and/or the prevention of disorders in which apoptosis is deleterious, including but not limited to immune deficiency syndromes (including AIDS), type I diabetes, pathogenic infections, cardiovascular and neurological injury, alopecia, aging, degenerative diseases such as Alzheimer's Disease, Parkinson's Disease, Huntington's disease, dystonia, Leber's hereditary optic neuropathy, schizophrenia, and myodegenerative disorders such as “mitochondrial encephalopathy, lactic acidosis, and stroke” (MELAS), and “myoclonic epilepsy ragged red fiber syndrome” (MERRF).

The invention further relates to methods and compositions using the protein of the invention or part thereof to diagnose, prevent and/or treat several disorders in which mitochondrial respiratory electron transport chain is impaired, or needs to be impaired, including but not limited to mitochondriocytopathies, necrosis, aging, neurodegenerative diseases, myopathies, and cancer. For diagnostic purposes, the expression of the protein of the invention could be investigated using any of the Northern blotting, RT-PCR or immunoblotting methods described herein and compared to the expression in control individuals. For prevention and/or treatment purposes, the protein of the invention may be used to enhance electron transport and increase energy delivery using any of the gene therapy methods described herein or known to those skilled in the art.

Moreover, antibodies to the protein of the invention or part thereof may be used for detection of mitochondria organelles and/or mitochondrial membranes using any techniques known to those skilled in the art.

Protein of SEQ ID NO: 443 (Internal Designation 108-013-5-0-H9-FL)

The protein of SEQ ID NO: 443 encoded by the extended cDNA SEQ ID NO:38 is homologous to the human IHLP lysophospholipase (Genseq accession number W88457) and to a family of lysophospholipases conserved among eukaryotes (yeast, rabbit, rodents and human). In addition, some members of this family (rat:Genbank accession number U97146, rabbit: Genbank accession number U97147) exhibit a calcium-independent phospholipase A2 activity (Portilla et al, J. Am. Soc. Nephro., 9:1178-1186 (1998)). All members of this family exhibit the active site consensus GXSXG motif of carboxylesterases that is also found in the protein of the invention (position 54 to 58). The protein of the invention also exhibits an emotif alpha/beta hydrolase fold signature from positions 52 to 66. In addition, this protein may be a membrane protein with one transmembrane domain as predicted by the software TopPred II (Claros and von Heijne, CABIOS applic. Notes, 10:685-686 (1994)).

Lysophospholipids are found in very low concentrations in biological membranes. Higher concentrations of lysophospholipids have been shown to disturb membrane conformation, affect the activities of many membrane-bound enzymes and may even lead to cell lysis. In addition, increased lysophospholipid levels were observed in atherosclerosis, inflammation, hyperlipidemia, lethal dysrhythmias in myocardial ischemia and segmental demyelination of peripheral nerves. Some lysophospholipids, such as lysophosphatidylcholine, may act as lipid second messengers, transducing signals eliciting from membrane receptors. They may also potentiate immune responses and exhibit anti-tumor effects as bactericidal activities (for a review see Wang and Dennis, Biochim Biophys Acta; 1439:1-16 (1999)).

Lysophospholipase is a widely distributed enzyme which regulates the level of lysophospholipids and occurs in numerous isoforms. These isoforms vary in molecular mass, substrate metabolized, and optimum pH required for activity. Small isoforms, approximately 15-30 kDa, function as hydrolases; large isoforms, those exceeding 60 kDa function both as transacylases and hydrolases. Lysophospholipases are regulated by lipid factors such as acylcamitine, arachidonic acid and phosphatidic acid. The expression of IHLP is associated with proliferation and differentiation of cells of the immune system.

The role of lysophospholipases in human tissues has been investigated in various research studies. Selle, H. et al. (1993; Eur. J. Biochem. 212:411-16) characterized the role of lysophopholipase in the hydrolysis of lysophosphatidylcholine which causes lysis in erythrocyte membranes. Similarly, Endresen, M. J. et al. (1993) Scand. J. Clin. Invest. 53:733-9 reported that the increased hydrolysis of lysophosphatidylcholine by lysophopholipase in pre-eclamptic women causes release of free fatty acids into the sera. In renal studies, lysophopholipase was shown to protect NA+,K+-ATPase from the cytotoxic and cytolytic effects of cyclosporin A (Anderson, R. et al. (1994) Toxicol. Appl. Pharmacol. 125:176-83).

It is believed that the protein of SEQ ID NO:443 or part thereof plays a role in fatty acid metabolism, probably as a phospholipase. Preferred polypeptides of the invention are polypeptides comprising the amino acids of SEQ ID NO:443 from positions 54 to 58, and 52 to 66. Other preferred polypeptides of the invention are fragments of SEQ ID NO:443 having any of the biological activities described herein. The hydrolytic activity of the protein of the invention or part thereof may be assayed using any of the assays known to those skilled in the art including those described in Portilla et al., J Am Soc Nephrol; 9:1178-1186 (1998) and in the U.S. Pat. No. 6,004,792.

The invention relates to methods and compositions using the protein of the invention or part thereof to hydrolyze one or several substrates, alone or in combination with other substances. Such substrates are glycerophospholipids, preferably containing an acyl ester bond at the sn-2 position, more preferably lysophosphatidylcholine, lysophosphatidylinositol, lysophosphatidylserine, 1-oleoyl-2-acetyl-sn-glycero-3-phosphocholine, lecithin and lysolecithin. For example, the protein of the invention or part thereof is added to a sample containing the substrate(s) in conditions allowing hydrolysis, and allowed to catalyze the hydrolysis of the substrate(s). In a preferred embodiment, the hydrolysis is carried out using a standard assay such as those described by Portilla et al., supra and in the U.S. Pat. No. 6,004,792.

In a preferred embodiment, the protein of the invention or part thereof may be used to hydrolyze undesirable phospholipids, both in vitro or in vivo. In particular, the protein of the invention or part thereof may be used as a food additive to improve fat digestibility and to promote growth in animals using methods described in U.S. Pat. No. 6,017,530. In another preferred embodiment, the protein of the invention or part thereof may be used to improve the filtration of starch syrup by hydrolyzing the turbidity consisting mainly from phospholipids and resulting from the production of highly concentrated solutions of glucose isomers using methods described in U.S. Pat. No. 5,965,422. In addition, the protein of the invention or part thereof may be used in an enzymatic degumming process to free vegetable oils from phospholipids in order to allow their refining using methods described in U.S. Pat. No. 6,001,640. In another preferred embodiment, compositions comprising the protein of the present invention or part thereof are added to samples as a “cocktail” with other hydrolytic enzymes, such as other phospholipases for example to improve feed utilization in animals (see U.S. Pat. No. 6,017,530). The advantage of using a cocktail of hydrolytic enzymes is that one is able to hydrolyze a wide range of substrates without knowing the specificity of any of the enzymes. Using a cocktail of hydrolytic enzymes also protects a sample from a wide range of future unknown contaminants from a vast number of sources. For example, the protein of the invention or part thereof is added to samples where contaminating substrates is undesirable. Alternatively, the protein of the invention or part thereof may be bound to a chromatographic support, either alone or in combination with other hydrolytic enzymes, using techniques well known in the art, to form an affinity chromatography column. A sample containing the undesirable substrate is run through the column to remove the substrate. Immobilizing the protein of the invention or part thereof on a support is particularly advantageous for those embodiments in which the method is to be practiced on a commercial scale. This immobilization facilitates the removal of the enzyme from the batch of product and subsequent reuse of the enzyme. Immobilization of the protein of the invention or part thereof can be accomplished, for example, by replacing the transmembrane region by a cellulose-binding domain in the protein. One of skill in the art will understand that other methods of immobilization could also be used and are described in the available literature. Alternatively, the same methods may be used to identify new substrates.

In another embodiment, the protein of the invention or part thereof may be used to identify or quantify the amount of a given substrate in a biological sample. In a preferred embodiment, the protein of the invention or part thereof is used in assays and diagnostic kits for the identification and quantification of substrates in a biological sample.

In still another embodiment, the protein of the invention or part thereof may be used to diagnose, treat and/or prevent disorders where the presence of substrates is undesirable or deleterious. Such disorders include but are not limited to, cancer, neurodegenerative disorders such as Parkinson's and Alzheimer's diseases, diabetes. In a preferred embodiment, the protein of the invention or part thereof may be administered to a subject to reduce immune response. Although the inventors do not wish to be limited to a particular mechanism of action, it is thought that reduction would at least protect against lysophospholipid toxicity, deacylate platelet activating factor, and hydrolyze lytic lysophospholipids such as lysophosphatidylcholine which contribute to immune response, and in particular hypersensitivity reactions and immune cell mediated injuries. Such injuries include, but are not limited to, adult respiratory distress syndrome, allergies, asthma, arteriosclerosis, bronchitis, emphysema, hypereosinophilia, myocardial or pericardial inflammation, rheumatoid arthritis, complications of heart attack, stroke, cancer, hemodialysis, infections, and trauma.

In addition, the protein of the invention or part thereof may be used to identify inhibitors for mechanistic and clinical applications. Such inhibitors may then be used to identify or quantify the protein of the invention in a sample, and to diagnose, treat or prevent any of the disorders where the protein's activity is undesirable and/or deleterious including but not limited to inflammation, disorders associated with cell proliferation, immune and inflammatory disorders. Disorders associated with cell proliferation include adenocarcinoma, sarcoma, lymphoma, leukemia, melanoma, myeloma, teratocarcinoma, and in particular, cancers of the adrenal gland, bladder, bone, brain, breast, gastrointestinal tract, heart, kidney, liver, lung, ovary, pancreas, paraganglia, parathyroid, prostate, salivary glands, skin, spleen, testis, thyroid, and uterus. Immune and inflammatory disorders include Addison's disease, AIDS, adult respiratory distress syndrome, allergies, anemia, asthma, atherosclerosis, bronchitis, cholecystitus, Crohn's disease, ulcerative colitis, atopic dermatitis, dernatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polycystic kidney disease, polymyositis, rheumatoid arthritis, scleroderma, Sjogren's syndrome, autoimmune thyroiditis.

Moreover, antibodies to the protein of the invention or part thereof may be used for detection of the Golgi apparatus using any techniques known to those skilled in the art.

Protein of SEQ ID NO: 408 (Internal Designation 105-095-1-0-D10-FLC)

The protein of SEQ ID NO:408 encoded by the cDNA of SEQ ID NO:3 is homologous to the human parotid secretory protein HPSP (Genseq accession number W60682). PSPs are leucine-rich glycoproteins well conserved among the murine, rat, bovine and human species which belongs to the PSP multigenic family with gland specific members which common traits are early and abundant expression. Because it is extremely abundant in saliva, PSP has been proposed as a marker for tissue-specific protein production of salivary glands and appears coordinately regulated with salivary amylase. PSP is also expressed although to a lesser extent in murine lacrimal glands. Although its function remains unknown, it was shown to bind to bacteria in exocrine secretions and was proposed to have antibacterial activity (Robinson et al., Am J Physiol 272:G863-G871 (1997)). Antagonists of this protein may be used to treat cancer and autoimmune diseases particularly of secretory or gastrointestinal tissue.

It is believed that the protein of SEQ ID NO:408 or part thereof plays a role in the defense against pathogens, preferably pathogens present in the oral and gastrointestinal tracts. Preferred polypeptides of the invention are fragments of SEQ ID NO:408 having any of the biological activity described herein. The activity of the protein of the invention or part thereof on pathogens may be assessed using techniques well known to those skilled in the art including those described in Robinson et al, supra.

In one embodiment, the present invention relates to methods and compositions using the protein of the invention or part thereof to detect bacteria in biological fluids, foods, water, air, solutions and the like. For example, the protein of the invention or part thereof is added to a sample containing bacteria and allowed to bind to such bacteria using any method known to those skilled in the art including those described in Robinson et al, supra. Then, the protein may be detected using any method known to those skilled including using an antibody able to bind to the protein of the invention or part thereof, or using another polypeptide fused to the protein of the invention or part thereof that may be detected directly, such as the green fluorescent protein, or though binding to a specific antibody. In a preferred embodiment, the protein of the invention or part thereof is used in assays and diagnostic kits for the detection of exogenous pathogens in bodily fluids, tissue samples or cell cultures. In another preferred embodiment, the protein of the invention or part thereof may be used to decontaminate samples. For example, the protein of the invention or part thereof may be bound to a chromatographic support using techniques well known in the art, to form an affinity chromatography column. A sample containing the undesirable contaminant is ran through the column in order to be removed. Immobilizing the protein of the invention or part thereof on a support advantageous is particularly for those embodiments in which the method is to be practiced on a commercial scale. This immobilization facilitates the removal of the protein of the invention from the batch of product and its subsequent reuse. Immobilization of the protein of the invention or part thereof can be accomplished, for example, by inserting a cellulose-binding domain in the protein. One of skill in the art will understand that other methods of immobilization could also be used and are described in the available literature.

In another embodiment, the invention related to methods and compositions using the protein of the invention or part thereof to retard and/or inhibit the growth of pathogens, preferably bacteria, more preferably Listeria and Streptococci, and Actinobacilli, either in vitro or in vivo using any methods and techniques known to those skilled in the art, alone or in combination with other antimicrobial substances. For example, the protein of the invention or part thereof may be used to disinfect aqueous samples or materials, or as a food preservative. In a preferred embodiment, compositions comprising the protein of the present invention or part thereof are added to samples or materials as a “cocktail” with other antimicrobial substances to decontaminate samples. The advantage of using such a cocktail is that one is able to decontaminate samples without knowing the specificity of any of the antimicrobial substances. Using such a cocktail also protects a sample or material from a wide range of future unknown contaminants from a vast number of sources.

In another embodiment, the invention relates to methods and compositions using the protein of the invention or part thereof as a marker protein to selectively identify tissues, preferably salivary glands and lacrimal glands. For example, the protein of the invention or part may be used to synthesize specific antibodies using any techniques known to those skilled in the art including those described therein. Such tissue-specific antibodies may then be used to identify tissues of unknown origin, for example, forensic samples, differentiated tumor tissue that has metastasized to foreign bodily sites, or to differentiate different tissue types in a tissue cross-section using immunochemistry.

Protein of SEQ ID NO: 452 (Internal Designation 108-019-5-0-F5-FLC)

The protein of SEQ ID NO:452 encoded by the cDNA of SEQ ID NO:47 is homologous to human proteins either thought to be a transmembrane proteolipid protein down regulated upon cell differentiation induced by sodium butyrate (Genbank accession number AF057306) or described as the alternatively spliced chemokine-like factor 2 (Genbank accession number AF135380).

Proteolipids are a class of hydrophobic membrane proteins characterized in part by their capacity to assume conformations compatible with solubility in organic solvents and in water (Sapirstein V. S. et al (1983) Biochemistry 22:3330-3335). This amphipathic character of proteolipids explains their participation in transmembrane ion movement. Proteolipids are components of ion channel and transport systems, such as H⁺ channels (Arai H. et al (1987) J Biol Chem 262:11006-11011), Ca²⁺ channels (Eytan G. D. et al (1977) J Biol Chem 252: 3208-3213) and the C (membrane channel) subunit of the vacuolar H⁺-ATPase (Nelson H. et al (1990) J Biol Chem 265: 20390-20393).

The latter proteolipid, also known as ductin, is also associated with gap junctions. Gap junctions are the relatively large pores which allow free diffusion of ions across biological membranes (Finbow M. E. et al (1995) Bioessays 17:247-255). Altered gap-junction intercellular communication (GJIC) may play an essential role in cancer development. A lack of GJIC has been observed between transformed and neighboring normal cells (Trosko et al (1990) Radiation Res 123:241-251). A decrease in GJIC has also been observed within tumor cells (Krutovskikh et al (1991) Carcinogenesis 12:1701-1706).

Proteolipids are also involved in membrane vesicular trafficking. Due to their lipid-like properties, proteolipids destabilize lipid bilayers and promote membrane vesicle fusion. Such proteolipid-assisted events may include the fusions and fissions of the nuclear membrane, endoplasmic reticulum, Golgi apparatus, and various inclusion bodies (peroxisomes, lysosomes, etc).

Human T-lymphocyte maturation-associated protein (MAL), a 153 amino acid proteolipid, has been localized to the endoplasmic reticulum (ER) of T-lymphocytes, where it mediates the fusion of ER-derived vesicles and Golgi cisterna (Rancano C. et al (1994) J Biol Chem 269:8159-8164). A canine MAL homologue, VIP17, is involved in the sorting and targeting of proteins between the Golgi complex and the apical plasma membrane (Zacchetti D. et al (1995) FEBS Left 377:465-469). A rat MAL homologue, rMAL, is expressed in the myelinating cells of the nervous system including oligodendrocytes and Schwann cells. The rMAL protein serves as a gap junction component and plays a role in myelin compaction (Schaeren-Wiemers N. et al (1995) J. Neurosci 5753-5764).

Plasmolipin from rat is a proteolipid localized to plasma membranes in kidney and brain. It has 157 amino acids and, based on hydropathy plots and secondary structure predictions, consists of four alpha-helical transmembrane domains (I through IV) of 20-22 amino acids in length. Transmembrane domains III and IV contain hydroxyl groups which may contribute to an aqueous channel. Domains I through III are connected by short hydrophilic segments of 9-11 amino acids in length, and domains III and IV are connected by a longer hydrophilic segment of 20 amino acids. The small size and high hydrophobicity of plasmolipin constrains the distribution of its transmembrane regions such that the four transmembrane alpha-helices form an antiparallel bundle, and both the amino- and carboxy-termini face the cytoplasm. This structural model defines the growing class of small hydrophobic transport-related proteolipids containing four-helix transmembrane segments, such as the MAL homologues (Rancano et al, supra), and the vacuolar H⁺-ATPase C subunit (Nelson et al, supra).

In rat brain, plasmolipin is localized to myelinated nerve tracts, and its expression increases markedly with the onset of myelination (Fischer I. et al (1991) Neurochem Res 28:81-89). The distribution of plasmolipin within myelin appears to include regions active in membrane recycling. Endocytotic coated vesicles isolated from myelinated tracts are enriched with plasmolipin (Sapirstein V. S. (1994) J Neurosci Res 37:348-358). Incorporation of the purified rat plasmolipin protein into lipid bilayers induces voltage-dependent K⁺ channel formation, suggesting it may function in vivo as a pore or channel (Tosteson M. T. et al (1981) J Membr Biol 63:77-84). Channel formation involved the trimerization of the plasmolipin molecule. The oligomerization model of the plasmolipin molecule portrays transmembrane domains III and IV as walls of the channel, consistent with the presence of hydroxyl groups in these domains (Sapirstein et al (1983) supra). The putative role of rat plasmolipin in transport suggests its function may be in the fluid volume regulation of the myelin complex (Fischer et al (1994), supra).

Proteolipids are involved in membrane trafficking, gap junction formation, ion transport and cellular fluid volume regulation. The selective modulation of their expression may provide a means for the regulation of vesicle trafficking or the formation of channels or gap junctions in normal as well as acute and chronic disease situations.

It is believed that the protein of SEQ ID NO: 452 or part thereof plays a role membrane trafficking, gap junction formation, ion transport and/or cellular fluid volume regulation. Preferred polypeptides of the invention are fragments of SEQ ID NO:452 having any of the biological activity described herein. The ability of the protein of the invention or part thereof to form pore and/or to destabilize lipid bilayers may be assessed using techniques well known to those skilled in the art including those described in U.S. Pat. No. 5,843,714.

The invention relates to methods and compositions using the protein of the invention or part thereof to promote membrane vesicle fusion both in vitro and in vivo.

In an embodiment, the protein of the invention or part thereof is used to facilitate exocytosis. For example, the protein of the invention or part thereof may be used to increase the release of chemokines involved in cell migration, proteases which are active in inflammation or other similar activities involving endothelial cells, fibroblasts, lymphocytes, etc. Accordingly, the protein of the invention or part thereof may be used to diagnose, treat and/or prevent disorders associated with abnormal membrane trafficking including but not limited to viral or other infections, traumatic tissue damage, hereditary diseases such as arthritis or asthma, invasive leukemias and lymphomas.

In another embodiment, the protein of the invention or part thereof may be used to promote vesicle fusion for drug delivery. The protein of the invention or part thereof may be incorporated into liposomes or artificial vesicles with a drug of interest and then used to promote vesicle fusion for drug delivery. [0252] In another embodiment, antibodies to the protein of the invention or part thereof may be used for detection of membranes and/or gap junctions using any techniques known to those skilled in the art. In a preferred embodiment, the protein of the invention or part thereof may be used to diagnose disorders associated with altered intercellular communication, more preferably altered gap junction communication, including but not limited to cardiac arrhythmia.

Protein of SEQ ID NO:406 (Internal Designation 105-016-3-0-E3-FLC)

The 325-amino-acid-long protein of SEQ ID NO:406 encoded by the cDNA of SEQ ID NO:1 shows homology over the whole length of the 332-amino-acid-long murine neural proliferation differentiation and control 1 protein or NPDC-1 (Genbank accession number 35×67209) which is thought to play an important role in the control of neural cell proliferation and differentiation as well as in cell survival by interacting with cell cycle regulators such as E2F-1 (Galiana et al., Proc. Natl. Acad. Sci. USA 92:1560-1564 (1995); Dupont et al., J. Neurosci. Res. 51:257-267 (1998)).

It is believed that the protein of SEQ ID NO:406 or part thereof plays a role in cell proliferation and differentiation. Preferred polypeptides of the invention are polypeptides comprising the amino acids of SEQ ID NO:406 from positions 1 to 81, and 129 to 308. Other preferred polypeptides of the invention are fragments of SEQ ID NO:406 having any of the biological activity described herein. The activity of the protein of the invention or part thereof on cellular proliferation and differentiation may be assessed using techniques well known to those skilled in the art including those described in Galiana et al, supra.

In one embodiment, the invention related to methods and compositions using the protein of the invention or part thereof to inhibit cellular proliferation, preferably neuronal cell proliferation, using any methods and techniques known to those skilled in the art including those described in Galiana et al, supra.

In another embodiment, the protein of the invention or part thereof, may be used to diagnose, treat and/or prevent several disorders linked to cell proliferation and differentiation including, but not limited to cancer and neurodegenerative disorders such as Parkinson's or Alzheimer's diseases. For diagnostic purposes, the expression of the protein of the invention could be investigated using any of the Northern blotting, RT-PCR or immunoblotting methods described herein and compared to the expression in control individuals.

Protein of SEQ ID NO:407 (Internal Designation 105-031-3-O-D6-FLC)

The protein of SEQ ID NO:407 encoded by the cDNA of SEQ ID. NO:2 exhibits homology to a murine putative sialyltransferase protein (TREMBL accession number 088725). Although sialyltransferases have virtually no sequence homology, they display the features of type II transmembrane proteins with a short N-terminal cytoplasmic tail, a 16-20 amino acid signal-anchor domain, and an extended stem region which is followed by the large C-terminal catalytic domain (Weinstein, J. et al., J. Biol. Chem. 262, 17735-17743, 1987; Paulson, J. C. et al., J. Biol. Chem. 264,17615-17618, 1989).

The protein of SEQ ID NO:407 displays the two conserved motifs of the sialyltransferase protein family, namely the centrally located sialylmotifL (positions 73 to 120) thought to be involved in the recognition of the sugar nucleotide donor common to all sialyltransferases and the sialylmotifS (positions 211 to 233) thought to be the catalytic site and located in the C-terminus of the protein. Furthermore, the 302-amino-acid long protein of SEQ ID NO:407 has a size similar to the one of the members of the sialyltransferase family. In addition, the protein of the invention has a predicted transmembrane structure. Indeed, it contains 2 potential transmembrane segments (positions 7 to 27 and 206 to 226, underlined in FIG. 12) as predicted by the software TopPred II (Claros and von Heijne, CABIOS applic. Notes, 10:685-686 (1994)).

Sialyltransferases are glycosyl transferases found primarily in the Golgi apparatus and also in body fluids such as breast milk, colustrum and blood. They are responsible for the terminal sialylation of carbohydrate groups of glycoproteins, glycolipids and oligosaccharides widely distributed in animal tissues. Sialic acids play important roles in the biological functions of carbohydrate structures because of their terminal position. Sialyltransferases are indeed involved in a large variety of biological processes such as cell-cell communication, cell-matrix interactions, maintenance of serum glycoproteins in the circulation, and so on (Sjoberg et al., J. Biol. Chem. 271:7450-7459 (1996); Tsuji, J. Biochem. 120:1-13 (1996)). A variety of biological phenomena are associated with recognition of sialosides, including viral replication, escape of immune detection, and cell adhesion (Schauer, R. Trends Biochem. Sci. 1985, 10, 357-360; Biology of the Sialic Acids ed. A. Rosenberg, Plenum Press, New York, 1995). For example, suppressed antibody production was observed in alpha-2,6-sialyltransferase knockout mice (Muramatsu, J. Biochem. 127:171-6 (2000). In addition, carbohydrate structures have been shown to influence proteins' stability, rate of in vivo clearance from blood stream, rate of proteolysis, thermal stability and solubility. Changes in the oligosaccharide portion of cell surface carbohydrates have been noted in cells which have become cancerous.

It is believed that the protein of SEQ ID NO:407 or part thereof plays a role in the biosynthesis of sialyl-glycoconjugates, probably as a sialyltransferase. Thus, the protein of the invention or part thereof is thought to be involved in cell-cell communication, cell-matrix interactions, maintenance of serum glycoproteins in the circulation, viral replication, escape of immune detection, and cell adhesion. Preferred polypeptides of the invention are polypeptides comprising the amino acids of SEQ ID NO:407 from positions 73 to 120, and from position 211 to 233. Other preferred polypeptides of the invention are fragments of SEQ ID NO:407 having any of the biological activity described herein. The sialyltransferase activity of the protein of the invention or part thereof may be assayed using any other technique known to those skilled in the art including those described in Sadler et al., J. Biol. Chem., 254:4434-4443 (1979) or U.S. Pat. Nos. 5,827,714 and 6,017,743.

One object of the present invention are compositions and methods of targeting heterologous polypeptides to the Golgi apparatus by recombinantly or chemically fusing a fragment of the protein of the invention to an heterologous polypeptide. Preferred fragments are signal peptide, transmembrane domains, the proline-rich region comprised between positions 31 and 67, tyrosine containing regions and/or any other fragments of the protein of the invention, or part thereof, that may contain targeting signals for the Golgi apparatus including but not limited to proline-rich regions (Ugur and Jones, Mol Cell Biol 11:1432-32 (2000), Picetti and Borrelli, Exp Cell Res 255:258-69 (2000)), tyrosine-based Golgi targeting signal region (Zhan et al., Cancer Immunol Immunother 46:55-60 (1998); Watson and Pessin J. Biol. Chem. 275:1261-8 (2000); Ward and Moss, J. Virol. 74:3771-80 (2000) or any other region as defined in Munro, Trends Cell Biol. 8:11-15 (1998); Luetterforst et al., J. Cell. Biol. 145:1443-59 (1999); Essl et al., FEBS Lett. 453:169-73 (1999).

Sialylated compounds have considerable potential both as therapeutics and as reagents for clinical assays. However, synthesis of glycosylated compounds of potential commercial and/or therapeutic interest is difficult because of the very nature of the saccharide subunits. A multitude of positional isomers in which different substituent groups on the sugars become involved in bond formation, along with the potential formation of different anomeric forms, are possible. As a result of these problems, large scale chemical synthesis of most carbohydrates is not possible due to economic considerations arising from the poor yields of desired products. Enzymatic synthesis using glycosyl transferases such as sialyltransferases provides an alternative to chemical synthesis of carbohydrates. Enzymatic synthesis using glycosidases, glycosyl transferases, or combinations thereof, have been considered as a possible approach to the synthesis of carbohydrates. As a matter of fact, enzyme-mediated catalytic synthesis would offer dramatic advantages over the classical synthetic organic pathways, producing very high yields of carbohydrates economically, under mild conditions in aqueous solutions, and without generating notable amounts of undesired side products. To date, such enzymes are however difficult to isolate, especially from eukaryotic, e.g., mammalian sources, because these proteins are only found in low concentrations, and tend to be membrane-bound. In addition to being difficult to isolate, the acceptor (peptide) specificity of glycosyl transferases is poorly understood. Thus, there is a need for obtaining recombinant glycosyl transferase, including sialyltransferases, that could be produced in very large amounts.

Thus, the invention related to methods and compositions using the protein of the invention or part thereof to synthesize glycosylated compounds, either glycoproteins, glycoplipids, or oligosaccharides, more particularly sialylated compounds. If necessary, the protein of the invention or part thereof may be produced in a soluble form by removing its transmembrane domains and/or its Golgi retention signal using any of the methods skilled in the art including those described in U.S. Pat. No. 5,776,772. For example, the protein of the invention or part thereof is added to a sample containing sialic acid and a substrate compound in conditions allowing glycosylation, more particularly sialylation and allowed to catalyze the glycosylation of this compound. In a preferred embodiment, the enzymatic reaction carried out by the protein of the invention is part of a series of other chemical and/or enzymatic reactions aiming at the synthesis of complex glycosylated compounds, such as the ones described in U.S. Pat. Nos. 5,409,817 and 5,374,541. In another preferred embodiment where the method is to be practiced on a commercial scale, it may be advantageous to immobilize the glycosyl transferase on a support. This immobilization facilitates the removal of the enzyme from the batch of product and subsequent reuse of the enzyme. Immobilization of glycosyl transferases can be accomplished, for example, by removing from the transferase its membrane-binding domain, and attaching in its place a cellulose-binding domain. One of skill in the art will understand that other methods of immobilization could also be used and are described in the available literature.

In another embodiment, the present invention relates to processes and compositions for producing glycosylated compounds, preferably sialylated compounds, wherein a cell is genetically engineered to produce the protein of the invention or part thereof and used in combination with one or several other cells able to produce the donor substrate for the protein of the invention. Preferably, a bacteria is engineered to express the protein of the invention and used with recombinant bacteria expressing enzymes able to synthesize cytidine 5′-monophospho-N-acetyl neuramininc acid (CMP-NeuAc). The methods for performing the above bacterial coupling process and making the above compositions are carried using the methods known in the art and described in Endo et al., Appl. Microbiol. Biotechnol. 53:257-61, (2000).

Another embodiment of the present invention relates to a process and compositions for controlling the glycosylation of proteins in a cell wherein an insect, plant, or animal cell is genetically engineered to produce one or more enzymes which provide internal control of the cell's glycosylation mechanism. Preferably, the invention relates to a Chinese hamster ovary (CHO) cell line that is genetically engineered to produce a sialyltransferase of the present invention either alone or in combination with other sialyltransferases. This supplemental sialyltransferase modifies the CHO glycosylation machinery to produce glycoproteins having carbohydrate structures which more closely resemble naturally occurring human glycoproteins. The methods for performing the above process and making the above compositions are carried using the methods known in the art and described in U.S. Pat. No. 5,047,335.

The invention further relates to glycosylated compounds, preferably sialylated compounds, obtained using any of the processes described herein using the protein of the invention or part thereof. Such compounds may be used in the diagnosing, prevention and/or treating of disorders in which the recognition of such compounds is impaired or needs to be impaired. These disorders include, but are not limited to, cancer, cystic fibrosis, ulcer, inflammation and immune based disorders, including autoimmune disorders such as arthritis, fertility disorders, and hypothyroidism. These conditions include infectious diseases where active infection exists at any body site, such as meningitis and salpingitis; complications of infections including septic shock, disseminated intravascular coagulation, and/or adult respiratory distress syndrome; acute or chronic inflammation due to antigen, antibody and/or complement deposition; inflammatory conditions including arthritis, chalangitis, colitis, encephalitis, endocarditis, glomerulonephritis, hepatitis, myocarditis, pancreatitis, pericarditis, reperfusion injury and vasculitis. Immune-based diseases include but are not limited to conditions involving T-cells and/or macrophages such as acute and delayed hypersensitivity, graft rejection, and graft-versus-host disease; auto-immune diseases including Type I diabetes mellitus and multiple sclerosis. In a preferred embodiment, these glycosylated compounds or derivatives thereof may be used as pharmacological agents to trap pathogens or endogenous ligands thus reducing the binding of pathogens or endogenous ligands to the endogenous glycosylated compounds. For example, such compounds may be used to prevent and/or inhibit the adhesion of cancer cells to inner wall of blood vessel or aggregation between cancer cells and platelets, thus reducing cancer metastasis, to prevent and/or inhibit the adhesion of neutrophils to blood vessels endothelial cells, thus reducing inflammation. Other disorders include infections in which recognition of a glycosylated product is essential to the development of the infection. Such infections include, but are not limited to, those caused by Vibrio cholerae, Escherichia Coli, Salmonella, and the influenza virus. In a preferred embodiment, such compounds, preferably sialyl lactose, are used as neutralizers for enterotoxins from bacteria such as Vibrio cholerae, Escherichia Coli, and Salmonella as described in U.S. Pat. No. 5,330,975. In another preferred embodiment, such compounds, preferably galactose oligosaccharides, are used to diagnose, identify and inhibit the adherence of uropathogenic bacteria to red blood cells (U.S. Pat. No. 4,657,849). In another preferred embodiment, such compound, preferably oligosaccharides, are used as gram positive antibiotics and disinfectants (U.S. Pat. Nos. 4,851,338 and 4,665,060). In another embodiment, such compounds, preferably sialyl lactose, may be used for the treatment of arthritis and related autoimmune diseases (see, U.S. Pat. No. 5,164,374). In another embodiment, such compounds, preferably sialylalpha (2,3) galactosides, sialyl lactose and sialyl lactosamine, may be used for the treatment of ulcers. Phase I clinical trials have began for the use of the former compound in this capacity. (Balkonen, et al., FEMS Immunology and Medical Microbiology 7:29 (1993) and BioWorld Today, p. 5, Apr. 4, 1995). In addition, such compounds, preferably sialyl lactose, may be used as food supplement, for instance in baby formula.

In addition, the protein of the invention or part thereof may be used in the development of inhibitors of glycosyl transferase, more particularly inhibitors of sialyltransferases and sialidases, for mechanistic and clinical applications (Taylor, G. Curr. Opin. Struc. Biol. 1996, 6, 830-837; Colman, P. M., Pure Appl. Chem. 1995, 67, 1683-1688; Bamford, M. J. J Enz. Inhib. 1995, 10, 1-16; Khan, S. H. & Matta, K. L. In Glycoconjugates, Composition, Structure, and Function. pp361-378. ed., Allen, H. J. & Kisailus, E. C. Marcel Dekker, Inc. New York, 1992, Thome-Tjomsland et al., Transplantation 69:806-8, (2000); Basset et al, Scand. J. Immunol. 51:307-11 (2000)).

The invention further relates to methods and compositions using the protein of the invention or part thereof to diagnose, prevent and/or treat several disorders in which recognition of glycosylated compounds, preferably of sialylated compounds, is impaired or needs to be impaired. For diagnostic purposes, the expression of the protein of the invention could be investigated using any of the Northern blotting, RT-PCR or immunoblotting methods described herein and compared to the expression in control individuals. For prevention and/or treatment purposes, inhibiting the endogenous expression of the protein of the invention using any of the antisense or triple helix methods described herein may be used to reduce the production of glycosylated compounds detrimental to the organism in any of the disorders described above.

Protein of SEQ ID NOs:436 (Internal Designation 108-008-5-0-C5-FL)

The protein of SEQ ID NO:436 encoded by the cDNA of SEQ ID NO:31 exhibits homology over the whole length to the murine recombination activating gene 1 inducing protein found in stromal cell (Genbank accession number X96618). The amino acid residues are identical except for the positions 6, 7, 10-13, 17, 25, 34-35, 42, 51, 56, 62, 68, 71, 74, 78, 91, 93, 95-96, 106, 121-122, 151-152, 159, 162-163, 170-171, 176-177, 188, 190, 192, 196, 199, 202-203, 206, 210, 215 and 217 of the 221 amino acid long matched protein. This protein with 4 potential transmembrane segments facilitates gene activation of RAG-1 which is involved in the recombination of V(D)J segments in T cells (Tagoh et al., Biochem Biophysic Res Comm 221:744-749 (1996); Muraguchi et al, Leuk Lymphoma, 30:73-85 (1998)).

It is believed that the protein of SEQ ID NO:436 may play a role in lymphocyte repertoire formation. Preferred polypeptides of the invention are fragments of SEQ ID NO:406 having any of the biological activity described herein. The activity of the protein of the invention or part thereof on the induction of RAG expression may be assessed using techniques well known to those skilled in the art including those described in Tagoh et al, supra.

In an embodiment, antibodies to the protein of the invention or part thereof may be used as markers for haematopoietic precursors, preferably precursors for B and T cells.

In another embodiment, the protein of the invention or part thereof, may be used to diagnose, treat and/or prevent immunological disorders including, but not limited to Ommen'syndrome, acute and delayed hypersensitivity, graft rejection, and graft-versus-host disease; auto-immune diseases including Type I diabetes mellitus and multiple sclerosis, lymphoid neoplasia including non Hodgkins' lymphoma, ALL and CLL. For diagnostic purposes, the expression of the protein of the invention could be investigated using any of the Northern blotting, RT-PCR or immunoblotting methods described herein and compared to the expression in control individuals. In another embodiment, the protein of the invention or part thereof may also be used to modulate the immune response to pathogens.

Protein of SEQ ID NO:419 (Internal Designation 116-073-4-0-C8-FLC)

The protein of SEQ ID NO:419 encoded by the cDNA of SEQ ID NO:14 shows homology over the whole length of the widely conserved family of lysozyme C precursors (fish, bird, and mammals). In particular, the protein of the invention displays 17 out of the 20 amino acids conserved among all known lysozyme C proteins at positions 115, 117, 123, 137, 141, 144, 146, 150, 151, 162, 166, 180, 181, 194, 197, 201 and 213 (Prager and Jollès, Lysozymes: model enzymes in biochemistry and biology, ed. Jollès, 9-321 (1996)). In addition, this protein displays the characteristic signature of the family 22 of glysosyl hydrolases (PROSITE signature from positions 162 to 185, eMotif signatures from positions 183 to 202 and from positions 111 to 120), which contain the evolutionary related alpha-lactalbumin, the regulatory subunit of lactose synthetase, and the bacteriolytic defensive enzymes lysozyme C (Qasba and Kumar, Crit. Rev. Biochem. Mol. Biol. 32:255-306 (1997)). Furthermore, the cDNA of SEQ ID NO:14 seems to be preferentially expressed in testis (Table VII) and in germ cells tumors (Table VIII).

Lysozyme, an ubiquitous protein secreted in most body secretions, is defined as 1,4-beta-N-acetylmuramidases which cleave the glycoside bond between the C-1 of N-acetyl-muramic acid and the C-4 of N-acetylglucosamine in the peptidoglycan of bacteria. It has various therapeutic properties, such as antiviral, antibacterial, anti-inflammatory and antihistaminic effects. The activity of the lysozyme as an anti-bacterial agent appears to be based on both its direct bacteriolytic activity and also on stimulatory effects in connection with phagocytosis of polymorphonuclear leucocytes and macrophages (Biggar and Sturgess, J. M. Infect Immunol. 16: 974-982 (1977); Thacore and Willet, Am. Rev. Resp. Dis. 93: 786-790 (1966); Klockars and Roberts, P. Acta Haematol 55: 289-292 (1976)). Lysozyme has proven to be not only a selective factor but also an effective factor against microorganisms of the mouth (Iacono et al, J. J. Infect. Immunol. 29: 623-632 (1980)). Lysozyme can also kill pathogens by acting synergistically with other proteins such as complement or antibody to lyse pathogenic cells. Lysozyme, also inhibits chemotaxis of polymorphonuclear leukocytes and limits the production of oxygen free radicals following an infection. This limits the degree of inflammation, while at the same time enhances phagocytosis by these cells. Other postulated functions of lysozyme include immune stimulation (Jolles, P. Biomedicine 25: 275-276 (1976) Ossermann, E. F. Adv. Pathobiol 4: 98-102 (1976)) and immunological and non-immunological monitoring of host membranes for any neoplastic transformation (Jolles, P. Biomedicine 25: 275-276 (1976); Ossermann, E. F. Adv. Pathobiol 4: 98-102 (1976)). Lysozyme may thus be used in a wide spectrum of applications (see U.S. Pat. No. 5,618,712). Determination of the lysozymes from serum and/or urine is used to diagnose various diseases or as an indicator for their development. In acute lymphoblastic leukaemia the lysozyme serum level is significantly reduced, whereas in chronic myelotic leukaemia and in acute monoblastic and myelomonocytic leukaemia the lysozyme concentration in the serum is greatly increased. The therapeutically effective use of lysozyme is possible in the treatment of various bacterial and virus infections (Zona, Herpes zoster), in colitis, various types of pain, in allergies, inflammation and in pediatrics (the conversion of cows milk into a form suitable for infants by the addition of lysozyme).

It is believed that the protein of SEQ ID NO:419 or part thereof plays a role in glycoprotein and/or peptidoglycan metabolism, probably as a glycosyl hydrolase of family 22. Thus, the protein of the invention or part thereof may be involved in immune and inflammatory responses and may have antiviral, antibacterial, anti-inflammatory and/or anti-histaminic functions. Preferred polypeptides of the invention are polypeptides comprising the amino acids of SEQ ID NO:419 from positions 70 to 215, 111 to 120, 183 to 202, and 162 to 185. Other preferred polypeptides of the invention are fragments of SEQ ID NO:419 having any of the biological activities described herein. The glycolytic activity of the protein of the invention or part thereof may be assayed using any of the assays known to those skilled in the art including those described in Gold and Schweiger, M. Methods in Enzymology, Vol. XX, Part C pp. 537-542, Ed. Moldave, Academic Press,New York and London, 1971 and in the U.S. Pat. No. 4,255,517.

The invention relates to methods and compositions using the protein of the invention or part thereof to hydrolyze one or several substrates, alone or in combination with other substances, preferably antiviral, antifungal and/or antibacterial substances including but not limited to immunoglobulins, lactoferrin, betalysin, fibronectin, and complement components. Such substrates are glycosylated compounds, preferably containing beta-1-4-glycoside bonds, more preferably containing beta-1-4-glycoside bonds between n-acetylomuraminic acid and n-acetyloglucosamine. For example, the protein of the invention or part thereof is added to a sample containing the substrate(s) in conditions allowing hydrolysis, and allowed to catalyze the hydrolysis of the substrate(s). In a preferred embodiment, the hydrolysis is carried out using a standard assay such as those described by Gold and Schweiger, supra, and U.S. Pat. Nos. 5,871,477 and 4,255,517. In a preferred embodiment, the protein of the invention or part thereof may be used to lyze recombinant bacteria in order to recover the recombinant DNA, the recombinant protein of interest, or both using, for example, any of the assays described in Sambrook, et al., Molecular Cloning: A Laboratory Manual, Second Edition, Cold Spring Harbor Laboratory Press (1989).

In an embodiment, the protein of the invention or part thereof is used to hydrolyze contaminating substrates in an aqueous sample or onto a material, preferably glassware and plasticware. In particular, the protein of the invention or part thereof may be used as a disinfectant in dental rinse, in protection of aqueous systems or in preparing material for medical applications using any of the methods and compositions described in U.S. Pat. Nos. 5,069,717, 4,355,022 and 5,001,062. In a preferred embodiment, the protein of the invention is used as a host resistance factor in infants' formulas to convert cow's milk into a form more suitable for infants as described in U.S. Pat. No. 6,020,015. In another preferred embodiment, the protein of the invention or part thereof may be used as a food preservative (see Hayashi et al., Agric. Biol. Chem. (European Edition of Japanese Journal of Agriculture, Biochemistry and Chemistry), Vol. 53, pp. 3173-3177, 1989). In addition, the protein of the invention or part thereof may be used to clarify xanthan gum fermented broth for applications in food and in cosmetic industries using the method described in U.S. Pat. No. 5,994,107. In another preferred embodiment, compositions comprising the protein of the present invention or part thereof are added to samples or materials as a “cocktail” with other antimicrobial substances, preferably antibiotics or hydrolytic enzymes such as those described in U.S. Pat. Nos. 5,458,876 and 5,041,326 to decontaminate the samples. For example, the protein of the invention or part thereof may be used in place or in combination with antibiotics in cell cultures. The advantage of using a cocktail of hydrolytic enzymes is that one is able to hydrolyze a wide range of substrates without knowing the specificity of any of the enzymes. Using a cocktail of hydrolytic enzymes also protects a sample or material from a wide range of future unknown contaminants from a vast number of sources. For example, the protein of the invention or part thereof is added to samples where contaminating substrates is undesirable. Alternatively, the protein of the invention or part thereof may be bound to a chromatographic support, either alone or in combination with other hydrolytic enzymes, using techniques well known in the art, to form an affinity chromatography column. A sample containing the undesirable substrate is run through the column to remove the substrate. Immobilizing the protein of the invention or part thereof on a support advantageous is particularly for those embodiments in which the method is to be practiced on a commercial scale. This immobilization facilitates the removal of the enzyme from the batch of product and subsequent reuse of the enzyme. Immobilization of the protein of the invention or part thereof can be accomplished, for example, by inserting a cellulose-binding domain in the protein. One of skill in the art will understand that other methods of immobilization could also be used and are described in the available literature. Alternatively, the same methods may be used to identify new substrates.

In addition, the protein of the invention or part thereof may be useful to identify or quantify the amount of a given substrate in biological fluids, foods, water, air, solutions and the like. In a preferred embodiment, the protein of the invention or part thereof is used in assays and diagnostic kits for the identification and quantification of exogenous substrates in bodily fluids including blood, lymph, saliva or other tissue samples, in addition to bacterial, fungal, plant, yeast, viral or mammalian cell cultures. In a preferred embodiment, the protein of the invention or part thereof is used to detect, identify, and or quantify eubacteria using reagents and assays described in U.S. Pat. No. 5,935,804. Briefly, the protein of the invention of part thereof is catalytically inactived, i.e. capable of binding but not cleaving a peptidoglycan comprising NAc-muramic acid in the eubacteria, using any of the methods known to those skilled in the art including those which produce a mutant enzyme, a recombinant-enzyme, or a chemically inactivated enzyme. The catalytically inactive protein of the invention is then incubated with an aliquot of a biological sample under conditions suitable for binding of the inactive enzyme to the peptidoglycan substrate. Then, the bound enzyme is detected to assess the presence or amount of the eubacteria in the biological sample.

In another embodiment, the nucleic acid of the invention or part thereof may be used to increase disease resistance of plants to bacterial, fungal and/or viral infections. A polynucleotide containing the nucleic acid of the invention or part thereof is introduced into the plant genome in conditions allowing correct expression of the transgenic protein using any methods known to those skilled in the art including those disclosed in U.S. Pat. Nos. 5,349,122 and 5,850,025.

In another preferred embodiment, the protein of the invention or part thereof may be useful to treat and/or prevent bacterial, fungal and viral infections in humans or in animals caused by various agents including but not limited to Streptococcus, Veillonella alcalescens, Actinomyces, Herpes simplex, Candida albicans, Micrococcus lysodeikticus and HIV by hydrolyzing the glycosylated compounds contained in such micro-organisms. In still a preferred embodiment, the protein of the invention or part thereof is used to prevent and/or treat bacterial, fungal and viral infections in immunocompromised individuals who lack fully functional immune systems, such as neonates or geriatric patients or HIV-infected individuals, or who suffer from a disease affecting the respiratory tract such as cystic fibrosis or the gastrointestinal tract such as ulcerative colitis or sprue.

In still another embodiment, the protein of the invention or part thereof may be used as a growth factor for in vitro cell culture, preferably for T cells and T cell lines, as described in U.S. Pat. No. 5,468,635.

In addition, the protein of the invention or part thereof may be used to identify inhibitors for mechanistic and clinical applications. Such inhibitors may then be used to identify or quantify the protein of the invention in a sample, and to diagnose, treat or prevent any of the disorders where the protein's hydrolytic, immunostimulatory and/or inflammatory activities is/are undesirable and/or deleterious including but not limited to amyloidosis, colitis, lysosomal diseases, inflammatory and immune disorders including allergies and leukaemia. The protein of the invention may also be used to monitor host cell membranes for neoplastic transformation.

In still another embodiment, the invention relates to methods and compositions using the protein of the invention or part thereof as a marker protein to selectively identify tissues, preferably germ cells, more preferably testis. For example, the protein of the invention or part may be used to synthesize specific antibodies using any techniques known to those skilled in the art including those described therein. Such tissue-specific antibodies may then be used to identify tissues of unknown origin, for example, forensic samples, differentiated tumor tissue that has metastasized to foreign bodily sites, or to differentiate different tissue types in a tissue cross-section using immunochemistry.

Protein of SEQ ID NO:433 (Internal Designation 108-005-5-0-F9-FL)

The protein of SEQ ID NO:433 encoded by the extended cDNA SEQ ID NO:28 shows homology with the Drosophila rhythmically expressed gene 2 protein (Genbank accession number U65492) and with a 2-haloalkanoic acid dehalogenase (Embl accession number AJ248288). In addition, the protein of SEQ ID NO:433 exhibits the pfam signature for haloacid dehalogenase-like hydrolase family from positions 7 to 214.

Expression of the mRNA coding for Dreg-2 is dependent on the interplay between light-dark cycle, feeding conditions and expression of the per gene which is essential to the function of the endogenous circadian pacemaker (Van Gelder et al., Curr. Biol., 5:1424-1436 (1995)). The matched pfam hydrolase family include proteins which are structurally different from the alpha/beta hydrolase family and which include L-2-haloacid dehalogenase, epoxide hydrolases and phosphatases (see Pfam accession number PF00702).

Organohalogen compounds are by-products in several industrial processes that are considered as environmental pollutants. The detection of trihalomethanes, halogenated acetic acids, halogenated acetonitriles and halogenated ketones in city water has become a great problem because of their liver toxicity and mutagenicity. Halogenated organic acids, for example halogenated acetic acids such as chloroacetic acid, dichloroacetic acid, trichloroacetic acid and bromoacetic acid have been designated as environment surveillance items in Japan since 1993. Increasing environmental concerns have created a demand for products that are free from such environmentally unsound byproducts. Physical methods of decontaminating aqueous reaction products containing unwanted nitrogen-free organohalogen byproducts are known, such as solvent extraction with a water-immiscible solvent, or adsorption on a solid adsorbent, such as charcoal. However, such known methods can result in depletion of the reaction product, as well as requiring costly measures to recover and purify the solvent or adsorbent. Furthermore, such methods still leave the problem of how to ultimately dispose of the contaminants such as undesired halogenated oxyalkylene compounds. As one of the countermeasures, for example, biodegradation treatment such as a bioreactor is very useful because treatment can be conducted under mild conditions and is relatively low in cost. The conversion of nitrogen-free organohalogen compounds with microorganisms containing a dehalogenase is also known. For example, C. E. Castro, et al. (“Biological Cleavage of Carbon-Halogen Bonds Metabolism of 3-Bromopropanol by Pseudomonas sp.”, Biochimica et Biophysica Acta, 100, 384-392, 1965) describe the use of Pseudomonas sp. isolated from soil that metabolizes 3-bromopropanol in sequence to 3-bromopropionic acid, 3-hydroxypropionic acid and CO₂. Various U.S. Patents also describe the use of microorganisms for dehalogenating halohydrins, e.g. U.S. Pat. Nos. 4,452,894; 4,477,570; and 4,493,895.

Epoxide hydrolases are a family of enzymes which hydrolyze a variety of exogenous and endogenous epoxides to their corresponding diols. Compounds containing the epoxide functionality have become common environmental contaminants because of their wide use as pesticides, sterilants, and industrial precursors. Such compounds also occur as products, by-products, or intermediates in normal metabolism and as the result of spontaneous oxidation of membrane lipids (i.e. see, Brash, et al., Proc. Natl. Acad. Sci., 85:3382-3386 (1988), and Sevanian, A., et al., Molecular Basis of Environmental Toxicology (Bhatnager, R. S., ed.) pp. 213-228, Ann Algor Science, Michigan (1980)). As three-membered cyclic ethers, epoxides are often very reactive and have been found to be cytotoxic, mutagenic and carcinogenic (i.e. see Sugiyama, S., et al., Life Sci. 40:225-231 (1987)). Cleavage of the ether bond in the presence of electrophiles often results in adduct formation. As a result, epoxides have been implicated as the proximate toxin or mutagen for a large number of xenobiotics. Reactions of detoxification using epoxide hydrolases typically decrease the hydrophobicity of a compound, resulting in a more polar and thereby excretable substance. In addition to degradation of potential toxic epoxides, dehalogenases are believed to play a role in the formation or degradation of endogenous chemical mediators (see U.S. Pat. No. 5,445,956).

Many eukaryotic cell functions, including signal transduction, cell adhesion, gene transcription, RNA splicing, apoptosis and cell proliferation, are controlled by protein phosphorylation which is in turn regulated by the dynamic relationship between kinases and phosphatases (see U.S. Pat. No. 6,040,323 for a short review). Thus, the protein phosphatases represent unique and attractive targets for small-molecule inhibition and pharmacological intervention. In addition, hydrolytic enzymes such as alkaline phosphatase are frequently used as markers or labels in enzyme-linked assays for biological molecules and other analytes of interest such as drugs, hormones, steroids and cancer markers.

It is believed that the protein of SEQ ID NO:433 or part thereof is an hydrolase, preferably a phosphatase, an ether hydrolase or an hydrolase acting on C-halide bonds. Preferred polypeptides of the invention are polypeptides comprising the amino acids of SEQ ID NO:433 from positions 7 to 214. Other preferred polypeptides of the invention are fragments of SEQ ID NO:433 having any of the biological activity described herein. The hydrolytic activity of the protein of the invention or part thereof may be assayed using any of the assays known to those skilled in the art including those described in U.S. Pat. Nos. 5,445,942; 5,445,956, 6,017,746 and 5,871,616.

The invention relates to methods and compositions using the protein of the invention or part thereof to hydrolyze one or several substrates, alone or in combination with other substances, either in vitro or in vivo. Such substrates are compounds containing phosphoric ester bonds, ether bonds or C-halide bonds. For example, the protein of the invention or part thereof is added to a sample containing the substrate(s) in conditions allowing hydrolysis, and allowed to catalyze the hydrolysis of the substrate(s). In a preferred embodiment, the hydrolysis is carried out using any assay known to those skilled in the art including those described by the U.S. Pat. Nos. 5,445,942; 5,445,956, 6,017,746 and 5,871,616. In a preferred embodiment, the protein of the invention is used to hydrolyze environmental pollutants, preferably organohalogen compounds and epoxide, such as those cited below using any of the methods and techniques described in U.S. Pat. Nos. 6,017,746 and 5,871,616.

The invention relates to methods and compositions using the protein of the invention or part thereof to diagnose, prevent and/or treat several disorders of the circadian rhythm including, but not limited to, insomnia, depression, stress, night work or jet lag. For diagnostic purposes, the overexpression or the improper temporal expression of the protein of the invention could be investigated using any of the Northern blotting, RT-PCR or immunoblotting methods described herein and compared to the expression in control individuals.

Protein of SEQ ID NO:427 (Internal Designation 122-005-2-0-F11-FLC)

The protein of SEQ ID NO:427 encoded by the cDNA of SEQ ID NO:22 exhibits homology with a fragment of NADH-cytochrome b5 reductases of rat, bovine and human species which are part of the mitochondrial electron transport chain (Genbank accession numbers J03867, M83104 and Y09501, respectively). This homology includes the flavin-adenine dinucleotide (FAD)-binding domain of this family of proteins from positions 118 to 148, and 157 to 192. Moreover, the 3 lysine residues shown to be implicated in the formation of charged ion pairs with carboxyl groups on NADH-cytochrome b5 reductase during interactions between the active sites of cytochrome b5 and NADH-cytochrome b5 reductase are conserved in the protein of the invention at positions 46, 112 and 150 (Strittmatter, P. et al. (1990) J. Biol. Chem. 265: 21709-13). In addition, the protein of the invention exhibits emotif signatures for cytochrome b5 reductase from positions 123 to 138, 163 to 180, and 256 to 265, emotif signatures for eukaryotic molybdopterin oxidoreductases from positions 256 to 266 and 256 to 268, and emotif signatures for flavoprotein pyridine nucleotide cytochrome reductases from positions 110 to 120, 163 to 177, and 163 to 179.

NADH-cytochrome b5 reductase proteins belong to a flavoenzyme family sharing common structural features and whose members (ferrodoxin-NADP+reductase, NADPH-cytochrome P450 reductase, NADPH-sulfite reductase, NADH-cytochrome b5 reductase and NADH-nitrate reductase) are involved in photosynthesis, in the assimilation of nitrogen and sulfur, in fatty-acid oxidation, in the reduction of methemoglobin and in the metabolism of many pesticides, drugs and carcinogens (Karplus et al., Science, 251:60-6 (1991)). In addition, cytochrome b5 reductase is thought to play a role in the prevention of apoptosis following oxidative stress (see review by Villalba et al., Mol Aspects Med 18 Suppl1:S7-13 (1997)).

It is believed that the protein of SEQ ID NO:427 may be an oxidoreductase. Thus it may play a role in electron transport and general aerobic metabolism and may be associated with mitochondrial membranes. In addition, the protein of the invention may be able to use FAD and/or molybdopterin as cofactors. It may be involved in photosynthesis, in the assimilation of nitrogen and sulfur, in fatty-acid oxidation, in the reduction of methemoglobin and in the metabolism of many pesticides, drugs and carcinogens. Preferred polypeptides of the SEQ ID NO:427 from positions 118 to 148, 157 to 192, 123 to 138, 163 to 180, 256 to 265, 256 to 266, 256 to 268, 110 to 120, 163 to 177, and 163 to 179. Other preferred polypeptides of the invention are fragments of SEQ ID NO:427 having any of the biological activity described herein. The oxidoreductase activity of the protein of the invention may be assayed using any technique known to those skilled in the art. The ability to bind a cofactor may also be assayed using any techniques well known to those skilled in the art including, for example, the assay for binding NAD described in U.S. Pat. No. 5,986,172.

An object of the present invention are compositions and methods of targeting heterologous compounds, either polypeptides or polynucleotides to mitochondria by recombinantly or chemically fusing a fragment of the protein of the invention to an heterologous polypeptide or polynucleotide. Preferred fragments are signal peptide, amphiphilic alpha helices and/or any other fragments of the protein of the invention, or part thereof, that may contain targeting signals for mitochondria including but not limited to matrix targeting signals as defined in Herrman and Neupert, Curr. Opinion Microbiol. 3:210-4 (2000); Bhagwat et al. J. Biol. Chem. 274:24014-22 (1999), Murphy Trends Biotechnol. 15:326-30 (1997); Glaser et al. Plant Mol Biol 38:311-38 (1998); Ciminale et al. Oncogene 18:4505-14 (1999). Such heterologous compounds may be used to modulate mitochondria's activities. For example, they may be used to induce and/or prevent mitochondrial-induced apoptosis or necrosis. In addition, heterologous polynucleotides may be used for mitochondrial gene therapy to replace a defective mitochondrial gene and/or to inhibit the deleterious expression of a mitochondrial gene.

In another embodiment, the protein of the invention or part thereof is used to prevent cells to undergo apoptosis. In a preferred embodiment, the apoptosis active polypeptide is added to an in vitro culture of mammalian cells in an amount effective to reduce apoptosis. Furthermore, the protein of the invention or part thereof may be useful in the diagnosis, the treatment and/or the prevention of disorders in which apoptosis is deleterious, including but not limited to immune deficiency syndromes (including AIDS), type I diabetes, pathogenic infections, cardiovascular and neurological injury, alopecia, aging, degenerative diseases such as Alzheimer's Disease, Parkinson's Disease, Huntington's disease, dystonia, Leber's hereditary optic neuropathy, schizophrenia, and myodegenerative disorders such as “mitochondrial encephalopathy, lactic acidosis, and stroke” (MELAS), and “myoclonic epilepsy ragged red fiber syndrome” (MERRF).

The invention further relates to methods and compositions using the protein of the invention or part thereof to diagnose, prevent and/or treat several disorders in which energy metabolism is impaired, or needs to be impaired, including but not limited to mitochondriocytopathies, necrosis, aging, neurodegenerative diseases, myopathies, methemoglobinemia, hyperlipidemia, obesity, cardiovascular disorders and cancer. For diagnostic purposes, the expression of the protein of the invention could be investigated using any of the Northern blotting, RT-PCR or immunoblotting methods described herein and compared to the expression in control individuals. For prevention and/or treatment purposes, the protein of the invention may be used to enhance electron transport and increase energy delivery using any of the gene therapy methods described herein.

Protein of SEQ ID NO:445 (Internal Designation 108-014-5-0-C7-FLC)

The protein of SEQ ID NO:445 encoded by the extended cDNA SEQ ID NO:40 shows homology with a fragment of a cold active protease isolated from Flavobacterium balustinum (Genseq accession number W23332) which degrades casein, gelatin, haemoglobin and albumin. This protease is able to degrade proteins at low temperatures or in presence of organic solvents that are volatile at normal processing temperature.

These data suggest that the protein of SEQ ID NO:445 or part thereof is an hydrolase, preferably a protease. Preferred polypeptides of the invention are polypeptides comprising the amino acids of SEQ ID NO:445 from positions 1 to 44. Other preferred polypeptides of the invention are fragments of SEQ ID NO:445 having any of the biological activity described herein. The hydrolytic activity of the protein of the invention or part thereof may be assayed using any of the assays known to those skilled in the art including those described in U.S. Pat. No. 6,069,229.

The invention relates to methods and compositions using the protein of the invention or part thereof to hydrolyze one or several substrates, alone or in combination with other substances. Such substrates are compounds containing peptide bonds. For example, the protein of the invention or part thereof is added to a sample containing the substrate(s) in conditions allowing hydrolysis, and allowed to catalyze the hydrolysis of the substrate(s). In a preferred embodiment, the hydrolysis is carried out using a standard assay such as those described by the U.S. Pat. No. 6,069,229.

In a preferred embodiment, compositions comprising the protein of the present invention or part thereof are added to samples as a “cocktail” with other hydrolytic enzymes such as those described in U.S. Pat. Nos. 5,458,876 and 5,041,326. The advantage of using a cocktail of hydrolytic enzymes is that one is able to hydrolyze a wide range of substrates without knowing the specificity of any of the enzymes. Using a cocktail of hydrolytic enzymes also protects a sample from a wide range of future unknown protein contaminants from a vast number of sources. For example, the protein of the invention or part thereof is added to samples where contaminating substrates is undesirable. For example, the protein of the invention or part thereof may be used to remove protein contaminants from nucleic acid preparations, to remove cells from cultureware. Alternatively, the protein of the invention or part thereof may be bound to a chromatographic support, either alone or in combination with other hydrolytic enzymes, using techniques well known in the art, to form an affinity chromatography column. A sample containing the undesirable substrate is run through the column to remove the substrate. Immobilizing the protein of the invention or part thereof on a support is particularly advantageous for those embodiments in which the method is to be practiced on a commercial scale. This immobilization facilitates the removal of the enzyme from the batch of product and subsequent reuse of the enzyme. Immobilization of the protein of the invention or part thereof can be accomplished, for example, by inserting a cellulose-binding domain in the protein. One of skill in the art will understand that other methods of immobilization could also be used and are described in the available literature. Alternatively, the same methods may be used to identify new substrates.

The protease of the invention may be used in many industrial processes, including in detergents and cleaning products, e.g., to degrade protein materials such as blood and stains or to clean contact lenses, in leather production, e.g., to remove hair, in baking, e.g., to break down glutens, in flavorings, e.g., soy sauce, in meat tenderizing, e.g., to break down collagen, in gelatin or food supplement production, in the textile industry, in waste treatment, and in the photographic industry. See, e.g., Gusek (1991) Inform 1:14-18; Zamost, et al. (1996) J. Industrial Microbiol. 8:71-82; James and Simpson (1996) CRC Critical Reviews in Food Science and Nutrition 36:437-463; Teichgraeber, et al. (1993) Trends in Food Science and Technology 4:145-149; Tjwan, et al. (1993) J. Dairy Research 60:269-286; Haard (1992) J. Aquatic Food Product Technology 1:17-35; van Dijk (1995) Laundry and Cleaning News 21:32-33; Nolte, et al. (1996) J. Textile Institute 87:212-226; Chikkodi, et al. (1995) Textile Res. J. 65:564-569; and Shih (1993) Poultry Science 72:1617-1620; PCT publication WO9925 848-Al.

In addition, the protein of the invention or part thereof may be used to identify inhibitors for mechanistic and clinical applications. Such inhibitors may then be used to identify or quantify the protein of the invention in a sample, and to diagnose, treat or prevent any of the disorders where the protein's hydrolytic activity is undesirable and/or deleterious such as disorders characterized by tissue degradation including but not limited to amyloidosis, colitis, lysosomal diseases, arthritis, muscular dystrophy, inflammation, tumor invasion, glomerulonephritis, parasite-borne infections, Alzheimer's disease, periodontal disease, and cancer metastasis.

Protein of SEQ ID NO:413 (Internal Designation 116-047-3-0-B1-FLC)

The protein of SEQ ID NO:413 encoded by the extended cDNA SEQ ID NO:8 shows homology with the ribokinase rbsk (Embl accession number Q9X4M5) which is part of the pfkb family of kinases. In addition, the protein of the invention exhibits the pfam signature for this family of carbohydrate and purine kinases from positions 28 to 94.

The pfkb family of carbohydrate kinase is composed of evolutionary related kinases including fructokinases, ribokinase, adenosine kinase, inosine-guanosine kinase, and phosphotagatokinase (for a short review see Prosite entry N° PD0C00504).

It is believed that the protein of SEQ ID NO:413 or part thereof is a carbohydrate or purine kinase. Preferred polypeptides of the invention are polypeptides comprising the amino acids of SEQ ID NO:413 from positions 28 to 94, and from 1 to 94. Other preferred polypeptides of the invention are fragments of SEQ ID NO:413 having any of the biological activity described herein. The kinase activity of the protein of the invention or part thereof may be assayed using any of the assays known to those skilled in the art including those described by the U.S. Pat. Nos. 5,756,315 and 5,861,294.

The invention relates to methods and compositions using the protein of the invention or part thereof to phosphorylate substrates, preferably carbohydrate or purine substrates. For example, the protein of the invention or part thereof is added to a sample containing the substrate(s) as well as a phosphate donor group in conditions allowing the transfer of the phosphorus group, and allowed to transfer the phosphorus group to the substrate(s). In a preferred embodiment, the kination is carried out using a standard assay including those described by the U.S. Pat. Nos. 5,756,315 and 5,861,294. Such phosphorylated purine substrates, such as 5′-IMP and 5′-GMP, have an enhanced flavor activity and may be used as seasoning agents.

In another embodiment, the present invention relates to processes and compositions for controlling the production of phosphorylated substrates, preferably carbohydrate and purine substrates, more preferably glucose, fructose, inosine, guanosine, adenosine, wherein a cell or an organism is an organism is genetically engineered either to produce the protein of the invention or part thereof or to inhibit the endogenous expression of the protein of the invention or part thereof using methods and techniques known to those skilled in the art including those described in U.S. Pat. No. 6,031,154. For example, a plant may be genetically engineered to express the protein of the invention or part thereof, thereby increasing the amount of phosphorylated carbohydrate substrates to be imported into plastids and ultimately enhancing starch biosynthesis. On the contrary, a fruit may also be genetically engineered to inhibit the endogenous expression of the protein of the invention in order to increase the concentration of non phosphorylated carbohydrates, ultimately leading to fruits with enhanced sweetness.

The invention further relates to methods and composition using the protein of the invention or part thereof to diagnose, prevent and/or treat disorders in which the availability of phosphorylated substrates, preferably carbohydrate and purine substrates, is impaired or needs to be impaired. In a preferred embodiment, the protein of the invention or part thereof may be used to activate pharmacologically active nucleosides including but not limited to tubercidin, formycin, ribavirin, pyrazofurin and 6-(methylmercapto) purine riboside which are antimetabolites with cytotoxic, anticancer and antiviral properties. In another preferred embodiment, the protein of the invention or part thereof may be used to compensate alterations observed in endogenous adenosine kinase activity observed in certain disorders including but not limited to hepatoma, hepatectomy, gout, and HIV infection. In still another preferred embodiment, the protein of the invention or part thereof may be used to modulate the concentration of adenosine which was shown to play important physiological roles. In the central nervous system, adenosine inhibits the release of certain neurotransmitters (Corradetti et al., Eur. J. Pharmacol. 1984, 104: 19-26), stabilizes membrane potential (Rudolphi et al., Cerebrovasc. Brain Metab. Rev. 1992, 4: 346-360), functions as an endogenous anticonvulsant (Dragunow, Trends Pharmacol. Sci. 1986, 7:128-130) and may have a role as an endogenous neuroprotective agent (Rudolphi et al., Trends Pharmacol. Sci. 1992, 13: 439-445). Adenosine has also been implicated in modulating transmission in pain pathways in the spinal cord (Sawynok et al., Br. J. Pharmacol. 1986, 88: 923-930), and in mediating the analgesic effects of morphine (Sweeney et al., J. Pharmacol. Exp. Ther. 1987, 243: 657-665). In the immune system, adenosine inhibits certain neutrophil functions and exhibits anti-inflammatory effects (Cronstein, J. Appl. Physiol. 1994, 76: 5-13). Adenosine also exerts a variety of effects on the cardiovascular system, including vasodilation, impairment of atrioventricular conduction and endogenous cardioprotection in myocardial ischemia and reperfusion (Mullane and Williams, in Adenosine and Adenosine Receptors 1990 (Williams, ed) Humana Press, New Jersey, pp. 289-334). The widespread actions of adenosine also include effects on the renal, respiratory, gastrointestinal and reproductive systems, as well as on blood cells and adipocytes. Endogenous adenosine release appears to have a role as a natural defense mechanism in various pathophysiologic conditions, including cerebral and myocardial ischemia, seizures, pain, inflammation and sepsis. While adenosine is normally present at low levels in the extracellular space, its release is locally enhanced at the site(s) of excessive cellular activity, trauma or metabolic stress. Once in the extracellular space, adenosine activates specific extracellular receptors to elicit a variety of responses which tend to restore cellular function towards normal (Bruns, Nucleosides Nucleotides, 1991, 10: 931-943; Miller and Hsu, J. Neurotrauma, 1992, 9: S563-S577). Adenosine has a half-life measured in seconds in extracellular fluids (Moser et al., Am. J. Physiol. 1989, 25: C799-C806), and its endogenous actions are therefore highly localized. The inhibition of adenosine kinase can result in augmentation of the local adenosine concentrations at foci of tissue injury, further enhancing cytoprotection. This effect is likely to be most pronounced at tissue sites where trauma results in increased adenosine production, thereby minimizing systemic toxicities. Pharmacological compounds directed towards adenosine kinase inhibition provide potential effective new therapies for disorders benefited by the site- and event-specific potentiation of adenosine.

Protein of SEQ ID NO:439 (Internal Designation 108-011-5-0-C7-FLC)

The protein of SEQ ID NO:439 encoded by the extended cDNA SEQ ID NO:34 shows homology with the chicken ribonuclease A (Embl accession number X61192) which is part of the pancreatic ribonuclease family. In addition, the protein of the invention exhibits the pfam signature for this family of pancreatic ribonucleases from positions 17 to 67.

Ribonucleases are proteins which catalyze the hydrolysis of phosphodiester bonds in RNA chains. Pancreatic ribonucleases are pyrimidic-specific ribonucleases present in high quantity in the pancreas of a number of mammalia taxa and of a few reptiles. In addition to their function in hydrolysis of RNA, ribonucleases have evolved to support a variety of other physiological activities. Such activities include anti-parasite, anti-bacterium, anti-virus, anti-neoplastic activities, neurotoxicity, and angiogenesis. For example, bovine seminal ribonuclease is anti-neoplastic (Laceetti, P. et al. (1992) Cancer Res. 52: 4582-4586). Some frog ribonucleases display both anti-viral and anti-neoplastic activity (Youle, R. J. et al. (1994) Proc. Natl. Acad. Sci. USA 91: 6012-6016; Mikulski, S. M. et al. (1990) J. Natl. Cancer Inst. 82: 151-152; and Wu, Y.-N. et al. (1993) J. Biol. Chem. 268: 10686-10693). Angiogenin is a tRNA-specific ribonuclease which binds actin on the surface of endothelial cells for endocytosis. Endocytosed angiogenin is translocated to the nucleus where it promotes endothelial invasiveness required for blood vessel formation (Moroianu, J. and Riordan, J. F. (1994) Proc. Natl. Acad. Sci. USA 91: 1217-1221). Eosinophil-derived neurotoxin (EDN) and eosinophil cationic protein (ECP) are related ribonucleases which possess neurotoxicity (Beintema, J. J. et al. (1988) Biochemistry 27: 4530-4538; Ackerman, S. J. (1993) In Makino, S. and Fukuda, T., Eosinophils: Biological and Clinical Aspects. CRC Press, Boca Raton, Fla., pp 33-74). In addition, ECP exhibits cytotoxic, anti-parasitic, and anti-bacterial activities. A EDN-related ribonuclease, named RNase k6, is shown to express in normal human monocytes and neutrophils, suggesting a role for this ribonuclease in host defense (Rosenberg, H. F. and Dyer, K. D. (1996) Nuc. Acid. Res. 24: 3507-3513).

It is believed that the protein of SEQ ID NO:439 or part thereof is a ribonuclease. Preferred polypeptides of the invention are polypeptides comprising the amino acids of SEQ ID NO:439 from positions 17 to 67. Other preferred polypeptides of the invention are fragments of SEQ ID NO:439 having any of the biological activity described herein. The ribonuclease activity of the protein of the invention or part thereof may be assayed using any of the assays known to those skilled in the art including those described in U.S. Pat. No. 5,866,119.

The invention relates to methods and compositions using the protein of the invention or part thereof to hydrolyze one or several substrates, preferably nucleic acids, more preferably RNA, alone or in combination with other substances. For example, the protein of the invention or part thereof is added to a sample containing the substrate(s) in conditions allowing hydrolysis, and allowed to catalyze the hydrolysis of the substrate(s).

In a preferred embodiment, the protein of the invention or part thereof may be used to remove contaminating RNA in a biological sample, alone or in combination with other nucleases. In a more preferred embodiment, the protein of the invention or part thereof may be used to purify DNA preparations from contaminating RNA, to remove RNA templates prior to second strand synthesis and prior to analysis of in vitro translation products. Compositions comprising the protein of the present invention or part thereof are added to biological samples as a “cocktail” with other nucleases. The advantage of using a cocktail of hydrolytic enzymes is that one is able to hydrolyze a wide range of substrates without knowing the specificity of any of the enzymes. Such cocktails of nucleases are commonly used in molecular biology assays, for example to remove unbound RNA in RNAse protection assays. Using a cocktail of hydrolytic enzymes also protects a sample from a wide range of future unknown RNA contaminants from a vast number of sources. For example, the protein of the invention or part thereof is added to samples where contaminating substrates is undesirable. Alternatively, the protein of the invention or part thereof may be bound to a chromatographic support, either alone or in combination with other hydrolytic enzymes, using techniques well known in the art, to form an affinity chromatography column. A sample containing the undesirable substrate is run through the column to remove the substrate. Immobilizing the protein of the invention or part thereof on a support is particularly advantageous for those embodiments in which the method is to be practiced on a commercial scale. This immobilization facilitates the removal of the enzyme from the batch of product and subsequent reuse of the enzyme. Immobilization of the protein of the invention or part thereof can be accomplished, for example, by inserting a cellulose-binding domain in the protein. One of skill in the art will understand that other methods of immobilization could also be used and are described in the available literature. Alternatively, the same methods may be used to identify new substrates.

In another embodiment, the protein of the invention or part thereof may be used to decontaminate or disinfect samples infected by undesirable parasite, bacteria and/or viruses using any of the methods known to those skilled in the art including those described in Youle et al, (1994), supra; Mikulski et al (1990) supra, Wu et al (1993) supra.

In another embodiment, the present invention relates to compositions and methods using the protein of the invention or part thereof to selectively kill cells. The protein of the invention or part thereof is linked to a recognition moiety capable of binding to a chosen cell, such as lectins, receptors or antibodies thus generating cytotoxic reagents using methods and techniques described in U.S. Pat. No. 5,955,073.

In another embodiment, the protein of the invention or part thereof may be used in the diagnosis, prevention and/or treatment of disorders associated with excessive cell proliferation such as cancer.

Protein of SEQ ID NO:409 (Internal Designation 105-118-4-0-E6-FLC)

The protein of SEQ ID NO:409 encoded by the extended cDNA SEQ ID NO:4 is homologous to a hepatocellular carcinoma associated ring finger protein (Embl accession number AF247565) and homology with a putative anaphase-promoting complex subunit from Drosophila (Embl accession number AJ251510). In addition, the protein of the invention exhibits the pfam PHD zinc finger signature from positions 33 to 79.

Zinc finger domains are found in numerous zinc binding proteins which are involved in protein-nucleic acid interactions. They are independently folded zinc-containing mini-domains which are used in a modular repeating fashion to achieve sequence-specific recognition of DNA (Klug 1993 Gene 135, 83-92). Such zinc binding proteins are commonly involved in the regulation of gene expression, and usually serve as transcription factors (see U.S. Pat. Nos. 5,866,325; 6,013,453 and 5,861,495). PHD fingers are C₄HC₃ zinc fingers spanning approximately 50-80 residues and distinct from RING fingers or LIM domains. They are thought to be mostly DNA or RNA binding domain but may also be involved in protein-protein interactions (for a review see Aasland et al, Trends Biochem Sci 20:56-59 (1995)).

It is believed that the protein of SEQ ID NO:409 or part thereof is a zinc binding protein, preferably able to bind nucleic acids, more preferably a transcription factor. Preferred polypeptides of the invention are polypeptides comprising the amino acids of SEQ ID NO:409 from positions 33 to 79. Other preferred polypeptides of the invention are fragments of SEQ ID NO:409 having any of the biological activity described herein. The nucleic acid binding activity of the protein of the invention or part thereof may be assayed using any of the assays known to those skilled in the art including those described in U.S. Pat. No. 6,013,453.

The invention relates to methods and compositions using the protein of the invention or part thereof to bind to nucleic acids, preferably DNA, alone or in combination with other substances. For example, the protein of the invention or part thereof is added to a sample containing nucleic acid in conditions allowing binding, and allowed to bind to nucleic acids. In a preferred embodiment, the protein of the invention or part thereof may be used to purify nucleic acids such as restriction fragments. In another preferred embodiment, the protein of the invention or part thereof may be used to visualize nucleic acids when the polypeptide is linked to an appropriate fusion partner, or is detected by probing with an antibody. Alternatively, the protein of the invention or part thereof may be bound to a chromatographic support, either alone or in combination with other DNA binding proteins, using techniques well known in the art, to form an affinity chromatography column. A sample containing nucleic acids to purify is run through the column. Immobilizing the protein of the invention or part thereof on a support advantageous is particularly for those embodiments in which the method is to be practiced on a commercial scale. This immobilization facilitates the removal of the protein from the batch of product and subsequent reuse of the protein. Immobilization of the protein of the invention or part thereof can be accomplished, for example, by inserting a cellulose-binding domain in the protein. One of skill in the art will understand that other methods of immobilization could also be used and are described in the available literature.

In another embodiment, the present invention relates to compositions and methods using the protein of the invention or part thereof, especially the zinc binding domain, to alter the expression of genes of interest in a target cells. Such genes of interest may be disease related genes, such as oncogenes or exogenous genes from pathogens, such as bacteria or viruses using any techniques known to those skilled in the art including those described in U.S. Pat. Nos. 5,861,495; 5,866,325 and 6,013,453.

In still another embodiment, the protein of the invention or part thereof may be used to diagnose, treat and/or prevent disorders linked to dysregulation of gene transcription such as cancer and other disorders relating to abnormal cellular differentiation, proliferation, or degeneration, including hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease.

Protein of SEQ ID NO:446 (Internal Designation 108-014-5-0-D12-FLC)

The protein of SEQ ID NO:446 encoded by the extended cDNA SEQ ID NO:41 shows homology with zinc binding proteins (Embl accession number Q9QZQ6 and Genseq accession number W69602). In addition, the protein of the invention exhibits the pfam RING zinc finger signature from positions 258 to 298.

Zinc binding (ZB) domains are found in numerous proteins which are involved in protein-nucleic acid or protein-protein interactions. ZB proteins are commonly involved in the regulation of gene expression, and may serve as transcription factors and signal transduction molecules. A ZB domain is generally composed of 25 to 30 amino acid residues which form one or more tetrahedral ion binding sites. The binding sites contain four ligands consisting of the sidechains of cysteine, histidine and occasionally aspartate or glutamate. The binding of zinc allows the relatively short stretches of polypeptide to fold into defined structural units which are well-suited to participate in macromolecular interactions (Berg, J. M. et al. (1996) Science 271:1081-1085). Zinc binding domains which contain a C₃HC₄ sequence motif are known as RING domains (Lovering, R. et al. (1993) Proc. Natl. Acad. Sci. USA 90:2112-2116). The RING domain consists of eight metal binding residues, and the sequences that bind the two metal ions overlap (Barlow, P. N. et al. (1994) J. Mol. Biol. 237:201-211). Functions of RING finger proteins are mediated through DNA binding and include the regulation of gene expression, DNA recombination, and DNA repair (see Borden and Freemont, Curr Opin Struct Biol 6:395-401 (1996) and U.S. Pat. No. 5,861,495).

It is believed that the protein of SEQ ID NO:446 or part thereof is a zinc binding protein, preferably able to bind nucleic acids or proteins, more preferably a transcription factor. Preferred polypeptides of the invention are polypeptides comprising the amino acids of SEQ ID NO:446 from positions 258 to 298. Other preferred polypeptides of the invention are fragments of SEQ ID NO:446 having any of the biological activity described herein. The nucleic acid binding activity of the protein of the invention or part thereof may be assayed using any of the assays known to those skilled in the art including those described in U.S. Pat. No. 6,013,453.

The invention relates to methods and compositions using the protein of the invention or part thereof to bind to nucleic acids, preferably DNA, alone or in combination with other substances. For example, the protein of the invention or part thereof is added to a sample containing nucleic acid in conditions allowing binding, and allowed to bind to nucleic acids. In a preferred embodiment, the protein of the invention or part thereof may be used to purify nucleic acids such as restriction fragments. In another preferred embodiment, the protein of the invention or part thereof may be used to visualize nucleic acids when the polypeptide is linked to an appropriate fusion partner, or is detected by probing with an antibody. Alternatively, the protein of the invention or part thereof may be bound to a chromatographic support, either alone or in combination with other DNA binding proteins, using techniques well known in the art, to form an affinity chromatography column. A sample containing nucleic acids to purify is run through the column. Immobilizing the protein of the invention or part thereof on a support advantageous is particularly for those embodiments in which the method is to be practiced on a commercial scale. This immobilization facilitates the removal of the protein from the batch of product and subsequent reuse of the protein. Immobilization of the protein of the invention or part thereof can be accomplished, for example, by inserting a cellulose-binding domain in the protein. One of skill in the art will understand that other methods of immobilization could also be used and are described in the available literature.

In another embodiment, the present invention relates to compositions and methods using the protein of the invention or part thereof, especially the zinc binding domain, to alter the expression of genes of interest in a target cells. Such genes of interest may be disease related genes, such as oncogenes or exogenous genes from pathogens, such as bacteria or viruses using any techniques known to those skilled in the art including those described in U.S. Pat. Nos. 5,861,495; 5,866,325 and 6,013,453.

In still another embodiment, the protein of the invention or part thereof may be used to diagnose, treat and/or prevent disorders linked to dysregulation of gene transcription such as cancer and other disorders relating to abnormal cellular differentiation, proliferation, or degeneration, including hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease.

Protein of SEQ ID NO:437 (Internal Designation 108-008-5-0-G5-FLC)

The protein of SEQ ID NO:437 encoded by the extended cDNA SEQ ID NO:32 shows homology with zinc binding proteins (Embl accession number Q9VZJ9). In addition, the protein of the invention exhibits the pfam RING zinc finger signature from positions 302 to 339.—

Zinc binding (ZB) domains are found in numerous proteins which are involved in protein-nucleic acid or protein-protein interactions. ZB proteins are commonly involved in the regulation of gene expression, and may serve as transcription factors and signal transduction molecules. A ZB domain is generally composed of 25 to 30 amino acid residues which form one or more tetrahedral ion binding sites. The binding sites contain four ligands consisting of the sidechains of cysteine, histidine and occasionally aspartate or glutamate. The binding of zinc allows the relatively short stretches of polypeptide to fold into defined structural units which are well-suited to participate in macromolecular interactions (Berg, J. M. et al. (1996) Science 271:1081-1085). Zinc binding domains which contain a C₃HC₄ sequence motif are known as RING domains (Lovering, R. et al. (1993) Proc. Natl. Acad. Sci. USA 90:2112-2116). The RING domain consists of eight metal binding residues, and the sequences that bind the two metal ions overlap (Barlow, P. N. et al. (1994) J. Mol. Biol. 237:201-211). Functions of RING finger proteins are mediated through DNA binding and include the regulation of gene expression, DNA recombination, and DNA repair (see Borden and Freemont, Curr Opin Struct Biol 6:395-401 (1996) and U.S. Pat. No. 5,861,495).

It is believed that the protein of SEQ ID NO:437 or part thereof is a zinc binding protein, preferably able to bind nucleic acids or proteins, more preferably a transcription factor. Preferred polypeptides of the invention are polypeptides comprising the amino acids of SEQ ID NO:437 from positions 302 to 339. Other preferred polypeptides of the invention are fragments of SEQ ID NO:437 having any of the biological activity described herein. The nucleic acid binding activity of the protein of the invention or part thereof may be assayed using any of the assays known to those skilled in the art including those described in U.S. Pat. No. 6,013,453.

The invention relates to methods and compositions using the protein of the invention or part thereof to bind to nucleic acids, preferably DNA, alone or in combination with other substances. For example, the protein of the invention or part thereof is added to a sample containing nucleic acid in conditions allowing binding, and allowed to bind to nucleic acids. In a preferred embodiment, the protein of the invention or part thereof may be used to purify nucleic acids such as restriction fragments. In another preferred embodiment, the protein of the invention or part thereof may be used to visualize nucleic acids when the polypeptide is linked to an appropriate fusion partner, or is detected by probing with an antibody. Alternatively, the protein of the invention or part thereof may be bound to a chromatographic support, either alone or in combination with other DNA binding proteins, using techniques well known in the art, to form an affinity chromatography column. A sample containing nucleic acids to purify is run through the column. Immobilizing the protein of the invention or part thereof on a support advantageous is particularly for those embodiments in which the method is to be practiced on a commercial scale. This immobilization facilitates the removal of the protein from the batch of product and subsequent reuse of the protein. Immobilization of the protein of the invention or part thereof can be accomplished, for example, by inserting a cellulose-binding domain in the protein. One of skill in the art will understand that other methods of immobilization could also be used and are described in the available literature.

In another embodiment, the present invention relates to compositions and methods using the protein of the invention or part thereof, especially the zinc binding domain, to alter the expression of genes of interest in a target cells. Such genes of interest may be disease related genes, such as oncogenes or exogenous genes from pathogens, such as bacteria or viruses using any techniques known to those skilled in the art including those described in U.S. Pat. Nos. 5,861,495; 5,866,325 and 6,013,453.

In still another embodiment, the protein of the invention or part thereof may be used to diagnose, treat and/or prevent disorders linked to dysregulation of gene transcription such as cancer and other disorders relating to abnormal cellular differentiation, proliferation, or degeneration, including hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease.

Protein of SEQ ID NO:438 (Internal Designation 108-011-5-0-B12-FL)

The protein of SEQ ID NO:438 encoded by the extended cDNA SEQ ID NO:33 shows homology to the predicted extracellular domain and part of transmembrane domain of interleukin-17 receptor of both human and murine species (Genbank accession numbers WO4185 and WO4184). These IL-17R proteins are thought to belong to a new family of receptors for cytokines which induce T cell proliferation, I-CAM expression and preferential maturation of haematopoietic precursors into neutrophils (Yao et al., Cytokine., 9:794-8001 (1997)). It is also thought to play a proinflammatory role and to induce nitric oxide. The protein of the invention has a 21 amino acid transmembrane domain (positions 172 to 192) as predicted by the software TopPred II (Claros and von Heijne, CABIOS applic. Notes, 10:685-686 (1994)) matching the 21 amino acid putative transmembrane domain of human interleukin-17 receptor.

It is believed that the protein of SEQ ID NO:438 plays a role in regulating immune and/or inflammatory responses. Preferred polypeptides of the invention are fragments of SEQ ID NO:438 having any of the biological activities described herein.

The present invention relates to methods and compositions using the protein of the invention or part thereof to inhibit the proliferation and/or the differentiation of lymphocytes or lymphocytic cell lines, both in vitro and in vivo. For example, soluble forms of the protein of the invention or part thereof may be added to cell culture medium in an amount effective to inhibit the proliferation and/or the differentiation of lymphocytes and/or lymphocytic cell lines.

Another embodiment relates to methods and compositions using the protein of the invention or part thereof to diagnose, treat and/or prevent several disorders including, but not limited to, cancer, inflammatory and immune disorders, septic shock and impotence. Immune and inflammatory disorders include Addison's disease, AIDS, acute or chronic inflammation due to antigen, antibody and/or complement deposition, acute and delayed hypersensitivity, adult respiratory distress syndrome, allergies, anemia, arthritis, asthma, atherosclerosis, bronchitis, chalangitis, cholecystitus, Crohn's disease, ulcerative colitis, atopic dermatitis, dermatomyositis, diabetes mellitus, emphysema, encephalitis, endocarditis, atrophic gastritis, glomerulonephritis, gout, graft rejection, graft-versus-host disease, Graves' disease, hepatitis, hypereosinophilia, irritable bowel syndrome, lupus erythematosus, multiple sclerosis, myasthenia gravis, myocardial or pericardial inflammation, osteoarthritis, osteoporosis, pancreatitis, polycystic kidney disease, polymyositis, reperfusion injury, rheumatoid arthritis, scleroderma, Sjogren's syndrome, and autoimmune thyroiditis.

In addition, this protein may also be useful to modulate immune and/or inflammatory responses to infectious responses and/or to suppress graft rejection. For example, soluble forms of the protein of the invention or blocking antibodies, or antagonists may be used to inhibit and/or reduce immune and/or inflammatory responses.

Protein of SEQ ID NO:429 (Internal Designation 108-004-5-0-B12-FLC)

The protein of SEQ ID NO:429 encoded by the extended cDNA SEQ ID NO:24 is homologous to a human protein either described as a maid-like gene (Embl accession number 35 AF132000) or a human secreted protein (Geneseq accession number Y41330).

Maid is a maternally transcribed gene encoding a putative regulator of basic helix-loop-helix transcription factor in the mouse egg and zygote. In vitro, maid is able to bind to DNA. When transfected, maid reduces the transcription of a CAT-reporter regulated by an E12/MyoD enhancer (Hwang et al, Dev Dyn, 209:217-26 (1997)).

It is believed that the protein of SEQ ID NO:429 or part thereof is involved in the regulation of gene transcription, probably through direct binding to DNA. Preferred polypeptides of the invention are fragments of SEQ ID NO:429 having any of the biological activity described herein. The nucleic acid binding activity of the protein of the invention or part thereof may be assayed using any of the assays known to those skilled in the art including those described in U.S. Pat. No. 6,013,453.

The invention relates to methods and compositions using the protein of the invention or part thereof to bind to nucleic acids, preferably DNA, alone or in combination with other substances. For example, the protein of the invention or part thereof is added to a sample containing nucleic acid in conditions allowing binding, and allowed to bind to nucleic acids. In a preferred embodiment, the protein of the invention or part thereof may be used to purify nucleic acids such as restriction fragments. In another preferred embodiment, the protein of the invention or part thereof may be used to visualize nucleic acids when the polypeptide is linked to an appropriate fusion partner, or is detected by probing with an antibody. Alternatively, the protein of the invention or part thereof may be bound to a chromatographic support, either alone or in combination with other DNA binding proteins, using techniques well known in the art, to form an affinity chromatography column. A sample containing nucleic acids to purify is run through the column. Immobilizing the protein of the invention or part thereof on a support advantageous is particularly for those embodiments in which the method is to be practiced on a commercial scale. This immobilization facilitates the removal of the protein from the batch of product and subsequent reuse of the protein. Immobilizing the protein of the invention or part thereof on a support advantageous is particularly for those embodiments in which the method is to be practiced on a commercial scale. This immobilization facilitates the removal of the protein from the batch of product and subsequent reuse of the protein. Immobilization of the protein of the invention or part thereof can be accomplished, for example, by inserting a cellulose-binding domain in the protein. One of skill in the art will understand that other methods of immobilization could also be used and are described in the available literature.

In another embodiment, the present invention relates to compositions and methods using the protein of the invention or part thereof to alter the expression of genes of interest in a target cell. Such genes of interest may be disease related genes, such as oncogenes or exogenous genes from pathogens, such as bacteria or viruses using any techniques known to those skilled in the art including those described in U.S. Pat. Nos. 5,861,495; 5,866,325 and 6,013,453.

In still another embodiment, the protein of the invention or part thereof may be used to diagnose, treat and/or prevent disorders linked to dysregulation of gene transcription such as cancer and other disorders relating to abnormal cellular differentiation, proliferation, or degeneration, including hyperaldosteronism, hypocortisolism (Addison's disease), hyperthyroidism (Grave's disease), hypothyroidism, colorectal polyps, gastritis, gastric and duodenal ulcers, ulcerative colitis, and Crohn's disease.

Protein of SEQ ID NO:454 (Internal Designation 108-020-5-O-D4-FLC)

The protein of SEQ ID NO:454 encoded by the extended cDNA SEQ ID NO:49 shows homology to a murine transmembrane protein (Genbank accession number BAA92746). When expressed in E. Coli, the matched which suppresses bacterial growth (Inoue et al, Biochem Biophys Res Commun 268:553-61 (2000)). In addition, a transmembrane domain is predicted for the protein of SEQ ID NO:454 from positions 36 to 56 by the software TopPred II (Claros and von Heijne, CABIOS applic. Notes, 10:685-686 (1994).

It is believed that the protein of SEQ ID NO:454 or part thereof is able to suppress bacterial growth. Preferred polypeptides of the invention are fragments of SEQ ID NO:429 having any of the biological activity described herein. The growth inhibiting activity of the protein of the invention or part thereof may be assayed using any of the assays known to those skilled in the art including those described in Inoue et al, supra.

The invention relates to methods and compositions using the protein of the invention or part thereof to suppress bacterial growth. For example, the protein of the invention may be expressed in a bacteria, preferably E. coli, using recombinant DNA technology methods known to those skilled in the art. The bacterial growth may then be assessed using any methods or techniques known to those skilled in the art.

Protein of SEQ ID NO:428 (Internal Designation 122-007-3-0-D10-FLC)

The protein of SEQ ID NO:428 encoded by the extended cDNA SEQ ID NO:23 shows homology to a human secreted protein highly expressed in testis (Genseq accession number Y06940). In addition, it exhibits an emotif signature for the flagellar biosynthetic protein fliR 30 family from positions 7 to 27.

FliR is an integral membrane protein located in the flagellar basal body and thought to be a component of the type III export apparatus (Fan et al, Mol Microbiol 26:1035-46 (1997)).

It is believed that the protein of SEQ ID NO:428 or part thereof plays a role in gametogenesis, maybe as a component of spermatozoids. Preferred polypeptides of the invention are polypeptides comprising the amino acids of SEQ ID NO:428 from positions 7 to 27. Other preferred polypeptides of the invention are fragments of SEQ ID NO:428 having any of the biological activity described herein.

The invention relates to methods and compositions using the protein of the invention or part thereof to diagnose, treat and/or prevent fertility disorders. For diagnostic purposes, the expression of the protein of the invention could be investigated using any of the Northern blotting, RT-PCR or immunoblotting methods described herein and compared to the expression in control individuals. For prevention and/or treatment purposes, the protein of the invention may be used to enhance gametogenesis using any of the gene therapy methods described herein or known to those skilled in the art.

Moreover, antibodies to the protein of the invention or part thereof may be used for detection of gametes using any techniques known to those skilled in the art.

Protein of SEQ ID NO:442 (Internal Designation 108-013-5-0-G5-FLC)

The protein of SEQ ID NO:442 encoded by the extended cDNA SEQ ID NO:37 displays the pfam signature for the N-terminus of the alpha-macroglobulin A2M family from positions 17 to 40. A2M-like proteins are able to inhibit all four classes of proteinases by a “trapping mechanism” (see Prosite entry PS00477 for a short review).

It is believed that the protein of SEQ ID NO:442 or part thereof is a member of the alpha-2-macroglobulin family, more preferably a protease inhibitor. Preferred polypeptides of the invention are polypeptides comprising the amino acids of SEQ ID NO:442 from positions 17 to 40. Other preferred polypeptides of the invention are fragments of SEQ ID NO:425 having any of the biological activity described herein. The protease inhibitor activity of the protein of the invention or part thereof may be assessed using any techniques known to those skilled in the art.

The invention relates to compositions and methods using the protein of the invention or part thereof to inhibit proteases, both in vitro or in vivo. Since proteases play an important role in the regulation of many biological processes in virtually all living organisms as well as a major role in diseases, inhibitors of proteases are useful in a wide variety of applications.

In one embodiment, the protein of the invention or part thereof may be useful to quantify the amount of a given protease in a biological sample, and thus used in assays and diagnostic kits for the quantification of proteases in bodily fluids or other tissue samples, in addition to bacterial, fungal, plant, yeast, viral or mammalian cell cultures. In a preferred embodiment, the sample is assayed using a standard protease substrate. A known concentration of protease inhibitor is added, and allowed to bind to a particular protease present. The protease assay is then rerun, and the loss of activity is correlated to the protease inhibitor activity using techniques well known to those skilled in the art.

In addition, the protein of the invention or part thereof may be used to remove, identify or inhibit contaminating proteases in a sample. Compositions comprising the polypeptides of the present invention may be added to biological samples as a “cocktail” with other protease inhibitors to prevent degradation of protein samples. The advantage of using a cocktail of protease inhibitors is that one is able to inhibit a wide range of proteases without knowing the specificity of any of the proteases. Using a cocktail of protease inhibitors also protects a protein sample from a wide range of future unknown proteases which may contaminate a protein sample from a vast number of sources. For example, the protein of the invention or part thereof are added to samples where proteolytic degradation by contaminating proteases is undesirable. Such protease inhibitor cocktails (see for example the ready to use cocktails sold by Sigma) are widely used in research laboratory assays to inhibit proteases susceptible of degrading a protein of interest for which the assay is to be performed. Alternatively, the protein of the invention or part thereof may be bound to a chromatographic support, either alone or in combination with other protease inhibitor, using techniques well known in the art, to form an affinity chromatography column. A sample containing the undesirable protease is run through the column to remove the protease. Alternatively, the same methods may be used to identify new proteases.

In a preferred embodiment, the protein of the invention or part thereof may be used to inhibit proteases implicated in a number of diseases where cellular proteolysis occur such as diseases characterized by tissue degradation including but not limited to arthritis, muscular dystrophy, inflammation, tumor invasion, glomerulonephritis, parasite-borne infections, Alzheimer's disease, periodontal disease, and cancer metastasis.

In another preferred embodiment, the protein of the invention or part thereof may be useful to inhibit exogenous proteases, both in vivo and in vitro, implicated in a number of infectious diseases including but not limited to gingivitis, malaria, leishmaniasis, filariasis, osteoporosis and osteoarthritis, and other bacterial, and parasite-borne or viral infections. In particular, the protein of the invention or part thereof may offer applications in viral diseases where the proteolysis of primary polypeptide precursors is essential to the replication of the virus, as for HIV and HCV.

Furthermore, the protease inhibitors of the present invention find use in drug potentiation applications. For example, therapeutic agents such as antibiotics or antitumor drugs can be inactivated through proteolysis by endogenous proteases, thus rendering the administered drug less effective or inactive. Accordingly, the protease inhibitors of the invention may be administered to a patient in conjunction with a therapeutic agent in order to potentiate or increase the activity of the drug. This co-administration may be by simultaneous administration, such as a mixture of the protease inhibitor and the drug, or by separate simultaneous or sequential administration.

In addition, protease inhibitors have been shown to inhibit the growth of microorganisms including human pathogenic bacteria. For example, protease inhibitors are able to inhibit growth of all strains of group A streptococci, including antibiotic-resistant strains (Merigan, T. et al (1996) Ann Intern Med 124:1039-1050; Stoka, V. (1995) FEBS. Lett 370:101-104; Vonderfecht, S. et al (1988) J Clin Invest 82:2011-2016; Collins, A. et al (1991) Antimicrob Agents Chemother 35:2444-2446). Accordingly, the protease inhibitors of the present invention may be used as antibacterial agents to retard or inhibit the growth of certain bacteria either in vitro or in vivo. Particularly, the polypeptides of the present invention may be used to inhibit the growth of group A streptococci on non-living matter such as instruments not conducive to other methods of preventing or removing contamination by group A streptococci, and in culture of living plant, fungi, and animal cells.

Protein of SEQ ID NO:693

The protein of SEQ ID NO: 693 is encoded by the extended cDNA SEQ ID NO: 51. The protein of SEQ ID NO: 693 is human strictosidine synthase. Strictodine synthase is a key enzyme in the production of, and therefore useful in making, the pharmaceutically important monoterpene indole alkaloids. Pathways for the production of monoterpene indole alkaloids can be reconstructed in various cell types, for example, insect cell cultures as described in Kutchan, T. M. et al. (1994) Phyochemistry 35(2):353-360. Strictodine synthase can also be produced E. coli and its activity measuring using methods described in, for example, Roessner, C. A. et al. (1992) Protein Expr. Purif. 3(4):295-300; Kutchan, T. M. (1989) FEBS Lett. 257(1):127-130; Pennings, E. J. et al. (1989) Anal. Biochem. 176(2):412-415; Walton, N. J. (1987) Anal. Biochem. 163(2):482-488. Preferred fragments of SEQ ID NO: 693 and the mature polypeptide encoded by the corresponding human cDNA of the deposited clone are those with strictodine synthase activity. Further preferred are fragments with not less then 100 fold less activity, not less than 10 fold activity, and not less than 5 fold activity when compared to mature protein.

Protein of SEQ ID NO: 695

The protein of SEQ ID NO: 695, encoded by the extended cDNA SEQ ID NO: 53, is human inositol hexakisphophate kinase-2. Inositol hexakisphophate kinase-2 phosphorylates inositol hexakisphosphate (InsP(6)) to diphosphoinositol pentakisphosphate/inositol heptakisphosphate (InsP(7)), a high energy regulator of cellular trafficking. Human inositol hexakisphophate kinase-2 also stimulates the uptake of inorganic phosphate and its products act as energy reserves. Therefore, hexakisphosphate kinase-2 is an ATP synthase, and its product, diphosphoinositol pentakisphosphate, acts as a high-energy phosphate donor. The human inositol hexakisphophate kinase-2 gene may be transfected into eukaryotic cells (preferably mammalian, yeast, and insect cells) and expressed to increase their growth, viability, and for more efficient secretions of polypeptides, including recombinant polypeptides. Preferred fragments of SEQ ID NO: 695 and the corresponding mature polypeptide encoded by the human cDNA of the deposited clone are those with inositol hexakisphophate kinase-2 activity. Further preferred are fragments with not less then 100 fold less activity, not less than 10 fold activity, and not less than 5 fold activity when compared to mature protein.

Proteins of SEQ ID NOs: 697 and 727:

The proteins of SEQ ID NOs: 697 and 727 encoded by the extended cDNA SEQ ID NOs: 55 and 85, respectively, are MEK binding partners. These proteins enhance enzymatic activation of mitogen-activated protein (MAP) kinase cascade. The MAP kinase pathway is one of the important enzymatic cascade that is conserved among all eukaryotes from yeast to human. This kind of pathway is involved in vital functions such as the regulation of growth, differentiation and apoptosis. These proteins are believed to act by facilitating the interaction of the two sequentially acting kinases MEKI and ERKI (Schaffer et al., Science, 281:1668-1671 (1998)).

Thus, the proteins of SEQ ID NO: 697 and 727 are involved in regulating protein-protein interaction in the signal transduction pathways. These proteins may be useful in diagnosing and/or treating several types of disorders including, but not limited to, cancer, neurodegenerative diseases, cardiovascular disorders, hypertension, renal injury and repair and septic shock. More specifically, over expression and mutant forms of this gene can serve as markers for cancer, such as ovarian cancer, using the nucleic acid as a probe or by using antibodies directed to the protein. Cells transfected with this gene have increased growth rate.

Protein of SEQ ID NO: 698

The protein of SEQ ID NO: 698, encoded by the extended cDNA SEQ ID NO: 56, is a new claudin named Claudin-50.

Cell adhesion is a complex process that is important for maintaining tissue integrity and generating physical and permeability barriers within the body. All tissues are divided into discrete compartments, each of which is composed of a specific cell type that adheres to similar cell types. Such adhesion triggers the formation of intercellular junctions (i.e., readily definable contact sites on the surfaces of adjacent cells that are adhering to one another), also known as tight junctions, gap junctions, spot desmosomes and belt desmosomes. The formation of such junctions gives rise to physical and permeability barriers that restrict the free passage of cells and other biological substances from one tissue compartment to another. For example, the blood vessels of all tissues are composed of endothelial cells. In order for components in the blood to enter a given tissue compartment, they must first pass from the lumen of a blood vessel through the barrier formed by the endothelial cells of that vessel. Similarly, in order for substances to enter the body via the gut, the substances must first pass through a barrier formed by the epithelial cells of that tissue. To enter the blood via the skin, both epithelial and endothelial cell layers must be crossed.

The transmembrane component of tight junctions that has been the most studied is occluding. Occludin is believed to be directly involved in cell adhesion and the formation of tight junctions (Furuse et al., J. Cell Sci. 109:429-435, 1996; Chen et al., J. 5 Cell Biol. 138:891-899, 1997). It has been proposed that occludin promotes cell adhesion through homophilic interactions (an occludin on the surface of one cell binds to an identical occludin on the surface of another cell). A detailed discussion of occludin structure and function is provided by Lampugnani and Dejana, Curr. Opin Cell Biol. 9:674-682, 1997.

More recently, a second family of tight junction components has been identified. Claudins are transmembrane proteins that appear to be directly involved in cell adhesion and the formation of tight junctions (Furuse et al., J. Cell Biology 141:1539-1550, 1998; Morita et al., Proc. Natl. Acad. Sci. USA 96:511-516, 1999). Other previously described proteins that appear to be members of the claudin family include RVP-1 (Briehl and Miesfeld, Molecular Endocrinology 5:1381-1388, 1991; Katahira et al., J. Biological Chemistry 272:26652-26656, 1997), the Clostridium perfringens enterotoxin receptor (CPE-R; see Katahira et al., J. Cell Biology 136:1239-1247, 1997; Katahira et al., J. Biological Chemistry 272:26652-26656, 1997) and TMVCF (transmembrane protein deleted in Velo-cardio-facial syndrome; Sirotkin et al., Genomics 42:245-51, 1997).

Based on hydrophobicity analysis, all claudins appear to be approximately 22 kD and contain four hydrophobic domains that transverse the plasma membrane. It has been proposed that claudins promote cell adhesion through homophilic interactions (a claudin on the surface of one cell binds to an identical claudin on the surface of another cell) or heterophilic interactions, possibly with occludin.

Although cell adhesion is required for certain normal physiological functions, there are situations in which the level of cell adhesion is undesirable. For example, many pathologies (such as autoimmune diseases and inflammatory diseases) involve abnormal cellular adhesion. Cell adhesion may also play a role in graft rejection. In such circumstances, modulation of cell adhesion may be desirable.

In addition, permeability barriers arising from cell adhesion create difficulties for the delivery of drugs to specific tissues and tumors within the body. For example, skin patches are a convenient tool for administering drugs through the skin. However, the use of skin patches has been limited to small, hydrophobic molecules because of the epithelial and endothelial cell barriers. Similarly, endothelial cells render the blood capillaries largely impermeable to drugs, and the blood/brain barrier has hampered the targeting of drugs to the central nervous system. In addition, many solid tumors develop internal barriers that limit the delivery of anti-tumor drugs and antibodies to inner cells.

Attempts to facilitate the passage of drugs across such barriers generally rely on specific receptors or carrier proteins that transport molecules across barriers in vivo. However, such methods are often inefficient, due to low endogenous transport rates or to the poor functioning of a carrier protein with drugs. While improved efficiency has been achieved using a variety of chemical agents that disrupt cell adhesion, such agents are typically associated with undesirable side-effects, may require invasive procedures for administration and may result in irreversible effects.

Accordingly, there is a need in the art for compounds that modulate cell adhesion and improve drug delivery across permeability barriers without such disadvantages. The present invention fulfills this need and further provides other related advantages.

The present invention provides compounds and methods for modulating claudin-mediated cell adhesion and the formation of permeability barriers. Within certain aspects, the present invention provides cell adhesion modulating agents that inhibit or enhance claudin-mediated cell adhesion. Certain modulating agents comprise the claudin CAR sequence WKTSSTVG. Other modulating agents comprise at least five or seven consecutive amino acid residues of a claudin CAR sequence: Comprising the sequence TSSY, wherein each permutation is an individual specie of the present invention.

The present invention further provides for polypeptides comprising amino acid residues 32 to 35 of SEQ. ID NO: 698, wherein said sequence comprises an additional 1 to 31 consecutive residues of N-terminal sequence of SEQ. ID NO: 698 and an additional 1 to 193 consecutive C-terminal residues of SEQ. ID NO: 698. Further included are polypeptides comprising additional consecutive residues at both the N-terminal, C-terminal. Each permutation of the above polypeptides comprising additional N-terminal, C-terminal & N— and C terminal residues are included in the present invention as individual species.

The present invention further provides, within other aspects, polynucleotides encoding a modulating agent as provided above, expression vectors comprising such a polynucleotide, and host cells transformed or transfected with such an expression vector.

Within further aspects, the present invention provides modulating agents that comprise an antibody or antigen-binding fragment thereof that specifically binds to a claudin CAR sequence and modulates a claudin-mediated function.

The present invention further provides modulating agents comprising a mimetic of a claudin CAR sequence that comprises at least three or five consecutive amino acid residues of the claudin CAR sequence WKTSSYVG.

Within other aspects, modulating agents as described above may be linked to one or more of a drug, a detectable marker, a targeting agent and/or a support material. Alternatively, or in addition, modulating agents as described above may further comprise one or more of: (a) a cell adhesion recognition sequence that is bound by an adhesion molecule other than a claudin, wherein the cell adhesion recognition sequence is separated from any claudin CAR sequence(s) by a linker; and/or (b) an antibody or antigen-binding fragment thereof that specifically binds to a cell adhesion recognition sequence bound by an adhesion molecule other than a claudin. Such adhesion molecules may be selected from the group consisting of integrins, cadherins, occludin, N-CAM, JAM, PE-CAM, desmogleins, desmocollins, fibronectin, lammin and other extracellular matrix proteins.

Within other aspects, a modulating agent may comprise an antibody or antigen-binding fragment thereof that specifically binds to the claudin-50 CAR sequence WKTSSYVG.

The present invention further provides pharmaceutical compositions comprising a cell adhesion modulating agent as described above, in combination with a pharmaceutically acceptable carrier. Such compositions may further comprise a drug. In addition, or alternatively, such compositions may further comprise one or more of: (a) a peptide comprising a cell adhesion recognition sequence that is bound by an adhesion molecule other than a claudin; and/or (b) an antibody or antigen-binding fragment thereof that specifically binds to a cell adhesion recognition sequence bound by an adhesion molecule other than a claudin.

Within further aspects, methods are provided for modulating cell adhesion, comprising contacting a claudin-expressing cell with a cell adhesion modulating agent as described above.

Within one such aspect, the present invention provides methods for increasing vasopermeability in a mammal, comprising administering to a mammal a cell adhesion modulating agent as provided above, wherein the modulating agent inhibits claudin-mediated cell adhesion.

Within another aspect, methods are provided for reducing unwanted cellular adhesion in a mammal, comprising administering to a mammal a cell adhesion modulating agent as provided above, wherein the modulating agent inhibits claudin-mediated cell adhesion.

In yet another aspect, the present invention provides methods for enhancing the delivery of a drug through the skin of a mammal, comprising contacting epithelial cells of a mammal with a cell adhesion modulating agent as provided above and a drug, wherein the modulating agent inhibits claudin-mediated cell adhesion, and wherein the step of contacting is performed under conditions and for a time sufficient to allow passage of the drug across the epithelial cells.

The present invention further provides methods for enhancing the delivery of a drug to a tumor in a mammal, comprising administering to a mammal a cell adhesion modulating agent as provided above and a drug, wherein the modulating agent inhibits claudin-mediated cell adhesion.

Within further aspects, the present invention provides methods for treating cancer in a mammal, comprising administering to a mammal a cell adhesion modulating agent as provided above, wherein the modulating agent inhibits claudin-mediated cell adhesion.

The present invention further provides methods for inhibiting angiogenesis in a mammal, comprising administering to a mammal a cell adhesion modulating agent as provided above, wherein the modulating agent inhibits claudin mediated cell adhesion.

Within further aspects, the present invention provides methods for enhancing drug delivery to the central nervous system of a mammal, comprising administering to a mammal a cell adhesion modulating agent as provided above, wherein the modulating agent inhibits claudin-mediated cell adhesion.

The present invention further provides methods for enhancing wound healing in a mammal, comprising contacting a wound in a mammal with a cell adhesion modulating agent as provided above, wherein the modulating agent enhances claudin mediated cell adhesion.

Within a related aspect, the present invention provides methods for enhancing adhesion of foreign tissue implanted within a mammal, comprising contacting a site of implantation of foreign tissue in a mammal with a cell adhesion modulating agent as provided above, wherein the modulating agent enhances claudin mediated cell adhesion.

The present invention further provides methods for inducing apoptosis in a claudin-expressing cell, comprising contacting a claudin-expressing cell with a cell adhesion modulating agent as provided above, wherein the modulating agent inhibits claudin-mediated cell adhesion.

The present invention further provides methods for identifying an agent capable of modulating claudin-mediated cell adhesion. One such method comprises the steps of (a) culturing cells that express a claudin in the presence and absence of a candidate agent, under conditions and for a time sufficient to allow cell adhesion; and (b) visually evaluating the extent of cell adhesion among the cells.

Within another embodiment, such methods may comprise the steps of: (a) culturing normal rat kidney cells in the presence and absence of a candidate agent, under conditions and for a time sufficient to allow cell adhesion; and (b) comparing the level of cell surface claudin and E-cadherin for cells cultured in the presence of candidate agent to the level for cells cultured in the absence of candidate agent.

Within a further embodiment, such methods may comprise the steps of: (a) culturing human aortic endothelial cells in the presence and absence of a candidate agent, under conditions and for a time sufficient to allow cell adhesion; and (b) comparing the level of cell surface claudin and N-cadherin for cells cultured in the presence of candidate agent to the level for cells cultured in the absence of candidate agent.

Within yet another embodiment, such methods comprise the steps of: (a) contacting an antibody that binds to a modulating agent comprising a claudin CAR sequence with a test compound; and (b) detecting the level of antibody that binds to the test compound.

The present invention further provides methods for detecting the presence of claudin-expressing cells in a sample, comprising: (a) contacting a sample with an antibody that binds to a claudin comprising a claudin CAR sequence under conditions and for a time sufficient to allow formation of an antibody-claudin complex; and (b) detecting the level of antibody-claudin complex, and there from detecting the presence of claudin-expressing cells in the sample.

Within further aspects, the present invention provides kits for detecting the presence of claudin-expressing cells in a sample, comprising: (a) an antibody that binds to a modulating agent comprising a claudin CAR sequence; and (b) a detection reagent.

The present invention further provides, within other aspects, kits for enhancing transdermal drug delivery, comprising: (a) a skin patch; and (b) a cell adhesion modulating agent, wherein the modulating agent comprises a claudin CAR sequence, and wherein the modulating agent inhibits claudin-mediated cell adhesion.

A detailed description of the above methods are described in PCT application WO 00/26360 (Blaschuck, O. W., et al.), incorporated herein in its entirety.

Further included in the present invention are methods of treating Clostridium perfringens or Clostridium difficile or Clostridium botulinum infections by targeting the enterotoxin, preferably Clostridium perfringens enterotoxin. Clostridium enterotoxin (CE) binds to Claudin-50. Purified Claudin-50 polypeptides can be used to absorb CE to prevent CE's cytotoxic effects on cells. Preferred CE binding Claudin-50 polypeptides include the full length and mature Claudin-50 polypeptide and fragments comprising the extracellular domains, amino acid residues 29 to 81 and 103 to 116. Further preferred CE binding Claudin-50 polypeptides include the extracellular domain 29 to 81 and fragments comprising the CAR sequence. CE binding Claudin-50 polypeptides may further be recombinantly fused or chemically coupled (covalently or non-covalently) to a heterologous polypeptide, molecule, or support. Means of administering CE binding Claudin-50 polypeptide compositions are those well known for administering biologically active polypeptides. Preferably, CE binding Claudin-50 polypeptide compositions are administered in at least equamolar concentration compared with CE. More preferably, CE binding Claudin-50 polypeptide compositions are administered in at least a 10 to 100 fold molar excess concentration compared with CE.

The above CE binding Claudin-50 polypeptides are also useful for affinity purification CE. For example, CE binding Claudin-50 polypeptides can be fixed or coupled to a solid support in a column and used to bind CE in a biological sample. CE can be released from the column for example, by using a salt gradient.

CE binding Claudin-50 polypeptide compositions are also useful in detecting and diagnosing Clostridium perfringens infection. The presence of CE indicates Clostridium perfringens infection. The level of CE is proportional to the level or degree of the disease or infection. Moreover, the degree of cellular disruption at tight junctions is also proportional to the level of CE. CE binding Claudin-50 polypeptides will preferentially bind endogenous claudins at the sites of tight junction disruptions. CE binding Claudin-50 polypeptides can therefore be used to detect or diagnose Clostridium perfringens infection by either binding CE or by binding sites of tight junction disruption. Biological samples including fluids and tissue samples can be assayed using methods well known in the art. Clostridium perfringens infections can further be localized in vivo using CE binding Claudin-50 polypeptides in in vivo imaging.

Protein of SEQ ID NO: 703

The protein of SEQ ID NO: 703 encoded by the extended cDNA SEQ ID NO: 61 and expressed in lymphocytes exhibits an extensive homology to a stretch of 91 amino acid of a human secreted protein expressed in peripheral blood mononucleocytes (Genpep accession number W36955 and Genseq accession number V00433). The amino acid residues are identical except for the substitution of asparagine to isoleucine at positions 94, and the conservative substitutions at positions 108, 109 and 110 of the 110 amino acids long matched protein.

Protein of SEQ ID NO: 704

The protein of SEQ ID NO: 704 encoded by the extended cDNA SEQ ID NO: 62 exhibits extensive homologies to stretches of proteins encoding vacuolar proton-ATPase subunits 9.2 of either human (Genbank accession number Y15286) or bovine species (Genbank accession umber Y15285). These two highly conserved proteins are extremely hydrophobic membrane roteins with two membrane-spanning helices and a potential metal-binding domain conserved in mammalian protein homologues (Ludwig et al., J. Biol. Chem., 273:10939-10947 (1998)). The amino acid residues are completely identical, the protein of SEQ ID NO: 704 is missing amino acids 1 to 92 from the Genbank sequences. The protein of SEQ ID NO: 704 contains the second putative transmembrane domain as well as the potential metal-binding site.

Taken together, these data suggest that the protein of SEQ ID NO: 704 may play a role in energy conservation, secondary active transport, acidification of intracellular compartments and/or cellular pH homeostasis. Preferred fragments of SEQ ID NO: 704 and the corresponding mature polypeptide encoded by the human cDNA of the deposited clone are those with inositol ATPase activity. Further preferred are fragments with not less then 100 fold less activity, not less than 10 fold activity, and not less than 5 fold activity when compared to mature protein.

Protein of SEQ ID NO: 705

The protein of SEQ ID NO: 705 encoded by the extended cDNA SEQ ID NO: 63 shows homology to short stretches of Drosophila, C. elegans and chloroplast proteins similar to E. coli ribosomal protein L16.

Taken together, these data suggest that the protein of SEQ ID NO: 705 may be a ribosomal protein.

Protein of SEQ ID NO: 706

The protein of SEQ ID NO: 706, encoded by the cDNA of SEQ ID NO:64, is a chemokine. The protein can be used to attract and activate monocytes and lymphocytes, especially to a site of infection or tumor. The protein can also be used in in vivo imaging to identify/locate/diagnose sites of infection or tumors. Preferred fragments of SEQ ID NO: 706 and the corresponding mature polypeptide encoded by the human cDNA of the deposited clone are those with the above activities. Further preferred are fragments with not less then 100 fold less activity, not less than 10 fold activity, and not less than 5 fold activity when compared to mature protein.

Protein of SEQ ID NO: 709

The protein of SEQ ID NO: 709, encoded by the extended cDNA SEQ ID NO: 67, is human Connexin 31.1. Connexins are a family of integral membrane proteins that oligomerize into clusters of intercellular channels called gap junctions, which join cells in virtually all metazoans. These channels permit exchange of ions between neurons and between neurons and excitable cells such as myocardiocytes (for review, see Goodenough et al., Ann. Rev. Biochem., 65:475-502 (1996)).

Human connexin 31.1 is expressed only in the skin, with Connexin 31.1 mRNA being 15-30 times more abundant in mature skin than in fetal skin. Within the skin layers, human Connexin 31.1 expression is localized to the keratinocyte layer. Human Connexin 31.1. is therefore useful as a marker for skin, particularly the keratinocyte layer, as well as keratinocytes, using either human Connexin 31.1 polynucleotides or antibodies made to human Connexin 31.1 polypeptides. Moreover, human Connexin 31.1 is useful as a marker for skin tumors because, whereas hyperplasia express Connexin 31.1, skin tumors at all stages do not. Hence, Connexin 31.1 polynucleotides and polupeptides are useful for differentiating between a skin hyperplasia and a tumor.

Human Connexin 31.1 is also useful in the methods for treating cancer, perferrably skin tumors, more preferably skin tumors involving keratinocytes. Preferred methods of using Human Connexin 31.1 for treating cancer includes the methods described in PCT application WO 97/28179 (Fick, J. R. et al.) incorporated herein in its entirety. Preferred fragments of SEQ ID NO: 709 and the corresponding mature polypeptide encoded by the human cDNA of the deposited clone are those with useful in the above methods, e.g., antigenic fragments and those fragments which form gap junctions.

Protein of SEQ ID NO: 710

The protein of SEQ ID NO: 710 encoded by the extended cDNA SEQ ID NO: 68 shows homologies with different DNA or RNA binding proteins such as the human Staf50 transcription factor (Genbank accession number X82200), the human Ro/SS-A ribonucleoprotein autoantigen (Swissprot accession number P19474) or the murine RPT1 transcription factor (Swissprot accession number P15533). The protein of SEQ ID NO: 710 exhibits a putative signal peptide and also a PROSITE signature for a RING type zinc finger domain located from positions 15 to 59. Secreted proteins may have nucleic acid binding domain as shown by a nematode protein thought to regulate gene expression which exhibits zinc fingers as well as a functional signal peptide (Holst and Zipfel, J. Biol. Chem., 271:16275-16733 (1996)).

Taken together, these data suggest that the protein of SEQ ID NO: 710 may play a role in protein-protein interaction in intracellular signaling and eventually may directly or indirectly bind to DNA and/or RNA, hence regulating gene expression.

Protein of SEQ ID NO: 712

The protein of SEQ ID NO: 712 encoded by the extended cDNA SEQ ID NO: 70 exhibits extensive homologies to proteins encoding RING zinc finger proteins of the human, chicken and rodent species, as well as an EGF-like domain. Two stretches of 341 and of 13 amino acids of the human RING zinc finger protein which might bind DNA (Genbank accession number AF037204). The amino acid residues are identical except for conservative substitutions at positions 18, 29, 156 and 282 of the 381 amino acid long human RING zinc finger. Such RING zinc finger proteins are thought to be involved in protein-protein interaction and are especially found in nucleic acid binding proteins. Secreted proteins may have nucleic acid binding domain as shown by a nematode protein thought to regulate gene expression which exhibits zinc fingers as well as a functional signal peptide (Holst and Zipfel, J. Biol. Chem., 271:16275-16733 (1996)).

Taken together, these data suggest that the protein of SEQ ID NO: 712 may play a role in protein-protein interaction or be a nucleic acid binding protein.

Proteins of SEQ ID NOs: 713 and 739

The proteins of SEQ ID NOs: 713 and 739 encoded by the extended cDNA SEQ ID NOs: 70 and 96, respectively, belong to the stomatin or band 7 family. The human stomatin is an integral membrane phosphoprotein thought to be involved to regulate the cation conductance by interacting with other proteins of the junctional complex of the membrane skeleton (Gallagher and Forget, J. Biol. Chem., 270:26358-26363 (1995)). The proteins of SEQ ID NOs: 713 and 739 exhibit the PROSITE signature typical for the band 7 family signature.

The proteins of SEQ ID NOs: 713 and 739 play a role in the regulation of ion transport, hence in the control of cellular volume. These proteins are useful in diagnosing and/or treating stomatocytosis and/or cryohydrocytosis by detecting a decreased level or absence of the proteins or alternatively by detecting a mutation or deletion affecting tertiary structure of the proteins.

Protein of SEQ ID NO: 725 and 740

The proteins of SEQ ID NO: 213 and 229, encoded by the cDNA of SEQ ID NO: 83 and 98, respectively, is human Glia Maturation Factor-gamma 2 (GMF-gamma 2). SEQ ID NO: 740 differs from SEQ ID NO: 725 in that SEQ ID NO: 740 has additional amino acids at the N-terminus. The following description applies equally to both SEQ ID NO: 725 and 740. A preferred use of GMF-gamma 2 is to stimulate neurite outgrowth or neurite re-sprouting. These methods include both in vitro and in vivo uses, but preferred uses are those for treating neural injuries and cancer as disclosed in WO9739133 and WO9632959, incorporated herein in their entireties.

GMF-gamma 2 may also be used as a neurotrophic and as a neuroprotective agent against toxic insults, such as ethonal and other neurotoxic agents. GMF-gamma2 may be used as a neurotrophic or neuroprotective agent either in vitro or in vivo. A preferred target of GMF-gamma 2 as a neurotrophic or neuroprotective agent are primary neurons.

GMF-gamma 2 may further be used to stimulate the expression and secretion of NGF and BDNF in glial cells both in vitro and in vivo. Conditioned media from cells treated with GMF-gamma 2 is useful as a source of NGF and BDNF. GMF-gamma 2 may further be used to target cells directly or by recombinantly fusing GMF-gamma 2 to a heterologous protein, such as a ligand or antibody specific to the target cell (e.g., glial cells). Alternatively, GMF-gamma 2 may be fused or covalently or non-covalently coupled to a heterologous protein or other biological or non-biological molecule wherein the heterologous protein or molecule is used as this targeting reagent.

Preferred fragments of SEQ ID NOs: 725 and 740 and the corresponding polypeptide encoded by the human cDNAs of the deposited clones are those with the above activities. Further preferred are fragments with not less then 100 fold less activity, not less than 10 fold activity, and not less than 5 fold activity when compared to the protein of SEQ ID NO: 740 or the protein encoded by the corresponding human cDNA of the deposited clone.

Protein of SEQ ID NO: 726:

The protein of SEQ ID NO: 726 encoded by the extended cDNA SEQ ID NO: 84 isolated from brain shows extensive homology to a human SH3 binding domain glutamic acid-rich like protein or SH3BGRL (Egeo et al, Biochem. Biophys. Res. Commun., 247:302-306 (1998)) with Genbank accession number is AF042081. The amino acid residues are identical to SH3BGRL except for positions 63 and 101 in the 114 amino acid long matched sequence. This SH3BRGL protein is itself homologous to the middle proline-rich region of a protein containing an SH3 binding domain, the SH3BGR protein (Scartezzini et al., Hum. Genet., 99:387-392 (1997)). This proline-rich region is also highly conserved in mice. Both SH3BGR and SH3BGRL proteins are thought to be involved in the Down syndrome pathogenesis. The protein SEQ ID NO: 726 also contains the proline-rich SH3 binding domain (bold) and a potential RGD cell attachment sequence (underlined).

SH3 domains are small important functional modules found in several proteins from all eukaryotic organisms that are involved in a whole range of regulation of protein-protein interaction, e.g. in regulating enzymatic activities, recruiting specific substrates to the enzyme in signal transduction pathways, in interacting with viral proteins and they are also thought to play a role in determining the localization of proteins to the plasma membrane or the cytoskeleton (for a review, see Cohen et al, Cell, 80:237-248 (1995)).

The Arg-Gly-Asp (RGD) attachment site promote cell adhesion of a large number of adhesive extracellular matrix, blood and cell surface proteins to their integrin receptors which have been shown to regulate cell migration, growth, differentiation and apoptosis. This cell adhesion activity is also maintained in short RGD containing synthetic peptides which were shown to exhibit anti-thrombolytic and anti-metastatic activities and to inhibit bone degradation in vivo (for review, see Ruoslahti, Annu. Rev. Cell Dev. Biol., 12:697-715 (1996)).

Taken together, these data suggest that the protein of SEQ ID NO: 726 may be important in regulating protein-protein interaction in signal transduction pathways, and/or may play a role of localization of proteins to the plasma membrane or cytoskeleton, and/or may play a role in cell adhesion. Moreover, this protein or part therein, especially peptides containing the RGD motif, may be useful in diagnosing and treating cancer, thrombosis, osteoporosis and/or in diagnosing and treating disorders associated with the Down syndrome.

Protein of SEQ ID NO: 728

The protein of SEQ ID NO: 728 found in testis encoded by the extended cDNA SEQ ID NO: 86 shows homologies to protein domains with a 4-disulfide core signature found in either an extracellular proteinase inhibitor named chelonianin (Swissprot accession number P00993) or in rabbit and human proteins specifically expressed in epididymes (Genbank accession numbers U26725 and R13329). The matched domain in red sea turtle chelonianin is known to inhibit subtilisin, a serine protease (Kato and Tominaga, Fed. Proc., 38:832 (1979)). All cysteines of the 4 disulfide core signature thought to be crucial for biological activity are present in the protein of SEQ ID NO: 728. The 4 disulfide core signature is present except for a conservative substitution of asparagine to glutamine.

Taken together, these data suggest that the protein of SEQ ID NO: 728 may play a role in protein-protein interaction, act as a protease inhibitor and/or may also be related to male fertility.

Protein of SEQ ID NO: 735

The protein of SEQ ID NO: 735 encoded by the extended cDNA SEQ ID NO: 93 shows homology to short stretches of a human protein called Tspan-1 (Genbank accession number AF054838) which belongs to the 4 transmembrane superfamily of molecular facilitators called tetraspanin (Meakers et al., FASEB J., 11:428-442 (1997)).

Taken together, these data suggest that the protein of SEQ ID NO: 735 may play a role in cell activation and proliferation, and/or adhesion and motility and/or differentiation and cancer.

Protein of SEQ ID NO: 532

The protein of SEQ ID NO: 532 encoded by the extended cDNA SEQ ID NO: 175 isolated from lymphocyte shows complete identity to a human protein TFAR19 that may play a role in apoptosis (Genbank accession number AF014955) as shown by the alignment in FIG. 10.

Taken together, these data suggest that the protein of SEQ ID NO: 532 may be involved in the control of development and homeostasis. Thus, this protein may be useful in diagnosis and/or treating several types of disorders including, but not limited to, cancer, autoimmune disorders, viral infections such as AIDS, neurodegenerative disorders, osteoporosis.

Proteins of SEQ ID NOs: 489, 490 and 547

The proteins of SEQ ID NOs: 174, 175 and 232 encoded by the extended cDNAs SEQ ID NOs:. 132, 133 and 190 respectively and isolated from lymphocytes shows complete extensive homologies to a human secreted protein (Genseq accession number W36955). As shown by the alignments of FIG. 11, the amino acid residues are identical to those of the 110 amino acid long matched protein except for positions 51 and 108-110 of the matched protein for the protein of SEQ ID NOs: 489, for positions 48, 94 and 108-110 of the matched protein of SEQ ID NOs:490 and for positions 94, and 108-110 of the matched protein for the protein of SEQ ID NOs: 547. Proteins of SEQ ID NOs: 489 and 547 may represent alternative forms issued from alternative use of polyadenylation signals.

Taken together, these data suggest that the proteins of SEQ ID NOs: 489, 490 and 547 may play a role in cell proliferation and/or differentiation, in immune responses and/or in haematopoeisis. Thus, this protein or part therein, may be useful in diagnosing and treating several disorders including, but not limited to, cancer, immunological, haematological and/or inflammatory disorders. It may also be useful in modulating the immune and inflammatory responses to infectious agents and/or to suppress graft rejection.

Proteins of SEQ ID NO: 546

The protein of SEQ ID NO: 546 encoded by the extended cDNA SEQ ID NO: 189 shows extensive homology with the human E25 protein (Genbank accession number AF038953). As shown by the alignments in FIG. 12, the amino acid residues are identical except for position 159 in the 263 amino acid long matched sequence. The matched protein might be involved in the development and differentiation of haematopoietic stem/progenitor cells. In addition, it is the human homologue of a murine protein thought to be involved in chondro-osteogenic differentiation and belonging to a novel multigene family of integral membrane proteins (Deleersnijder et al, J. Biol. Chem., 271: 19475-19482 (1996)).

The protein of invention contains two short segments from positions 1 to 21 and from 100 to 120 as predicted by the software TopPred II (Claros and von Heijne, CABIOS applic. Notes, 10: 685-686 (1994)). The first transmembrane domains matches exactly those predicted for the murine E25 protein.

Taken together, these data suggest that the protein of SEQ ID NO: 546 may be involved in cellular proliferation and differentiation. Thus, this protein may be useful in diagnosing and/or treating several types of disorders including, but not limited to, cancer and embryogenesis disorders.

Protein of SEQ ID NO: 511

The protein of SEQ ID NO: 511 encoded by the extended cDNA SEQ ID NO: 154 shows extensive homology with the human seventransmembrane protein (Genbank accession number Y11395) and its murine homologue (Genbank accession number Y11550). As shown by the alignments in FIG. 13, the amino acid residues are identical except for position 174 in the 399 amino acid long human matched sequence. The matched protein potentially associated to stomatin may act as a G-protein coupled receptor and is likely to be important for the signal transduction in neurons and haematopoietic cells (Mayer et al, Biochem. Biophys. Acta., 1395: 301-308 (1998)).

Taken together, these data suggest that the protein of SEQ ID NOs: 511 may be involved in signal transduction. Thus, this protein may be useful in diagnosing and/or treating several types of disorders including, but not limited to, cancer, neurodegenerative diseases cardiovascular disorders, hypertension, renal injury and repair and septic shock.

Protein of SEQ ID NO: 473

The protein of SEQ ID NOs: 473 encoded by the extended cDNA SEQ ID NO: 116 shows homology with the murine subunit 7a of the COP9 complex (Genbank accession number AF071316). As shown by the alignments in FIG. 14, the amino acid residues are identical except for positions 90, 172 and 247 in the 275 amino acid long matched sequence. This complex is highly conserved between mammals and higher plants where it has been shown to act as a repressor of photomorphogenesis All the components of the mammalian COP9 complex contain structural features also present in components of the proteasome regulatory complex and the translation initiation complex eIF3 complex, suggesting that the mammalian COP9 complex is an important cellular regulator modulating multiple signaling pathways (Wei et al, Curr. Biol., 8 919-922 (1998)).

Taken together, these data suggest that the protein of SEQ ID NO: 473 may be involved in cellular signaling, probably as a subunit of the human COP9 complex. Thus, this protein may be useful in diagnosing and/or treating several types of disorders including, but not limited to, cancer, neurodegenerative diseases, cardiovascular disorders, hypertension, renal injury and repair and septic shock.

Protein of SEQ ID NO: 541

The protein of SEQ ID NO:541 encoded by the extended cDNA SEQ ID NO: 184 shows homology with the bovine subunit B14.5B of the NADH-ubiquinone oxidureductase complex (Arizmendi et al, FEBS Lett., 313: 80-84 (1992) and Swissprot accession-number Q02827, SEQ ID NO: 514). As shown by the alignments in FIG. 15, the amino acid residues are identical except for positions 3-4,6-12, 32-34, 47, 53-55, 67 and 69-74 in the 120 amino acid long matched sequence. This complex is the first of four complexes located in the inner mitochondrial membrane and composing the mitochondrial electron transport chain. Complex I is involved in the dehydrogenation of NADH and the transportation of electrons to coenzyme Q. It is composed of 7 subunits encoded by the mitochondrial genome and 34 subunits encoded by the nuclear genome. It is also thought to play a role in the regulation of apoptosis and necrosis. Mitochondriocytopathies due to complex I deficiency are frequently encountered and affect tissues with a high energy demand such as brain (mental retardation, convulsions, movement disorders), heart (cardiomyopathy, conduction disorders), kidney (Fanconi syndrome), skeletal muscle (exercise intolerance, muscle weakness, hypotonia) and/or eye (opthmaloplegia, ptosis, cataract and retinopathy). For a review on complex I see Smeitink et al., Hum. Mol. Gent., 7: 1573-1579 (1998).

Taken together, these data suggest that the protein of SEQ ID NO:541 may be part of the mitochondrial energy-generating system, probably as a subunit of the NADH-ubiquinone oxidoreductase complex. Thus, this protein or part therein, may be useful in diagnosing and/or treating several disorders including, but not limited to, brain disorders (mental retardation, convulsions, movement disorders), ‘heart disorders (cardiomyopathy, conduction disorders), kidney disorders (Fanconi syndrome), skeletal muscle disorders (exercise intolerance, muscle weakness, hypotonia) and/or eye disorders opthmalmoplegia, ptosis, cataract and retinopathy).

Proteins of SEQ ID NOs: 464, 465 and 526

The proteins of SEQ ID NOs: 464, 465 and 526 encoded by the extended cDNAs SEQ ID NOs: 107, 108 and 169 respectively and found in, skeletal muscle shows homologies with T1/ST2 ligand polypeptide of either human (Genbank accession number U41804 and Genseq accession number WO9639) or rodent species (Genbank accession number U41805 and Genseq accession number WO9640). These polypeptides are thought to be cytokines that bind to the ST2 receptor, a member of the immunoglobulin family homologous to the interleukin-1 receptor and present on some lymphoma cells. They are predicted to be cell-surface proteins containing a short transmembrane domain. (Gayle et al, J. Biol. Chem., 271: 5784-5789 (1996)). Proteins of SEQ ID NOs: 464, 465 and 526 may represent alternative forms issued from alternative use of polyadenylation signals.

The protein of invention contains two short transmembrane segments from positions 5 to 25 and from 195 to 215 as predicted by the software TopPred II (Claros and von Heijne, CABIOS applic. Notes, 10:685-686 (1994)). The second transmembrane domain matches exactly those of the matched cell-surface protein.

Taken together, these data suggest that the protein of SEQ ID NOs: 464, 465 and 526 may act as a cytokine, thus may play a role in the regulation of cell growth and differentiation and/or in the regulation of the immune response. Thus, this protein or part therein, may be useful in diagnosing and treating several disorders including, but not limited to, cancer, immunological, haematological and/or inflammatory disorders. It may also be useful in modulating the immune and inflammatory responses to infectious agents such as HIV and/or to suppress graft rejection.

Protein of SEQ ID NO: 492

The protein SEQ ID NO: 492 found in testis encoded by the extended cDNA SEQ ID NO: 135 shows homologies to serine protease inhibitor proteins belonging to the pancreatic trypsin inhibitor family (Kunitz) such as the extracellular proteinase inhibitor named chelonianin (Swissprot accession number P00993). The characteristic PROSITE signature of this family is conserved in the protein of the invention (positions 69 to 87) except for a drastic change of the last cysteine residue into an arginine residue.

Taken together, these data suggest that the protein of SEQ ID NO: 492 may be a protease inhibitor, probably of the Kunitz family. Thus, this protein or part therein, may be useful in diagnosing and treating several disorders including but not limited to, cancer and neurodegenerative disorders such as Alzheimer's disease.

Protein of SEQ ID NO: 461

The protein SEQ ID NO: 461 encoded by the extended cDNA SEQ ID NO: 104 shows homology to human apolipoprotein L (Genbank accession number AF019225). The matched protein is a secreted high density lipoprotein associated with apoA-1-containing lipoproteins which play a key role in reverse cholesterol transport.

Taken together, these data suggest that the protein of SEQ ID NO. 461 may play a role in lipid metabolism. Thus, this protein may be useful in diagnosing and/or treating several types of disorders including, but not limited to, hyperlipidemia, hypercholesterolemia, atherosclerosis, cardiovascular disorders such as, coronary heart disease, and neurodegenerative disorders such as Alzheimer's disease or dementia.

Protein of SEQ ID NO: 478

The protein SEQ ID NO: 478 encoded by the extended cDNA SEQ ID NO: 121 shows homology to the yeast autophagocytosis protein AUT1 (SwissProt accession number P40344). The matched protein is required for starvation-induced non-specific bulk transport of cytoplasmic proteins to the vacuole.

Taken together, these data suggest that the protein of SEQ ID NO: 478 may play a role in protein transport. Thus, this protein may be useful in diagnosing and/or treating several types of disorders including, but not limited to, autoimmune disorders and immune disorders due to dysfunction of antigen presentation.

Protein of SEQ ID NO: 529

The protein of SEQ ID NO: 529 encoded by the extended cDNA SEQ ID NO: 172 and expressed in adult brain shows extensive homology to part of the murine SHYC protein (Genbank accession number AF072697) which is expressed in the developing and embryonic nervous system as well as along the olfactory pathway in adult brains (Koster et al., Neuroscience Letters., 252: 69-71 (1998)).

Taken together, these data suggest that the protein of SEQ ID NO: 529 may play a role in nervous system development and function. Thus, this protein may be useful in diagnosing and/or treating cancer and/or brain disorders, including neurodegenerative disorders such as Alzheimer's and Parkinson's diseases.

Protein of SEQ ID NO: 540

The protein of SEQ ID NO: 540 encoded by the extended cDNA SEQ ID NO: 183 and expressed in adult prostate belong to the phosphatidylethanolainin-binding protein from which it exhibits the characteristic PROSITE signature from positions 90 to 112 (see table VIII). Proteins from this widespread family, from nematodes to fly, yeast, rodent and primate species, bind hydrophobic ligands such as phospholipids and nucleotides. They are mostly expressed in brain and in testis and are thought to play a role in cell growth and/or maturation, in regulation of the sperm maturation, motility and ‘in membrane remodeling. They may act either through signal transduction or through oxidoreduction reactions (for a review see Schoentgen and Jollès, FEBS Letters, 369: 22-26 (1995)).

Taken together, these data suggest that the protein of SEQ ID NO: 540 may play a role in cell. Thus, these growth, maturation and in membrane remodeling and/or may be related to male fertility. Thus, this protein may be useful in diagnosing and/or treating cancer, neurodegenerative diseases, and/of, disorders related to male fertility and sterility.

Protein of SEQ ID NO: 468

The protein of SEQ ID NO: 468 encoded by the extended cDNA SEQ ID NO. 111 and expressed in brain exhibits homology to different integral membrane proteins. These membrane proteins include the nematode protein SRE-2 (Swissprot accession number Q09273) that belongs to the multigene SRE family of C. elegans receptor-like proteins and a family of tricarboxylate carriers conserved between flies and mammals. One member of this matched family is the rat tricarboxylate carrier (Genbank accession number S70011), an anion transporter localized in the inner membrane of mitochondria and involved in the biosynthesis of fatty acids and cholesterol. The protein of the invention contains a short transmembrane segments from positions 5 to 25 as predicted by the software TopPred II (Claros and von Heijne, CABIOS applic. Notes, 10:685-686 (1994)).

Taken together, these data suggest that the protein of SEQ ID NO: 468 may play a role in signal transduction and/or in molecule transport. Thus, this protein may be useful in diagnosing and/or treating several types of disorders including, but not limited to, cancer, neurodegenerative diseases, immune disorders, cardiovascular disorders, hypertension, renal injury and repair and septic shock.

Protein of SEQ ID NO: 528

The protein of SEQ ID NO: 528 encoded by the extended cDNA SEQ ID NO: 171 and expressed in brain exhibits homology with part of the tRNA pseudouridine 55 synthase found in Escherichia Coli (Swissprot accession number P09171). This bacterial protein belongs to the NAP57/CBF5/TRUB family of nuclieolar proteins found in bacteria, yeasts and mammals involved in rRNA or tRNA biosynthesis, ribosomal subunit assembly and/or centromere/mircotubule binding.

Taken together, these data suggest that the protein of SEQ ID NO:528 may play a role in rRNA or tRNA biogensis and function. Thus, this protein may be useful in diagnosing and/or treating several types of disorders including, but not limited to, cancer, hearing loss or disorders linked to chromosomal instability such as dyskeratosis.

Protein of SEQ ID NO: 555

The protein of SEQ ID NO: 555 encoded by the extended cDNA SEQ ID NO: 198 and expressed in brain exhibits homology with a family of eukaryotic cell surface antigens containing 4 transmembrane domains. The PROSITE signature for this family is conserved in the protein of the invention except for a substitution of an alanine residue in place of any of the following hydrophic residues: leucine, valine, isoleucine or methionine (positions 21 to 36).

The protein of the invention contains three short transmembrane segments from positions 6 to 26, 32 to 52 and from 56 to 76 as predicted by the software TopPred II (Claros and von Heijne, CABIOS applic. Notes, 10: 685-686 (1994)). These transmembrane domains match the last three transmembrane domains of the matched protein family.

Taken together, these data suggest that the protein of SEQ ID NO: 555 may play a role in immunological and/or inflammatory responses, probably as a cell surface antigen. Thus, this protein or part therein, may be useful in diagnosing and treating several disorders including, but not limited to, cancer, immunological, haematological and/or inflammatory disorders. It may also be useful in modulating the immune and inflammatory responses to infectious agents and/or to suppress graft rejection.

Protein of SEQ ID NO: 554

The protein of SEQ ID NO: 554 encoded by the extended cDNA SEQ ID NO: 197 exhibits homology with a conserved region in a family of NA+/H+ exchanger conserved in yeast, nematode and mammals. These cation/proton exchangers are integral membrane proteins with 5 transmembrane segments involved in intracellular pH regulation, maintenance of cell volume, reabsorption of sodium across specialized epithelia, vectorial transport and are also thought to play a role in signal transduction and especially in the induction of cell proliferation and in the induction of apoptosis.

The protein of invention contains four short transmembrane segments from positions 21 to 41, 48 to 68 and from 131 to 151 as predicted by the software TopPred II (Claros and von Heijne, CABIOS applic. Notes, 10: 685-686 (1994)). The third and fourth transmembrane domains match the fourth and fifth transmembrane segments of the matched family of proteins.

Taken together, these data suggest that the protein of SEQ ID NO: 554 may play a role in membrane permeability and/or in signal transduction. Thus, this protein may be useful in diagnosing and/or treating several types of disorders including, but not limited to, cancer, neurodegenerative diseases, cardiovascular disorders, hypertension, renal injury and repair, septic shock as well as disorders of membrane permeability such as diarrhea.

Protein of SEQ ID NO: 515

The protein of SEQ ID NO:515 encoded by the extended cDNA SEQ ID NO: 158 and expressed in brain exhibits extensive homology to the N-terminus of cell division cycle protein 23 (Genbank accession number AF053977) and also to a lesser extent to its homologue in Saccharomyces cerevisiae. The matched protein is required for chromosome segregation and is part of the anaphae-promoting complex necessary for cell cycle progression to mitosis.

Taken together, these data suggest that the protein of SEQ ID NO: 515 may play a role in cellular mitosis. Thus, this protein may be useful in diagnosing and/or treating several types of disorders including, but not limited to, cancer and leukemia.

Protein of SEQ ID NO: 545

The protein of SEQ ID NO: 545 encoded by the extended cDNA SEQ ID NO: 188 exhibits extensive homology to the C-terminus of the eta subunit of T-complex polypeptide 1 conserved from yeasts to mammals, and even complete identity with the last 54 amino acid residues of the human protein (Genbank accession number AF026292). The matched protein is a chaperonin which assists the folding of actins and tubulins in eukaryotic cells upon ATP hydrolysis.

Taken together, these data suggest that the protein of SEQ ID NO:545 may play a role in the folding, transport, assembly and degradation of proteins. Thus, this protein may be useful in diagnosing and/or treating several types of disorders including, but not limited to, cancer, cardiovascular disorders, immune disorders, neurodegenerative disorders, osteoporosis and arthritis.

Protein of SEQ ID NO: 482

The protein of SEQ ID NO: 482 encoded by the extended cDNA SEQ ID NO: 125 exhibits homology to a monkey pepsinogen A-4 precursor (Swissprot accession number P27678) and to related members of the aspartyl protease family. The matched protein belongs to a family of widely distributed proteolytic enzymes known to exist in vertebrate, fungi, plants, retroviruses and some plant viruses.

Taken together, these data suggest that the protein of SEQ ID NO: 482 may play a role in the degradation of proteins. Thus, this protein may be useful in diagnosing and/or treating several types of disorders including, but not limited to, cancer, autoimmune disorders and immune disorders due to dysfunction of antigen presentation.

Protein of SEQ ID NO: 494

The protein of SEQ ID NO: 494 encoded by the extended cDNA SEQ ID NO: 137 found in testis exhibits homology to part of mammalian colipase precursors. Colipases are secreted cofactors for pancreatic lipases that allow the lipase to anchor at the water-lipid interface. Colipase plays a crucial role in the intestinal digestion and absorption of dietary fats. The 5 cysteines characteristic for this protein family are conserved in the protein of the invention although the colipase PROSITE signature is not.

Taken together, these data suggest that the protein of SEQ ID NO: 494 may play a role in the lipid metabolism and/or in male fertility. Thus, this protein may be useful in diagnosing and/or treating several types of disorders including, but not limited to, hyperlipidemia, hypercholesterolemia, atherosclerosis, cardiovascular disorders such as coronary heart disease, and neurodegenerative disorders such as Alzheimer's disease or dementia, and disorders linked to male fertility.

Protein of SEQ ID NO: 542

The protein of SEQ ID NO: 542 encoded by the extended cDNA SEQ ID NO: 185 exhibits extensive homology to the ATP binding region of a whole family of serine/threonine protein kinases belonging to the CDC2/CDC28 subfamily. The PROSITE signature characteristic for this domain is present in the protein of the invention from positions 10 to 34.

Taken together, these data suggest that the protein of SEQ ID NO: 542 may bind ATP, and even be a protein kinase. Thus, this protein may be useful in diagnosing and/or treating several types of disorders including, but not limited to, cancer, neurodegenerative diseases, cardiovascular disorders, hypertension, renal injury and repair and septic shock.

Protein of SEQ ID NO: 776 (Internal Designation 26-44-1-B5-CL31)

The protein of SEQ ID NO: 776 encoded by the extended cDNA SEQ ID NO: 371 isolated from ovary shows extensive homology to a human protein called phospholemman or PLM and its homologues in rodent and canine species. PLM is encoded by the nucleic acid sequence of Genbank accession number U72245. Phospholemman is a prominent plasma membrane protein whose phosphorylation correlates with an increase in contractility of myocardium and skeletal muscle. Initially described as a simple chloride channel, it has recently been shown to be a channel for taurine that acts as an osmolyte in the regulation of cell volume (Moorman et al, Adv Exp. Med. Biol., 442:219-228 (1998)).

As shown by the alignment in FIG. 10 between tha protein of SEQ ID NO:776 and PLM, the amino acid residues are identical except for positions 3 and 5 in the 92 amino acid long matched protein. The substitution of a proline residue at position 3 par another neutral residue, serine, is conservative. In addition, the protein of the invention also exhibits the typical ATP1G/PLM/MAT8 PROSITE signature (position 27 to 40 in bold in FIG. 10) for a family containing mostly proteins known to be either chloride channels or chloride channel regulators In addition, the protein of invention contains 2 short transmembrane segments from positions 1 to 21 and from 37 to 57 as predicted by the software TopPred II (Claros and von Heijne, CABIOS applic. Notes, 10:685-686 (1994)). The first segment (in italic) corresponds to the signal peptide of PLM and the second transmembrane domains (underlined) matches the transmembrane region (double-underlined) shown to be the chloride channel itself (Chen et al., Circ. Res., 82:367-374 (1998)).

Taken together, these data suggest that the protein of SEQ ID NO: 776 may be involved in the regulation of cell volume and in tissue contractility. Thus, this protein may be useful in diagnosing and/or treating several types of disorders including, but not limited to, cancer, diarrhea, fertility disorders, and in contractility disorders including muscle disorders, pulmonary disorders and myocardial disorders.

Proteins of SEQ ID NOs: 777 (Internal Designation 47-4-4-C6-CL23)

The protein of SEQ ID NO: 777 encoded by the extended cDNA SEQ ID NO: 372 found in substantia nigra shows extensive homology with the human E25 protein. The E25 protein 35 is encoded by the nucleic acid sequence of Genbank accession number AF038953. The matched protein might be involved in the development and differentiation of haematopoietic stem/progenitor cells. In addition, it is the human homologue of a murine protein thought to be involved in chondro-osteogenic differentiation and belonging to a novel multigene family of integral membrane proteins (Deleersnijder et al, J. Biol. Chem., 271:19475-19482 (1996)).

As shown by the alignments in FIG. 11 between the protein of SEQ ID NO:777 and E25, the amino acid residues are identical except for positions 9, 24 and 121 in the 263 amino acid long matched sequence. All these substitutions are conservative. In addition, the protein of invention contains one short transmembrane segment from positions 1 to 21 (underlined in FIG. 11) matching the one predicted for the murine E25 protein as predicted by the software TopPred II (Claros and von Heijne, CABIOS applic. Notes, 10:685-686 (1994)).

Taken together, these data suggest that the protein of SEQ ID NO:777 may be involved in cellular proliferation and differentiation, and/or in haematopoiesis. Thus, this protein may be useful in diagnosing and/or treating several types of disorders including, but not limited to, cancer, hematological, chondro-osteogenic and embryogenetic disorders.

Proteins of SEQ ID NO: 784 (internal designation 58-34-2-H8-CL1_{—}3)

The protein of SEQ ID NO: 784 encoded by the extended cDNA SEQ ID NO: 379 isolated from kidney shows extensive homology to the murine WW-domain binding protein 1 or WWBP-1. WWBP-1 is encoded by the nucleic acid sequence of Genbank accession number U40825. This protein is expressed in placenta, lung, liver and kidney is thought to play a role in intracellular signaling by binding to the WW domain of the Yes protooncogene-associated protein via its so-called PY domain (Chen and Sudol, Proc. Natl. Acad. Sci., 92:7819-7823 (1995)). The WW—PY domains are thought to represent a new set of modular protein-binding sequences just like the SH3—PXXP domains (Sudol et al., FEBS Lett., 369:67-71 (1995)).

As shown by the alignments of FIG. 12 between the protein of SEQ ID NO:784 and WWBP-1, the amino acid residues are identical to those of the 305 amino acid long matched protein except for positions 53, 66, 78, 89, 92, 94, 96, 100, 102, 106, 110, 113, 124, 128, 136, 139, 140, 142-144, 166, 168, 173, 176, 178, 181, 182, 188, 196, 199, 201, 202, 207 and 210 of the matched protein. 68% of these substitutions are conservative. Indeed the histidine-rich PY domain is present in the protein of the invention (positions 82-86 in bold in FIG. 12).

Taken together, these data suggest that the protein of SEQ ID NO: 784 may play a role in intracellular signaling. Thus, this protein may be useful in diagnosing and/or treating several types of disorders including, but not limited to, cancer, neurodegenerative diseases, cardiovascular disorders, hypertension, renal injury and repair and septic shock.

Protein of SEQ ID NO: 753 (Internal Designation 108-004-5-0-G6-FL)

The protein SEQ ID NO: 753 found in liver encoded by the extended cDNA SEQ ID NO:348 shows homology to a lectin-like oxidized LDL receptor (LOX-1) found in human, bovine and murine species. Such type II proteins with a C-lectin-like domain, expressed in vascular endothelium and vascular-rich organs, bind and internalize oxidatively modified low-density lipoproteins (Sawamura et al, Nature, 386:73-77, (1997)). The oxidized lipoproteins have been implicated in the pathogenesis of atherosclerosis, a leading cause of death in industrialized countries (see review by Parthasarathy et al, Biochem. Pharmacol. 56:279-284 (1998)). In addition, type II membrane proteins with a C-terminus C-type lectin domain, also known as carbohydrate-recognition domains, also include proteins involved in target-cell recognition and cell activation.

The protein of invention has the typical structure of a type II protein belonging to the C-type lectin family. Indeed, it contains a short 31-amino-acid-long N-terminal tail, a transmembrane segment from positions 32 to 52 matching the one predicted for human LOX-I and a large 177-amino-acid-long C-terminal tail as predicted by the software TopPred II (Claros and von Heijne, CABIOS applic. Notes, 10:685-686 (1994)). All six cysteines of LOX-1 C-type lectin domain are also conserved in the protein of the invention (positions 102, 113, 130, 195, 208 and 216) although the characteristic PROSITE signature of this family is not. The LOX-1 protein is encoded by the nucleic acid sequence of Genbank accession number: AB010710.

Taken together, these data suggest that the protein of SEQ ID NO:753 may be involved in the metabolism of lipids and/or in cell-cell or cell-matrix interactions and/or in cell activation. Thus, this protein or part therein, may be useful in diagnosing and treating several disorders including, but not limited to, cancer, hyperlipidaemia, cardiovascular disorders and neurodegenerative disorders.

Protein of SEQ ID NO: 767 (Internal Designation 108-008-5-O-G12-FL)

The protein SEQ ID NO: 767 encoded by the extended cDNA SEQ ID NO:362 shows homology to a mitochondrial protein found in Saccharomyces Cerevisiae (PIR:S72254) which is similar to E. Coli ribosomal protein L36. The typical PROSITE signature for ribosomal L36 is present in the protein of the invention (positions 76-102) except for a substitution of a tryptophane residue instead of a valine, leucine, isoleucine, methionine or asparagine residue.

Taken together, these data suggest that the protein of SEQ ID NO:767 may be involved in protein biosynthesis. Thus, this protein may be useful in diagnosing and/or treating several types of disorders including, but not limited to, cancer.

Protein of SEQ ID NO: 750 (Internal Designation 108-004-5-0-D10-FL)

The protein SEQ ID NO: 750 encoded by the extended cDNA SEQ ID NO: 345 shows remote homology to a subfamily of beta4-galactosyltransferases widely conserved in animals (human, rodents, cow and chicken). Such enzymes, usually type II membrane proteins located in the endoplasmic reticulum or in the Golgi apparatus, catalyzes the biosynthesis of glycoproteins, glycolipid glycans and lactose. Their characteristic features defined as those of subfamily A in Breton et al, J. Biochem., 123:1000-1009 (1998) are pretty well conserved in the protein of the invention, especially the region I containing the DVD motif (positions 163-165) thought to be involved either in UDP binding or in the catalytic process itself.

In addition, the protein of invention has the typical structure of a type II protein. Indeed, it contains a short 28-amino-acid-long N-terminal tail, a transmembrane segment from positions 29 to 49 and a large 278-amino-acid-long C-terminal tail as predicted by the software TopPred II (Claros and von Heijne, CABIOS applic. Notes, 10:685-686 (1994)).

Taken together, these data suggest that the protein of SEQ ID NO: 750 may play a role in the biosynthesis of polysaccharides, and of the carbohydrate moieties of glycoproteins and glycolipids and/or in cell-cell recognition. Thus, this protein may be useful in diagnosing and/or treating several types of disorders including, but not limited to, cancer, atherosclerosis, cardiovascular disorders, autoimmune disorders and rheumatic diseases including rheumatoid arthritis.

Protein of SEQ ID NO: 760 (Internal Designation 108-006-5-0-G2-FL)

The protein of SEQ ID NO: 760 encoded by the extended cDNA SEQ ID NO: 355 shows homology to a neuronal murine protein NP15.6 whose expression is developmentally regulated. NP15.6 protein is encoded by the nucleic acid sequence of Genbank accession number Y08702.

Taken together, these data suggest that the protein of SEQ ID NO: 760 may be involved in cellular proliferation and differentiation. Thus, this protein may be useful in diagnosing and/or treating several types of disorders including, but not limited to, cancer, neurodegenerative disorders and embryogenetic disorders.

Protein of SEQ ID NO: 769 (Internal Designation 108-009-5-0-A2-FL)

The protein of SEQ ID NO: 769 encoded by the extended cDNA SEQ ID NO: 364 shows extensive homology to the bZIP family of transcription factors, and especially to the human luman protein. (Lu et al., Mol. Cell. Biol., 17:5117-5126 (1997)). The human luman protein is encoded by the nucleic acid sequence of Genbank accession number: AF009368. The match include the whole bZIP domain composed of a basic DNA-binding domain and of a leucine zipper allowing protein dimerization. The basic domain is conserved in the protein of the invention as shown by the characteristic PROSITE signature (positions 224-237) except for a conservative substitution of a glutamic acid with an aspartic acid in position 233. The typical PROSITE signature for leucine zipper is also present (positions 259 to 280). Secreted proteins may have nucleic acid binding domain as shown by a nematode protein thought to regulate gene expression which exhibits zinc fingers as well as a functional signal peptide (Holst and Zipfel, J. Biol. Chem., 271:16275-16733, 1996).

Taken together, these data suggest that the protein of SEQ ID NO: 113 may bind to DNA, hence regulating gene expression as a transcription factor. Thus, this protein may be useful in diagnosing and/or treating several types of disorders including, but not limited to, cancer.

Proteins of SEQ ID NO:785 (Internal Designation 76-13-3-A9-CL1_{—}1)

The protein of SEQ ID NO: 785 encoded by the extended cDNA SEQ ID NO:380 shows homology with part of a human seven transmembrane protein. The human seven transmembrane protein is encoded by the nucleic acid sequence of Genbank accession number Y11395. The matched protein potentially associated to stomatin may act as a G-protein coupled receptor and is likely to be important for the signal transduction in neurons and haematopoietic cells (Mayer et al, Biochem. Biophys. Acta., 1395:301-308 (1998)).

Taken together, these data suggest that the protein of SEQ ID NO:785 may be involved in signal transduction. Thus, this protein may be useful in diagnosing and/or treating several types of disorders including, but not limited to, cancer, neurodegenerative diseases, cardiovascular disorders, hypertension, renal injury and repair and septic shock.

Proteins of SEQ ID NO: 751 (Internal Designation 108-004-5-0-E8-FL)

The protein of SEQ ID NO: 751 encoded by the extended cDNA SEQ ID NO: 346 exhibit the typical PROSITE signature for amino acid permeases (positions 5 to 66) which are integral membrane proteins involved in the transport of amino acids into the cell. In addition, the protein of invention has a transmembrane segment from positions 9 to 29 as predicted by the software TopPred II (Claros and von Heijne, CABIOS applic. Notes, 10:685-686 (1994)).

Taken together, these data suggest that the protein of SEQ ID NO: 751 may be involved in amino acid transport. Thus, this protein may be useful in diagnosing and/or treating several types of disorders including, but not limited to, cancer, aminoacidurias, neurodegenerative diseases, anorexia, chronic fatigue, coronary vascular disease, diphtheria, hypoglycemia, male infertility, muscular and myopathies.

As discussed above, the extended cDNAs of the present invention or portions thereof can be used for various purposes. The polynucleotides can be used to express recombinant protein for use for therapeutic use or research (not limited to research on the gene itself); as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; for selecting and making oligomers for attachment to a “gene chip” or other support (e.g., microarrays), including for examination for expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins or polypeptides provided by the present invention can similarly be used in assays to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation Molecular Cloning; A Laboratory Manual, 2d ed., Cole Spring Harbor Laboratory Press, Sambrook J., E. F. Fritsch and T. Maniatis eds., (1989), and Methods in Enzymology; Guide to Molecular Cloning Techniques, Academic Press, Berger, S. L. and A. R. Kimmel eds., (1987).

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. Throughout this application, various publications, patents, and published patent applications are cited.

Some of the disclosures of the publications, patents, and published patent specifications referenced in this application may not have been incorporated into the present disclosure at the point of reference. Regardless of this, all of the disclosures of the publications, patents, and published patent specifications referenced in this application are hereby incorporated by reference in their entireties into the present disclosure to more fully describe the state of the art to which this invention pertains.

The nucleic acid sequences of SEQ ID NOs: 1-405 or fragments thereof may also be used to construct fusion proteins in which the polypeptide sequences of SEQ ID NOs: 406-810 or fragments thereof are fused to heterologous polypeptides. For example, the fragments of the polypeptides of SEQ ID NOs. 406-810 which are included in the fusion proteins may comprise at least 5, 10, 15, 20, 25, 30, 35, 40, 50, 75, 100, or 150 consecutive amino acids of the polypeptides of SEQ ID NOs.406-810 or may be of any length suitable for the intended purpose of the fusion protein. Nucleic acids encoding the desired fusion protein are produced by cloning a nucleic acid of SEQ ID NOs. 1-405 in frame with a nucleic acid encoding the heterologous polypeptide. The nucleic acid encoding the desired fusion protein is operably linked to a promoter in an appropriate vector, such as any of the vectors described above, and introduced into a host capable of expressing the fusion protein.

Antibodies against the polypeptides of SEQ ID NOs. 406-810 or fragments thereof may be used in immunoaffinity chromatography to isolate the polypeptides of SEQ ID NOs. 406-810 or fragments thereof or to isolate fusion proteins containing the polypeptides of SEQ ID NOs. 406-810 or fragments thereof.

The invention further relates to methods and compositions using the protein of the invention or part thereof to diagnose, prevent and/or treat several disorders in which the activity of the protein of the invention is deleterious. For diagnostic purposes, the expression of the protein of the invention could be investigated using any of the Northern blotting, RT-PCR or immunoblotting methods described herein and compared to the expression in control individuals. For prevention and/or treatment purposes, inhibiting the endogenous expression of the protein of the invention using any of the antisense or triple helix methods described herein may be used. Alternatively, inhibitors for the protein's activity may be developed and use to inhibit and/or reduce its activity using any methods known to those skilled in the art.

Chromosomal localization of the cDNA of the present invention were also determined using information from public and proprietary databases. Table XI lists the putative chromosomal location of the polynucleotides of the present invention. Column 1 lists the sequence identification number with the corresponding chromosomal location listed in column two.

The present invention also relates to methods and compositions using the chromosomal location of the polynucleotides of the invention to construct a human high resolution map or to identify a given chromosome in a sample using any techniques to those skilled in the art including those disclosed in Example 43.

Alternatively, the cDNA clone obtained by the process described in Examples 1 through 13 may not include the entire coding sequence of the protein encoded by the corresponding mRNA, although they do include sequences derived from the 5′ends of their corresponding mRNA. Such 5′EST can be used to isolate extended cDNAs which contain sequences adjacent to the 5′ ESTs. Such obtained extended cDNAs may include the entire coding sequence of the protein encoded by the corresponding mRNA, including the authentic translation start site. Examples 16 and 17 below describe methods for obtaining extended cDNAs using 5′ ESTs. Example 17 also describes methods to obtain cDNA, mRNA or genomic DNA homologous to cDNA, 5′ESTs, or fragment thereof.

The methods of Examples 16 and 17 can also be used to obtain cDNAs which encode less than the entire coding sequence of proteins encoded by the genes corresponding to the 5′ ESTs. In some embodiments, the cDNAs isolated using these methods encode at least 5, 8, 10, 12, 15, 20, 25, 30, 35, 40, 50, 60, 75, 100, 150 or 200 consecutive amino acids of one of the proteins encoded by the sequences of SEQ ID NOs. 406-810.

### EXAMPLE 16

General Method for using 5′ ESTs to Clone and Sequence cDNAs which Include the Entire Coding Region and the Authentic 5′End of the Corresponding mRNA

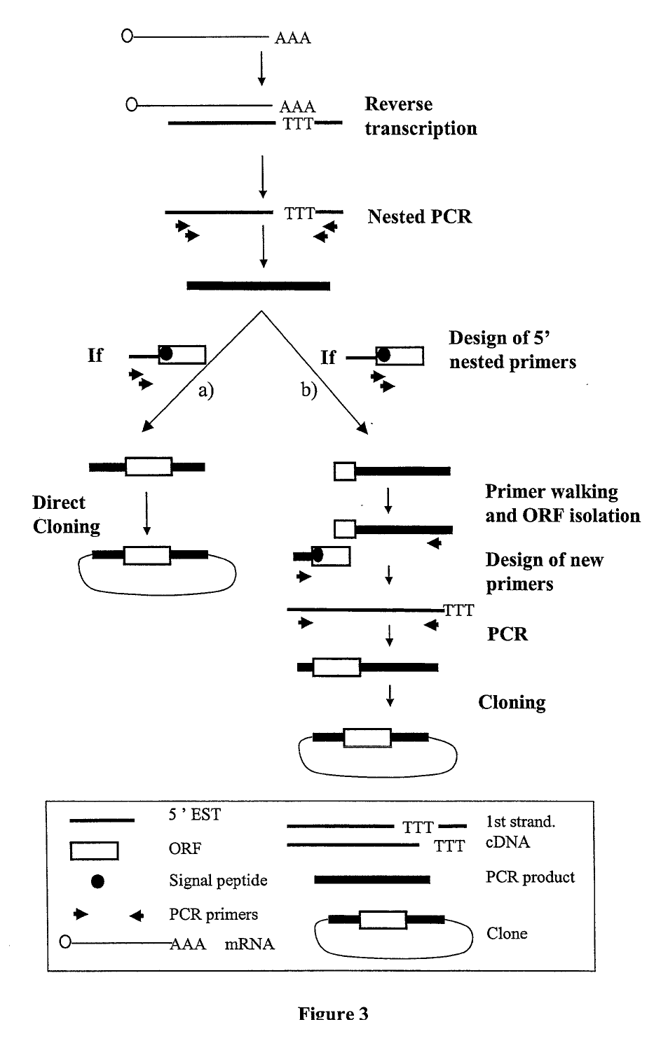

The following general method may be used to quickly and efficiently isolate cDNAs including sequence adjacent to the sequences of the 5′ ESTs used to obtain them. This method, ilustrated in FIG. 3, may be applied to obtain cDNAs for any 5′ EST.

The method takes advantage of the known 5′ sequence of the mRNA. A reverse transcription reaction is conducted on purified mRNA with a poly dT primer containing a nucleotide sequence at its 5′ end allowing the addition of a known sequence at the end of the cDNA which corresponds to the 3′ end of the mRNA. Such a primer and a commercially-available reverse transcriptase enzyme are added to a buffered mRNA sample yielding a reverse transcript anchored at the 3′ polyA site of the RNAs. Nucleotide monomers are then added to complete the first strand synthesis. After removal of the mRNA hybridized to the first cDNA strand by alkaline hydrolysis, the products of the alkaline hydrolysis and the residual poly dT primer can be eliminated with an exclusion column.

Subsequently, a pair of nested primers on each end is designed based on the known 5′ sequence from the 5′ EST and the known 3′ end added by the poly dT primer used in the first strand synthesis. Software used to design primers is either based on GC content and melting temperatures of oligonucleotides, such as OSP (Illier and Green, PCR Meth. Appl. 1:124-128, 1991), or based on the octamer frequency disparity method (Griffais et al., Nucleic Acids Res. 19: 3887-3891, 1991) such as PC-Rare (http://bioinformatics.weizmann.ac.il/software/PC-Rare/doc/manuel.html). Preferably, the nested primers at the 5′ end and the nested primers at the 3′ end are separated from one another by four to nine bases. These primer sequences may be selected to have melting temperatures and specificities suitable for use in PCR.

A first PCR run is performed using the outer primer from each of the nested pairs. A second PCR run using the inner primer from each of the nested pairs is then performed on a small aliquot of the first PCR product. Thereafter, the primers and remaining nucleotide monomers are removed.

Due to the lack of position constraints on the design of 5′ nested primers compatible for PCR use using the OSP software, amplicons of two types are obtained. Preferably, the second 5′ primer is located upstream of the translation initiation codon thus yielding a nested PCR product containing the entire coding sequence. Such a cDNA may be used in a direct cloning procedure such as the one described in example 4.

However, in some cases, the second 5′ primer is located downstream of the translation initiation codon, thereby yielding a PCR product containing only part of the ORF. For such amplicons which do not contain the complete coding sequence, intermediate steps are necessary to obtain both the complete coding sequence and a PCR product containing the full coding sequence. The complete coding sequence can be assembled from several partial sequences determined directly from different PCR products. Once the full coding sequence has been completely determined, new primers compatible for PCR use are then designed to obtain amplicons containing the whole coding region. However, in such cases, 3′ primers compatible for PCR use are located inside the 3′ UTR of the corresponding mRNA, thus yielding amplicons which lack part of this region, i.e. the polyA tract and sometimes the polyadenylation signal, as illustrated in FIG. 3. Such obtained cDNAs are then cloned into an appropriate vector using a procedure essentially similar to the one described in example 4.

Full-length PCR products are then sequenced using a procedure similar to the one described in example 11. Completion of the sequencing of a given cDNA fragment may be assessed by comparing the sequence length to the size of the corresponding nested PCR product. When Northern blot data are available, the size of the mRNA detected for a given PCR product may also be used to finally assess that the sequence is complete. Sequences which do not fulfill these criteria are discarded and will undergo a new isolation procedure.

Full-length PCR products are then cloned in an appropriate vector. For example, the cDNAs can be cloned into a vector using a procedure similar to the one described in example 4. Such full-length cDNA clones are then double-sequenced and submitted to computer analyses using procedure essentially similar to the ones described in Examples 11 through 13. However, it will be appreciated that full-length cDNA clones obtained from amplicons lacking part of the 3′UTR may lack polyadenylations sites and signals.

### EXAMPLE 17

25 Methods for Obtaining cDNAs or Nucleic Acids Homologous to cDNAs or Fragments Thereof

In addition to PCR based methods for obtaining cDNAs, traditional hybridization based methods may also be employed. These methods may also be used to obtain the genomic DNAs which encode the mRNAs from which the cDNA is derived, mRNAs corresponding to the cDNAs, or nucleic acids which are homologous to cDNAs or fragments thereof. Indeed, cDNAs of the present invention or fragments thereof, including 5′ESTs, may also be used to isolate cDNAs or nucleic acids homologous to cDNAs from a cDNA library or a genomic DNA library as follows. Such cDNA libraries or genomic DNA libraries may be obtained from a commercial source or made using techniques familiar to those skilled in the art such as the one described in Examples 1 through 5. An example of such hybridization-based methods is provided below.

Techniques for identifying cDNA clones in a cDNA library which hybridize to a given probe sequence are disclosed in Sambrook et al., Molecular Cloning: A Laboratory Manual 2d Ed., Cold Spring Harbor Laboratory Press, 1989, the disclosure of which is incorporated herein by reference. The same techniques may be used to isolate genomic DNAs.

Briefly, cDNA or genomic DNA clones which hybridize to the detectable probe are identified and isolated for further manipulation as follows. A probe comprising at least 10 consecutive nucleotides from the cDNA or fragment thereof is labeled with a detectable label such as a radioisotope or a fluorescent molecule. Preferably, the probe comprises at least 12, 15, or 17 consecutive nucleotides from the cDNA or fragment thereof. More preferably, the probe comprises 20 to 30 consecutive nucleotides from the cDNA or fragment thereof. In some embodiments, the probe comprises more than 30 nucleotides from the cDNA or fragment thereof.

Techniques for labeling the probe are well known and include phosphorylation with polynucleotide kinase, nick translation, in vitro transcription, and non radioactive techniques. The cDNAs or genomic DNAs in the library are transferred to a nitrocellulose or nylon filter and denatured. After blocking of non specific sites, the filter is incubated with the labeled probe for an amount of time sufficient to allow binding of the probe to cDNAs or genomic DNAs containing a sequence capable of hybridizing thereto.

By varying the stringency of the hybridization conditions used to identify cDNAs or genomic DNAs which hybridize to the detectable probe, cDNAs or genomic DNAs having different levels of identity to the probe can be identified and isolated as described below.

1. Isolation of cDNA or Genomic DNA Sequences Having a High Degree of Identity to the Labeled Probe

To identify cDNAs or genomic DNAs having a high degree of identity to the probe sequence, the melting temperature of the probe may be calculated using the following formulas:

For probes between 14 and 70 nucleotides in length the melting temperature (Tm) is calculated using the formula: Tm=81.5+16.6(log (Na+))+0.41(fraction G+C)−(600/N) where N is the length of the probe.

If the hybridization is carried out in a solution containing formamide, the melting temperature may be calculated using the equation T=81.5+16.6(log (Na+))+0.41(fraction G+C)−(0.63% formamide)−(600/N) where N is the length of the probe.

Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% formamide. The formulas for SSC and Denhardt's solutions are listed in Sambrook et al., supra.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 15-25° C. below the Tm. Preferably, for hybridizations in 6×SSC, the hybridization is conducted at approximately 68° C. Preferably, for hybridizations in 50% formamide containing solutions, the hybridization is conducted at approximately 42° C.

All of the foregoing hybridizations would be considered to be under “stringent” conditions.

Following hybridization, the filter is washed in 2×SSC, 0.1% SDS at room temperature for 15 minutes. The filter is then washed with 0.1×SSC, 0.5% SDS at room temperature for 30 minutes to 1 hour. Thereafter, the solution is washed at the hybridization temperature in 0.1×SSC, 0.5% SDS. A final wash is conducted in 0.1×SSC at room temperature.

cDNAs or genomic DNAs which have hybridized to the probe are identified by autoradiography or other conventional techniques.

2. Isolation of cDNA or Genomic DNA Sequences Having Lower Degrees of Identity to the Labeled Probe

The above procedure may be modified to identify cDNAs or genomic DNAs having decreasing levels of identity to the probe sequence. For example, to obtain cDNAs or genomic DNAs of decreasing identity to the detectable probe, less stringent conditions may be used. For example, the hybridization temperature may be decreased in increments of 5° C. from 68° C. to 42° C. in a hybridization buffer having a sodium concentration of approximately 1M. Following hybridization, the filter may be washed with 2×SSC, 0.5% SDS at the temperature of hybridization. These conditions are considered to be “moderate” conditions above 50° C. and “low” conditions below 50° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of identity to the probe. Following hybridization, the filter may be washed with 6×SSC, 0.5% SDS at 50° C. These conditions are considered to be “moderate” conditions above 25% formamide and “low” conditions below 25% formamide. cDNAs or genomic DNAs which have hybridized to the probe are identified by autoradiography or other conventional techniques.

3. Determination of the Degree of Identity between the Obtained cDNAs or Genomic DNAs and cDNAs or Fragments thereof used as the Labeled Probe or Between the Polypeptides Encoded by the Obtained cDNAs or Genomic DNAs and the Polypeptides Encoded by the cDNAs or Fragment Thereof Used as the Labeled Probe

To determine the level of identity between the hybridized cDNA or genomic DNA and the cDNA or fragment thereof from which the probe was derived, the nucleotide sequences of the hybridized nucleic acid and the cDNA or fragment thereof from which the probe was derived are compared. The sequences of the cDNA or fragment thereof from which the probe was derived and the sequences of the cDNA or genomic DNA which hybridized to the detectable probe may be stored on a computer readable medium as described below and compared to one another using any of a variety of algorithms familiar to those skilled in the art such as those described below.

To determine the level of identity between the polypeptide encoded by the hybridizing cDNA or genomic DNA and the polypeptide encoded by the cDNA or fragment thereof from which the probe was derived, the polypeptide sequence encoded by the hybridized nucleic acid and the polypeptide sequence encoded by the cDNA or fragment thereof from which the probe was derived are compared. The sequences of the polypeptide encoded by the cDNA or fragment thereof from which the probe was derived and the polypeptide sequence encoded by the cDNA or genomic DNA which hybridized to the detectable probe may be stored on a computer readable medium as described below and compared to one another using any of a variety of algorithms familiar to those skilled in the art such as those described below.

Protein and/or nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al., 1996, Methods Enzymol. 266:383-402; Altschul et al., 1990, J. Mol. Biol. 215(3):403-410; Altschul et al., 1993, Nature Genetics 3:266-272).

In a particularly preferred embodiment, protein and nucleic acid sequence homologies are evaluated using the Basic Local Alignment Search Tool (“BLAST”) which is well known in the art (see, e.g., Karlin and Altschul, 1990, Proc. Natl. Acad. Sci. USA 87:2267-2268; Altschul et al., 1990, J. Mol. Biol. 215:403-410; Altschul et al., 1993, Nature Genetics 3:266-272; Altschul et al., 1997, Nuc. Acids Res. 25:3389-3402). In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992, Science 256:1443-1445; Henikoff and Henikoff, 1993, Proteins 17:49-61). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation)

The BLAST programs evaluate the statistical significance of all high-scoring segment pairs identified, and preferably selects those segments which satisfy a user-specified threshold of significance, such as a user-specified percent identity. Preferably, the statistical significance of a high-scoring segment pair is evaluated using the statistical significance formula of Karlin (see, e.g., Karlin and Altschul, 1990, Proc. Natl. Acad. Sci. USA 87:2267-2268).

The parameters used with the above algorithms may be adapted depending on the sequence length and degree of identity studied. In some embodiments, the parameters may be the default parameters used by the algorithms in the absence of instructions from the user.

In some embodiments, the level of identity between the hybridized nucleic acid and the cDNA or fragment thereof from which the probe was derived may be determined using the FASTDB algorithm described in Brutlag et al. Comp. App. Biosci. 6:237-245, 1990. In such analyses the parameters may be selected as follows: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the sequence which hybridizes to the probe, whichever is shorter. Because the FASTDB program does not consider 5′ or 3′ truncations when calculating identity levels, if the sequence which hybridizes to the probe is truncated relative to the sequence of the cDNA or fragment thereof from which the probe was derived the identity level is manually adjusted by calculating the number of nucleotides of the cDNA or fragment thereof which are not matched or aligned with the hybridizing sequence, determining the percentage of total nucleotides of the hybridizing sequence which the non-matched or non-aligned nucleotides represent, and subtracting this percentage from the identity level. For example, if the hybridizing sequence is 700 nucleotides in length and the cDNA or fragment thereof sequence is 1000 nucleotides in length wherein the first 300 bases at the 5′end of the cDNA or fragment thereof are absent from the hybridizing sequence, and wherein the overlapping 700 nucleotides are identical, the identity level would be adjusted as follows. The non-matched, non-aligned 300 bases represent 30% of the length of the cDNA or fragment thereof. If the overlapping 700 nucleotides are 100% identical, the adjusted identity level would be 100−30=70% identity. It should be noted that the preceding adjustments are only made when the non-matched or non-aligned nucleotides are at the 5′ or 3′ends. No adjustments are made if the non-matched or non-aligned sequences are internal or under any other conditions.

For example, using the above methods, nucleic acids having at least 95% nucleic acid identity, at least 96% nucleic acid identity, at least 97% nucleic acid identity, at least 98% nucleic acid identity, at least 99% nucleic acid identity, or more than 99% nucleic acid identity to the cDNA or fragment thereof from which the probe was derived may be obtained and identified. Such nucleic acids may be allelic variants or related nucleic acids from other species. Similarly, by using progressively less stringent hybridization conditions one can obtain and identify nucleic acids having at least 90%, at least 85%, at least 80% or at least 75% identity to the cDNA or fragment thereof from which the probe was derived.

Using the above methods and algorithms such as FASTA with parameters depending on the sequence length and degree of identity studied, for example the default parameters used by the algorithms in the absence of instructions from the user, one can obtain nucleic acids encoding proteins having at least 99%, at least 98%, at least 97%, at least 96%, at least 95%, at least 90%, at least 85%, at least 80% or at least 75% identity to the protein encoded by the cDNA or fragment thereof from which the probe was derived. In some embodiments, the identity levels can be determined using the “default” opening penalty and the “default” gap penalty, and a scoring matrix such as PAM 250 (a standard scoring matrix; see Dayhoff et al., in: Atlas of Protein Sequence and Structure, Vol. 5, Supp. 3 (1978)).

Alternatively, the level of polypeptide identity may be determined using the FASTDB algorithm described by Brutlag et al. Comp. App. Biosci. 6:237-245, 1990. In such analyses the parameters may be selected as follows: Matrix=PAM 0, k-tuple=2, Mismatch Penalty=1, Joining Penalty-20, Randomization Group Length=0, Cutoff Score=1, Window Size=Sequence Length, Gap Penalty=5, Gap Size Penalty=0.05, Window Size=500 or the length of the homologous sequence, whichever is shorter. If the homologous amino acid sequence is shorter than the amino acid sequence encoded by the cDNA or fragment thereof as a result of an N terminal and/or C terminal deletion the results may be manually corrected as follows. First, the number of amino acid residues of the amino acid sequence encoded by the cDNA or fragment thereof which are not matched or aligned with the homologous sequence is determined. Then, the percentage of the length of the sequence encoded by the cDNA or fragment thereof which the non-matched or non-aligned amino acids represent is calculated. This percentage is subtracted from the identity level. For example wherein the amino acid sequence encoded by the cDNA or fragment thereof is 100 amino acids in length and the length of the homologous sequence is 80 amino acids and wherein the amino acid sequence encoded by the cDNA or fragment thereof is truncated at the N terminal end with respect to the homologous sequence, the identity level is calculated as follows. In the preceding scenario there are 20 non-matched, non-aligned amino acids in the sequence encoded by the cDNA or fragment thereof. This represents 20% of the length of the amino acid sequence encoded by the cDNA or fragment thereof. If the remaining amino acids are 100% identical between the two sequences, the identity level would be 100%-20%=80% identity. No adjustments are made if the non-matched or non-aligned sequences are internal or under any other conditions.

In addition to the above described methods, other protocols are available to obtain homologous cDNAs using cDNA of the present invention or fragment thereof as outlined in the following paragraphs.
cDNAs may be prepared by obtaining mRNA from the tissue, cell, or organism of interest using mRNA preparation procedures utilizing polyA selection procedures or other techniques known to those skilled in the art. A first primer capable of hybridizing to the polyA tail of the mRNA is hybridized to the mRNA and a reverse transcription reaction is performed to generate a first cDNA strand.

The first cDNA strand is hybridized to a second primer containing at least 10 consecutive nucleotides of the sequences of SEQ ID NOs 1-405. Preferably, the primer comprises at least 10, 12, 15, 17, 18, 20, 23, 25, or 28 consecutive nucleotides from the sequences of SEQ ID NOs 1-405. In some embodiments, the primer comprises more than 30 nucleotides from the sequences of SEQ ID NOs 1-405. If it is desired to obtain cDNAs containing the full protein coding sequence, including the authentic translation initiation site, the second primer used contains sequences located upstream of the translation initiation site. The second primer is extended to generate a second cDNA strand complementary to the first cDNA strand. Alternatively, RT-PCR may be performed as described above using primers from both ends of the cDNA to be obtained.

cDNAs containing 5′ fragments of the mRNA may be prepared by hybridizing an mRNA comprising the sequences of SEQ ID NOs. 1-405 with a primer comprising a complementary to a fragment of the known cDNA, genomic DNA or fragment thereof hybridizing the primer to the mRNAs, and reverse transcribing the hybridized primer to make a first cDNA strand from the mRNAs. Preferably, the primer comprises at least 10, 12, 15, 17, 18, 20, 23, 25, or 28 consecutive nucleotides of the sequences complementary to SEQ ID NOs. 1-405.

Thereafter, a second cDNA strand complementary to the first cDNA strand is synthesized. The second cDNA strand may be made by hybridizing a primer complementary to sequences in the first cDNA strand to the first cDNA strand and extending the primer to generate the second cDNA strand.

The double stranded cDNAs made using the methods described above are isolated and cloned. The cDNAs may be cloned into vectors such as plasmids or viral vectors capable of replicating in an appropriate host cell. For example, the host cell may be a bacterial, mammalian, avian, or insect cell.

Techniques for isolating mRNA, reverse transcribing a primer hybridized to mRNA to generate a first cDNA strand, extending a primer to make a second cDNA strand complementary to the first cDNA strand, isolating the double stranded cDNA and cloning the double stranded cDNA are well known to those skilled in the art and are described in Current Protocols in Molecular Biology, John Wiley & Sons, Inc. 1997 and Sambrook et al., Molecular Cloning: A Laboratory Manual, Second Edition, Cold Spring Harbor Laboratory Press, 1989.

Alternatively, other procedures may be used for obtaining full-length cDNAs or homologous cDNAs. In one approach, cDNAs are prepared from mRNA and cloned into double stranded phagemids as follows. The cDNA library in the double stranded phagemids is then rendered single stranded by treatment with an endonuclease, such as the Gene II product of the phage Fl and an exonuclease (Chang et al., Gene 127:95-8, 1993). A biotinylated oligonucleotide comprising the sequence of a fragment of a known cDNA, genomic DNA or fragment thereof is hybridized to the single stranded phagemids. Preferably, the fragment comprises at least 10, 12, 15, 17, 18, 20, 23, 25, or 28 consecutive nucleotides of the sequences of SEQ ID NOs. 1-405.

Hybrids between the biotinylated oligonucleotide and phagemids are isolated by incubating the hybrids with streptavidin coated paramagnetic beads and retrieving the beads with a magnet (Fry et al., Biotechniques, 13: 124-131, 1992). Thereafter, the resulting phagemids are released from the beads and converted into double stranded DNA using a primer specific for the cDNA or fragment thereof used to design the biotinylated oligonucleotide. Alternatively, protocols such as the Gene Trapper kit (Gibco BRL) may be used. The resulting double stranded DNA is transformed into bacteria. Homologous cDNAs or full length cDNAs containing the cDNA or fragment thereof sequence are identified by colony PCR or colony hybridization.

Using any of the above described methods, a plurality of cDNAs containing full-length protein coding sequences or fragments of the protein coding sequences may be provided as cDNA libraries for subsequent evaluation of the encoded proteins or use in diagnostic assays as described below.

cDNAs prepared by any method described therein may be subsequently engineered to obtain nucleic acids which include desired fragments of the cDNA using conventional techniques such as subcloning, PCR, or in vitro oligonucleotide synthesis. For example, nucleic acids which include only the full coding sequences (i.e. the sequences encoding the signal peptide and the mature protein remaining after the signal peptide peptide is cleaved off) may be obtained using techniques known to those skilled in the art. Alternatively, conventional techniques may be applied to obtain nucleic acids which contain only the coding sequence for the mature protein remaining after the signal peptide is cleaved off or nucleic acids which contain only the coding sequences for the signal peptides.

Similarly, nucleic acids containing any other desired fragment of the coding sequences for the encoded protein may be obtained. For example, the nucleic acid may contain at least 8, 10, 12, 15, 18, 20, 25, 28, 30, 35, 40, 50, 75, 100, 150, 200, 300, 400, 500, 1000 or 2000 consecutive bases of a cDNA.

Once a cDNA has been obtained, it can be sequenced to determine the amino acid sequence it encodes. Once the encoded amino acid sequence has been determined, one can create and identify any of the many conceivable cDNAs that will encode that protein by simply using the degeneracy of the genetic code. For example, allelic variants or other homologous nucleic acids can be identified as described below. Alternatively, nucleic acids encoding the desired amino acid sequence can be synthesized in vitro.

In a preferred embodiment, the coding sequence may be selected using the known codon or codon pair preferences for the host organism in which the cDNA is to be expressed.

IV. Use of cDNA or Fragments thereof to Express Proteins and uses of those Expressed Proteins

Using any of the above described methods, cDNAs containing the full protein coding sequences of their corresponding mRNAs or portions thereof, such as cDNAs encoding the mature protein, may be used to express the secreted proteins or portions thereof which they encode as described below. If desired, the cDNAs may contain the sequences encoding the signal peptide to facilitate secretion of the expressed protein. It will be appreciated that a plurality of extended cDNAs containing the full protein coding sequences or portions thereof may be simultaneously cloned into expression vectors to create an expression library for analysis of the encoded proteins as described below.

### EXAMPLE 18

Expression of the Proteins Encoded by cDNAs or Fragments thereof

To express the proteins encoded by the cDNAs or fragments thereof, nucleic acids containing the coding sequence for the proteins or fragments thereof to be expressed are obtained as described above and cloned into a suitable expression vector. If desired, the nucleic acids may contain the sequences encoding the signal peptide to facilitate secretion of the expressed protein. For example, the nucleic acid may comprise the sequence of one of SEQ ID NOs: 1-405 listed in Table I and in the accompanying sequence listing. Alternatively, the nucleic acid may comprise those nucleotides which make up the full coding sequence of one of the sequences of SEQ ID NOs: 1-405 as defined in Table I above.

It will be appreciated that should the extent of the full coding sequence (i.e. the sequence encoding the signal peptide and the mature protein resulting from cleavage of the signal peptide) differ from that listed in Table I as a result of a sequencing error, reverse transcription or amplification error, mRNA splicing, post-translational modification of the encoded protein, enzymatic cleavage of the encoded protein, or other biological factors, one skilled in the art would be readily able to identify the extent of the full coding sequences in the sequences of SEQ ID NOs. 1-405. Accordingly, the scope of any claims herein relating to nucleic acids containing the full coding sequence of one of SEQ ID NOs. 1-405 is not to be construed as excluding any readily identifiable variations from or equivalents to the full coding sequences listed in Table I. Similarly, should the extent of the fall length polypeptides differ from those indicated in Table II as a result of any of the preceding factors, the scope of claims relating to polypeptides comprising the amino acid sequence of the full length polypeptides is not to be construed as excluding any readily identifiable variations from or equivalents to the sequences listed in Table II.

Alternatively, the nucleic acid used to express the protein or fragment thereof may comprise those nucleotides which encode the mature protein (i.e. the protein created by cleaving the signal peptide off) encoded by one of the sequences of SEQ ID NOs: 1-405 as defined in Table I above.

It will be appreciated that should the extent of the sequence encoding the mature protein differ from that listed in Table I as a result of a sequencing error, reverse transcription or amplification error, mRNA splicing, post-translational modification of the encoded protein, enzymatic cleavage of the encoded protein, or other biological factors, one skilled in the art would be readily able to identify the extent of the sequence encoding the mature protein in the sequences of SEQ ID NOs. 1405. Accordingly, the scope of any claims herein relating to nucleic acids containing the sequence encoding the mature protein encoded by one of SEQ ID NOs. 1-405 is not to be construed as excluding any readily identifiable variations from or equivalents to the sequences listed in Table I. Thus, claims relating to nucleic acids containing the sequence encoding the mature protein encompass equivalents to the sequences listed in Table I, such as sequences encoding biologically active proteins resulting from post-translational modification, enzymatic cleavage, or other readily identifiable variations from or equivalents to the secreted proteins in addition to cleavage of the signal peptide. Similarly, should the extent of the mature polypeptides differ from those indicated in Table II as a result of any of the preceding factors, the scope of claims relating to polypeptides comprising the sequence of a mature protein included in the sequence of one of SEQ ID NOs. 406-810 is not to be construed as excluding any readily identifiable variations from or equivalents to the sequences listed in Table II. Thus, claims relating to polypeptides comprising the sequence of the mature protein encompass equivalents to the sequences listed in Table II, such as biologically active proteins resulting from post-translational modification, enzymatic cleavage, or other readily identifiable variations from or equivalents to the secreted proteins in addition to cleavage of the signal peptide. It will also be appreciated that should the biologically active form of the polypeptides included in the sequence of one of SEQ ID NOs. 406-810 or the nucleic acids encoding the biologically active form of the polypeptides differ from those identified as the mature polypeptide in Table II or the nucleotides encoding the mature polypeptide in Table I as a result of a sequencing error, reverse transcription or amplification error, mRNA splicing, post-translational modification of the encoded protein, enzymatic cleavage of the encoded protein, or other biological factors, one skilled in the art would be readily able to identify the amino acids in the biologically active form of the polypeptides and the nucleic acids encoding the biologically active form of the polypeptides. In such instances, the claims relating to polypetides comprising the mature protein included in one of SEQ ID NOs. 406-810 or nucleic acids comprising the nucleotides of one of SEQ ID NOs. 1405 encoding the mature protein shall not be construed to exclude any readily identifiable variations from the sequences listed in Table I and Table II.

In some embodiments, the nucleic acid used to express the protein or fragment thereof may comprise those nucleotides which encode the signal peptide encoded by one of the sequences of SEQ ID NOs: 1-405 as defined in Table I above.

It will be appreciated that should the extent of the sequence encoding the signal peptide differ from that listed in Table I as a result of a sequencing error, reverse transcription or amplification error, mRNA splicing, post-translational modification of the encoded protein, enzymatic cleavage of the encoded protein, or other biological factors, one skilled in the art would be readily able to identify the extent of the sequence encoding the signal peptide in the sequences of SEQ ID NOs. 1-405. Accordingly, the scope of any claims herein relating to nucleic acids containing the sequence encoding the signal peptide encoded by one of SEQ ID NOs.1-405 is not to be construed as excluding any readily identifiable variations from the sequences listed in Table I. Similarly, should the extent of the signal peptides differ from those indicated in Table II as a result of any of the preceding factors, the scope of claims relating to polypeptides comprising the sequence of a signal peptide included in the sequence of one of SEQ ID NOs. 406-810 is not to be construed as excluding any readily identifiable variations from the sequences listed in Table II.

Alternatively, the nucleic acid may encode a polypeptide comprising at least 5 consecutive amino acids of one of the sequences of SEQ ID NOs: 406-810. In some embodiments, the nucleic acid may encode a polypeptide comprising at least 8, 10, 12, 15, 20, 25, 30, 35, 40, 50, 60, 75, 100, 150 or 200 consecutive amino acids of one of the sequences of SEQ ID NOs: 406-810.

The nucleic acids inserted into the expression vectors may also contain sequences upstream of the sequences encoding the signal peptide, such as sequences which regulate expression levels or sequences which confer tissue specific expression.

The nucleic acid encoding the protein or polypeptide to be expressed is operably linked to a promoter in an expression vector using conventional cloning technology. The expression vector may be any of the mammalian, yeast, insect or bacterial expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon context and codon pairing of the sequence may be optimized for the particular expression organism in which the expression vector is introduced, as explained by Hatfield, et al., U.S. Pat. No. 5,082,767, incorporated herein by this reference.

The following is provided as one exemplary method to express the proteins encoded by the cDNAs or the nucleic acids described above. First, the methionine initiation codon for the gene and the poly A signal of the gene are identified. If the nucleic acid encoding the polypeptide to be expressed lacks a methionine to serve as the initiation site, an initiating methionine can be introduced next to the first codon of the nucleic acid using conventional techniques. Similarly, if the cDNA lacks a poly A signal, this sequence can be added to the construct by, for example, splicing out the Poly A signal from pSG5 (Stratagene) using BglI and SalI restriction endonuclease enzymes and incorporating it into the mammalian expression vector pXT1 (Stratagene). pXT1 contains the LTRs and a fragment of the gag gene from Moloney Murine Leukemia Virus. The position of the LTRs in the construct allow efficient stable transfection. The vector includes the Herpes Simplex Thymidine Kinase promoter and the selectable neomycin gene. The cDNA or fragment thereof encoding the polypeptide to be expressed is obtained by PCR from the bacterial vector using oligonucleotide primers complementary to the cDNA or fragment thereof and containing restriction endonuclease sequences for Pst I incorporated into the 5′primer and BglII at the 5′ end of the corresponding cDNA 3′ primer, taking care to ensure that the cDNA is positioned in frame with the poly A signal. The purified fragment obtained from the resulting PCR reaction is digested with PstI, blunt ended with an exonuclease, digested with Bgl II, purified and ligated to pXT1, now containing a poly A signal and digested with BglII.

The ligated product is transfected into mouse NIH 3T3 cells using Lipofectin (Life Technologies, Inc., Grand Island, N.Y.) under conditions outlined in the product specification. Positive transfectants are selected after growing the transfected cells in 600 ug/ml G418 (Sigma, St. Louis, Mo.). Preferably the expressed protein is released into the culture medium, thereby facilitating purification.

Alternatively, the cDNAs may be cloned into pED6dpc2 (DiscoverEase, Genetics Institute, Cambridge, Mass.). The resulting pED6dpc2 constructs may be transfected into a suitable host cell, such as COS 1 cells. Methotrexate resistant cells are selected and expanded. Preferably, the protein expressed from the cDNA is released into the culture medium thereby facilitating purification.

Proteins in the culture medium are separated by gel electrophoresis. If desired, the proteins may be ammonium sulfate precipitated or separated based on size or charge prior to electrophoresis.

As a control, the expression vector lacking a cDNA insert is introduced into host cells or organisms and the proteins in the medium are harvested. The secreted proteins present in the medium are detected using techniques such as Coomassie or silver staining or using antibodies against the protein encoded by the cDNA. Coomassie and silver staining techniques are familiar to those skilled in the art.

Antibodies capable of specifically recognizing the protein of interest may be generated using synthetic 15-mer peptides having a sequence encoded by the appropriate 5′ EST, cDNA, or fragment thereof. The synthetic peptides are injected into mice to generate antibody to the polypeptide encoded by the 5′ EST, cDNA, or fragment thereof.

Secreted proteins from the host cells or organisms containing an expression vector which contains the cDNA or a fragment thereof are compared to those from the control cells or organism. The presence of a band in the medium from the cells containing the expression vector which is absent in the medium from the control cells indicates that the cDNA encodes a secreted protein. Generally, the band corresponding to the protein encoded by the cDNA will have a mobility near that expected based on the number of amino acids in the open reading frame of the cDNA. However, the band may have a mobility different than that expected as a result of modifications such as glycosylation, ubiquitination, or enzymatic cleavage.

Alternatively, if the protein expressed from the above expression vectors does not contain sequences directing its secretion, the proteins expressed from host cells containing an expression vector containing an insert encoding a secreted protein or fragment thereof can be compared to the proteins expressed in host cells containing the expression vector without an insert. The presence of a band in samples from cells containing the expression vector with an insert which is absent in samples from cells containing the expression vector without an insert indicates that the desired protein or fragment thereof is being expressed. Generally, the band will have the mobility expected for the secreted protein or fragment thereof. However, the band may have a mobility different than that expected as a result of modifications such as glycosylation, ubiquitination, or enzymatic cleavage.

The protein encoded by the cDNA may be purified using standard immunochromatography techniques. In such procedures, a solution containing the secreted protein, such as the culture medium or a cell extract, is applied to a column having antibodies against the secreted protein attached to the chromatography matrix. The secreted protein is allowed to bind the immunochromatography column. Thereafter, the column is washed to remove non-specifically bound proteins. The specifically bound secreted protein is then released from the column and recovered using standard techniques.

If antibody production is not possible, the cDNA sequence or fragment thereof may be incorporated into expression vectors designed for use in purification schemes employing chimeric polypeptides. In such strategies the coding sequence of the cDNA or fragment thereof is inserted in frame with the gene encoding the other half of the chimera. The other half of the chimera may be β-globin or a nickel binding polypeptide encoding sequence. A chromatography matrix having antibody to β-globin or nickel attached thereto is then used to purify the chimeric protein. Protease cleavage sites may be engineered between the β-globin gene or the nickel binding polypeptide and the cDNA or fragment thereof. Thus, the two polypeptides of the chimera may be separated from one another by protease digestion.

One useful expression vector for generating β-globin chimerics is pSG5 (Stratagene), which encodes rabbit β-globin. Intron II of the rabbit β-globin gene facilitates splicing of the expressed transcript, and the polyadenylation signal incorporated into the construct increases the level of expression. These techniques as described are well known to those skilled in the art of molecular biology. Standard methods are published in methods texts such as Davis et al., (Basic Methods in Molecular Biology, L. G. Davis, M. D. Dibner, and J. F. Battey, ed., Elsevier Press, NY, 1986) and many of the methods are available from Stratagene, Life Technologies, Inc., or Promega. Polypeptide may additionally be produced from the construct using in vitro translation systems such as the In vitro Express™ Translation Kit (Stratagene).

Following expression and purification of the secreted proteins encoded by the 5′ ESTs, cDNAs, or fragments thereof, the purified proteins may be tested for the ability to bind to the surface of various cell types as described below. It will be appreciated that a plurality of proteins expressed from these cDNAs may be included in a panel of proteins to be simultaneously evaluated for the activities specifically described below, as well as other biological roles for which assays for determining activity are available.

Alternatively, the polypeptide to be expressed may also be a product of transgenic animals, i.e., as a component of the milk of transgenic cows, goats, pigs or sheeps which are characterized by somatic or germ cells containing a nucleotide sequence encoding the protein of interest.

### EXAMPLE 19

Analysis of Secreted Proteins to Determine Whether they Bind to the Cell Surface

The proteins encoded by the cDNAs, or fragments thereof are cloned into expression vectors such as those described in the previous example. The proteins are purified by size, charge, immunochromatography or other techniques familiar to those skilled in the art. Following purification, the proteins are labeled using techniques known to those skilled in the art. The labeled proteins are incubated with cells or cell lines derived from a variety of organs or tissues to allow the proteins to bind to any receptor present on the cell surface. Following the incubation, the cells are washed to remove non-specifically bound protein. The labeled proteins are detected by autoradiography. Alternatively, unlabeled proteins may be incubated with the cells and detected with antibodies having a detectable label, such as a fluorescent molecule, attached thereto.

Specificity of cell surface binding may be analyzed by conducting a competition analysis in which various amounts of unlabeled protein are incubated along with the labeled protein. The amount of labeled protein bound to the cell surface decreases as the amount of competitive unlabeled protein increases. As a control, various amounts of an unlabeled protein unrelated to the labeled protein is included in some binding reactions. The amount of labeled protein bound to the cell surface does not decrease in binding reactions containing increasing amounts of unrelated unlabeled protein, indicating that the protein encoded by the cDNA binds specifically to the cell surface.

As discussed above, secreted proteins have been shown to have a number of important physiological effects and, consequently, represent a valuable therapeutic resource. The secreted proteins encoded by the cDNAs or fragments thereof made using any of the methods described therein may be evaluated to determine their physiological activities as described below.

### EXAMPLE 20

Assaying the Proteins Expressed from cDNAs or Fragments Thereof for Cytokine, Cell Proliferation or Cell Differentiation Activity

As discussed above, secreted proteins may act as cytokines or may affect cellular proliferation or differentiation. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7c and CMK. The proteins encoded by the above cDNAs or fragments thereof may be evaluated for their ability to regulate T cell or thymocyte proliferation in assays such as those described above or in the following references, which are incorporated herein by reference: Current Protocols in Immunology, Ed. by J. E. Coligan et al., Greene Publishing Associates and Wiley-Interscience; Takai et al. J. Immunol. 137:3494-3500, 1986. Bertagnolli et al. J. Immunol. 145:1706-1712, 1990. Bertagnolli et al., Cellular Immunology 133:327-341, 1991. Bertagnolli, et al. J. Immunol. 149:3778-3783, 1992; Bowman et al., J. Immunol. 152:1756-1761, 1994.

In addition, numerous assays for cytokine production and/or the proliferation of spleen cells, lymph node cells and thymocytes are known. These include the techniques disclosed in Current Protocols in Immunology. J. E. Coligan et al. Eds., Vol 1 pp. 3.12.1-3.12.14 John Wiley and Sons, Toronto. 1994; and Schreiber, R. D. Current Protocols in Immunology, supra Vol 1 pp. 6.8.1-6.8.8, John Wiley and Sons, Toronto. 1994.

The proteins encoded by the cDNAs may also be assayed for the ability to regulate the proliferation and differentiation of hematopoietic or lymphopoietic cells. Many assays for such activity are familiar to those skilled in the art, including the assays in the following references, which are incorporated herein by reference: Bottomly, K., Davis, L. S. and Lipsky, P. E., Measurement of Human and Murine Interleukin 2 and Interleukin 4, Current Protocols in Immunology., J. E. Coligan et al. Eds. Vol 1 pp. 6.3.1-6.3.12, John Wiley and Sons, Toronto. 1991; deVries et al., J. Exp. Med. 173:1205-1211, 1991; Moreau et al., Nature 36:690-692, 1988; Greenberger et al., Proc. Natl. Acad. Sci. U.S.A. 80:2931-2938, 1983; Nordan, R., Measurement of Mouse and Human Interleukin 6 Current Protocols in Immunology. J. E. Coligan et al. Eds. Vol 1 pp. 6.6.1-6.6.5, John Wiley and Sons, Toronto. 1991; Smith et al., Proc. Natl. Acad. Sci. U.S.A. 83:1857-1861, 1986; Bennett, F., Giannotti, J., Clark, S. C. and Turner, K. J., Measurement of Human Interleukin 11 Current Protocols in Immunology. J. E. Coligan et al. Eds. Vol 1 pp. 6.15.1 John Wiley and Sons, Toronto. 1991; Ciarlefta, A., Giannotti, J., Clark, S. C. and Turner, K. J., measurement of Mouse and Human Interleukin 9 Current Protocols in Immunology. J. E. Coligan et al., Eds. Vol 1 pp. 6.13.1, John Wiley and Sons, Toronto. 1991.

The proteins encoded by the cDNAs may also be assayed for their ability to regulate T-cell responses to antigens. Many assays for such activity are familiar to those skilled in the art, including the assays described in the following references, which are incorporated herein by reference: Chapter 3 (In vitro Assays for Mouse Lymphocyte Function), Chapter 6 (Cytokines and Their Cellular Receptors) and Chapter 7, (Immunologic Studies in Humans) in Current Protocols in Immunology, J. E. Coligan et al. Eds. Greene Publishing Associates and Wiley-Interscienc; Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Those proteins which exhibit cytokine, cell proliferation, or cell differentiation activity may then be formulated as pharmaceuticals and used to treat clinical conditions in which induction of cell proliferation or differentiation is beneficial. Alternatively, as described in more detail below, genes encoding these proteins or nucleic acids regulating the expression of these proteins may be introduced into appropriate host cells to increase or decrease the expression of the proteins as desired.

### EXAMPLE 21

Assaying the Proteins Expressed from cDNAs or Fragments Thereof for Activity as Immune System Regulators

The proteins encoded by the cDNAs may also be evaluated for their effects as immune regulators. For example, the proteins may be evaluated for their activity to influence thymocyte or splenocyte cytotoxicity. Numerous assays for such activity are familiar to those skilled in the art including the assays described in the following references, which are incorporated herein by reference: Chapter 3 (In vitro Assays for Mouse Lymphocyte Function 3.1-3.19) and Chapter 7 (Immunologic studies in Humans) in Current Protocols in Immunology, J. E. Coligan et al. Eds, Greene Publishing Associates and Wiley-Interscience; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; akai et al., J. Immunol. 140:508-512, 1988; Bertagnolli et al., Cellular Immunology 133:327-341, 1991; Brown et al., J. Immunol. 153:3079-3092, 1994.

The proteins encoded by the cDNAs may also be evaluated for their effects on T-cell dependent immunoglobulin responses and isotype switching. Numerous assays for such activity are familiar to those skilled in the art, including the assays disclosed in the following eferences, which are incorporated herein by reference: Maliszewski, J. Immunol. 144:3028-3033, 1990; Mond, J. J. and Brunswick, M Assays for B Cell Function: In vitro Antibody Production, Vol 1 pp. 3.8.1-3.8.16 in Current Protocols in Immunology. J. E. Coligan et al Eds., John Wiley and Sons, Toronto. 1994.

The proteins encoded by the cDNAs may also be evaluated for their effect on immune effector cells, including their effect on Th1 cells and cytotoxic lymphocytes. Numerous assays for such activity are familiar to those skilled in the art, including the assays disclosed in the following references, which are incorporated herein by reference: Chapter 3 (In vitro Assays for Mouse Lymphocyte Function 3.1-3.19) and Chapter 7 (Immunologic Studies in Humans) in Current Protocols in Immunology, J. E. Coligan et al. Eds., Greene Publishing Associates and Wiley-Interscience; Takai et al., J. Immunol. 137:3494-3500, 1986; Takai et al.; J. Immunol. 140:508-512, 1988; Bertagnolli et al., J. Immunol. 149:3778-3783, 1992.

The proteins encoded by the cDNAs may also be evaluated for their effect on dendritic cell mediated activation of naive T-cells. Numerous assays for such activity are familiar to those skilled in the art, including the assays disclosed in the following references, which are incorporated herein by reference: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:40624069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

The proteins encoded by the cDNAs may also be evaluated for their influence on the lifetime of lymphocytes. Numerous assays for such activity are familiar to those skilled in the art, including the assays disclosed in the following references, which are incorporated herein by reference: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal oflmmunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and evelopment include, without limitation, those described in: Antica et al., Blood 84:111-117, 1994; Fine et al., Cellular immunology 155:111-122, 1994; Galy et al., Blood 85:2770-2778, 1995; Toki et al., Proc. Nat. Acad. Sci. USA 88:7548-7551, 1991.

Those proteins which exhibit activity as immune system regulators activity may hen be formulated as pharmaceuticals and used to treat clinical conditions in which regulation of mmune activity is beneficial. For example, the protein may be useful in the treatment of various immune deficiencies and disorders (including severe combined immunodeficiency (SCID)), e.g., in egulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases caused by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to regulate immune responses, in a number of ways. Down regulation may be in the form of inhibiting or blocking an immune response already in progress or may involve preventing the induction of an immune response. The functions of activated T-cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al, Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/pr/pr mice or NZB hybrid mice, murine autoimmuno collagen arthritis, diabetes mellitus in OD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory form of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. The infected cells would now be capable of delivering a costimulatory signal to T cells in vivo, thereby activating the T cells.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acids encoding all or a fragment of (e.g., a cytoplasmic-domain truncated fragment) of an MHC class I α chain protein and P2 microglobulin protein or an MHC class II α chain protein and an MHC class II β chain protein to thereby express MHC class I or MHC class II proteins on the cell surface. Expression of the appropriate class II or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject. Alternatively, as described in more detail below, genes encoding these proteins or nucleic acids regulating the expression of these proteins may be introduced into appropriate host cells to increase or decrease the expression of the proteins as desired.

### EXAMPLE 22

Assaying the Proteins Expressed from cDNAs or Fragments thereof for Hematopoiesis Regulating Activity

The proteins encoded by the cDNAs or fragments thereof may also be evaluated for their hematopoiesis regulating activity. For example, the effect of the proteins on embryonic stem cell differentiation may be evaluated. Numerous assays for such activity are familiar to those skilled in the art, including the assays disclosed in the following references, which are incorporated herein by reference: Johansson et al. Cellular Biology 15:141-151, 1995; Keller et al., Molecular and Cellular Biology 13:473-486, 1993; McClanahan et al., Blood 81:2903-2915, 1993.

The proteins encoded by the cDNAs or fragments thereof may also be evaluated for their influence on the lifetime of stem cells and stem cell differentiation. Numerous assays for such activity are familiar to those skilled in the art, including the assays disclosed in the following references, which are incorporated herein by reference: Freshney, M. G. Methylcellulose Colony Forming Assays, in Culture of Hematopoietic Cells. R. I. Freshney, et al. Eds. pp. 265-268, Wiley-Liss, Inc., New York, N.Y. 1994; Hirayama et al., Proc. Natl. Acad. Sci. USA 89:5907-5911, 1992; McNiece, I. K. and Briddell, R. A. Primitive Hematopoietic Colony Forming Cells with High Proliferative Potential, in Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Ploemacher, R. E. Cobblestone Area Forming Cell Assay, In Culture of Hematopoietic Cells. R. I. Freshney, et al. Eds. pp. 1-21, Wiley-Liss, Inc., New York, N.Y. 1994; Spooncer, E., Dexter, M. and Allen, T. Long Term Bone Marrow Cultures in the Presence of Stromal Cells, in Culture of Hematopoietic Cells. R. I. Freshney, et al. Eds. pp. 163-179, Wiley-Liss, Inc., New York, N.Y. 1994; and Sutherland, H. J. Long Term Culture Initiating Cell Assay, in Culture of Hematopoietic Cells. R. I. Freshney, et al. Eds. pp. 139-162, Wiley-Liss, Inc., New York, N.Y. 1994.

Those proteins which exhibit hematopoiesis regulatory activity may then be formulated as pharmaceuticals and used to treat clinical conditions in which regulation of hematopoeisis is beneficial. For example, a protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantion, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy. Alternatively, as described in more detail below, genes encoding these proteins or nucleic acids regulating the expression of these proteins may be introduced into appropriate host cells to increase or decrease the expression of the proteins as desired.

### EXAMPLE 23

Assaying the Proteins Expressed from cDNAs or Fragments thereof for Regulation of Tissue Growth

The proteins encoded by the cDNAs or fragments thereof may also be evaluated for their effect on tissue growth. Numerous assays for such activity are familiar to those skilled in the art, including the assays disclosed in International Patent Publication No. WO95/16035, International Patent Publication No. WO95/05846 and International Patent Publication No. WO91/07491, which are incorporated herein by reference.

Assays for wound healing activity include, without limitation, those described in: Winter, Epidermal Wound Healing, pps. 71-112 (Maibach, H1 and Rovee, D T, eds.), Year Book Medical Publishers, Inc., Chicago, as modified by Eaglstein and Mertz, J. Invest. Dermatol 71:382-84 (1978) which are incorporated herein by reference.

Those proteins which are involved in the regulation of tissue growth may then be formulated as pharmaceuticals and used to treat clinical conditions in which regulation of tissue growth is beneficial. For example, a protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e., for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium) muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to generate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokinc damage.

A protein of the present invention may also be useful for promoting or inhibiting differentiation of tissues described above from precursor tissues or cells; or for inhibiting the growth of tissues described above.

Alternatively, as described in more detail below, genes encoding these proteins or nucleic acids regulating the expression of these proteins may be introduced into appropriate host cells to increase or decrease the expression of the proteins as desired.

### EXAMPLE 24

Assaying the Proteins Expressed from cDNAs or Fragments Thereof for Regulation of Reproductive Hormones or Cell Movement

The proteins encoded by the cDNAs or fragments thereof may also be evaluated for their ability to regulate reproductive hormones, such as follicle stimulating hormone. Numerous assays for such activity are familiar to those skilled in the art, including the assays disclosed in the following references, which are incorporated herein by reference: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986. Chapter 6.12 (Measurement of Alpha and Beta Chemokines) Current Protocols in Immunology, J. E. Coligan et al. Eds. Greene Publishing Associates and Wiley-Intersciece; Taub et al. J. Clin. Invest. 95:1370-1376, 1995; Lind et al. APMIS 103:140-146, 1995; Muller et al. Eur. J. Immunol. 25:1744-1748; Gruber et al. J. of Immunol. 152:5860-5867, 1994; Johnston et al. J. of Immunol 153:1762-1768, 1994.

Those proteins which exhibit activity as reproductive hormones or regulators of cell movement may then be formulated as pharmaceuticals and used to treat clinical conditions in which regulation of reproductive hormones or cell movement are beneficial. For example, a protein of the present invention may also exhibit activin- or inhibin-related activities. Inhibins are characterized by their ability to inhibit the release of follicle stimulating hormone (FSH), while activins are characterized by their ability to stimulate the release of folic stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-B group, may be useful as a fertility inducing therapeutic, based upon the ability of activin molecules in stimulating FSH release from cells of the anterior pituitary. See, for example, U.S. Pat. No. 4,798,885, the disclosure of which is incorporated herein by reference. A protein of the invention may also be useful for advancement of the onset of fertility in sexually immature mammals, so as to increase the lifetime reproductive performance of domestic animals such as cows, sheep and pigs.

Alternatively, as described in more detail below, genes encoding these proteins or nucleic acids regulating the expression of these proteins may be introduced into appropriate host cells to increase or decrease the expression of the proteins as desired.

### EXAMPLE 25

Assaying the Proteins Expressed from cDNAs or Fragments thereof for Chemotactic/Chemokinetic Activity

The proteins encoded by the cDNAs or fragments thereof may also be evaluated for chemotactic/chemokinetic activity. For example, a protein of the present invention may have chemotactic or chemokinetic activity (e.g., act as a chemokine) for mammalian cells, including, for example, monocytes, fibroblasts, neutrophils, T-cells, mast cells, cosinophils, epithelial and/or endothelial cells. Chemotactic and chmokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhension of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokincs 6.12.1-6.12.28; Taub et al. J. Clin. Invest. 95:1370-1376, 1995; Lind et al. APMIS 103:140-146, 1995; Mueller et al Eur. J. Immunol. 25:1744-1748; Gruber et al. J. of Immunol. 152:5860-5867, 1994; Johnston et al. J. of Immunol, 153:1762-1768, 1994.

### EXAMPLE 26

Assaying the Proteins Expressed from cDNAs or Fragments Thereof for Regulation of Blood Clotting

The proteins encoded by the cDNAs or fragments thereof may also be evaluated for their effects on blood clotting. Numerous assays for such activity are familiar to those skilled in the art, including the assays disclosed in the following references, which are incorporated herein by reference: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Those proteins which are involved in the regulation of blood clotting may then be formulated as pharmaceuticals and used to treat clinical conditions in which regulation of blood clotting is beneficial. For example, a protein of the invention may also exhibit hemostatic or thrombolytic activity. As a result, such a protein is expected to be useful in treatment of various coagulations disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke)). Alternatively, as described in more detail below, genes encoding these proteins or nucleic acids regulating the expression of these proteins may be introduced into appropriate host cells to increase or decrease the expression of the proteins as desired.

### EXAMPLE 27

Assaying the Proteins Expressed from cDNAs or Fragments Thereof for Involvement in Receptor/Ligand Interactions

The proteins encoded by the cDNAs or a fragment thereof may also be evaluated for their involvement in receptor/ligand interactions. Numerous assays for such involvement are familiar to those skilled in the art, including the assays disclosed in the following references, which are incorporated herein by reference: Chapter 7.28 (Measurement of Cellular Adhesion under Static Conditions 7.28.1-7.28.22) in Current Protocols in Immunology, J. E. Coligan et al. Eds. Greene Publishing Associates and Wiley-Interscience; Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160, 1989; Stoltenborg et al., J. Immunol. Methods 175:59-68, 1994; Stitt et al., Cell 80:661-670, 1995; Gyuris et al., Cell 75:791-803, 1993.

For example, the proteins of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune respones). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

### EXAMPLE 28

Assaying the Proteins Expressed from cDNAs or Fragments Thereof for Anti-Inflammatory Activity

The proteins encoded by the cDNAs or a fragment thereof may also be evaluated for anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusioninury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

### EXAMPLE 29

Assaying the Proteins Expressed from cDNAs or Fragments Thereof for Tumor Inhibition Activity

The proteins encoded by the cDNAs or a fragment thereof may also be evaluated for tumor inhibition activity. In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

### EXAMPLE 30

Identification of Proteins which Interact with Polypeptides Encoded by cDNAs

Proteins which interact with the polypeptides encoded by cDNAs or fragments thereof, such as receptor proteins, may be identified using two hybrid systems such as the Matchmaker Two Hybrid System 2 (Catalog No. K1604-1, Clontech). As described in the manual accompanying the Matchmaker Two Hybrid System 2 (Catalog No. K1604-1, Clontech), which is incorporated herein by reference, the cDNAs or fragments thereof, are inserted into an expression vector such that they are in frame with DNA encoding the DNA binding domain of the yeast transcriptional activator GAL4. cDNAs in a cDNA library which encode proteins which might interact with the polypeptides encoded by the cDNAs or fragments thereof are inserted into a second expression vector such that they are in frame with DNA encoding the activation domain of GAL4. The two expression plasmids are transformed into yeast and the yeast are plated on selection medium which selects for expression of selectable markers on each of the expression vectors as well as GAL4 dependent expression of the HIS3 gene. Transformants capable of growing on medium lacking histidine are screened for GAL4 dependent lacZ expression. Those cells which are positive in both the histidine selection and the lacZ assay contain plasmids encoding proteins which interact with the polypeptide encoded by the cDNAs or fragments thereof.

Alternatively, the system described in Lustig et al., Methods in Enzymology 283: 83-99 (1997), the disclosure of which is incorporated herein by reference, may be used for identifying molecules which interact with the polypeptides encoded by cDNAs. In such systems, in vitro transcription reactions are performed on a pool of vectors containing cDNA inserts cloned downstream of a promoter which drives in vitro transcription. The resulting pools of mRNAs are introduced into Xenopus laevis oocytes. The oocytes are then assayed for a desired acitivity.

Alternatively, the pooled in vitro transcription products produced as described above may be translated in vitro. The pooled in vitro translation products can be assayed for a desired activity or for interaction with a known polypeptide.

Proteins or other molecules interacting with polypeptides encoded by cDNAs can be found by a variety of additional techniques. In one method, affinity columns containing the polypeptide encoded by the cDNA or a fragment thereof can be constructed. In some versions, of this method the affinity column contains chimeric proteins in which the protein encoded by the cDNA or a fragment thereof is fused to glutathione S-transferase. A mixture of cellular proteins or pool of expressed proteins as described above and is applied to the affinity column. Proteins interacting with the polypeptide attached to the column can then be isolated and analyzed on 2-D electrophoresis gel as described in Ramunsen et al. Electrophoresis, 18, 588-598 (1997), the disclosure of which is incorporated herein by reference. Alternatively, the proteins retained on the affinity column can be purified by electrophoresis based methods and sequenced. The same method can be used to isolate antibodies, to screen phage display products, or to screen phage display human antibodies.

Proteins interacting with polypeptides encoded by cDNAs or fragments thereof can also be screened by using an Optical Biosensor as described in Edwards & Leatherbarrow, Analytical Biochemistry, 246, 1-6 (1997), the disclosure of which is incorporated herein by reference. The main advantage of the method is that it allows the determination of the association rate between the protein and other interacting molecules. Thus, it is possible to specifically select interacting molecules with a high or low association rate. Typically a target molecule is linked to the sensor surface (through a carboxymethl dextran matrix) and a sample of test molecules is placed in contact with the target molecules. The binding of a test molecule to the target molecule causes a change in the refractive index and/or thickness. This change is detected by the Biosensor provided it occurs in the evanescent field (which extend a few hundred manometers from the sensor surface). In these screening assays, the target molecule can be one of the polypeptides encoded by cDNAs or a fragment thereof and the test sample can be a collection of proteins extracted from tissues or cells, a pool of expressed proteins, combinatorial peptide and/or chemical libraries, or phage displayed peptides. The tissues or cells from which the test proteins are extracted can originate from any species.

In other methods, a target protein is immobilized and the test population is a collection of unique polypeptides encoded by the cDNAs or fragments thereof.

To study the interaction of the proteins encoded by the cDNAs or fragments thereof with drugs, the microdialysis coupled to HPLC method described by Wang et al., Chromatographia, 44, 205-208(1997) or the affinity capillary electrophoresis method described by Busch et al, J. Chromatogr. 777:311-328 (1997), the disclosures of which are incorporated herein by reference can be used.

The system described in U.S. Pat. No. 5,654,150, the disclosure of which is incorporated herein by reference, may also be used to identify molecules which interact with the polypeptides encoded by the cDNAs. In this system, pools of cDNAs are transcribed and translated in vitro and the reaction products are assayed for interaction with a known polypeptide or antibody.

It will be appreciated by those skilled in the art that the proteins expressed from the cDNAs or fragments may be assayed for numerous activities in addition to those specifically enumerated above. For example, the expressed proteins may be evaluated for applications involving control and regulation of inflammation, tumor proliferation or metastasis, infection, or other clinical conditions. In addition, the proteins expressed from the cDNAs or fragments thereof may be useful as nutritional agents or cosmetic agents.

The proteins expressed from the cDNAs or fragments thereof may be used to generate antibodies capable of specifically binding to the expressed protein or fragments thereof as described below. The antibodies may capable of binding a full length protein encoded by one of the sequences of SEQ ID NOs. 1-405, a mature protein encoded by one of the sequences of SEQ ID NOs. 1-405, or a signal peptide encoded by one of the sequences of SEQ ID Nos. 1-405. Alternatively, the antibodies may be capable of binding fragments of the proteins expressed from the cDNAs which comprise at least 10 amino acids of the sequences of SEQ ID NOs: 406-810. In some embodiments, the antibodies may be capable of binding fragments of the proteins expressed from the cDNAs which comprise at least 15 amino acids of the sequences of SEQ ID NOs: 406-810. In other embodiments, the antibodies may be capable of binding fragments of the proteins expressed from the cDNAs which comprise at least 25 amino acids of the sequences of SEQ ID NOs: 406-810. In further embodiments, the antibodies may be capable of binding fragments of the proteins expressed from the cDNAs which comprise at least 40 amino acids of the sequences of SEQ ID NOs: 406-810.

### EXAMPLE 31

Epitopes and Antibody Fusions

A preferred embodiment of the present invention is directed to eiptope-bearing polypeptides and epitope-bearing polypeptide fragments. These epitopes may be “antigenic epitopes” or both an “antigenic epitope” and an “immunogenic epitope”. An “immunogenic epitope” is defined as a part of a protein that elicits an antibody response in vivo when the polypeptide is the immunogen. On the other hand, a region of polypeptide to which an antibody binds is defined as an “antigenic determinant” or “antigenic epitope.” The number of immunogenic epitopes of a protein generally is less than the number of antigenic epitopes (See, e.g., Geysen, et al., 1983). It is particularly noted that although a particular epitope may not be immunogenic, it is nonetheless useful since antibodies can be made to both immunogenic and antigenic epitopes.

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes may be produced by any conventional means (See, e.g., Houghten, R. A., 1985), also, further described in U.S. Pat. No. 4,631,211. Methods for determining the amino acids which make up an epitope include x-ray crystallography, 2-dimensional nuclear magnetic resonance, and epitope mapping, e.g., the Pepscan method described by Mario H. Geysen et al. (1984); PCT Publication No. WO 84/03564; and PCT Publication No. WO 84/03506. Epitopes may also be delineated using an algorithm, such as the algorithm of Jameson and Wolf, (Jameson and Wolf, Comp. Appl. Biosci. 4:181-186 (1988). The Jameson-Wolf antigenic analysis, for example, may be performed using the computer program PROTEAN, using default parameters (Version 4.0 Windows, DNASTAR, Inc., 1228 South Park Street Madison, Wis.

Table X lists antigenic peaks of predicted antigenic epitopes identified by the Jameson-Wolf algorithm. For each polypeptide referred to by its sequence identification number in the first column, the second colmun gives a list of antigenic peaks separated by a coma. Preferred antigenic epitopes of the present invention comprise an additional 6 amino acid residues both N-terminal and C-terminal to the positions listed in the Table. For example, for SEQ ID NO:406, the first preferred immunogenic epitope comprises amino acid residues 52 to 64. Note that for the purposes of this Table, position 1 is the N-terminal methionine residue, i.e., the leader sequence is not numbered negatively.

It is pointed out that the immunogenic epitope list describe only amino acid residues comprising epitopes predicted to have the highest degree of immunogenicity by a particular algorithm. Polypeptides of the present invention that are not specifically described as immunogenic are not considered non-antigenic. This is because they may still be antigenic in vivo but merely not recognized as such by the particular algorithm used. Alternatively, the polypeptides are probably antigenic in vitro using methods such a phage display. In fact, all fragments of the polypeptides of the present invention, at least 6 amino acids residues in length, are included in the present invention as being useful as antigenic epitope. Moreover, listed in Table IX are only the critical residues of the epitopes determined by the Jameson-Wolf analysis. Thus, additional flanking residues on either the N-terminal, C-terminal, or both N- and C-terminal ends may be added to the sequences listed to generate an epitope-bearing portion at least 6 residues in length. Amino acid residues comprising other immunogenic epitopes may be determined by algorithms similar to the Jameson-Wolf analysis or by in vivo testing for an antigenic response using the methods described herein or those known in the art.

The epitope-bearing fragments of the present invention preferably comprises 6 to 50 amino acids (i.e. any integer between 6 and 50, inclusive) of a polypeptide of the present invention. Also, included in the present invention are antigenic fragments between the integers of 6 and the full length polypeptide sequence of the sequence listing. All combinations of sequences between the integers of 6 and the full-length sequence of a polypeptide are included. The epitope-bearing fragments may be specified by either the number of contiguous amino acid residues (as a sub-genus) or by specific N-terminal and C-terminal positions (as species) as described above for the polypeptide fragments of the present invention. Any number of epitope-bearing fragments of the present invention may also be excluded in the same manner.

Antigenic epitopes are useful, for example, to raise antibodies, including monoclonal antibodies that specifically bind the epitope (See, Wilson et al., 1984; and Sutcliffe, J. G. et al., 1983). The antibodies are then used in various techniques such as diagnostic and tissue/cell identification techniques, as described herein, and in purification methods.

Similarly, immunogenic epitopes can be used to induce antibodies according to methods well known in the art (See, Sutcliffe et al., supra; Wilson et al., supra; Chow, M. et al.; (1985) and Bittle, F. J. et al., (1985)). The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.).

Epitope-bearing polypeptides of the present invention are used to induce antibodies according to methods well known in the art including, but not limited to, in vivo immunization, in vitro immunization, and phage display methods (See, e.g., Sutcliffe, et al., supra; Wilson, et al., supra, and Bittle, et al., 1985). If in vivo immunization is used, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μgs of peptide or carrier protein and Freund's adjuvant. Several booster injections may be needed, for instance, at intervals of about two weeks, to provide a useful titer of anti-peptide antibody, which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and discussed above, the polypeptides of the present invention comprising an immunogenic or antigenic epitope can be fused to heterologous polypeptide sequences. For example, the polypeptides of the present invention may be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo. This has been shown, e.g., for chimeric proteins consisting of the first two domains of the human CD4-polypeptide and various domains of the constant regions of the heavy or light chains of mammalian immunoglobulins (See, e.g., EPA 0,394,827; and Traunecker et al., 1988). Fusion proteins that have a disulfide-linked dimeric structure due to the IgG portion can also be more efficient in binding and neutralizing other molecules than monomeric polypeptides or fragments thereof alone (See, e.g., Fountoulakis et al., 1995). Nucleic acids encoding the above epitopes can also be recombined with a gene of interest as an epitope tag to aid in detection and purification of the expressed polypeptide.

Additonal fusion proteins of the invention may be generated through the techniques of gene-shuffling, motif-shuffling, exon-shuffling, or codon-shuffling (collectively referred to as “DNA shuffling”). DNA shuffling may be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; and Patten, P. A., et al., (1997); Harayama, S., (1998); Hansson, L. O., et al (1999); and Lorenzo, M. M. and Blasco, R., (1998). In one embodiment, one or more components, motifs, sections, parts, domains, fragments, etc., of coding polynucleotides of the invention, or the polypeptides encoded thereby may be recombined with one or more components, motifs, sections, parts, domains, fragments, etc. of one or more heterologous molecules.

Antibodies:

The present invention further relates to antibodies and T-cell antigen receptors (TCR), which specifically bind the polypeptides, and more specifically, the epitopes of the polyepeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen binding fragments thereof. In a preferred embodiment the antibodies are human antigen binding antibody fragments of the present invention include, but are not limited to, Fab, Fab′F(ab)2 and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies, which specifically bind the polypeptides of the present invention. The present invention further includes antibodies that are anti-idiotypic to the antibodies of the present invention.

The antibodies of the present invention may be monospecific, bispecific, and trispecific or have greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, A. et al. (1991); U.S. Pat. Nos. 5,573,920, 4,474,893, 5,601,819, 4,714,681, 4,925,648; Kostelny, S. A. et al. (1992).

Antibodies of the present invention may be described or specified in terms of the epitope(s) or epitope-bearing portion(s) of a polypeptide of the present invention, which are recognized or specifically bound by the antibody. In the case of proteins of the present invention secreted proteins, the antibodies may specifically bind a full-length protein encoded by a nucleic acid of the present invention, a mature protein (i.e., the protein generated by cleavage of the signal peptide) encoded by a nucleic acid of the present invention, a signal peptide encoded by a nucleic acid of the present invention, or any other polypeptide of the present invention. Therefore, the epitope(s) or epitope bearing polypeptide portion(s) may be specified as described herein, e.g., by N-terminal and C-terminal positions, by size in contiguous amino acid residues, or otherwise described herein (including the squence listing). Antibodies which specifically bind any epitope or polypeptide of the present invention may also be excluded as individual species. Therefore, the present invention includes antibodies that specifically bind specified polypeptides of the present invention, and allows for the exclusion of the same.

Antibodies of the present invention may also be described or specified in terms of their cross-reactivity. Antibodies that do not specifically bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein, eg., using FASTDB and the parameters set forth herein) to a polypeptide of the present invention are also included in the present invention. Further included in the present invention are antibodies, which only bind polypeptides encoded by polynucleotides, which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10¹⁵ M, and 10⁻¹⁵ M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples (See, e.g., Harlow et al., 1988).

The antibodies of the present invention may be used either alone or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the resent invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not limited to antibodies produced through hybridoma technology. The term “antibody” refers to a polypeptide or group of polypeptides which are comprised of at least one binding domain, where a binding domain is formed from the folding of variable domains of an antibody molecule to form three-dimensional binding spaces with an internal surface shape and charge distribution complementary to the features of an antigenic determinant of an antigen, which allows an immunological reaction with the antigen. The term “monoclonal antibody” refers to an antibody that is derived from a single clone, including eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art (See, e.g., Harlow et al. 1988); Hammerling, et al, 1981). (Said references incorporated by reference in their entireties). Fab and F(ab′)2 fragments may be produced, for example, from hybridoma-produced antibodies by proteolytic cleavage, using enzymes such as papain (to produce Fab fragments) or pepsin (to produce F(ab′)2 fragments).

Alternatively, antibodies of the present invention can be produced through the application of recombinant DNA technology or through synthetic chemistry using methods known in the art. For example, the antibodies of the present invention can be prepared using various phage display methods known in the art. In phage display methods, functional antibody domains are displayed on the surface of a phage particle, which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman U. et al. (1995); Ames, R. S. et al. (1995); Kettleborough, C. A. et al. (1994); Persic, L. et al. (1997); Burton, D. R. et al. (1994); PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ F(ab)2 and F(ab′)2 fragments can also be employed using methods known in the art such as those disclosed in WO 92/22324; Mullinax, R. L. et al. (1992); and Sawai, H. et al. (1995); and Better, M. et al. (1988).

Examples of techniques which can be used to produce single-chain Fvs and antibodies include those described in U.S. Pat. Nos. 4,946,778 and 5,258,498; Huston et al. (1991); Shu, L. et al. (1993); and Skerra, A. et al. (1988). For some uses, including in vivo use of antibodies in humans and in vitro detection assays, it may be preferable to use chimeric, humanized, or human antibodies. Methods for producing chimeric antibodies are known in the art. See e.g., Morrison, (1985); Oi et al., (1986); Gillies, S. D. et al. (1989); and U.S. Pat. No. 5,807,715. Antibodies can be humanized using a variety of techniques including CDR-grafting (EP 0 239 400; WO 91/09967; U.S. Pat. No. 5,530,101; and 5,585,089), veneering or resurfacing, (EP 0 592 106; EP 0 519 596; Padlan E. A., 1991; Studnicka G. M. et al., 1994; Roguska M. A. et al., 1994), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; WO 98/46645; WO 98/50433; WO 98/24893; WO 96/34096; WO 96/33735; and WO 91/10741.

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art (See e.g., Harbor et al. supra; WO 93/21232; EP 0 439 095; Naramura, M. et al. 1994; U.S. Pat. No. 5,474,981; Gillies, S. O. et al., 1992; Fell, H. P. et al., 1991).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half-life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al. (1991); Zheng, X.X. et al. (1995); and Vil, H. et al. (1992).

The invention further relates to antibodies that act as agonists or antagonists of the polypeptides of the present invention. For example, the present invention includes antibodies that disrupt the receptor/ligand interactions with the polypeptides of the invention either partially or fully. Included are both receptor-specific antibodies and ligand-specific antibodies. Included are receptor-specific antibodies, which do not prevent ligand binding but prevent receptor activation. Receptor activation (i.e., signaling) may be determined by techniques described herein or otherwise known in the art. Also include are receptor-specific antibodies which both prevent ligand binding and receptor activation. Likewise, included are neutralizing antibodies that bind the ligand and prevent binding of the ligand to the receptor, as well as antibodies that bind the ligand, thereby preventing receptor activation, but do not prevent the ligand from binding the receptor. Further included are antibodies that activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al. (1998); Chen, Z. et al. (1998); Harrop, J. A. et al. (1998); Zhu, Z. et al. (1998); Yoon, D. Y. et al. (1998); Prat, M. et al. (1998) J.; Pitard, V. et al. (1997); Liautard, J. et al. (1997); Carlson, N. G. et al. (1997) J.; Taryman, R. E. et al. (1995); Muller, Y. A. et al. (1998); Bartunek, P. et al. (1996).

As discussed above, antibodies of the polypeptides of the invention can, in turn, be utilized to generate anti-idiotypic antibodies that “mimic” polypeptides of the invention using techniques well known to those skilled in the art (See, e.g. Greenspan and Bona (1989); and Nissinoff (1991). For example, antibodies which bind to and competitively inhibit polypeptide multimerization or binding of a polypeptide of the invention to ligand can be used to generate anti-idiotypes that “mimic” the polypeptide multimerization or binding domain and, as a consequence, bind to and neutralize polypeptide or its ligand. Such neutralization anti-idiotypic antibodies can be used to bind a polypeptide of the invention or to bind its ligands/receptors, and therby block its biological activity,

The invention also concerns a purified or isolated antibody capable of specifically binding to a mutated full length or mature polypeptide of the present invention or to a fragment or variant thereof comprising an epitope of the mutated polypeptide. In another preferred embodiment, the present invention concerns an antibody capable of binding to a polypeptide comprising at least 10 consecutive amino acids of a polypeptide of the present invention and including at least one of the amino acids which can be encoded by the trait causing mutations.

Non-human animals or mammals, whether wild-type or transgenic, which express a different species of a polypeptide of the present invention than the one to which antibody binding is desired, and animals which do not express a polypeptide of the present invention (i.e. a knock out animal) are particularly useful for preparing antibodies. Gene knock out animals will recognize all or most of the exposed regions of a polypeptide of the present invention as foreign antigens, and therefore produce antibodies with a wider array of epitopes. Moreover, smaller polypeptides with only 10 to 30 amino acids may be useful in obtaining specific binding to any one of the polypeptides of the present invention. In addition, the humoral immune system of animals which produce a species of a polypeptide of the present invention that resembles the antigenic sequence will preferentially recognize the differences between the animal's native polypeptide species and the antigen sequence, and produce antibodies to these unique sites in the antigen sequence. Such a technique will be particularly useful in obtaining antibodies that specifically bind to any one of the polypeptides of the present invention.

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

The antibodies of the invention may be labeled by any one of the radioactive, fluorescent or enzymatic labels known in the art.

Consequently, the invention is also directed to a method for detecting specifically the presence of a polypeptide of the present invention according to the invention in a biological sample, said method comprising the following steps:
a) bringing into contact the biological sample with a polyclonal or monoclonal antibody that specifically binds a polypeptide of the present invention; and
b) detecting the antigen-antibody complex formed.

The invention also concerns a diagnostic kit for detecting in vitro the presence of a polypeptide of the present invention in a biological sample, wherein said kit comprises:
a) a polyclonal or monoclonal antibody that specifically binds a polypeptide of the present invention, optionally labeled;
b) a reagent allowing the detection of the antigen-antibody complexes formed, said reagent carrying optionally a label, or being able to be recognized itself by a labeled reagent, more particularly in the case when the above-mentioned monoclonal or polyclonal antibody is not labeled by itself.
   A. Monoclonal Antibody Production by Hybridoma Fusion

Monoclonal antibody to epitopes of any of the peptides identified and isolated as described can be prepared from murine hybridomas according to the classical method of Kohler, G. and Milstein, C., Nature 256:495 (1975) or derivative methods thereof. Briefly, a mouse is repetitively inoculated with a few micrograms of the selected protein or peptides derived therefrom over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as Elisa, as originally described by Engvall, E., Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

B. Polyclonal Antibody Production by Immunization

Polyclonal antiserum containing antibodies to heterogenous epitopes of a single protein can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than others and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991(1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980).

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies may also be used in therapeutic compositions for killing cells expressing the protein or reducing the levels of the protein in the body.

V. Use of cDNAs or Fragments thereof as Reagents

The cDNAs of the present invention may be used as reagents in isolation procedures, diagnostic assays, and forensic procedures. For example, sequences from the cDNAs (or genomic DNAs obtainable therefrom) may be detectably labeled and used as probes to isolate other sequences capable of hybridizing to them. In addition, sequences from the cDNAs (or genomic DNAs obtainable therefrom) may be used to design PCR primers to be used in isolation, diagnostic, or forensic procedures.

### EXAMPLE 32

Preparation of PCR Primers and Amplification of DNA

The cDNAs (or genomic DNAs obtainable therefrom) may be used to prepare PCR primers for a variety of applications, including isolation procedures for cloning nucleic acids capable of hybridizing to such sequences, diagnostic techniques and forensic techniques. The PCR primers are at least 10 bases, and preferably at least 12, 15, or 17 bases in length. More preferably, the PCR primers are at least 20-30 bases in length. In some embodiments, the PCR primers may be more than 30 bases in length. It is preferred that the primer pairs have approximately the same G/C ratio, so that melting temperatures are approximately the same. A variety of PCR techniques are familiar to those skilled in the art. For a review of PCR technology, see Molecular Cloning to Genetic Engineering White, B. A. Ed. in Methods in Molecular Biology 67: Humana Press, Totowa 1997. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites.

### EXAMPLE 33

Use of cDNAs as Probes

Probes derived from cDNAs or fragments thereof (or genomic DNAs obtainable therefrom) may be labeled with detectable labels familiar to those skilled in the art, including radioisotopes and non-radioactive labels, to provide a detectable probe. The detectable probe may be single stranded or double stranded and may be made using techniques known in the art, including in vitro transcription, nick translation, or kinase reactions. A nucleic acid sample containing a sequence capable of hybridizing to the labeled probe is contacted with the labeled probe. If the nucleic acid in the sample is double stranded, it may be denatured prior to contacting the probe. In some applications, the nucleic acid sample may be immobilized on a surface such as a nitrocellulose or nylon membrane. The nucleic acid sample may comprise nucleic acids obtained from a variety of sources, including genomic DNA, cDNA libraries, RNA, or tissue samples.

Procedures used to detect the presence of nucleic acids capable of hybridizing to the detectable probe include well known techniques such as Southern blotting, Northern blotting, dot blotting, colony hybridization, and plaque hybridization. In some applications, the nucleic acid capable of hybridizing to the labeled probe may be cloned into vectors such as expression vectors, sequencing vectors, or in vitro transcription vectors to facilitate the characterization and expression of the hybridizing nucleic acids in the sample. For example, such techniques may be used to isolate and clone sequences in a genomic library or cDNA library which are capable of hybridizing to the detectable probe as described in example 17 above.

PCR primers made as described in example 32 above may be used in forensic analyses, such as the DNA fingerprinting techniques described in Examples 34-38 below. Such analyses may utilize detectable probes or primers based on the sequences of the cDNAs or fragments thereof (or genomic DNAs obtainable therefrom).

### EXAMPLE 34

Forensic Matching by DNA Sequencing

In one exemplary method, DNA samples are isolated from forensic specimens of, for example, hair, semen, blood or skin cells by conventional methods. A panel of PCR primers based on a number of the cDNAs (or genomic DNAs obtainable therefrom), is then utilized in accordance with example 32 to amplify DNA of approximately 100-200 bases in length from the forensic specimen. Corresponding sequences are obtained from a test subject. Each of these identification DNAs is then sequenced using standard techniques, and a simple database comparison determines the differences, if any, between the sequences from the subject and those from the sample. Statistically significant differences between the suspect's DNA sequences and those from the sample conclusively prove a lack of identity. This lack of identity can be proven, for example, with only one sequence. Identity, on the other hand, should be demonstrated with a large number of sequences, all matching. Preferably, a minimum of 50 statistically identical sequences of 100 bases in length are used to prove identity between the suspect and the sample.

### EXAMPLE 35

Positive Identification by DNA Sequencing

The technique outlined in the previous example may also be used on a larger scale to provide a unique fingerprint-type identification of any individual. In this technique, primers are prepared from a large number of sequences from Table I and the appended sequence listing. Preferably, 20 to 50 different primers are used. These primers are used to obtain a corresponding number of PCR-generated DNA segments from the individual in question in accordance with example 32. Each of these DNA segments is sequenced, using the methods set forth in example 34. The database of sequences generated through this procedure uniquely identifies the individual from whom the sequences were obtained. The same panel of primers may then be used at any later time to absolutely correlate tissue or other biological specimen with that individual.

### EXAMPLE 36

Southern Blot Forensic Identification

The procedure of example 35 is repeated to obtain a panel of at least 10 amplified sequences from an individual and a specimen. Preferably, the panel contains at least 50 amplified sequences. More preferably, the panel contains 100 amplified sequences. In some embodiments, the panel contains 200 amplified sequences. This PCR-generated DNA is then digested with one or a combination of, preferably, four base specific restriction enzymes. Such enzymes are commercially available and known to those of skill in the art. After digestion, the resultant gene fragments are size separated in multiple duplicate wells on an agarose gel and transferred to nitrocellulose using Southern blotting techniques well known to those with skill in the art. For a review of Southern blotting see Davis et al. (Basic Methods in Molecular Biology, 1986, Elsevier Press. pp 62-65).

A panel of probes based on the sequences of the cDNAs (or genomic DNAs obtainable therefrom), or fragments thereof of at least 10 bases, are radioactively or calorimetrically labeled using methods known in the art, such as nick translation or end labeling, and hybridized to the Southern blot using techniques known in the art (Davis et al., supra). Preferably, the probe comprises at least 12, 15, or 17 consecutive nucleotides from the cDNA (or genomic DNAs obtainable therefrom). More preferably, the probe comprises at least 20-30 consecutive nucleotides from the cDNA (or genomic DNAs obtainable therefrom). In some embodiments, the probe comprises more than 30 nucleotides from the cDNA (or genomic DNAs obtainable therefrom). In other embodiments, the probe comprises at least 40, at least 50, at least 75, at least 100, at least 150, or at least 200 consecutive nucleotides from the cDNA (or genomic DNAs obtainable therefrom).

Preferably, at least 5 to 10 of these labeled probes are used, and more preferably at least about 20 or 30 are used to provide a unique pattern. The resultant bands appearing from the hybridization of a large sample of cDNAs (or genomic DNAs obtainable therefrom) will be a 20 unique identifier. Since the restriction enzyme cleavage will be different for every individual, the band pattern on the Southern blot will also be unique. Increasing the number of cDNA probes will provide a statistically higher level of confidence in the identification since there will be an increased number of sets of bands used for identification.

### EXAMPLE 37

Dot Blot Identification Procedure

Another technique for identifying individuals using the cDNA sequences disclosed herein utilizes a dot blot hybridization technique.

Genomic DNA is isolated from nuclei of subject to be identified. Oligonucleotide probes of approximately 30 bp in length are synthesized that correspond to at least 10, preferably 50 sequences from the cDNAs or genomic DNAs obtainable therefrom. The probes are used to hybridize to the genomic DNA through conditions known to those in the art. The oligonucleotides are end labeled with p³² using polynucleotide kinase (Pharmacia). Dot Blots are created by spotting the genomic DNA onto nitrocellulose or the like using a vacuum dot blot manifold (BioRad, Richmond Calif.). The nitrocellulose filter containing the genomic sequences is baked or UV linked to the filter, prehybridized and hybridized with labeled probe using techniques known in the art (Davis et al. supra). The ³²p labeled DNA fragments are sequentially hybridized with successively stringent conditions to detect minimal differences between the 30 bp sequence and the DNA. Tetramethylammonium chloride is useful for identifying clones containing small numbers of nucleotide mismatches (Wood et al., Proc. Natl. Acad. Sci. USA 82(6):1585-1588 (1985)) which is hereby incorporated by reference. A unique pattern of dots distinguishes one individual from another individual.
cDNAs or oligonucleotides containing at least 10 consecutive bases from these sequences can be used as probes in the following alternative fingerprinting technique. Preferably, the probe comprises at least 12, 15, or 17 consecutive nucleotides from the cDNA (or genomic DNAs obtainable therefrom). More preferably, the probe comprises at least 20-30 consecutive nucleotides from the cDNA (or genomic DNAs obtainable therefrom). In some embodiments, the probe comprises more than 30 nucleotides from the cDNA (or genomic DNAs obtainable therefrom). In other embodiments, the probe comprises at least 40, at least 50, at least 75, at least 100, at least 150, or at least 200 consecutive nucleotides from the cDNA (or genomic DNAs obtainable therefrom).

Preferably, a plurality of probes having sequences from different genes are used in the alternative fingerprinting technique. Example 38 below provides a representative alternative fingerprinting procedure in which the probes are derived from cDNAs.

### EXAMPLE 38

Alternative “Fingerprint” Identification Technique

20-mer oligonucleotides are prepared from a large number, e.g. 50, 100, or 200, of cDNA sequences (or genomic DNAs obtainable therefrom) using commercially available oligonucleotide services such as Genset, Paris, France. Cell samples from the test subject are processed for DNA using techniques well known to those with skill in the art. The nucleic acid is digested with restriction enzymes such as EcoRI and XbaI. Following digestion, samples are applied to wells for electrophoresis. The procedure, as known in the art, may be modified to accommodate polyacrylamide electrophoresis, however in this example, samples containing 5 ug of DNA are loaded into wells and separated on 0.8% agarose gels. The gels are transferred onto nitrocellulose using standard Southern blotting techniques.

ng of each of the oligonucleotides are pooled and end-labeled with p³². The nitrocellulose is prehybridized with blocking solution and hybridized with the labeled probes. Following hybridization and washing, the nitrocellulose filter is exposed to X-Omat AR X-ray film. The resulting hybridization pattern will be unique for each individual.

It is additionally contemplated within this example that the number of probe sequences used can be varied for additional accuracy or clarity.

The antibodies generated in Examples 18 and 31 above may be used to identify the tissue type or cell species from which a sample is derived as described above.

### EXAMPLE 39

Identification of Tissue Types or Cell Species by Means of Labeled Tissue Specific Antibodies

Identification of specific tissues is accomplished by the visualization of tissue specific antigens by means of antibody preparations according to Examples 18 and 31 which are conjugated, directly or indirectly to a detectable marker. Selected labeled antibody species bind to their specific antigen binding partner in tissue sections, cell suspensions, or in extracts of soluble proteins from a tissue sample to provide a pattern for qualitative or semi-qualitative interpretation.

Antisera for these procedures must have a potency exceeding that of the native preparation, and for that reason, antibodies are concentrated to a mg/ml level by isolation of the gamma globulin fraction, for example, by ion-exchange chromatography or by ammonium sulfate fractionation. Also, to provide the most specific antisera, unwanted antibodies, for example to common proteins, must be removed from the gamma globulin fraction, for example by means of insoluble immunoabsorbents, before the antibodies are labeled with the marker. Either monoclonal or heterologous antisera is suitable for either procedure.

A. Immunohistochemical Techniques

Purified, high-titer antibodies, prepared as described above, are conjugated to a detectable marker, as described, for example, by Fudenberg, H., Chap. 26 in: Basic 503 Clinical Immunology, 3rd Ed. Lange, Los Altos, Calif. (1980) or Rose, N. et al., Chap. 12 in: Methods in Immunodiagnosis, 2d Ed. John Wiley 503 Sons, New York (1980).

A fluorescent marker, either fluorescein or rhodamine, is preferred, but antibodies can also be labeled with an enzyme that supports a color producing reaction with a substrate, such as horseradish peroxidase. Markers can be added to tissue-bound antibody in a second step, as described below. Alternatively, the specific antitissue antibodies can be labeled with ferritin or other electron dense particles, and localization of the ferritin coupled antigen-antibody complexes achieved by means of an electron microscope. In yet another approach, the antibodies are radiolabeled, with, for example ¹²⁵I, and detected by overlaying the antibody treated preparation with photographic emulsion.

Preparations to carry out the procedures can comprise monoclonal or polyclonal antibodies to a single protein or peptide identified as specific to a tissue type, for example, brain tissue, or antibody preparations to several antigenically distinct tissue specific antigens can be used in panels, independently or in mixtures, as required.

Tissue sections and cell suspensions are prepared for immunohistochemical examination according to common histological techniques. Multiple cryostat sections (about 4 μm, unfixed) of the unknown tissue and known control, are mounted and each slide covered with different dilutions of the antibody preparation. Sections of known and unknown tissues should also be treated with preparations to provide a positive control, a negative control, for example, pre-immune sera, and a control for non-specific staining, for example, buffer.

Treated sections are incubated in a humid chamber for 30 min at room temperature, rinsed, then washed in buffer for 30-45 min. Excess fluid is blotted away, and the marker developed.

If the tissue specific antibody was not labeled in the first incubation, it can be labeled at this time in a second antibody-antibody reaction, for example, by adding fluorescein- or enzyme-conjugated antibody against the immunoglobulin class of the antiserum-producing species, for example, fluorescein labeled antibody to mouse IgG. Such labeled sera are commercially available.

The antigen found in the tissues by the above procedure can be quantified by measuring the intensity of color or fluorescence on the tissue section, and calibrating that signal using appropriate standards.

B. Identification of Tissue Specific Soluble Proteins

The visualization of tissue specific proteins and identification of unknown tissues from that procedure is carried out using the labeled antibody reagents and detection strategy as described for immunohistochemistry; however the sample is prepared according to an electrophoretic technique to distribute the proteins extracted from the tissue in an orderly array on the basis of molecular weight for detection.

A tissue sample is homogenized using a Virtis apparatus; cell suspensions are disrupted by Dounce homogenization or osmotic lysis, using detergents in either case as required to disrupt cell membranes, as is the practice in the art. Insoluble cell components such as nuclei, microsomes, and membrane fragments are removed by ultracentrifugation, and the soluble protein-containing fraction concentrated if necessary and reserved for analysis.

A sample of the soluble protein solution is resolved into individual protein species by conventional SDS polyacrylamide electrophoresis as described, for example, by Davis, L. et al., Section 19-2 in: Basic Methods in Molecular Biology (P. Leder, ed), Elsevier, New York (1986), using a range of amounts of polyacrylamide in a set of gels to resolve the entire molecular weight range of proteins to be detected in the sample. A size marker is run in parallel for purposes of estimating molecular weights of the constituent proteins. Sample size for analysis is a convenient volume of from 5 to 55 μl, and containing from about 1 to 100 μg protein. An aliquot of each of the resolved proteins is transferred by blotting to a nitrocellulose filter paper, a process that maintains the pattern of resolution. Multiple copies are prepared. The procedure, known as Western Blot Analysis, is well described in Davis, L. et al., (above) Section 19-3. One set of nitrocellulose blots is stained with Coomassie Blue dye to visualize the entire set of proteins for comparison with the antibody bound proteins. The remaining nitrocellulose filters are then incubated with a solution of one or more specific antisera to tissue specific proteins prepared as described in Examples 18 and 31. In this procedure, as in procedure A above, appropriate positive and negative sample and reagent controls are run.

In either procedure A or B, a detectable label can be attached to the primary tissue antigen-primary antibody complex according to various strategies and permutations thereof. In a straightforward approach, the primary specific antibody can be labeled; alternatively, the unlabeled complex can be bound by a labeled secondary anti-IgG antibody. In other approaches, either the primary or secondary antibody is conjugated to a biotin molecule, which can, in a subsequent step, bind an avidin conjugated marker. According to yet another strategy, enzyme labeled or radioactive protein A, which has the property of binding to any IgG, is bound in a final step to either the primary or secondary antibody.

The visualization of tissue specific antigen binding at levels above those seen in control tissues to one or more tissue specific antibodies, prepared from the gene sequences identified from cDNA sequences, can identify tissues of unknown origin, for example, forensic samples, or differentiated tumor tissue that has metastasized to foreign bodily sites.

In addition to their applications in forensics and identification, cDNAs (or genomic DNAs obtainable therefrom) may be mapped to their chromosomal locations. example 40 below describes radiation hybrid (RH) mapping of human chromosomal regions using cDNAs. example 41 below describes a representative procedure for mapping a cDNA (or a genomic DNA obtainable therefrom) to its location on a human chromosome. example 42 below describes mapping of cDNAs (or genomic DNAs obtainable therefrom) on metaphase chromosomes by Fluorescence In Situ Hybridization (FISH).

### EXAMPLE 40

Radiation Hybrid Mapping of cDNAs to the Human Genome

Radiation hybrid (RH) mapping is a somatic cell genetic approach that can be used for high resolution mapping of the human genome. In this approach, cell lines containing one or more human chromosomes are lethally irradiated, breaking each chromosome into fragments whose size depends on the radiation dose. These fragments are rescued by fusion with cultured rodent cells, yielding subclones containing different fragments of the human genome. This technique is described by Benham et al. (Genomics 4:509-517, 1989) and Cox et al., (Science 250:245-250, 1990), the entire contents of which are hereby incorporated by reference. The random and independent nature of the subclones permits efficient mapping of any human genome marker. Human DNA isolated from a panel of 80-100 cell lines provides a mapping reagent for ordering cDNAs (or genomic DNAs obtainable therefrom). In this approach, the frequency of breakage between markers is used to measure distance, allowing construction of fine resolution maps as has been done using conventional ESTs (Schuler et al., Science 274:540-546, 1996, hereby incorporated by reference).

RH mapping has been used to generate a high-resolution whole genome radiation hybrid map of human chromosome 17q22-q25.3 across the genes for growth hormone (GH) and thymidine kinase (TK) (Foster et al., Genomics 33:185-192, 1996), the region surrounding the Gorlin syndrome gene (Obermayr et al., Eur. J. Hum. Genet. 4:242-245, 1996), 60 loci covering the entire short arm of chromosome 12 (Raeymaekers et al., Genomics 29:170-178, 1995), the region of human chromosome 22 containing the neurofibromatosis type 2 locus (Frazer et al., Genomics 14:574-584, 1992) and 13 loci on the long arm of chromosome 5 (Warrington et al., Genomics 11:701-708, 1991).

### EXAMPLE 41

Mapping of cDNAs to Human Chromosomes using PCR Techniques

cDNAs (or genomic DNAs obtainable therefrom) may be assigned to human chromosomes using PCR based methodologies. In such approaches, oligonucleotide primer pairs are designed from the cDNA sequence (or the sequence of a genomic DNA obtainable therefrom) to minimize the chance of amplifying through an intron. Preferably, the oligonucleotide primers are 18-23 bp in length and are designed for PCR amplification. The creation of PCR primers from known sequences is well known to those with skill in the art. For a review of PCR technology see Erlich, H. A., PCR Technology; Principles and Applications for DNA Amplification. 1992. W. H. Freeman and Co., New York.

The primers are used in polymerase chain reactions (PCR) to amplify templates from total human genomic DNA. PCR conditions are as follows: 60 ng of genomic DNA is used as a template for PCR with 80 ng of each oligonucleotide primer, 0.6 unit of Taq polymerase, and 1 μCu of a ³²P-labeled deoxycytidine triphosphate. The PCR is performed in a microplate thermocycler (Techne) under the following conditions: 30 cycles of 94° C., 1.4 min; 55° C., 2 min; and 72° C., 2 min; with a final extension at 72° C. for 10 min. The amplified products are analyzed on a 6% polyacrylamide sequencing gel and visualized by autoradiography. If the length of the resulting PCR product is identical to the distance between the ends of the primer sequences in the cDNA from which the primers are derived, then the PCR reaction is repeated with DNA templates from two panels of human-rodent somatic cell hybrids, BIOS PCRable DNA (BIOS Corporation) and NIGMS Human-Rodent Somatic Cell Hybrid Mapping Panel Number 1 (NIGMS, Camden, N.J.).

PCR is used to screen a series of somatic cell hybrid cell lines containing defined sets of human chromosomes for the presence of a given cDNA (or genomic DNA obtainable therefrom). DNA is isolated from the somatic hybrids and used as starting templates for PCR reactions using the primer pairs from the cDNAs (or genomic DNAs obtainable therefrom). Only those somatic cell hybrids with chromosomes containing the human gene corresponding to the cDNA (or genomic DNA obtainable therefrom) will yield an amplified fragment. The cDNAs (or genomic DNAs obtainable therefrom) are assigned to a chromosome by analysis of the segregation pattern of PCR products from the somatic hybrid DNA templates. The single human chromosome present in all cell hybrids that give rise to an amplified fragment is the chromosome containing that cDNA (or genomic DNA obtainable therefrom). For a review of techniques and analysis of results from somatic cell gene mapping experiments. (See Ledbetter et al., Genomics 6:475-481 (1990).)

Alternatively, the cDNAs (or genomic DNAs obtainable therefrom) may be mapped to individual chromosomes using FISH as described in example 42 below.

### EXAMPLE 42

Mapping of cDNAs to Chromosomes using Fluorescence in Situ Hybridization

Fluorescence in situ hybridization allows the cDNA (or genomic DNA obtainable therefrom) to be mapped to a particular location on a given chromosome. The chromosomes to be used for fluorescence in situ hybridization techniques may be obtained from a variety of sources including cell cultures, tissues, or whole blood.

In a preferred embodiment, chromosomal localization of a cDNA (or genomic DNA obtainable therefrom) is obtained by FISH as described by Cherif et al. (Proc. Natl. Acad. Sci. U.S.A., 87:6639-6643, 1990). Metaphase chromosomes are prepared from phytohemagglutinin (PHA)-stimulated blood cell donors. PHA-stimulated lymphocytes from healthy males are cultured for 72 h in RPMI-1640 medium. For synchronization, methotrexate (10 μM) is added for 17 h, followed by addition of 5-bromodeoxyuridine (5-BudR, 0.1 mM) for 6 h. Colcemid (1 μg/ml) is added for the last 15 min before harvesting the cells. Cells are collected, washed in RPMI, incubated with a hypotonic solution of KCl (75 MM) at 37° C. for 15 min and fixed in three changes of methanol:acetic acid (3:1). The cell suspension is dropped onto a glass slide and air dried. The cDNA (or genomic DNA obtainable therefrom) is labeled with biotin-16 dUTP by nick translation according to the manufacturer's instructions (Bethesda Research Laboratories, Bethesda, Md.), purified using a Sephadex G-50 column (Pharmacia, Upssala, Sweden) and precipitated. Just prior to hybridization, the DNA pellet is dissolved in hybridization buffer (50% formamide, 2×SSC, 10% dextran sulfate, 1 mg/ml sonicated salmon sperm DNA, pH 7) and the probe is denatured at 70° C. for 5-10 min.

Slides kept at −20° C. are treated for 1 h at 37° C. with RNase A (100 μg/ml), rinsed three times in 2×SSC and dehydrated in an ethanol series. Chromosome preparations are denatured in 70% formamide, 2×SSC for 2 min at 70° C., then dehydrated at 4° C. The slides are treated with proteinase K (10 μg/100 ml in 20 mM Tris-HCl, 2 mM CaCl₂) at 37° C. for 8 min and dehydrated. The hybridization mixture containing the probe is placed on the slide, covered with a coverslip, sealed with rubber cement and incubated overnight in a humid chamber at 37° C. After hybridization and post-hybridization washes, the biotinylated probe is detected by avidin-FITC and amplified with additional layers of biotinylated goat anti-avidin and avidin-FITC. For chromosomal localization, fluorescent R-bands are obtained as previously described (Cherif et al., supra.). The slides are observed under a LEICA fluorescence microscope (DMRXA). Chromosomes are counterstained with propidium iodide and the fluorescent signal of the probe appears as two symmetrical yellow-green spots on both chromatids of the fluorescent R-band chromosome (red). Thus, a particular cDNA (or genomic DNA obtainable therefrom) may be localized to a particular cytogenetic R-band on a given chromosome.

### EXAMPLE 43

Use of cDNAs to Construct or Expand Chromosome Maps

Once the cDNAs (or genomic DNAs obtainable therefrom) have been assigned to particular chromosomes using the techniques described in Examples 40-42 above, they may be utilized to construct a high resolution map of the chromosomes on which they are located or to identify the chromosomes in a sample.

Chromosome mapping involves assigning a given unique sequence to a particular chromosome as described above. Once the unique sequence has been mapped to a given chromosome, it is ordered relative to other unique sequences located on the same chromosome. One approach to chromosome mapping utilizes a series of yeast artificial chromosomes (YACs) bearing several thousand long inserts derived from the chromosomes of the organism from which the cDNAs (or genomic DNAs obtainable therefrom) are obtained. This approach is described in Ramaiah Nagaraja et al. Genome Research 7:210-222, March 1997. Briefly, in this approach each chromosome is broken into overlapping pieces which are inserted into the YAC vector. The YAC inserts are screened using PCR or other methods to determine whether they include the cDNA (or genomic DNA obtainable therefrom) whose position is to be determined. Once an insert has been found which includes the cDNA (or genomic DNA obtainable therefrom), the insert can be analyzed by PCR or other methods to determine whether the insert also contains other sequences known to be on the chromosome or in the region from which the cDNA (or genomic DNA obtainable therefrom) was derived. This process can be repeated for each insert in the YAC library to determine the location of each of the cDNAs (or genomic DNAs obtainable therefrom) relative to one another and to other known chromosomal markers. In this way, a high resolution map of the distribution of numerous unique markers along each of the organisms chromosomes may be obtained.

As described in example 44 below cDNAs (or genomic DNAs obtainable therefrom) may also be used to identify genes associated with a particular phenotype, such as hereditary disease or drug response.

### EXAMPLE 44

Identification of Genes Associated with Hereditary Diseases or Drug Response

This example illustrates an approach useful for the association of cDNAs (or genomic DNAs obtainable therefrom) with particular phenotypic characteristics. In this example, a particular cDNA (or genomic DNA obtainable therefrom) is used as a test probe to associate that cDNA (or genomic DNA obtainable therefrom) with a particular phenotypic characteristic.

cDNAs (or genomic DNAs obtainable therefrom) are mapped to a particular location on a human chromosome using techniques such as those described in Examples 40 and 41 or other techniques known in the art. A search of Mendelian Inheritance in Man (V. McKusick, Mendelian Inheritance in Man (available on line through Johns Hopkins University Welch Medical Library) reveals the region of the human chromosome which contains the cDNA (or genomic DNA obtainable therefrom) to be a very gene rich region containing several known genes and several diseases or phenotypes for which genes have not been identified. The gene corresponding to this cDNA (or genomic DNA obtainable therefrom) thus becomes an immediate candidate for each of these genetic diseases.

Cells from patients with these diseases or phenotypes are isolated and expanded in culture. PCR primers from the cDNA (or genomic DNA obtainable therefrom) are used to screen genomic DNA, mRNA or cDNA obtained from the patients. cDNAs (or genomic DNAs obtainable therefrom) that are not amplified in the patients can be positively associated with a particular disease by further analysis. Alternatively, the PCR analysis may yield fragments of different lengths when the samples are derived from an individual having the phenotype associated with the disease than when the sample is derived from a healthy individual, indicating that the gene containing the cDNA may be responsible for the genetic disease.

VI. Use of cDNAs (or Genomic DNAs Obtainable Therefrom) to Construct Vectors

The present cDNAs (or genomic DNAs obtainable therefrom) may also be used to construct secretion vectors capable of directing the secretion of the proteins encoded by genes inserted in the vectors. Such secretion vectors may facilitate the purification or enrichment of the proteins encoded by genes inserted therein by reducing the number of background proteins from which the desired protein must be purified or enriched. Exemplary secretion vectors are described below.

### EXAMPLE 45

Construction of Secretion Vectors

The secretion vectors of the present invention include a promoter capable of directing gene expression in the host cell, tissue, or organism of interest. Such promoters include the Rous Sarcoma Virus promoter, the SV40 promoter, the human cytomegalovirus promoter, and other promoters familiar to those skilled in the art.

A signal sequence from a cDNA (or genomic DNA obtainable therefrom), such as one of the signal sequences in SEQ ID NOs: 1-405 as defined in Table I above, is operably linked to the promoter such that the mRNA transcribed from the promoter will direct the translation of the signal peptide. The host cell, tissue, or organism may be any cell, tissue, or organism which recognizes the signal peptide encoded by the signal sequence in the cDNA (or genomic DNA obtainable therefrom). Suitable hosts include mammalian cells, tissues or organisms, avian cells, tissues, or organisms, insect cells, tissues or organisms, or yeast.

In addition, the secretion vector contains cloning sites for inserting genes encoding the proteins which are to be secreted. The cloning sites facilitate the cloning of the insert gene in frame with the signal sequence such that a fusion protein in which the signal peptide is fused to the protein encoded by the inserted gene is expressed from the mRNA transcribed from the promoter. The signal peptide directs the extracellular secretion of the fusion protein.

The secretion vector may be DNA or RNA and may integrate into the chromosome of the host, be stably maintained as an extrachromosomal replicon in the host, be an artificial chromosome, or be transiently present in the host. Preferably, the secretion vector is maintained in multiple copies in each host cell. As used herein, multiple copies means at least 2, 5, 10, 20, 25, 50 or more than 50 copies per cell. In some embodiments, the multiple copies are maintained extrachromosomally. In other embodiments, the multiple copies result from amplification of a chromosomal sequence.

Many nucleic acid backbones suitable for use as secretion vectors are known to those skilled in the art, including retroviral vectors, SV40 vectors, Bovine Papilloma Virus vectors, yeast integrating plasmids, yeast episomal plasmids, yeast artificial chromosomes, human artificial chromosomes, P element vectors, baculovirus vectors, or bacterial plasmids capable of being transiently introduced into the host.

The secretion vector may also contain a polyA signal such that the polyA signal is located downstream of the gene inserted into the secretion vector.

After the gene encoding the protein for which secretion is desired is inserted into the secretion vector, the secretion vector is introduced into the host cell, tissue, or organism using calcium phosphate precipitation, DEAE-Dextran, electroporation, liposome-mediated transfection, viral particles or as naked DNA. The protein encoded by the inserted gene is then purified or enriched from the supernatant using conventional techniques such as ammonium sulfate precipitation, immunoprecipitation, immunochromatography, size exclusion chromatography, ion exchange chromatography, and hplc. Alternatively, the secreted protein may be in a sufficiently enriched or pure state in the supernatant or growth media of the host to permit it to be used for its intended purpose without further enrichment.

The signal sequences may also be inserted into vectors designed for gene therapy. In such vectors, the signal sequence is operably linked to a promoter such that mRNA transcribed from the promoter encodes the signal peptide. A cloning site is located downstream of the signal sequence such that a gene encoding a protein whose secretion is desired may readily be inserted into the vector and fused to the signal sequence. The vector is introduced into an appropriate host cell. The protein expressed from the promoter is secreted extracellularly, thereby producing a therapeutic effect.

The cDNAs or 5′ ESTs may also be used to clone sequences located upstream of the cDNAs or 5′ ESTs which are capable of regulating gene expression, including promoter sequences, enhancer sequences, and other upstream sequences which influence transcription or translation levels. Once identified and cloned, these upstream regulatory sequences may be used in expression vectors designed to direct the expression of an inserted gene in a desired spatial, temporal, developmental, or quantitative fashion. The next example describes a method for cloning sequences upstream of the cDNAs or 5′ ESTs.

### EXAMPLE 46

Use of cDNAs or Fragments thereof to Clone Upstream Sequences from Genomic DNA

Sequences derived from cDNAs or 5′ ESTs may be used to isolate the promoters of the corresponding genes using chromosome walking techniques. In one chromosome walking technique, which utilizes the GenomeWalker™ kit available from Clontech, five complete genomic DNA samples are each digested with a different restriction enzyme which has a 6 base recognition site and leaves a blunt end. Following digestion, oligonucleotide adapters are ligated to each end of the resulting genomic DNA fragments.

For each of the five genomic DNA libraries, a first PCR reaction is performed according to the manufacturer's instructions (which are incorporated herein by reference) using an outer adaptor primer provided in the kit and an outer gene specific primer. The gene specific primer should be selected to be specific for the cDNA or 5′ EST of interest and should have a melting temperature, length, and location in the cDNA or 5′ EST which is consistent with its use in PCR reactions. Each first PCR reaction contains 5ng of genomic DNA, 5 μl of 10×Tth reaction buffer, 0.2 mM of each dNTP, 0.2 μM each of outer adaptor primer and outer gene specific primer, 1.1 mM of Mg(OAc)₂, and 1 μl of the Tth polymerase 50× mix in a total volume of 50 μl. The reaction cycle for the first PCR reaction is as follows: 1 min at 94° C./2 sec at 94° C., 3 min at 72° C. (7 cycles)/2 sec at 94° C., 3 min at 67° C. (32 cycles)/5 min at 67° C.

The product of the first PCR reaction is diluted and used as a template for a second PCR reaction according to the manufacturer's instructions using a pair of nested primers which are located internally on the amplicon resulting from the first PCR reaction. For example, 5 μl of the reaction product of the first PCR reaction mixture may be diluted 180 times. Reactions are made in a 50 μl volume having a composition identical to that of the first PCR reaction except the nested primers are used. The first nested primer is specific for the adaptor, and is provided with the GenomeWalker™ kit. The second nested primer is specific for the particular cDNA or 5′ EST for which the promoter is to be cloned and should have a melting temperature, length, and location in the cDNA or 5′ EST which is consistent with its use in PCR reactions. The reaction parameters of the second PCR reaction are as follows: 1 min at 94° C./2 sec at 94° C., 3 min at 72° C. (6 cycles)/2 sec at 94° C., 3 min at 67° C. (25 cycles)/5 min at 67° C.

The product of the second PCR reaction is purified, cloned, and sequenced using standard techniques. Alternatively, two or more human genomic DNA libraries can be constructed by using two or more restriction enzymes. The digested genomic DNA is cloned into vectors which can be converted into single stranded, circular, or linear DNA. A biotinylated oligonucleotide comprising at least 15 nucleotides from the cDNA or 5′ EST sequence is hybridized to the single stranded DNA. Hybrids between the biotinylated oligonucleotide and the single stranded DNA containing the cDNA or EST sequence are isolated as described in example 17 above. Thereafter, the single stranded DNA containing the cDNA or EST sequence is released from the beads and converted into double stranded DNA using a primer specific for the cDNA or 5′ EST sequence or a primer corresponding to a sequence included in the cloning vector. The resulting double stranded DNA is transformed into bacteria. DNAs containing the 5′ EST or cDNA sequences are identified by colony PCR or colony hybridization.

Once the upstream genomic sequences have been cloned and sequenced as described above, prospective promoters and transcription start sites within the upstream sequences may be identified by comparing the sequences upstream of the cDNAs or 5′ ESTs with databases containing known transcription start sites, transcription factor binding sites, or promoter sequences.

In addition, promoters in the upstream sequences may be identified using promoter reporter vectors as described below.

### EXAMPLE 47

Identification of Promoters in Cloned Upstream Sequences

The genomic sequences upstream of the cDNAs or fragment thereof are cloned into a suitable promoter reporter vector, such as the pSEAP-Basic, pSEAP-Enhancer, pβgal-Basic, pβgal-Enhancer, or pEGFP-1 Promoter Reporter vectors available from Clontech. Briefly, each of these promoter reporter vectors include multiple cloning sites positioned upstream of a reporter gene encoding a readily assayable protein such as secreted alkaline phosphatase, β galactosidase, or green fluorescent protein. The sequences upstream of the cDNAs or 5′ ESTs are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for augmenting transcription levels from weak promoter sequences. A significant level of expression above that observed with the vector lacking an insert indicates that a promoter sequence is present in the inserted upstream sequence.

Appropriate host cells for the promoter reporter vectors may be chosen based on the results of the above described determination of expression patterns of the cDNAs and ESTs. For example, if the expression pattern analysis indicates that the mRNA corresponding to a particular cDNA or fragment thereof is expressed in fibroblasts, the promoter reporter vector may be introduced into a human fibroblast cell line.

Promoter sequences within the upstream genomic DNA may be further defined by constructing nested deletions in the upstream DNA using conventional techniques such as Exonuclease III digestion. The resulting deletion fragments can be inserted into the promoter reporter vector to determine whether the deletion has reduced or obliterated promoter activity. In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into the cloning sites in the promoter reporter vectors.

### EXAMPLE 48

Cloning and Identification of Promoters

Using the method described in example 47 above with 5′ ESTs, sequences upstream of several genes were obtained.

The promoters and other regulatory sequences located upstream of the cDNAs or 5′ ESTs may be used to design expression vectors capable of directing the expression of an inserted gene in a desired spatial, temporal, developmental, or quantitative manner. A promoter capable of directing the desired spatial, temporal, developmental, and quantitative patterns may be selected using the results of the expression analysis described in example 10 above. For example, if a promoter which confers a high level of expression in muscle is desired, the promoter sequence upstream of a cDNA or 5′ EST derived from an mRNA which is expressed at a high level in muscle, as determined by the method of example 10, may be used in the expression vector.

Preferably, the desired promoter is placed near multiple restriction sites to facilitate the cloning of the desired insert downstream of the promoter, such that the promoter is able to drive expression of the inserted gene. The promoter may be inserted in conventional nucleic acid backbones designed for extrachromosomal replication, integration into the host chromosomes or transient expression. Suitable backbones for the present expression vectors include retroviral backbones, backbones from eukaryotic episomes such as SV40 or Bovine Papilloma Virus, backbones from bacterial episomes, or artificial chromosomes.

Preferably, the expression vectors also include a polyA signal downstream of the multiple restriction sites for directing the polyadenylation of mRNA transcribed from the gene inserted into the expression vector.

Following the identification of promoter sequences using the procedures of Examples 4648, proteins which interact with the promoter may be identified as described in example 49 below.

### EXAMPLE 49

Identification of Proteins which Interact with Promoter Sequences, Upstream Regulatory Sequences, or mRNA

Sequences within the promoter region which are likely to bind transcription factors may be identified by identity to known transcription factor binding sites or through conventional mutagenesis or deletion analyses of reporter plasmids containing the promoter sequence. For example, deletions may be made in a reporter plasmid containing the promoter sequence of interest operably linked to an assayable reporter gene. The reporter plasmids carrying various deletions within the promoter region are transfected into an appropriate host cell and the effects of the deletions on expression levels is assessed. Transcription factor binding sites within the regions in which deletions reduce expression levels may be further localized using site directed mutagenesis, linker scanning analysis, or other techniques familiar to those skilled in the art. Nucleic acids encoding proteins which interact with sequences in the promoter may be identified using one-hybrid systems such as those described in the manual accompanying the Matchmaker One-Hybrid System kit avalilabe from Clontech (Catalog No. K1603-1), the disclosure of which is incorporated herein by reference. Briefly, the Matchmaker One-hybrid system is used as follows. The target sequence for which it is desired to identify binding proteins is cloned upstream of a selectable reporter gene and integrated into the yeast genome. Preferably, multiple copies of the target sequences are inserted into the reporter plasmid in tandem.

A library comprised of fusions between cDNAs to be evaluated for the ability to bind to the promoter and the activation domain of a yeast transcription factor, such as GAL4, is transformed into the yeast strain containing the integrated reporter sequence. The yeast are plated on selective media to select cells expressing the selectable marker linked to the promoter sequence. The colonies which grow on the selective media contain genes encoding proteins which bind the target sequence. The inserts in the genes encoding the fusion proteins are further characterized by sequencing. In addition, the inserts may be inserted into expression vectors or in vitro transcription vectors. Binding of the polypeptides encoded by the inserts to the promoter DNA may be confirmed by techniques familiar to those skilled in the art, such as gel shift analysis or DNAse protection analysis.

VII. Use of cDNAs (or Genomic DNAs Obtainable therefrom) in Gene Therapy

The present invention also comprises the use of cDNAs (or genomic DNAs obtainable therefrom) in gene therapy strategies, including antisense and triple helix strategies as described in Examples 50 and 51 below. In antisense approaches, nucleic acid sequences complementary to an mRNA are hybridized to the mRNA intracellularly, thereby blocking the expression of the protein encoded by the mRNA. The antisense sequences may prevent gene expression through a variety of mechanisms. For example, the antisense sequences may inhibit the ability of ribosomes to translate the mRNA. Alternatively, the antisense sequences may block transport of the mRNA from the nucleus to the cytoplasm, thereby limiting the amount of mRNA available for translation. Another mechanism through which antisense sequences may inhibit gene expression is by interfering with mRNA splicing. In yet another strategy, the antisense nucleic acid may be incorporated in a ribozyme capable of specifically cleaving the target mRNA.

### EXAMPLE 50

Preparation and use of Antisense Oligonucleotides

The antisense nucleic acid molecules to be used in gene therapy may be either DNA or RNA sequences. They may comprise a sequence complementary to the sequence of the cDNA (or genomic DNA obtainable therefrom). The antisense nucleic acids should have a length and melting temperature sufficient to permit formation of an intracellular duplex having sufficient stability to inhibit the expression of the mRNA in the duplex. Strategies for designing antisense nucleic acids suitable for use in gene therapy are disclosed in Green et al., Ann. Rev. Biochem., 55:569-597 (1986) and Izant and Weintraub, Cell, 36:1007-1015 (1984), which are hereby incorporated by reference.

In some strategies, antisense molecules are obtained from a nucleotide sequence encoding a protein by reversing the orientation of the coding region with respect to a promoter so as to transcribe the opposite strand from that which is normally transcribed in the cell. The antisense molecules may be transcribed using in vitro transcription systems such as those which employ T7 or SP6 polymerase to generate the transcript. Another approach involves transcription of the antisense nucleic acids in vivo by operably linking DNA containing the antisense sequence to a promoter in an expression vector.

Alternatively, oligonucleotides which are complementary to the strand normally transcribed in the cell may be synthesized in vitro. Thus, the antisense nucleic acids are complementary to the corresponding mRNA and are capable of hybridizing to the mRNA to create a duplex. In some embodiments, the antisense sequences may contain modified sugar phosphate backbones to increase stability and make them less sensitive to RNase activity. Examples of modifications suitable for use in antisense strategies include 2′ O-methyl RNA oligonucleotides and Protein-nucleic acid (PNA) oligonucleotides. Further examples are described by Rossi et al., Pharmacol. Ther., 50(2):245-254, (1991).

Various types of antisense oligonucleotides complementary to the sequence of the cDNA (or genomic DNA obtainable therefrom) may be used. In one preferred embodiment, stable and semi-stable antisense oligonucleotides described in International Application No. PCT WO94/23026, hereby incorporated by reference, are used. In these moleucles, the 3′ end or both the 3′ and 5′ ends are engaged in intramolecular hydrogen bonding between complementary base pairs. These molecules are better able to withstand exonuclease attacks and exhibit increased stability compared to conventional antisense oligonucleotides.

In another preferred embodiment, the antisense oligodeoxynucleotides against herpes simplex virus types 1 and 2 described in International Application No. WO 95/04141, hereby incorporated by reference, are used.

In yet another preferred embodiment, the covalently cross-linked antisense oligonucleotides described in International Application No. WO 96/31523, hereby incorporated by reference, are used. These double- or single-stranded oligonucleotides comprise one or more, respectively, inter- or intra-oligonucleotide covalent cross-linkages, wherein the linkage consists of an amide bond between a primary amine group of one strand and a carboxyl group of the other strand or of the same strand, respectively, the primary amine group being directly substituted in the 2′ position of the strand nucleotide monosaccharide ring, and the carboxyl group being carried by an aliphatic spacer group substituted on a nucleotide or nucleotide analog of the other strand or the same strand, respectively.

The antisense oligodeoxynucleotides and oligonucleotides disclosed in International Application No. WO 92/18522, incorporated by reference, may also be used. These molecules are stable to degradation and contain at least one transcription control recognition sequence which binds to control proteins and are effective as decoys therefor. These molecules may contain “hairpin” structures, “dumbbell” structures, “modified dumbbell” structures, “cross-linked” decoy structures and “loop” structures.

In another preferred embodiment, the cyclic double-stranded oligonucleotides described in European Patent Application No. 0 572 287 A2, hereby incorporated by reference are used. These ligated oligonucleotide “dumbbells” contain the binding site for a transcription factor and inhibit expression of the gene under control of the transcription factor by sequestering the factor.

Use of the closed antisense oligonucleotides disclosed in International Application No. WO 92/19732, hereby incorporated by reference, is also contemplated. Because these molecules have no free ends, they are more resistant to degradation by exonucleases than are conventional oligonucleotides. These oligonucleotides may be multifunctional, interacting with several regions which are not adjacent to the target mRNA.

The appropriate level of antisense nucleic acids required to inhibit gene expression may be determined using in vitro expression analysis. The antisense molecule may be introduced into the cells by diffusion, injection, infection or transfection using procedures known in the art. For example, the antisense nucleic acids can be introduced into the body as a bare or naked oligonucleotide, oligonucleotide encapsulated in lipid, oligonucleotide sequence encapsidated by viral protein, or as an oligonucleotide operably linked to a promoter contained in an expression vector. The expression vector may be any of a variety of expression vectors known in the art, including retroviral or viral vectors, vectors capable of extrachromosomal replication, or integrating vectors. The vectors may be DNA or RNA.

The antisense molecules are introduced onto cell samples at a number of different concentrations preferably between 1×10⁻¹⁰M to 1×1⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg bodyweight. Levels of oligonucleotide approaching 100 mg/kg bodyweight or higher may be possible after testing the toxicity of the oligonucleotide in laboratory animals. It is additionally contemplated that cells from the vertebrate are removed, treated with the antisense oligonucleotide, and reintroduced into the vertebrate.

It is further contemplated that the antisense oligonucleotide sequence is incorporated into a ribozyme sequence to enable the antisense to specifically bind and cleave its target mRNA. For technical applications of ribozyme and antisense oligonucleotides see Rossi et al., supra.

In a preferred application of this invention, the polypeptide encoded by the gene is first identified, so that the effectiveness of antisense inhibition on translation can be monitored using techniques that include but are not limited to antibody-mediated tests such as RIAs and ELISA, functional assays, or radiolabeling.

The cDNAs of the present invention (or genomic DNAs obtainable therefrom) may also be used in gene therapy approaches based on intracellular triple helix formation. Triple helix oligonucleotides are used to inhibit transcription from a genome. They are particularly useful for studying alterations in cell activity as it is associated with a particular gene. The cDNAs (or genomic DNAs obtainable therefrom) of the present invention or, more preferably, a fragment of those sequences, can be used to inhibit gene expression in individuals having diseases associated with expression of a particular gene. Similarly, a fragment of the cDNA (or genomic DNA obtainable therefrom) can be used to study the effect of inhibiting transcription of a particular gene within a cell. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences from the cDNA or from the gene corresponding to the cDNA are contemplated within the scope of this invention.

### EXAMPLE 51

Preparation and use of Triple Helix Probes

The sequences of the cDNAs (or genomic DNAs obtainable therefrom) are scanned to identify 10-mer to 20-mer homopyrimidine or homopurine stretches which could be used in triple-helix based strategies for inhibiting gene expression. Following identification of candidate homopyrimidine or homopurine stretches, their efficiency in inhibiting gene expression is assessed by introducing varying amounts of oligonucleotides containing the candidate sequences into tissue culture cells which normally express the target gene. The oligonucleotides may be prepared on an oligonucleotide synthesizer or they may be purchased commercially from a company specializing in custom oligonucleotide synthesis, such as GENSET, Paris, France.

The oligonucleotides may be introduced into the cells using a variety of methods known to those skilled in the art, including but not limited to calcium phosphate precipitation, DEAE-Dextran, electroporation, liposome-mediated transfection or native uptake.

Treated cells are monitored for altered cell function or reduced gene expression using techniques such as Northern blotting, RNase protection assays, or PCR based strategies to monitor the transcription levels of the target gene in cells which have been treated with the oligonucleotide. The cell functions to be monitored are predicted based upon the homologies of the target gene corresponding to the cDNA from which the oligonucleotide was derived with known gene sequences that have been associated with a particular function. The cell functions can also be predicted based on the presence of abnormal physiologies within cells derived from individuals with a particular inherited disease, particularly when the cDNA is associated with the disease using techniques described in example 44.

The oligonucleotides which are effective in inhibiting gene expression in tissue culture cells may then be introduced in vivo using the techniques described above and in example 50 at a dosage calculated based on the in vitro results, as described in example 50.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al. (Science, 245:967-971 (1989), which is hereby incorporated by this reference).

### EXAMPLE 52

Use of cDNAs to Express an Encoded Protein in a Host Organism

The cDNAs of the present invention may also be used to express an encoded protein in a host organism to produce a beneficial effect. In such procedures, the encoded protein may be transiently expressed in the host organism or stably expressed in the host organism. The encoded protein may have any of the activities described above. The encoded protein may be a protein which the host organism lacks or, alternatively, the encoded protein may augment the existing levels of the protein in the host organism.

A full length cDNA encoding the signal peptide and the mature protein, or a cDNA encoding only the mature protein is introduced into the host organism. The cDNA may be introduced into the host organism using a variety of techniques known to those of skill in the art. For example, the cDNA may be injected into the host organism as naked DNA such that the encoded protein is expressed in the host organism, thereby producing a beneficial effect.

Alternatively, the cDNA may be cloned into an expression vector downstream of a promoter which is active in the host organism. The expression vector may be any of the expression vectors designed for use in gene therapy, including viral or retroviral vectors.

The expression vector may be directly introduced into the host organism such that the encoded protein is expressed in the host organism to produce a beneficial effect. In another approach, the expression vector may be introduced into cells in vitro. Cells containing the expression vector are thereafter selected and introduced into the host organism, where they express the encoded protein to produce a beneficial effect.

### EXAMPLE 53

Use of Signal Peptides to Import Proteins into Cells

The short core hydrophobic region (h) of signal peptides encoded by the cDNAs of the present invention or fragment thereof may also be used as a carrier to import a peptide or a protein of interest, so-called cargo, into tissue culture cells (Lin et al., J. Biol. Chem., 270: 14225-14258 (1995); Du et al., J. Peptide Res., 51: 235-243 (1998); Rojas et al., Nature Biotech., 16: 370-375 (1998)).

When cell permeable peptides of limited size (approximately up to 25 amino acids) are to be translocated across cell membrane, chemical synthesis may be used in order to add the h region to either the C-terminus or the N-terminus to the cargo peptide of interest. Alternatively, when longer peptides or proteins are to be imported into cells, nucleic acids can be genetically engineered, using techniques familiar to those skilled in the art, in order to link the cDNA sequence or fragment thereof encoding the h region to the 5′ or the 3′ end of a DNA sequence coding for a cargo polypeptide. Such genetically engineered nucleic acids are then translated either in vitro or in vivo after transfection into appropriate cells, using conventional techniques to produce the resulting cell permeable polypeptide. Suitable hosts cells are then simply incubated with the cell permeable polypeptide which is then translocated across the membrane.

This method may be applied to study diverse intracellular functions and cellular processes. For instance, it has been used to probe functionally relevant domains of intracellular proteins and to examine protein-protein interactions involved in signal transduction pathways (Lin et al., supra; Lin et al., J. Biol. Chem., 271: 5305-5308 (1996); Rojas et al., J. Biol. Chem., 271: 27456-27461 (1996); Liu et al., Proc. Natl. Acad. Sci. USA, 93: 11819-11824 (1996); Rojas et al., Bioch. Biophys. Res. Commun., 234: 675-680 (1997)).

Such techniques may be used in cellular therapy to import proteins producing therapeutic effects. For instance, cells isolated from a patient may be treated with imported therapeutic proteins and then re-introduced into the host organism.

Alternatively, the h region of signal peptides of the present invention could be used in combination with a nuclear localization signal to deliver nucleic acids into cell nucleus. Such oligonucleotides may be antisense oligonucleotides or oligonucleotides designed to form triple helixes, as described in examples 50 and 51 respectively, in order to inhibit processing and maturation of a target cellular RNA.

### EXAMPLE 54

Computer Embodiments

As used herein the term “cDNA codes of SEQ ID NOs. 1-405” encompasses the nucleotide sequences of SEQ ID NOs. 1-405, fragments of SEQ ID NOs. 1-405, nucleotide sequences homologous to SEQ ID NOs. 1-405 or homologous to fragments of SEQ ID NOs. 1-405, and sequences complementary to all of the preceding sequences. The fragments include fragments of SEQ ID NOs. 1-405 comprising at least 8, 10, 12, 15, 18, 20, 25, 28, 30, 35, 40, 50, 75, 100, 150, 200, 300, 400, 500, 1000 or 2000 consecutive nucleotides of SEQ ID NOs. 1-405. Preferably, the fragments are novel fragments. Preferably the fragments include polynucleotides described in Table III or fragments thereof comprising at least 8, 10, 12, 15, 18, 20, 25, 28, 30, 35, 40, 50, 75, 100, 150, 200, 300, 400, 500, 1000 or 2000 consecutive nucleotides of the polynucleotides described in Table III. Homologous sequences and fragments of SEQ ID NOs. 1-405 refer to a sequence having at least 99%, 98%, 97%, 96%, 95%, 90%, 85%, 80%, or 75% identity to these sequences. Identity may be determined using any of the computer programs and parameters described in example 17, including BLAST2N with the default parameters or with any modified parameters. Homologous sequences also include RNA sequences in which uridines replace the thymines in the cDNA codes of SEQ ID NOs. 1-405. The homologous sequences may be obtained using any of the procedures described herein or may result from the correction of a sequencing error as described above. Preferably the homologous sequences and fragments of SEQ ID NOs. 1-405 include polynucleotides described in Table III or fragments comprising at least 8, 10, 12, 15, 18, 20, 25, 28, 30, 35, 40, 50, 75, 100, 150, 200, 300, 400, 500, 1000 or 2000 consecutive nucleotides of the polynucleotides described in Table III. It will be appreciated that the cDNA codes of SEQ ID NOs. 1-405 can be represented in the traditional single character format (See the inside back cover of Styer, Lubert. Biochemistry, 3^{rd} edition. W. H Freeman & Co., New York.) or in any other format which records the identity of the nucleotides in a sequence.

As used herein the term “polypeptide codes of SEQ ID NOS. 406-810” encompasses the polypeptide sequences of SEQ ID NOs. 406-810 which are encoded by the cDNAs of SEQ ID NOs. 1-405, polypeptide sequences homologous to the polypeptides of SEQ ID NOS. 406-810, or fragments of any of the preceding sequences. Homologous polypeptide sequences refer to a polypeptide sequence having at least 99%, 98%, 97%, 96%, 95%, 90%, 85%, 80%, 75% identity to one of the polypeptide sequences of SEQ ID NOS. 406-810. Identity may be determined using any of the computer programs and parameters described herein, including FASTA with the default parameters or with any modified parameters. The homologous sequences may be obtained using any of the procedures described herein or may result from the correction of a sequencing error as described above. The polypeptide fragments comprise at least 5, 8, 10, 12, 15, 20, 25, 30, 35, 40, 50, 60, 75, 100, 150 or 200 consecutive amino acids of the polypeptides of SEQ ID NOS. 406-810. Preferably, the fragments are novel fragments. Preferably, the fragments include polypeptides encoded by the polynucleotides described in Table III, or fragments thereof comprising at least 5, 10, 15, 20, 25, 30, 35, 40, 50, 75, 100, or 150 consecutive amino acids of the polypeptides encoded by the polynucleotides described in Table III. It will be appreciated that the polypeptide codes of the SEQ ID NOS. 406-810 can be represented in the traditional single character format or three letter format (See the inside back cover of Starrier, Lubert. Biochemistry, 3^{rd} edition. W. H Freeman & Co., New York.) or in any other format which relates the identity of the polypeptides in a sequence.

It will be appreciated by those skilled in the art that the cDNA codes of SEQ ID NOs. 1-405 and polypeptide codes of SEQ ID NOS. 406-810 can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the cDNA codes of SEQ ID NOs. 1-405, one or more of the polypeptide codes of SEQ ID NOS. 406-810. Another aspect of the present invention is a computer readable medium having recorded thereon at least 2, 5, 10, 15, 20, 25, 30, or 50 cDNA codes of SEQ ID NOs. 1-405. Another aspect of the present invention is a computer readable medium having recorded thereon at least 2, 5, 10, 15, 20, 25, 30, or 50 polypeptide codes of SEQ ID NOS. 406-810.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

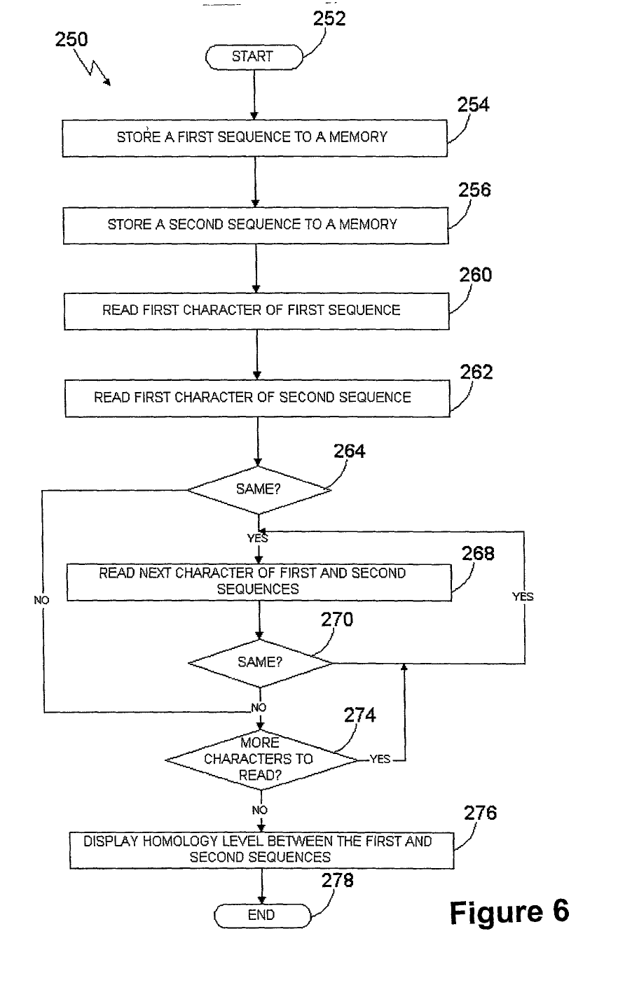

Embodiments of the present invention include systems, particularly computer systems which store and manipulate the sequence information described herein. One example of a computer system 100 is illustrated in block diagram form in FIG. 6. As used herein, “a computer system” refers to the hardware components, software components, and data storage components used to analyze the nucleotide sequences of the cDNA codes of SEQ ID NOs. 1-405, or the amino acid sequences of the polypeptide codes of SEQ ID NOS. 406-810. In one embodiment, the computer system 100 is a Sun Enterprise 1000 server (Sun Microsystems, Palo Alto, Calif.). The computer system 100 preferably includes a processor for processing, accessing and manipulating the sequence data. The processor 105 can be any well-known type of central processing unit, such as the Pentium III from Intel Corporation, or similar processor from Sun, Motorola, Compaq or International Business Machines.

Preferably, the computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one particular embodiment, the computer system 100 includes a processor 105 connected to a bus which is connected to a main memory 115 (preferably implemented as RAM) and one or more internal data storage devices 110, such as a hard drive and/or other computer readable media having data recorded thereon. In some embodiments, the computer system 100 further includes one or more data retrieving device 118 for reading the data stored on the internal data storage devices 110.

The data retrieving device 118 may represent, for example, a floppy disk drive, a compact disk drive, a magnetic tape drive, etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device.

The computer system 100 includes a display 120 which is used to display output to a computer user. It should also be noted that the computer system 100 can be linked to other computer systems 125a-c in a network or wide area network to provide centralized access to the computer system 100.

Software for accessing and processing the nucleotide sequences of the cDNA codes of SEQ ID NOs. 1-405, or the amino acid sequences of the polypeptide codes of SEQ ID NOS. 406-810 (such as search tools, compare tools, and modeling tools etc.) may reside in main memory 115 during execution.

In some embodiments, the computer system 100 may further comprise a sequence comparer for comparing the above-described cDNA codes of SEQ ID NOs. 1-405 or polypeptide codes of SEQ ID NOS. 406-810 stored on a computer readable medium to reference nucleotide or polypeptide sequences stored on a computer readable medium. A “sequence comparer” refers to one or more programs which are implemented on the computer system 100 to compare a nucleotide or polypeptide sequence with other nucleotide or polypeptide sequences and/or compounds including but not limited to peptides, peptidomimetics, and chemicals stored within the data storage means. For example, the sequence comparer may compare the nucleotide sequences of the cDNA codes of SEQ ID NOs1-405, or the amino acid sequences of the polypeptide codes of SEQ ID NOS. 406-810 stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention.

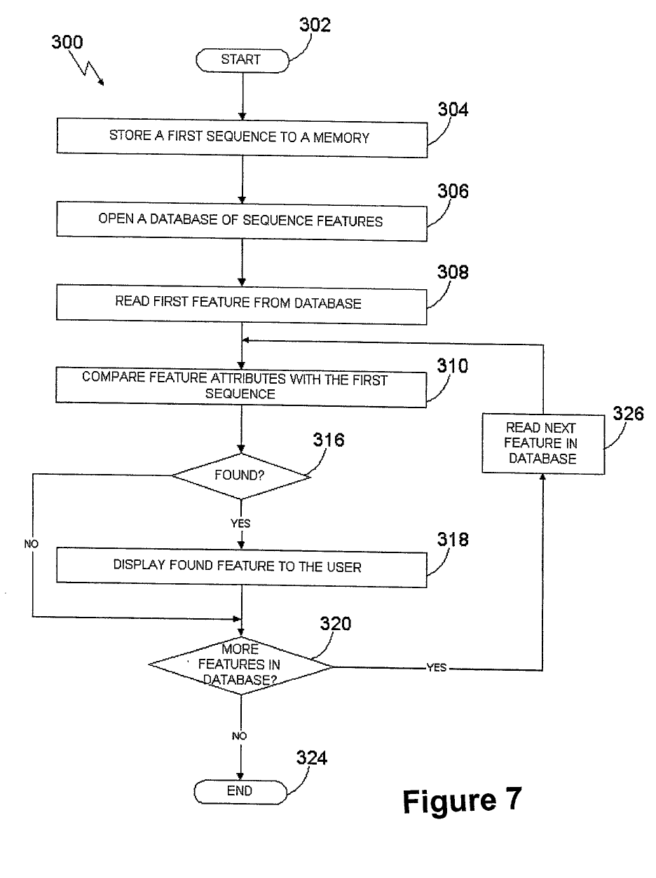

FIG. 7 is a flow diagram illustrating one embodiment of a process 200 for comparing a new nucleotide or protein sequence with a database of sequences in order to determine the identity levels between the new sequence and the sequences in the database. The database of sequences can be a private database stored within the computer system 100, or a public database such as GENBANK, PIR or SWISSPROT that is available through the Internet.

The process 200 begins at a start state 201 and then moves to a state 202 wherein the new sequence to be compared is stored to a memory in a computer system 100. As discussed above, the memory could be any type of memory, including RAM or an internal storage device.

The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the identity level between the two tested sequences. The parameters that control whether gaps or other features are introduced into a sequence during comparison are normally entered by the user of the computer system.

Once a comparison of the two sequences has been performed at the state 210, a determination is made at a decision state 210 whether the two sequences are the same. Of course, the term “same” is not limited to sequences that are absolutely identical. Sequences that are within the identity parameters entered by the user will be marked as “same” in the process 200.

If a determination is made that the two sequences are the same, the process 200 moves to a state 214 wherein the name of the sequence from the database is displayed to the user. This state notifies the user that the sequence with the displayed name fulfills the identity constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database.

It should be noted that if a determination had been made at the decision state 212 that the sequences were not homologous, then the process 200 would move immediately to the decision state 218 in order to determine if any other sequences were available in the database for comparison.

Accordingly, one aspect of the present invention is a computer system comprising a processor, a data storage device having stored thereon a nucleic acid code of SEQ ID NOs. 1-405 or a polypeptide code of SEQ ID NOS. 406-810, a data storage device having retrievably stored thereon reference nucleotide sequences or polypeptide sequences to be compared to the nucleic acid code of SEQ ID NOs. 1-405 or polypeptide code of SEQ ID NOS. 406-810 and a sequence comparer for conducting the comparison. The sequence comparer may indicate a identity level between the sequences compared or identify structural motifs in the above described nucleic acid code of SEQ ID NOs. 1-405 and polypeptide codes of SEQ ID NOS. 406-810 or it may identify structural motifs in sequences which are compared to these cDNA codes and polypeptide codes. In some embodiments, the data storage device may have stored thereon the sequences of at least 2, 5, 10, 15, 20, 25, 30, or 50 of the cDNA codes of SEQ ID NOs.1-405 or polypeptide codes of SEQ ID NOS. 406-810.

Another aspect of the present invention is a method for determining the level of identity between a nucleic acid code of SEQ ID NOs. 1-405 and a reference nucleotide sequence, comprising the steps of reading the nucleic acid code and the reference nucleotide sequence through the use of a computer program which determines identity levels and determining identity between the nucleic acid code and the reference nucleotide sequence with the computer program. The computer program may be any of a number of computer programs for determining identity levels, including those specifically enumerated herein, including BLAST2N with the default parameters or with any modified parameters. The method may be implemented using the computer systems described above. The method may also be performed by reading 2, 5, 10, 15, 20, 25, 30, or 50 of the above described cDNA codes of SEQ ID NOs. 1-405 through use of the computer program and determining identity between the cDNA codes and reference nucleotide sequences.

FIG. 8 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it should be in the single letter amino acid code so that the first and sequence sequences can be easily compared.

A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read.

If there aren't any more characters to read, then the process 250 moves to a state 276 wherein the level of identity between the first and second sequences is displayed to the user. The level of identity is determined by calculating the profragment of characters between the sequences that were the same out of the total number of sequences in the first sequence. Thus, if every character in a first 100 nucleotide sequence aligned with a every character in a second sequence, the identity level would be 100%.

Alternatively, the computer program may be a computer program which compares the nucleotide sequences of the cDNA codes of the present invention, to reference nucleotide sequences in order to determine whether the nucleic acid code of SEQ ID NOs. 1-405 differs from a reference nucleic acid sequence at one or more positions. Optionally such a program records the length and identity of inserted, deleted or substituted nucleotides with respect to the sequence of either the reference polynucleotide or the nucleic acid code of SEQ ID NOs. 1-405. In one embodiment, the computer program may be a program which determines whether the nucleotide sequences of the cDNA codes of SEQ ID NOs. 1-405 contain a biallelic marker or single nucleotide polymorphism (SNP) with respect to a reference nucleotide sequence. This single nucleotide polymorphism may comprise a single base substitution, insertion, or deletion, while this biallelic marker may comprise about one to ten consecutive bases substituted, inserted or deleted.

Another aspect of the present invention is a method for determining the level of identity between a polypeptide code of SEQ ID NOS. 406-810 and a reference polypeptide sequence, comprising the steps of reading the polypeptide code of SEQ ID NOS. 406-810 and the reference polypeptide sequence through use of a computer program which determines identity levels and determining identity between the polypeptide code and the reference polypeptide sequence using the computer program.

Accordingly, another aspect of the present invention is a method for determining whether a nucleic acid code of SEQ ID NOs. 1-405 differs at one or more nucleotides from a reference nucleotide sequence comprising the steps of reading the nucleic acid code and the reference nucleotide sequence through use of a computer program which identifies differences between nucleic acid sequences and identifying differences between the nucleic acid code and the reference nucleotide sequence with the computer program. In some embodiments, the computer program is a program which identifies single nucleotide polymorphisms. The method may be implemented by the computer systems described above and the method illustrated in FIG. 8. The method may also be performed by reading at least 2, 5, 10, 15, 20, 25, 30, or 50 of the cDNA codes of SEQ ID NOs. 1-405 and the reference nucleotide sequences through the use of the computer program and identifying differences between the cDNA codes and the reference nucleotide sequences with the computer program.

In other embodiments the computer based system may further comprise an identifier for identifying features within the nucleotide sequences of the cDNA codes of SEQ ID NOs. 1-405 or the amino acid sequences of the polypeptide codes of SEQ ID NOS. 406-810.

An “identifier” refers to one or more programs which identifies certain features within the above-described nucleotide sequences of the cDNA codes of SEQ ID NOs. 1-405 or the amino acid sequences of the polypeptide codes of SEQ ID NOS. 406-810. In one embodiment, the identifier may comprise a program which identifies an open reading frame in the cDNAs codes of SEQ ID NOs. 1-405.

FIG. 9 is a flow diagram illustrating one embodiment of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group (www.gcg.com).

Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user.

The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence.

It should be noted, that if the feature attribute is not found in the first sequence at the decision state 316, the process 300 moves directly to the decision state 320 in order to determine if any more features exist in the database.

In another embodiment, the identifier may comprise a molecular modeling program which determines the 3-dimensional structure of the polypeptides codes of SEQ ID NOS. 406-810. In some embodiments, the molecular modeling programidentifies target sequences that are most compatible with profiles representing the structural environments of the residues in known three-dimensional protein structures. (See, e.g., Eisenberg et al., U.S. Pat. No. 5,436,850 issued Jul. 25, 1995). In another technique, the known three-dimensional structures of proteins in a given family are superimposed to define the structurally conserved regions in that family. This protein modeling technique also uses the known three-dimensional structure of a homologous protein to approximate the structure of the polypeptide codes of SEQ ID NOS. 406-810. (See e.g., Srinivasan, et al., U.S. Pat. No. 5,557,535 issued Sep. 17, 1996). Conventional identity modeling techniques have been used routinely to build models of proteases and antibodies. (Sowdhamini et al., Protein Engineering 10:207, 215 (1997)). Comparative approaches can also be used to develop three-dimensional protein models when the protein of interest has poor sequence identity to template proteins. In some cases, proteins fold into similar three-dimensional structures despite having very weak sequence identities. For example, the three-dimensional structures of a number of helical cytokines fold in similar three-dimensional topology in spite of weak sequence identity.

The recent development of threading methods now enables the identification of likely folding patterns in a number of situations where the structural relatedness between target and template(s) is not detectable at the sequence level. Hybrid methods, in which fold recognition is performed using Multiple Sequence Threading (MST), structural equivalencies are deduced from the threading output using a distance geometry program DRAGON to construct a low resolution model, and a full-atom representation is constructed using a molecular modeling package such as QUANTA.

According to this 3-step approach, candidate templates are first identified by using the novel fold recognition algorithm MST, which is capable of performing simultaneous threading of multiple aligned sequences onto one or more 3-D structures. In a second step, the structural equivalencies obtained from the MST output are converted into inter-residue distance restraints and fed into the distance geometry program DRAGON, together with auxiliary information obtained from secondary structure predictions. The program combines the restraints in an unbiased manner and rapidly generates a large number of low resolution model confirmations. In a third step, these low resolution model confirmations are converted into full-atom models and subjected to energy minimization using the molecular modeling package QUANTA. (See e.g., Asz6di et al., Proteins:Structure, Function, and Genetics, Supplement 1:38-42 (1997)).

The results of the molecular modeling analysis may then be used in rational drug design techniques to identify agents which modulate the activity of the polypeptide codes of SEQ ID NOS. 74-123.

Accordingly, another aspect of the present invention is a method of identifying a feature within the cDNA codes of SEQ ID NOs. 1-405 or the polypeptide codes of SEQ ID NOS. 406-810 comprising reading the nucleic acid code(s) or the polypeptide code(s) through the use of a computer program which identifies features therein and identifying features within the nucleic acid code(s) or polypeptide code(s) with the computer program. In one embodiment, computer program comprises a computer program which identifies open reading frames. In a further embodiment, the computer program comprises a computer program which identifies linear or structural motifs in a polypeptide sequence. In another embodiment, the computer program comprises a molecular modeling program. The method may be performed by reading a single sequence or at least 2, 5, 10, 15, 20, 25, 30, or 50 of the cDNA codes of SEQ ID NOs. 1-405 or the polypeptide codes of SEQ ID NOS. 406-810 through the use of the computer program and identifying features within the cDNA codes or polypeptide codes with the computer program.

The cDNA codes of SEQ ID NOs. 1-405 or the polypeptide codes of SEQ ID NOS. 406-810 may be stored and manipulated in a variety of data processor programs in a variety of formats. For example, the cDNA codes of SEQ ID NOs. 1-405 or the polypeptide codes of SEQ ID NOS. 406-810 may be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparers, identifiers, or sources of reference nucleotide or polypeptide sequences to be compared to the cDNA codes of SEQ ID NOs.1-405 or the polypeptide codes of SEQ ID NOS406-810. The following list is intended not to limit the invention but to provide guidance to programs and databases which are useful with the cDNA codes of SEQ ID NOs. 1-405 or the polypeptide codes of SEQ ID NOS. 406-810. The programs and databases which may be used include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCB1), BLASTN and BLASTX (Altschul et al, J. Mol. Biol. 215: 403 (1990)), FASTA (Pearson and Lipman, Proc. Natl. Acad. Sci. USA, 85: 2444 (1988)), FASTDB (Brutlag et al. Comp. App. Biosci. 6:237-245, 1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the EMBL/Swissprotein database, the MDL Available Chemicals Directory database, the MDL Drug Data Report data base, the Comprehensive Medicinal Chemistry database, Derwents's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

### EXAMPLE 55

Methods of Making Nucleic Acids

The present invention also comprises methods of making the cDNA of SEQ ID Nos. 406-810, genomic DNA obtainable therefrom, or fragment thereof. The methods comprise sequentially linking together nucleotides to produce the nucleic acids having the preceding sequences. A variety of methods of synthesizing nucleic acids are known to those skilled in the art.

In many of these methods, synthesis is conducted on a solid support. These included the 3′ phosphoramidite methods in which the 3′ terminal base of the desired oligonucleotide is immobilized on an insoluble carrier. The nucleotide base to be added is blocked at the 5′ hydroxyl and activated at the 3′ hydroxyl so as to cause coupling with the immobilized nucleotide base. Deblocking of the new immobilized nucleotide compound and repetition of the cycle will produce the desired polynucleotide. Alternatively, polynucleotides may be prepared as described in U.S. Pat. No. 5,049,656. In some embodiments, several polynucleotides prepared as described above are ligated together to generate longer polynucleotides having a desired sequence.

### EXAMPLE 56

Methods of Making Polypeptides

The present invention also comprises methods of making the polynucleotides encoded by the cDNA of SEQ ID Nos. 1-405, genomic DNA obtainable therefrom, or fragments thereof and methods of making the polypeptides of SEQ ID Nos. 406-810 or fragments thereof. The methods comprise sequentially linking together amino acids to produce the nucleic polypeptides having the preceding sequences. In some embodiments, the polypeptides made by these methods are 150 amino acids or less in length. In other embodiments, the polypeptides made by these methods are 120 amino acids or less in length.

A variety of methods of making polypeptides are known to those skilled in the art, including methods in which the carboxyl terminal amino acid is bound to polyvinyl benzene or another suitable resin. The amino acid to be added possesses blocking groups on its amino moiety and any side chain reactive groups so that only its carboxyl moiety can react. The carboxyl group is activated with carbodiimide or another activating agent and allowed to couple to the immobilized amino acid. After removal of the blocking group, the cycle is repeated to generate a polypeptide having the desired sequence. Alternatively, the methods described in U.S. Pat. No. 5,049,656 may be used.

### EXAMPLE 57

Immunoaffinity Chromatography

Antibodies prepared as described above are coupled to a support. Preferably, the antibodies are monoclonal antibodies, but polyclonal antibodies may also be used. The support may be any of those typically employed in immunoaffinity chromatography, including Sepharose CL-4B (Pharmacia, Piscataway, N.J.), Sepharose CL-2B (Pharmacia, Piscataway, N.J.), Affi-gel 10 (Biorad, Richmond, Calif.), or glass beads.

The antibodies may be coupled to the support using any of the coupling reagents typically used in immunoaffinity chromatography, including cyanogen bromide. After coupling the antibody to the support, the support is contacted with a sample which contains a target polypeptide whose isolation, purification or enrichment is desired. The target polypeptide may be a polypeptide of SEQ ID NOs. 406-810, a fragment thereof, or a fusion protein comprising a polypeptide of SEQ ID NOs. 406-810 or a fragment thereof.

Preferably, the sample is placed in contact with the support for a sufficient amount of time and under appropriate conditions to allow at least 50% of the target polypeptide to specifically bind to the antibody coupled to the support.

Thereafter, the support is washed with an appropriate wash solution to remove polypeptides which have non-specifically adhered to the support. The wash solution may be any of those typically employed in immunoaffinity chromatography, including PBS, Tris-lithium chloride buffer (0.1M lysine base and 0.5M lithium chloride, pH 8.0), Tris-hydrochloride buffer (0.05M Tris-hydrochloride, pH 8.0), or Tris/Triton/NaCl buffer (50 mM Tris.cl, pH 8.0 or 9.0, 0.1% Triton X-100, and 0.5 MNaCl).

After washing, the specifically bound target polypeptide is eluted from the support using the high pH or low pH elution solutions typically employed in immunoaffinity chromatography. In particular, the elution solutions may contain an eluant such as triethanolamine, diethylamine, calcium chloride, sodium thiocyanate, potasssium bromide, acetic acid, or glycine. In some embodiments, the elution solution may also contain a detergent such as Triton X-100 or octyl-β-D-glucoside.

As discussed above, the cDNAs of the present invention or fragments thereof can be used for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; for selecting and making oligomers for attachment to a “gene chip” or other support, including for examination for expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, that described in Gyuris et al., Cell 75:791-803 (1993)) to identify polynucleotides encoding the other protein with which binding occurs or to identify inhibitors of the binding interaction.

The proteins or polypeptides provided by the present invention can similarly be used in assays to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning; A Laboratory Manual”, 2d ed., Cole Spring Harbor Laboratory Press, Sambrook, J., E. F. Fritsch and T. Maniatis eds., 1989, and “Methods in Enzymology; Guide to Molecular Cloning Techniques”, Academic Press, Berger, S. L. and A. R. Kimmel eds., 1987.

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. All documents cited herein are incorporated herein by reference in their entirety.

TABLE I

| | | | Mature | | | |
|---|---|---|---|---|---|---|
| | FCS | SigPep | Polypeptide | Stop Codon | PolyA Signal | PolyA Site |
| Id | Location | Location | Location | Location | Location | Location |
| 1 | 153/1127 | 153/230 | 231/1127 | 1128 | 1415/1420 | 1434/1450 |
| 2 | 261/1166 | 261/314 | 315/1166 | 1167 | — | 1524/1556 |
| 3 | 67/813 | 67/111 | 112/813 | 814 | 1023/1028 | 1042/1058 |
| 4 | 187/438 | — | 187/438 | 439 | 612/617 | 632/648 |
| 5 | 92/1753 | 92/130 | 131/1753 | 1754 | 2070/2075 | 2090/2104 |
| 6 | 144/440 | 144/287 | 288/440 | 441 | 457/462 | 500/515 |
| 7 | 174/443 | 174/269 | 270/443 | 444 | 623/628 | 647/661 |
| 8 | 55/399 | 55/192 | 193/399 | 400 | 654/659 | 680/694 |
| 9 | 90/287 | 90/146 | 147/287 | 288 | 1078/1083 | 1096/1110 |
| 10 | 49/447 | 49/111 | 112/447 | 448 | 579/584 | 602/623 |
| 11 | 199/618 | 199/408 | 409/618 | 619 | 626/631 | 643/657 |
| 12 | 271/969 | 271/366 | 367/969 | 970 | 1092/1097 | 1123/1137 |
| 13 | 192/440 | 192/278 | 279/440 | 441 | 590/595 | 622/636 |
| 14 | 59/703 | 59/181 | 182/703 | 704 | 783/788 | 804/818 |
| 15 | 139/1389 | 139/198 | 199/1389 | 1390 | 1854/1859 | 1873/1888 |
| 16 | 21/1118 | 21/89 | 90/1118 | 1119 | 1858/1863 | 1879/1894 |
| 17 | 143/592 | 143/277 | 278/592 | 593 | 1877/1882 | 1899/1913 |
| 18 | 76/999 | 76/279 | 280/999 | 1000 | 1711/1716 | 1729/1744 |
| 19 | 123/464 | 123/269 | 270/464 | 465 | 908/913 | 931/946 |
| 20 | 85/1230 | 85/129 | 130/1230 | 1231 | 1589/1594 | 1607/1622 |
| 21 | 29/664 | 29/619 | 620/664 | 665 | 657/662 | 699/715 |
| 22 | 18/878 | 18/95 | 96/878 | 879 | 1500/1505 | 1533/1549 |
| 23 | 73/1008 | 73/147 | 148/1008 | 1009 | 1286/1291 | 1312/1328 |
| 24 | 165/842 | 165/251 | 252/842 | 843 | 1474/1479 | 1500/1515 |
| 25 | 31/1248 | 31/135 | 136/1248 | 1249 | 1580/1585 | 1607/1622 |
| 26 | 131/490 | 131/301 | 302/490 | 491 | 1411/1416 | 1434/1448 |
| 27 | 61/690 | 61/168 | 169/690 | 691 | 858/863 | 879/894 |
| 28 | 501/1253 | 501/1229 | 1230/1253 | 1254 | 1392/1397 | 1432/1447 |
| 29 | 25/402 | 25/96 | 97/402 | 403 | 1500/1505 | 1525/1540 |
| 30 | 280/678 | 280/411 | 412/678 | 679 | 1606/1611 | 1628/1643 |
| 31 | 64/726 | 64/147 | 148/726 | 727 | 1279/1284 | 1300/1314 |
| 32 | 42/1097 | 42/110 | 111/1097 | 1098 | 2323/2328 | 2341/2356 |
| 33 | 245/1399 | 245/796 | 797/1399 | 1400 | 1669/1674 | 1687/1701 |
| 34 | 235/441 | 235/303 | 304/441 | 442 | — | 758/772 |
| 35 | 88/411 | 88/234 | 235/411 | 412 | 938/943 | 964/987 |
| 36 | 129/452 | 129/212 | 213/452 | 453 | 1290/1295 | 1309/1324 |
| 37 | 238/612 | 238/348 | 349/612 | 613 | 1885/1890 | 1905/1918 |
| 38 | 229/735 | 229/492 | 493/735 | 736 | 816/821 | 841/852 |
| 39 | 168/413 | 168/335 | 336/413 | 414 | 684/689 | 708/726 |
| 40 | 100/852 | 100/159 | 160/852 | 853 | 998/1003 | 1019/1039 |
| 41 | 238/1152 | 238/339 | 340/1152 | 1153 | 1298/1303 | 1324/1355 |
| 42 | 187/369 | 187/312 | 313/369 | 370 | 489/494 | 558/572 |
| 43 | 121/459 | 121/165 | 166/459 | 460 | 497/502 | 521/535 |
| 44 | 34/336 | 34/123 | 124/336 | 337 | 536/541 | 556/572 |
| 45 | 119/409 | 119/388 | 389/409 | 410 | 769/774 | 789/804 |
| 46 | 232/534 | 232/306 | 307/534 | 535 | 595/600 | 615/629 |
| 47 | 140/595 | 140/442 | 443/595 | 596 | 630/635 | 655/669 |
| 48 | 32/658 | 32/289 | 290/658 | 659 | 936/941 | 959/973 |
| 49 | 14/280 | 14/76 | 77/280 | 281 | — | 776/791 |
| 50 | 93/290 | 93/149 | 150/290 | 291 | 1078/1083 | 1096/1110 |
| 51 | 131-1042 | 131-169 | 170-1042 | — | — | 1042-1053 |
| 52 | 100-276 | — | 100-276 | 277 | 638-643 | 662-675 |
| 53 | 111-401 | 111-194 | 195-401 | 402 | 1080-1085 | 1101-1112 |
| 54 | 359-514 | 359-454 | 455-514 | 515 | — | 536-547 |
| 55 | 26-397 | 26-316 | 317-397 | 398 | 1164-1169 | 1187-1198 |
| 56 | 36-725 | 36-107 | 108-725 | 726 | 1302-1307 | 1389-1400 |
| 57 | 35-250 | 35-130 | 131-250 | 251 | 505-510 | 526-538 |
| 58 | 169-432 | 169-267 | 268-432 | 433 | 1132-1137 | 1155-1167 |
| 59 | 143-460 | 143-238 | 239-460 | 461 | 697-702 | 721-730 |
| 60 | 108-908 | 108-170 | 171-908 | 909 | 1141-1146 | 1161-1174 |
| 61 | 209-532 | — | 209-532 | 533 | 1133-1138 | 1146-1158 |
| 62 | 5-211 | 5-142 | 143-211 | 212 | 716-721 | 742-754 |
| 63 | 98-850 | 98-181 | 182-850 | 851 | 1035-1040 | 1060-1073 |
| 64 | 46-342 | 46-189 | 190-342 | 343 | 377-382 | 402-413 |
| 65 | 139-381 | 139-231 | 232-381 | 382 | 579-584 | 598-609 |
| 66 | 72-512 | — | 72-512 | — | — | 512-522 |
| 67 | 126-944 | 126-260 | 261-944 | 945 | 1283-1288 | 1309-1322 |
| 68 | 50-1279 | 50-160 | 161-1279 | — | — | 1280-1290 |
| 69 | 83-1261 | 83-139 | 140-1261 | 1262 | — | — |
| 70 | 57-1199 | 57-95 | 96-1199 | 1200 | 1438-1443 | 1458-1470 |
| 71 | 72-944 | 72-197 | 198-944 | 945 | — | 970-982 |
| 72 | 4-279 | — | 4-279 | 280 | 425-430 | 443-455 |
| 73 | 90-470 | 90-278 | 279-470 | 471 | 704-709 | 724-738 |
| 74 | 88-339 | 88-147 | 148-339 | 340 | 619-624 | 637-649 |
| 75 | 33-578 | 33-92 | 93-578 | 579 | — | 703-714 |
| 76 | 33-245 | 33-107 | 108-245 | 246 | 546-551 | 584-596 |
| 77 | 125-343 | — | 125-343 | 344 | 375-380 | 390-403 |
| 78 | 126-632 | 126-575 | 576-632 | 633 | 670-675 | 721-727 |
| 79 | 90-317 | 90-155 | 156-317 | 318 | 913-918 | 932-944 |
| 80 | 126-410 | 126-287 | 288-410 | 411 | 561-566 | 587-598 |
| 81 | 85-348 | 85-150 | 151-348 | — | — | 349-360 |
| 82 | 77-343 | 77-124 | 125-343 | 344 | 461-466 | 477-490 |
| 83 | 38-364 | — | 38-364 | 365 | 458-463 | 475-488 |
| 84 | 48-389 | 48-356 | 357-389 | 390 | 742-747 | 760-771 |
| 85 | 69-440 | 69-359 | 360-440 | 441 | 927-932 | 947-959 |
| 86 | 33-311 | 33-98 | 99-311 | 312 | 437-442 | 455-464 |
| 87 | 110-730 | 110-235 | 236-730 | 731 | 764-769 | 787-799 |
| 88 | 38-214 | — | 38-214 | 215 | — | 308-320 |
| 89 | 129-296 | 129-209 | 209-296 | 297 | — | 318-331 |
| 90 | 78-563 | 78-359 | 340-563 | 564 | 1042-1047 | 1063-1075 |
| 91 | 62-523 | 62-265 | 266-523 | 524 | 602-607 | 621-632 |
| 92 | 24-320 | — | 24-320 | 321 | 402407 | 419-430 |
| 93 | 42-170 | 42-113 | 114-170 | 171 | — | 172-185 |
| 94 | 108-314 | 108-170 | 171-314 | 315 | 550-555 | 574-585 |
| 95 | 118-351 | 118-171 | 172-351 | 352 | 583-588 | 602-613 |
| 96 | 128-367 | 128-268 | 269-367 | 368 | 410-415 | 424-427 |
| 97 | 149-871 | 149-457 | 458-871 | 872 | — | 893-912 |
| 99 | 7-471 | 7-99 | 100-471 | 472 | 537-542 | 554-568 |
| 100 | 168 / 332 | — | 168 / 332 | 333 | — | — |
| 101 | 51 / 251 | 51 / 110 | 111 / 251 | 252 | 849 / 854 | 882 / 895 |
| 102 | 20 / 613 | 20 / 82 | 83 / 613 | 614 | — | — |
| 103 | 12 / 416 | 12 / 86 | 87 / 416 | 417 | 425 / 430 | 445 / 458 |
| 104 | 276 / 1040 | 276 / 485 | 486 / 1040 | 1041 | — | 2024 / 2036 |
| 105 | 443 / 619 | 443 / 589 | 590 / 619 | 620 | — | 1267 / 1276 |
| 106 | 206 / 747 | — | 206 / 747 | — | — | — |
| 107 | 36 / 521 | 36 / 104 | 105 / 521 | 522 | 528 / 533 | 548 / 561 |
| 108 | 36 / 395 | 36 / 104 | 105 / 395 | 396 | 599 / 604 | 619 / 632 |
| 109 | 21 / 41 | — | 21 / 41 | 42 | 328 / 333 | 357 / 370 |
| 110 | 35 / 631 | 35 / 160 | 161 / 631 | 632 | 901 / 906 | 979 / 994 |
| 111 | 271 / 399 | — | 271 / 399 | 400 | — | — |
| 112 | 103 / 252 | 103 / 213 | 214 / 252 | 253 | — | 588 / 597 |
| 113 | 2 / 460 | — | 2 / 460 | 461 | 713 / 718 | 735 / 748 |
| 114 | 31 / 231 | — | 31 / 231 | 232 | 769 / 774 | 690 / 703 |
| 115 | 305 / 565 | — | 305 / 565 | 566 | 694 / 699 | 713 / 725 |
| 116 | 124 / 873 | 124 / 378 | 379 / 873 | 874 | 1673 / 1678 | 1694 / 1705 |
| 117 | 135 / 206 | — | 135 / 206 | 207 | 850 / 855 | 1056 / 1069 |
| 118 | 135 / 818 | — | 135 / 818 | 819 | 909 / 914 | 1071 / 1084 |
| 119 | 33 / 290 | 33 / 92 | 93 / 290 | 291 | — | — |
| 120 | 485 / 616 | — | 485 / 616 | 617 | — | 669 / 682 |
| 121 | 54 / 995 | 54 / 227 | 228 / 995 | 996 | 1130 / 1135 | 1181 / 1191 |
| 122 | 657 / 923 | 657 / 896 | 897 / 923 | 924 | 957 / 962 | 974 / 1008 |
| 123 | 18 / 311 | 18 / 62 | 63 / 311 | 312 | — | — |
| 124 | 151 / 426 | 151 / 258 | 259 / 426 | 427 | 505 / 510 | 527 / 538 |
| 125 | 10 / 1062 | 10 / 57 | 58 / 1062 | 1063 | 1710 / 1715 | 1735 / 1747 |
| 126 | 78 / 491 | 78 / 218 | 219 / 491 | 492 | 1652 / 1657 | 1673 / 1686 |
| 127 | 69 / 371 | 69 / 287 | 288 / 371 | 372 | 510 / 515 | 530 / 542 |
| 128 | 2 / 757 | 2 / 205 | 206 / 757 | 758 | — | 1160 / 1174 |
| 129 | 2 / 1051 | 2 / 205 | 206 / 1051 | 1052 | 1248 / 1253 | 1272 / 1285 |
| 130 | 2 / 1171 | 2 / 205 | 206 / 1171 | 1172 | 1368 / 1373 | 1386 / 1398 |
| 131 | 42 / 611 | 42 / 287 | 288 / 611 | 612 | 787 / 792 | 808 / 821 |
| 132 | 62 / 916 | 62 / 757 | 758 / 916 | — | — | 904 / 916 |
| 133 | 62 / 520 | — | 62 / 520 | 521 | 1124 / 1129 | 1141 / 1153 |
| 134 | 21 / 167 | — | 21 / 167 | 168 | — | — |
| 135 | 22 / 318 | 22 / 93 | 94 / 318 | 319 | 497 / 502 | 516 / 526 |
| 136 | 8 / 292 | 8 / 118 | 119 / 292 | 293 | 317 / 322 | 339 / 352 |
| 137 | 16 / 378 | 16 / 84 | 85 / 378 | 379 | 502 / 507 | 522 / 542 |
| 138 | 57 / 233 | — | 57 / 233 | — | — | — |
| 139 | 83 / 340 | 83 / 124 | 125 / 340 | 341 | 573 / 578 | 607 / 660 |
| 140 | 47 / 541 | 47 / 220 | 221 / 541 | 542 | — | 597 / 605 |
| 141 | 46 / 285 | 46 / 150 | 151 / 285 | 286 | 364 / 369 | 385 / 396 |
| 142 | 22 / 240 | 22 / 84 | 85 / 240 | 241 | 397 / 402 | 421 / 432 |
| 143 | 89 / 382 | — | 89 / 382 | 383 | — | 408 / 420 |
| 144 | 80 / 415 | 80 / 142 | 143 / 415 | — | 471 / 476 | 488 / 501 |
| 145 | 152 / 361 | 152 / 283 | 284 / 361 | 362 | — | — |
| 146 | 32 / 307 | 32 / 70 | 71 / 307 | 308 | 1240 / 1245 | 1261 / 1272 |
| 147 | 114 / 734 | 114 / 239 | 240 / 734 | 735 | 768 / 773 | 793 / 804 |
| 148 | 199 / 802 | — | 199 / 802 | — | 780 / 785 | 791 / 802 |
| 149 | 38 / 1174 | 38 / 148 | 149 / 1174 | 1175 | 1452 / 1457 | 1478 / 1490 |
| 150 | 26 / 361 | — | 26 / 361 | — | — | 350 / 361 |
| 151 | 3 / 131 | — | 3 / 131 | 132 | — | 591 / 605 |
| 152 | 33 / 185 | 33 / 80 | 81 / 185 | 186 | 570 / 575 | 586 / 591 |
| 153 | 184 / 915 | 184 / 237 | 238 / 915 | 916 | 1119 / 1124 | 1139 / 1150 |
| 154 | 58 / 1116 | 58 / 159 | 160 / 1116 | 1117 | 1486 / 1491 | 1504 / 1513 |
| 155 | 327 / 417 | — | 327 / 417 | — | — | 404 / 417 |
| 156 | 63 / 398 | 63 / 206 | 207 / 398 | 399 | — | — |
| 157 | 2 / 163 | — | 2 / 163 | | 488 / 493 | 511 / 522 |
| 158 | 13 / 465 | 13 / 75 | 76 / 465 | 466 | — | — |
| 159 | 20 / 703 | 20 / 94 | 95 / 703 | 704 | 1000 / 1005 | 1023 / 1041 |
| 160 | 103 / 294 | 103 / 243 | 244 / 294 | 295 | — | — |
| 161 | 81 / 518 | 81 / 173 | 174 / 518 | 519 | — | — |
| 162 | 66 / 326 | — | 66 / 326 | 327 | 1066 / 1071 | 1087 / 1098 |
| 163 | 170 / 289 | 170 / 250 | 251 / 289 | 290 | — | — |
| 164 | 36 / 497 | — | 36 / 497 | 498 | 650 / 655 | 663 / 685 |
| 165 | 18 / 320 | — | 18 / 320 | 321 | 539 / 544 | 542 / 554 |
| 166 | 71 / 1438 | 71 / 136 | 137 / 1438 | 1439 | 1644 / 1649 | 1665 / 1678 |
| 167 | 25 / 318 | 25 / 75 | 76 / 318 | 319 | 452 / 457 | 482 / 494 |
| 168 | 84 / 332 | 84 / 170 | 171 / 332 | 333 | — | 702 / 714 |
| 169 | 32 / 718 | 32 / 100 | 101 / 718 | 719 | 770 / 775 | 793 / 805 |
| 170 | 26 / 481 | 26 / 88 | 89 / 481 | 482 | 755 / 760 | 775 / 787 |
| 171 | 26 / 562 | 26 / 187 | 188 / 562 | 563 | — | — |
| 172 | 4 / 810 | 4 / 279 | 280 / 810 | 811 | 858 / 863 | 881 / 893 |
| 173 | 55 / 459 | 55 / 120 | 121 / 459 | 460 | 1444 / 1449 | 1462 / 1475 |
| 174 | 48 / 248 | 48 / 161 | 162 / 248 | 249 | 283 / 288 | 308 / 321 |
| 175 | 25 / 399 | 25 / 186 | 187 / 399 | 400 | — | — |
| 176 | 10 / 1137 | 10 / 72 | 73 / 1137 | 1138 | 1144 / 1149 | 1162 / 1173 |
| 177 | 72 / 704 | 72 / 161 | 162 / 704 | 705 | 772 / 777 | — |
| 178 | 44 / 505 | 44 / 223 | 224 / 505 | 506 | — | — |
| 179 | 25 / 393 | 25 / 150 | 151 / 393 | 394 | 734 / 739 | 757 / 770 |
| 180 | 58 / 1095 | 58 / 114 | 115 / 1095 | 1096 | — | 1202 / 1213 |
| 181 | 31 / 660 | 31 / 90 | 91 / 660 | 661 | 1288 / 1293 | 1307 / 1318 |
| 182 | 31 / 582 | 31 / 90 | 91 / 582 | 583 | 816 / 821 | 840 / 853 |
| 183 | 15 / 695 | 15 / 80 | 81 / 695 | 696 | 795 / 800 | 814 / 826 |
| 184 | 74 / 295 | 74 / 196 | 197 / 295 | 296 | 545 / 550 | 561 / 571 |
| 185 | 440 / 659 | — | 440 / 659 | — | 601 / 606 | — |
| 186 | 38 / 283 | 38 / 85 | 86 / 283 | 284 | 257 / 262 | — |
| 187 | 121 / 477 | 121 / 288 | 289 / 477 | — | — | — |
| 188 | 2 / 163 | — | 2 / 163 | 164 | 292 / 297 | 310 / 323 |
| 189 | 46 / 675 | 46 / 87 | 88 / 675 | | 1364 / 1369 | 1383 / 1392 |
| 190 | 62 / 385 | — | 62 / 385 | 386 | 974 / 979 | 987 / 999 |
| 191 | 422 / 550 | 422 / 475 | 476 / 550 | 551 | — | 714 / 725 |
| 192 | 124 / 231 | — | 124 / 231 | 232 | — | 387 / 400 |
| 193 | 131 /1053 | 131 / 169 | 170 / 1053 | — | 1019 / 1024 | — |
| 194 | 86 / 403 | 86 / 181 | 182 / 403 | 404 | 1097 / 1102 | 1117 / 1128 |
| 195 | 37 / 162 | 37 / 93 | 94 / 162 | 163 | 224 / 229 | 243 / 254 |
| 196 | 31 / 381 | 31 / 90 | 91 / 381 | 382 | — | 875 / 886 |
| 197 | 46 / 579 | 46 / 156 | 157 / 579 | 580 | — | — |
| 198 | 92 / 471 | 92 / 172 | 173 / 471 | — | 454 / 459 | 458 / 471 |
| 199 | 154 / 675 | 154 / 498 | 499 / 675 | 676 | 819 / 824 | 838 / 849 |
| 200 | 18 / 173 | 18 / 77 | 78 / 173 | 174 | 864 / 869 | 882 / 893 |
| 201 | 17 / 595 | 17 / 85 | 86 / 595 | 596 | 820 / 825 | 840 / 851 |
| 202 | 89 / 334 | 89 / 130 | 131 / 334 | 335 | 462 / 467 | 484 / 495 |
| 203 | 21 / 614 | 21 / 83 | 84 / 614 | 615 | 849 / 854 | 873 / 884 |
| 204 | 94 / 573 | 94 / 258 | 259 / 573 | 574 | 862 / 867 | 886 / 897 |
| 205 | 74 / 397 | 74 / 127 | 128 / 397 | 398 | 472 / 477 | 507 / 518 |
| 206 | 51 / 242 | 51 / 116 | 117 / 242 | 243 | 319 / 324 | 339 / 350 |
| 207 | 111 / 191 | 111/ 155 | 156 / 191 | 192 | 965 / 970 | 986 / 996 |
| 208 | 45 / 602 | 45 / 107 | 108 / 602 | 603 | 828 / 833 | 850 / 860 |
| 209 | 24 / 560 | 24 / 101 | 102 / 560 | 561 | 563 / 568 | 583 / 593 |
| 210 | 109 / 558 | 109/ 273 | 274 / 558 | 559 | — | 1104 / 1114 |
| 211 | 128 / 835 | 128/ 220 | 221 / 835 | 836 | 1145 / 1150 | 1170 / 1181 |
| 212 | 59 / 505 | 59 / 358 | 359 / 505 | 506 | 1042 / 1047 | 1062 / 1073 |
| 213 | 1 / 207 | 1 / 147 | 148 / 207 | 208 | 784 / 789 | 807 / 818 |
| 214 | 12 / 734 | 12 / 101 | 102 / 734 | 735 | 914 / 919 | 961 / 971 |
| 215 | 378 / 518 | 378/ 467 | 468 / 518 | 519 | 607 / 612 | 628 / 640 |
| 216 | 110 / 304 | 110/ 193 | 194 / 304 | 305 | 708 / 713 | 732 / 743 |
| 217 | 201 / 419 | 201/ 272 | 273 / 419 | 420 | 601 / 606 | 627 / 637 |
| 218 | 123 / 302 | 123/ 176 | 177 / 302 | 303 | 1279 / 1284 | 1301 / 1312 |
| 219 | 98 / 673 | 98 / 376 | 377 / 673 | 674 | — | 1025 / 1035 |
| 220 | 17 / 463 | 17 / 232 | 233 / 463 | 464 | 657 / 662 | 684 / 696 |
| 221 | 263 / 481 | 263/ 322 | 323 / 481 | 482 | — | 858 / 868 |
| 222 | 42 / 299 | 42 / 101 | 102 / 299 | 300 | — | 762 / 775 |
| 223 | 198 / 431 | 198/ 260 | 261 / 431 | 432 | — | 1064 / 1074 |
| 224 | 279 / 473 | 279/ 362 | 363 / 473 | 474 | 944 / 949 | 970 / 981 |
| 225 | 12 / 644 | 12 / 92 | 93 / 644 | 645 | 1002 / 1007 | 1020 / 1031 |
| 226 | 91 / 459 | 91 / 330 | 331 / 459 | 460 | — | 1271 / 1281 |
| 227 | 70 / 327 | 70 / 147 | 148 / 327 | 328 | 1741 / 1746 | 1763 / 1774 |
| 228 | 12 / 497 | 12 / 104 | 105 / 497 | 498 | 935 / 940 | 955 / 967 |
| 229 | 90 / 383 | 90 / 200 | 201 / 383 | 384 | 609 / 614 | 632 / 643 |
| 230 | 332 / 541 | 332/ 376 | 377 / 541 | 542 | 739 / 744 | 761 / 773 |
| 231 | 43 / 222 | 43 / 177 | 178 / 222 | 223 | 530 / 535 | 555 / 566 |
| 232 | 115 / 231 | 115/ 180 | 181 / 231 | 232 | 419 / 424 | 445 / 455 |
| 233 | 232 / 384 | 232/ 300 | 301 / 384 | 385 | 650 / 655 | 662 / 673 |
| 234 | 143 / 427 | 143/ 286 | 287 / 427 | 428 | 606 / 611 | 628 / 639 |
| 235 | 284 / 463 | 284/ 379 | 380 / 463 | 464 | — | 762 / 772 |
| 236 | 162 / 671 | 162/ 398 | 399 / 671 | 672 | 805 / 810 | 830 / 840 |
| 237 | 63 / 632 | 63 / 308 | 309 / 632 | 633 | 808 / 813 | 829 / 840 |
| 238 | 21 / 362 | 21 / 200 | 201 / 362 | 363 | 821 / 826 | 838 / 849 |
| 239 | 21 / 503 | 21 / 344 | 345 / 503 | 504 | 1305 / 1310 | 1330 / 1341 |
| 240 | 1 / 201 | 1 / 63 | 64 / 201 | 202 | 637 / 642 | 660 / 671 |
| 241 | 39 / 1034 | 39 / 134 | 135 / 1034 | 1035 | 1566 / 1571 | 1587 / 1597 |
| 242 | 69 / 263 | 69 / 125 | 126 / 263 | 264 | 1173 / 1178 | 1196 / 1205 |
| 243 | 115 / 285 | 115/ 204 | 205 / 285 | 286 | 505 / 510 | 525 / 536 |
| 244 | 90 / 344 | 90 / 140 | 141 / 344 | 345 | 500 / 505 | 515 / 527 |
| 245 | 57 / 311 | 57 / 107 | 108 / 311 | 312 | 467 / 472 | 482 / 493 |
| 246 | 96 / 302 | 96 / 182 | 183 / 302 | 303 | — | 501 / 514 |
| 247 | 161 / 526 | 161/ 328 | 329 / 526 | 527 | — | 799 / 811 |
| 248 | 210 / 332 | 210/ 299 | 300 / 332 | 333 | 594 / 599 | 613 / 625 |
| 249 | 212 / 361 | 212/ 319 | 320 / 361 | 362 | 650 / 655 | 673 / 684 |
| 250 | 75 / 482 | 75 / 128 | 129 / 482 | 483 | 595 / 600 | 618 / 627 |
| 251 | 50 / 631 | 50 / 244 | 245 / 631 | 632 | 777 / 782 | 801 / 812 |
| 252 | 154 / 576 | 154/ 360 | 361 / 576 | 577 | 737 / 742 | 763 / 775 |
| 253 | 154 / 897 | 154/ 360 | 361 / 897 | 898 | 1017 / 1022 | 1044 / 1054 |
| 254 | 146 / 292 | 146/ 253 | 254 / 292 | 293 | 395 / 400 | 433 / 444 |
| 255 | 126 / 383 | 126/ 167 | 168 / 383 | 384 | 726 / 731 | 743 / 754 |
| 256 | 66 / 497 | 66 / 239 | 240 / 497 | 498 | 594 / 599 | 618 / 629 |
| 257 | 49 / 411 | 49 / 96 | 97 / 411 | 412 | 732 / 737 | 750 / 763 |
| 258 | 49 / 534 | 49 / 96 | 97 / 534 | 535 | 593 / 598 | 612 / 623 |
| 259 | 86 / 415 | 86 / 145 | 146 / 415 | 416 | 540 / 545 | 560 / 571 |
| 260 | 56 / 268 | 56 / 100 | 101 / 268 | 269 | 584 / 589 | 601 / 612 |
| 261 | 32 / 328 | 32 / 103 | 104 / 328 | 329 | 508 / 513 | 528 / 539 |
| 262 | 21 / 527 | 21 / 95 | 96 / 527 | 528 | 921 / 926 | 953 / 963 |
| 263 | 147 / 647 | 147/ 374 | 375 / 647 | 648 | — | 668 / 681 |
| 264 | 262 / 471 | 262/ 306 | 307 / 471 | 472 | 663 / 668 | 682 / 693 |
| 265 | 74 / 1216 | 74 / 172 | 173 / 1216 | 1217 | 1627 / 1632 | 1640 / 1652 |
| 266 | 48 / 164 | 48 / 89 | 90 / 164 | 165 | 482 / 487 | 505 / 517 |
| 267 | 185 / 334 | 185/ 295 | 296 / 334 | 335 | 355 / 360 | 392 / 405 |
| 268 | 195 / 347 | 195/ 272 | 273 / 347 | 348 | 1037 / 1042 | 1071 / 1082 |
| 269 | 90 / 815 | 90 / 179 | 180 / 815 | 816 | 883 / 888 | 905 / 916 |
| 270 | 52 / 513 | 52 / 231 | 232 / 513 | 514 | 553 / 558 | 572 / 583 |
| 271 | 172 / 438 | 172/ 354 | 355 / 438 | 439 | 682 / 687 | 685 / 697 |
| 272 | 148 / 366 | 148/ 225 | 226 / 366 | 367 | 770 / 775 | 792 / 803 |
| 273 | 175 / 336 | 17 / 276 | 277 / 336 | 337 | — | 812 / 823 |
| 274 | 191 / 553 | 191/ 304 | 305 / 553 | 554 | 766 / 771 | 804 / 817 |
| 275 | 106 / 603 | 106/ 216 | 217 / 603 | 604 | — | 1102 / 1112 |
| 276 | 47 / 586 | 47 / 124 | 125 / 586 | 587 | 1583 / 1588 | 1614 / 1623 |
| 277 | 99 / 371 | 99 / 290 | 291 / 371 | 372 | 491 / 496 | 513 / 524 |
| 278 | 44 / 814 | 44 / 112 | 113 / 814 | 815 | — | 978 / 989 |
| 279 | 3 / 581 | 3 / 182 | 183 / 581 | 582 | — | 1006 / 1016 |
| 280 | 107 / 427 | 107/ 190 | 191 / 427 | 428 | 499 / 504 | 516 / 529 |
| 281 | 45 / 407 | 45 / 83 | 84 / 407 | 408 | 1008 / 1013 | 1032 / 1042 |
| 282 | 201 / 332 | 201/ 251 | 252 / 332 | 333 | — | 869 / 880 |
| 283 | 217 / 543 | 217/ 255 | 256 / 543 | 544 | — | 1206 / 1217 |
| 284 | 18 / 446 | 18 / 140 | 141 / 446 | 447 | 930 / 935 | 948 / 959 |
| 285 | 29 / 724 | 29 / 118 | 119 / 724 | 725 | 886 / 891 | 910 / 920 |
| 286 | 404 / 586 | 404/ 466 | 467 / 586 | 587 | 1304 / 1309 | 1334 / 1344 |
| 287 | 331 / 432 | 331/ 387 | 388 / 432 | 433 | 548 / 553 | 573 / 585 |
| 288 | 59 / 703 | 59 / 220 | 221 / 703 | 704 | 886 / 891 | 903 / 914 |
| 289 | 672 / 752 | 672/ 722 | 723 / 752 | 753 | — | 1150 / 1161 |
| 290 | 57 / 311 | 57 / 128 | 129 / 311 | 312 | 332 / 337 | 351 / 363 |
| 291 | 80 / 232 | 80 / 127 | 128 / 232 | 233 | 617 / 622 | 634 / 645 |
| 292 | 91 / 291 | 91 / 219 | 220 / 291 | 292 | 367 / 372 | 389 / 400 |
| 293 | 196 / 384 | 196/ 240 | 241 / 384 | 385 | 461 / 466 | 485 / 496 |
| 294 | 54 / 590 | 54 / 227 | 228 / 590 | 591 | — | 955 / 965 |
| 295 | 133 / 846 | 133/ 345 | 346 / 846 | 847 | — | 890 / 901 |
| 296 | 138 / 671 | 138/ 248 | 249 / 671 | 672 | 1319 / 1324 | 1338 / 1347 |
| 297 | 124 / 411 | 124/ 186 | 187 / 411 | 412 | 948 / 953 | 971 / 983 |
| 298 | 372 / 494 | 372/ 443 | 444 / 494 | 495 | 708 / 713 | 732 / 745 |
| 299 | 112 / 450 | 112/ 192 | 193 / 450 | 451 | 1053 / 1058 | 1095 / 1106 |
| 300 | 117 / 866 | 117/ 170 | 171 / 866 | 867 | 1159 / 1164 | 1178 / 1190 |
| 301 | 13 / 465 | 13 / 75 | 76 / 465 | 466 | 1035 / 1040 | 1060 / 1070 |
| 302 | 2 / 718 | 2 / 76 | 77 / 718 | 719 | 1170 / 1175 | 1203 / 1213 |
| 303 | 86 / 709 | 86 / 361 | 362 / 709 | 710 | 943 / 948 | 963 / 973 |
| 304 | 63 / 320 | 63 / 179 | 180 / 320 | 321 | 771 / 776 | 799 / 810 |
| 305 | 299 / 418 | 299/ 379 | 380 / 418 | 419 | 739 / 744 | 762 / 771 |
| 306 | 186 / 380 | 186/ 233 | 234 / 380 | 381 | 383 / 388 | 396 / 409 |
| 307 | 69 / 458 | 69 / 233 | 234 / 458 | 459 | 564 / 569 | 602 / 613 |
| 308 | 12 / 638 | 12 / 263 | 264 / 638 | 639 | 951 / 956 | 975 / 985 |
| 309 | 282 / 389 | 282/ 332 | 333 / 389 | 390 | 1413 / 1418 | 1437 / 1447 |
| 310 | 208 / 339 | 208/ 294 | 295 / 339 | 340 | — | 1631 / 1641 |
| 311 | 69 / 557 | 69 / 224 | 225 / 557 | 558 | 849 / 854 | 870 / 883 |
| 312 | 134 / 325 | 134/ 274 | 275 / 325 | 326 | — | 718 / 729 |
| 313 | 78 / 731 | 78 / 227 | 228 / 731 | 732 | — | 1002 / 1013 |
| 314 | 46 / 693 | 46 / 90 | 91 / 693 | 694 | 937 / 942 | 962 / 973 |
| 315 | 126 / 527 | 126/ 182 | 183 / 527 | 528 | 834 / 839 | 856 / 867 |
| 316 | 66 / 320 | 66 / 113 | 114 / 320 | 321 | 490 / 495 | 508 / 519 |
| 317 | 73 / 948 | 73 / 159 | 160 / 948 | 949 | — | 1016 / 1028 |
| 318 | 69 / 434 | 69 / 236 | 237 / 434 | 435 | 419 / 424 | 441 / 452 |
| 319 | 628 / 804 | 628/ 711 | 712 / 804 | 805 | — | 864 / 875 |
| 320 | 70 / 366 | 70 / 108 | 109 / 366 | 367 | 496 / 501 | 521 / 531 |
| 321 | 70 / 366 | 70 / 108 | 109 / 366 | 367 | — | 1233 / 1244 |
| 322 | 111 / 434 | 111/ 185 | 186 / 434 | 435 | — | 618 / 631 |
| 323 | 19 / 567 | 19 / 63 | 64 / 567 | 568 | 749 / 754 | 771 / 781 |
| 324 | 19 / 312 | 19 / 63 | 64 / 312 | 313 | 896 / 901 | 921 / 931 |
| 325 | 64 / 612 | 64 / 234 | 235 / 612 | 613 | — | 839 / 849 |
| 326 | 39 / 458 | 39 / 80 | 81 / 458 | 459 | 613 / 618 | 633 / 644 |
| 327 | 9 / 185 | 9 / 50 | 51 / 185 | 186 | — | 906 / 918 |
| 328 | 14 / 316 | 14 / 121 | 122 / 316 | 317 | 442 / 447 | 458 / 471 |
| 329 | 70 / 1092 | 70 / 234 | 235 / 1092 | 1093 | 1475 / 1480 | 1493 / 1504 |
| 330 | 274 / 597 | 274/ 399 | 400 / 597 | 598 | 731 / 736 | 754 / 765 |
| 331 | 230 / 469 | 230/ 307 | 308 / 469 | 470 | 1004 / 1009 | 1027 / 1040 |
| 332 | 72 / 545 | 72 / 203 | 204 / 545 | 546 | — | 1151 / 1162 |
| 333 | 36 / 425 | 36 / 119 | 120 / 425 | 426 | 1215 / 1220 | 1240 / 1250 |
| 334 | 155 / 751 | 155/ 340 | 341 / 751 | 752 | 912 / 917 | 937 / 947 |
| 335 | 46 / 585 | 46 / 120 | 121 / 585 | 586 | 584 / 589 | 606 / 619 |
| 336 | 35 / 568 | 35 / 100 | 101 / 568 | 569 | 667 / 672 | 685 / 699 |
| 337 | 68 / 337 | 68 / 124 | 125 / 337 | 338 | 462 / 467 | 482 / 497 |
| 338 | 39 / 413 | 39 / 83 | 84 / 413 | 414 | 566 / 571 | 583 / 598 |
| 339 | 235 / 642 | 235 / 336 | 337 / 642 | 643 | 1540 / 1545 | 1564 / 1579 |
| 340 | 42 / 755 | 42 / 200 | 201 / 755 | 756 | 860 / 865 | 878 / 893 |
| 341 | 23 / 340 | 23 / 235 | 236 / 340 | 341 | 611 / 616 | 629 / 644 |
| 342 | 12 / 380 | 12 / 263 | 264 / 380 | 381 | — | 523 / 538 |
| 343 | 8 / 232 | 8 / 154 | 155 / 232 | 233 | — | 737 / 752 |
| 344 | 183 / 422 | 183 / 302 | 303 / 422 | 423 | 505 / 510 | 523 / 537 |
| 345 | 24 / 1004 | 24 / 170 | 171 / 1004 | 1005 | — | 1586 / 1602 |
| 346 | 80 / 784 | 80 / 139 | 140 / 784 | 785 | 910 / 915 | 933 / 948 |
| 347 | 67 / 222 | 67 / 159 | 160 / 222 | 223 | — | 673 / 687 |
| 348 | 46 / 732 | 46 / 186 | 187 / 732 | 733 | 781 / 786 | 806 / 821 |
| 349 | 81 / 356 | 81 / 152 | 153 / 356 | 357 | 406 / 411 | 429 / 445 |
| 350 | 72 / 1346 | 72 / 140 | 141 / 1346 | 1347 | 1482 / 1487 | 1502 / 1517 |
| 351 | 194 / 454 | 194 / 379 | 380 / 454 | 455 | — | 1545 / 1560 |
| 352 | 48 / 494 | 48 / 347 | 348 / 494 | 495 | 1031 / 1036 | 1051 / 1066 |
| 353 | 111 / 671 | 111 / 215 | 216 / 671 | 672 | 990 / 995 | 1045 / 1061 |
| 354 | 5 / 373 | 5 / 82 | 83 / 373 | 374 | 1986 / 1991 | 2010 / 2025 |
| 355 | 14 / 472 | 14 / 319 | 320 / 472 | 473 | 555 / 560 | 576 / 591 |
| 356 | 2 / 217 | — | 2 / 217 | 218 | 489 / 494 | 529 / 544 |
| 357 | 51 / 575 | 51 / 110 | 111 / 575 | 576 | 1653 / 1658 | 1674 / 1689 |
| 358 | 69 / 977 | 69 / 128 | 129 / 977 | 978 | 1076 / 1081 | 1096 / 1111 |
| 359 | 44 / 238 | 44 / 160 | 161 / 238 | 239 | 443 / 448 | 540 / 554 |
| 360 | 114 / 524 | 114 / 164 | 165 / 524 | 525 | 1739 / 1744 | 1758 / 1773 |
| 361 | 26 / 487 | 26 / 64 | 65 / 487 | 488 | 883 / 888 | 901 / 917 |
| 362 | 80 / 388 | 80 / 187 | 188 / 388 | 389 | 609 / 614 | 627 / 641 |
| 363 | 186 / 443 | 186 / 407 | 408 / 443 | 444 | 827 / 832 | 839 / 854 |
| 364 | 75 / 1259 | 75 / 1004 | 1005 / 1259 | 1260 | 1536 / 1541 | 1553 / 1568 |
| 365 | 98 / 376 | 98 / 151 | 152 / 376 | 377 | 471 / 476 | 491 / 506 |
| 366 | 72 / 254 | 72 / 134 | 135 / 254 | 255 | 506 / 511 | 528 / 542 |
| 367 | 148 / 1140 | 148 / 240 | 241 / 1140 | 1141 | 1590 / 1595 | 1614 / 1629 |
| 368 | 109 / 738 | 109 / 405 | 406 / 738 | 739 | 1633 / 1638 | 1650 / 1665 |
| 369 | 55 / 291 | 55 / 255 | 256 / 291 | 292 | 390 / 395 | 410 / 425 |
| 370 | 25 / 276 | — | 25 / 276 | 277 | 508 / 513 | 533 / 546 |
| 371 | 32 / 307 | 32 / 91 | 92 / 307 | 308 | 452 / 457 | 472 / 485 |
| 372 | 46 / 675 | 46 / 87 | 88 / 675 | 676 | 1363 / 1368 | 1382 / 1394 |
| 373 | 329 / 943 | 329 / 745 | 746 / 943 | 944 | — | 1322 / 1333 |
| 374 | 27 / 281 | 27 / 77 | 78 / 281 | 282 | — | — |
| 375 | 61 / 405 | 61 / 213 | 214 / 405 | 406 | 675 / 680 | 692 / 703 |
| 376 | 137 / 379 | 137 / 229 | 230 / 379 | 380 | 728 / 733 | 755 / 768 |
| 377 | 37 / 741 | 37 / 153 | 154 / 741 | 742 | 969 / 974 | 994 / 1007 |
| 378 | 80 / 265 | 80 / 142 | 143 / 265 | 266 | 491 / 496 | 517 / 527 |
| 379 | 612 / 644 | — | 612 / 644 | 645 | 829 / 834 | 850 / 861 |
| 380 | 61 / 228 | 61 / 162 | 163 / 228 | 229 | 208 / 213 | — |
| 381 | 15 / 311 | 15 / 110 | 111 / 311 | 312 | 507 / 512 | 531 / 542 |
| 382 | 50 / 529 | 50 / 130 | 131 / 529 | 530 | 877 / 882 | 899 / 909 |
| 383 | 240 / 416 | 240 / 305 | 306 / 416 | 417 | 1117 / 1122 | 1139 / 1149 |
| 384 | 111 / 446 | 111 / 254 | 255 / 446 | 447 | 890 / 895 | 909 / 921 |
| 385 | 123 / 455 | 123 / 290 | 291 / 455 | 456 | 886 / 891 | 904 / 916 |
| 386 | 2 / 433 | 2 / 232 | 233 / 433 | 434 | 488 / 493 | 510 / 520 |
| 387 | 34 / 363 | 34 / 87 | 88 / 363 | 364 | 536 / 541 | 558 / 568 |
| 388 | 50 / 286 | 50 / 157 | 158 / 286 | 287 | 385 / 390 | 405 / 416 |
| 389 | 50 / 637 | 50 / 151 | 152 / 637 | 638 | — | 1277 / 1289 |
| 390 | 72 / 602 | 72 / 125 | 126 / 602 | 603 | — | 704 / 715 |
| 391 | 120 / 434 | 120 / 185 | 186 / 434 | 435 | 899 / 904 | 918 / 931 |
| 392 | 4 / 447 | 4 / 147 | 148 / 447 | 448 | 858 / 863 | 880 / 891 |
| 393 | 28 / 804 | 28 / 96 | 97 / 804 | 805 | — | 806 / 817 |
| 394 | 27 / 359 | 27 / 212 | 213 / 359 | 360 | 988 / 993 | 1009 / 1020 |
| 395 | 25 / 957 | 25 / 93 | 94 / 957 | 958 | 1368 / 1373 | 1388 / 1399 |
| 396 | 47 / 319 | 47 / 226 | 227 / 319 | 320 | — | 656 / 666 |
| 397 | 80 / 940 | 80 / 130 | 131 / 940 | 941 | 1101 / 1106 | 1119 / 1130 |
| 398 | 146 / 457 | 146 / 292 | 293 / 457 | 458 | 442 / 447 | 465 / 475 |
| 399 | 100 / 351 | 100 / 207 | 208 / 351 | 352 | — | 940 / 949 |
| 400 | 177 / 569 | 177 / 236 | 237 / 569 | 570 | — | 931 / 939 |
| 401 | 67 / 459 | 67 / 135 | 136 / 459 | 460 | 856 / 861 | 875 / 887 |
| 402 | 65 / 1069 | 65 / 112 | 113 / 1069 | 1070 | 1978 / 1983 | 1999 / 2010 |
| 403 | 70 / 321 | 70 / 234 | 235 / 321 | 322 | 364 / 369 | 375 / 387 |
| 404 | 38 / 877 | 38 / 91 | 92 / 877 | 878 | 947 / 952 | 974 / 983 |
| 405 | 51 / 470 | 51 / 203 | 204 / 470 | 471 | 1585 / 1590 | 1604 / 1614 |

TABLE II

| | Full Length | Signal | Mature |
|---|---|---|---|
| | Polypeptide | Peptide | Polypeptide |
| Seq Id No | Location | Location | Location |
| 406 | −26 / 299 | −26 / −1 | 1 / 299 |
| 407 | −18 / 284 | −18 / −1 | 1 / 284 |
| 408 | −15 / 234 | −15 / −1 | 1 / 234 |
| 409 | 1 / 84 | — | 1 / 84 |
| 410 | −13 / 541 | −13 / −1 | 1 / 541 |
| 411 | −48 / 51 | −48 / −1 | 1 / 51 |
| 412 | −32 / 58 | −32 / −1 | 1 / 58 |
| 413 | −46 / 69 | −46 / −1 | 1 / 69 |
| 414 | −19 / 47 | −19 / −1 | 1 / 47 |
| 415 | −21 / 112 | −21 / −1 | 1 / 112 |
| 416 | −70 / 70 | −70 / −1 | 1 / 70 |
| 417 | −32 / 201 | −32 / −1 | 1 / 201 |
| 418 | −29 / 54 | −29 / −1 | 1 / 54 |
| 419 | −41 / 174 | −41 / −1 | 1 / 174 |
| 420 | −20 / 397 | −20 / −1 | 1 / 397 |
| 421 | −23 / 343 | −23 / −1 | 1 / 343 |
| 422 | −45 / 105 | −45 / −1 | 1 / 105 |
| 423 | −68 / 240 | −68 / −1 | 1 / 240 |
| 424 | −49 / 65 | −49 / −1 | 1 / 65 |
| 425 | −15 / 367 | −15 / −1 | 1 / 367 |
| 426 | −197 / 15 | −197 / −1 | 1 / 15 |
| 427 | −26 / 261 | −26 / −1 | 1 / 261 |
| 428 | −25 / 287 | −25 / −1 | 1 / 287 |
| 429 | −29 / 197 | −29 / −1 | 1 / 197 |
| 430 | −35 / 371 | −35 / −1 | 1 / 371 |
| 431 | −57 / 63 | −57 / −1 | 1 / 63 |
| 432 | −36 / 174 | −36 / −1 | 1 / 174 |
| 433 | −243 / 8 | −243 / −1 | 1 / 8 |
| 434 | −24 / 102 | −24 / −1 | 1 / 102 |
| 435 | −44 / 89 | −44 / −1 | 1 / 89 |
| 436 | −28 / 193 | −28 / −1 | 1 / 193 |
| 437 | −23 / 329 | −23 / −1 | 1 / 329 |
| 438 | −184 / 201 | −184 / −1 | 1 / 201 |
| 439 | −23 / 46 | −23 / −1 | 1 / 46 |
| 440 | −49 / 59 | −49 / −1 | 1 / 59 |
| 441 | −28 / 80 | −28 / −1 | 1 / 80 |
| 442 | −37 / 88 | −37 / −1 | 1 / 88 |
| 443 | −88 / 81 | −88 / −1 | 1 / 81 |
| 444 | −56 / 26 | −56 / −1 | 1 / 26 |
| 445 | −20 / 231 | −20 / −1 | 1 / 231 |
| 446 | −34 / 271 | −34 / −1 | 1 / 271 |
| 447 | −42 / 19 | −42 / −1 | 1 / 19 |
| 448 | −15 / 98 | −15 / −1 | 1 / 98 |
| 449 | −30 / 71 | −30 / −1 | 1 / 71 |
| 450 | −90 / 7 | −90 / −1 | 1 / 7 |
| 451 | −25 / 76 | −25 / −1 | 1 / 76 |
| 452 | −101 / 51 | −101 / −1 | 1 / 51 |
| 453 | −86 / 123 | −86 / −1 | 1 / 123 |
| 454 | −21 / 68 | −21 / −1 | 1 / 68 |
| 455 | −19 / 47 | −19 / −1 | 1 / 47 |
| 693 | −13 / 291 | −13 / −1 | 1 / 291 |
| 694 | 1 / 59 | — | 1 / 59 |
| 695 | −28 / 69 | −28 / −1 | 1 / 69 |
| 696 | −32 / 20 | −32 / −1 | 1 / 20 |
| 697 | −97 / 27 | −97 / −1 | 1 / 27 |
| 698 | −24 / 206 | −24 / −1 | 1 / 206 |
| 699 | −32 / 40 | −32 / −1 | 1 / 40 |
| 700 | −33 / 55 | −33 / −1 | 1 / 55 |
| 701 | −32 / 74 | −32 / −1 | 1 / 74 |
| 702 | −21 / 246 | −21 / −1 | 1 / 246 |
| 703 | 1 / 108 | — | 1 / 108 |
| 704 | −46 / 23 | −46 / −1 | 1 / 23 |
| 705 | −28 / 223 | −28 / −1 | 1 / 223 |
| 706 | −48 / 51 | −48 / −1 | 1 / 51 |
| 707 | −31 / 50 | −31 / −1 | 1 / 50 |
| 708 | 1 / 147 | — | 1 / 147 |
| 709 | −45 / 228 | −45 / −1 | 1 / 228 |
| 710 | −37 / 373 | −37 / −1 | 1 / 373 |
| 711 | −19 / 374 | −19 / −1 | 1 / 374 |
| 712 | −13 / 368 | −13 / −1 | 1 / 368 |
| 713 | −42 / 249 | −42 / −1 | 1 / 249 |
| 714 | 1 / 92 | — | 1 / 92 |
| 715 | −63 / 64 | −63 / −1 | 1 / 64 |
| 716 | −20 / 64 | −20 / −1 | 1 / 64 |
| 717 | −20 / 162 | −20 / −1 | 1 / 162 |
| 718 | −25 / 46 | −25 / −1 | 1 / 46 |
| 719 | 1 / 73 | — | 1 / 73 |
| 720 | −150 / 19 | −150 / −1 | 1 / 19 |
| 721 | −22 / 54 | −22 / −1 | 1 / 54 |
| 722 | −54 / 41 | −54 / −1 | 1 / 41 |
| 723 | −22 / 66 | −22 / −1 | 1 / 66 |
| 724 | −16 / 73 | −16 / −1 | 1 / 73 |
| 725 | 1 / 109 | — | 1 / 109 |
| 726 | −103 / 11 | −103 / −1 | 1 / 11 |
| 727 | −97 / 27 | −97 / −1 | 1 / 27 |
| 728 | −22 / 71 | −22 / −1 | 1 / 71 |
| 729 | −42 / 165 | −42 / −1 | 1 / 165 |
| 730 | 1 / 59 | — | 1 / 59 |
| 731 | −27 / 29 | −27 / −1 | 1 / 29 |
| 732 | −94 / 68 | −94 / −1 | 1 / 68 |
| 733 | −68 / 86 | −68 / −1 | 1 / 86 |
| 734 | 1 / 99 | — | 1 / 99 |
| 735 | −24 / 19 | −24 / −1 | 1 / 19 |
| 736 | −21 / 48 | −21 / −1 | 1 / 48 |
| 737 | −18 / 60 | −18 / −1 | 1 / 60 |
| 738 | −47 / 33 | −47 / −1 | 1 / 33 |
| 739 | −103 / 138 | −103 / −1 | 1 / 138 |
| 456 | −31 / 124 | −31 / −1 | 1 / 124 |
| 456 | −31 / 124 | −31 / −1 | 1 / 124 |
| 457 | 1 / 55 | — | 1 / 55 |
| 458 | −20 / 47 | −20 / −1 | 1 / 47 |
| 459 | −21 / 177 | −21 / −1 | 1 / 177 |
| 460 | −25 / 110 | −25 / −1 | 1 / 110 |
| 461 | −70 / 185 | −70 / −1 | 1 / 185 |
| 462 | −49 / 10 | −49 / −1 | 1 / 10 |
| 463 | 1 / 180 | — | 1 / 180 |
| 464 | −23 / 139 | −23 / −1 | 1 / 139 |
| 465 | −23 / 97 | −23 / −1 | 1 / 97 |
| 466 | 1 / 7 | — | 1 / 7 |
| 467 | −42 / 157 | −42 / −1 | 1 / 157 |
| 468 | 1 / 43 | — | 1 / 43 |
| 469 | −37 / 13 | −37 / −1 | 1 / 13 |
| 470 | 1 / 153 | — | 1 / 153 |
| 471 | 1 / 67 | — | 1 / 67 |
| 472 | 1 / 87 | — | 1 / 87 |
| 473 | −85 / 165 | −85 / −1 | 1 / 165 |
| 474 | 1 / 24 | — | 1 / 24 |
| 475 | 1 / 228 | — | 1 / 228 |
| 476 | −20 / 66 | −20 / −1 | 1 / 66 |
| 477 | 1 / 44 | — | 1 / 44 |
| 478 | −58 / 256 | −58 / −1 | 1 / 256 |
| 479 | −80 / 9 | −80 / −1 | 1 / 9 |
| 480 | −15 / 83 | −15 / −1 | 1 / 83 |
| 481 | −36 / 56 | −36 / −1 | 1 / 56 |
| 482 | −16 / 335 | −16 / −1 | 1 / 335 |
| 483 | −47 / 91 | −47 / −1 | 1 / 91 |
| 484 | −73 / 28 | −73 / −1 | 1 / 28 |
| 485 | −68 / 184 | −68 / −1 | 1 / 184 |
| 486 | −68 / 282 | −68 / −1 | 1 / 282 |
| 487 | −68 / 322 | −68 / −1 | 1 / 322 |
| 488 | −82 / 108 | −82 / −1 | 1 / 108 |
| 489 | −232 / 53 | −232 / −1 | 1 / 53 |
| 490 | 1 / 153 | — | 1 / 153 |
| 491 | 1 / 49 | — | 1 / 49 |
| 492 | −24 / 75 | −24 / −1 | 1 / 75 |
| 493 | −37 / 58 | −37 / −1 | 1 / 58 |
| 494 | −23 / 98 | −23 / −1 | 1 / 98 |
| 495 | 1 / 59 | — | 1 / 59 |
| 496 | −14 / 72 | −14 / −1 | 1 / 72 |
| 497 | −58 / 107 | −58 / −1 | 1 / 107 |
| 498 | −35 / 45 | −35 / −1 | 1 / 45 |
| 499 | −21 / 52 | −21 / −1 | 1 / 52 |
| 500 | 1 / 98 | — | 1 / 98 |
| 501 | −21 / 91 | −21 / −1 | 1 / 91 |
| 502 | −44 / 26 | −44 / −1 | 1 / 26 |
| 503 | −13 / 79 | −13 / −1 | 1 / 79 |
| 504 | −42 / 165 | −42 / −1 | 1 / 165 |
| 505 | 1 / 201 | — | 1 / 201 |
| 506 | −37 / 342 | −37 / −1 | 1 / 342 |
| 507 | 1 / 112 | — | 1 / 112 |
| 508 | 1 / 43 | — | 1 / 43 |
| 509 | −16 / 35 | −16 / −1 | 1 / 35 |
| 510 | −18 / 226 | −18 / −1 | 1 / 226 |
| 511 | −34 / 319 | −34 / −1 | 1 / 319 |
| 512 | 1 / 30 | — | 1 / 30 |
| 513 | −48 / 64 | −48 / −1 | 1 / 64 |
| 514 | 1 / 54 | — | 1 / 54 |
| 515 | −21 / 130 | −21 / −1 | 1 / 130 |
| 516 | −25 / 203 | −25 / −1 | 1 / 203 |
| 517 | −47 / 17 | −47 / −1 | 1 / 17 |
| 518 | −31 / 115 | −31 / −1 | 1 / 115 |
| 519 | 1 / 87 | — | 1 / 87 |
| 520 | −27 / 13 | −27 / −1 | 1 / 13 |
| 521 | 1 / 154 | — | 1 / 154 |
| 522 | 1 / 101 | — | 1 / 101 |
| 523 | −22 / 434 | −22 / −1 | 1 / 434 |
| 524 | −17 / 81 | −17 / −1 | 1 / 81 |
| 525 | −29 / 54 | −29 / −1 | 1 / 54 |
| 526 | −23 / 206 | −23 / −1 | 1 / 206 |
| 527 | −21 / 131 | −21 / −1 | 1 / 131 |
| 528 | −54 / 125 | −54 / −1 | 1 / 125 |
| 529 | −92 / 177 | −92 / −1 | 1 / 177 |
| 530 | −22 / 113 | −22 / −1 | 1 / 113 |
| 531 | −38 / 29 | −38 / −1 | 1 / 29 |
| 532 | −54 / 71 | −54 / −1 | 1 / 71 |
| 533 | −21 / 355 | −21 / −1 | 1 / 355 |
| 534 | −30 / 181 | −30 / −1 | 1 / 181 |
| 535 | −60 / 94 | −60 / −1 | 1 / 94 |
| 536 | −42 / 81 | −42 / −1 | 1 / 81 |
| 537 | −19 / 327 | −19 / −1 | 1 / 327 |
| 538 | −20 / 190 | −20 / −1 | 1 / 190 |
| 539 | −20 / 164 | −20 / −1 | 1 / 164 |
| 540 | −22 / 205 | −22 / −1 | 1 / 205 |
| 541 | −41 / 33 | −41 / −1 | 1 / 33 |
| 542 | 1 / 73 | — | 1 / 73 |
| 543 | −16 / 66 | −16 / −1 | 1 / 66 |
| 544 | −56 / 63 | −56 / −1 | 1 / 63 |
| 545 | 1 / 54 | — | 1 / 54 |
| 546 | −14 / 196 | −14 / −1 | 1 / 196 |
| 547 | 1 / 108 | — | 1 / 108 |
| 548 | −18 / 25 | −18 / −1 | 1 / 25 |
| 549 | 1 / 36 | — | 1 / 36 |
| 550 | −13 / 294 | −13 / −1 | 1 / 294 |
| 551 | −32 / 74 | −32 / −1 | 1 / 74 |
| 552 | −19 / 23 | −19 / −1 | 1 / 23 |
| 553 | −20 / 97 | −20 / −1 | 1 / 97 |
| 554 | −37 / 141 | −37 / −1 | 1 / 141 |
| 555 | −27 / 99 | −27 / −1 | 1 / 99 |
| 556 | −115 / 59 | −115 / −1 | 1 / 59 |
| 557 | −20 / 32 | −20 / −1 | 1 / 32 |
| 558 | −23 / 170 | −23 / −1 | 1 / 170 |
| 559 | −14 / 68 | −14 / −1 | 1 / 68 |
| 560 | −21 / 177 | −21 / −1 | 1 / 177 |
| 561 | −55 / 105 | −55 / −1 | 1 / 105 |
| 562 | −18 / 90 | −18 / −1 | 1 / 90 |
| 563 | −22 / 42 | −22 / −1 | 1 / 42 |
| 564 | −15 / 12 | −15 / −1 | 1 / 12 |
| 565 | −21 / 165 | −21 / −1 | 1 / 165 |
| 566 | −26 / 153 | −26 / −1 | 1 / 153 |
| 567 | −55 / 95 | −55 / −1 | 1 / 95 |
| 568 | −31 / 205 | −31 / −1 | 1 / 205 |
| 569 | −100 / 49 | −100 / −1 | 1 / 49 |
| 570 | −49 / 20 | −49 / −1 | 1 / 20 |
| 571 | −30 / 211 | −30 / −1 | 1 / 211 |
| 572 | −30 / 17 | −30 / −1 | 1 / 17 |
| 573 | −28 / 37 | −28 / −1 | 1 / 37 |
| 574 | −24 / 49 | −24 / −1 | 1 / 49 |
| 575 | −18 / 42 | −18 / −1 | 1 / 42 |
| 576 | −93 / 99 | −93 / −1 | 1 / 99 |
| 577 | −72 / 77 | −72 / −1 | 1 / 77 |
| 578 | −20 / 53 | −20 / −1 | 1 / 53 |
| 579 | −20 / 66 | −20 / −1 | 1 / 66 |
| 580 | −21 / 57 | −21 / −1 | 1 / 57 |
| 581 | −28 / 37 | −28 / −1 | 1 / 37 |
| 582 | −27 / 184 | −27 / −1 | 1 / 184 |
| 583 | −80 / 43 | −80 / −1 | 1 / 43 |
| 584 | −26 / 60 | −26 / −1 | 1 / 60 |
| 585 | −31 / 131 | −31 / −1 | 1 / 131 |
| 586 | −37 / 61 | −37 / −1 | 1 / 61 |
| 587 | −15 / 55 | −15 / −1 | 1 / 55 |
| 588 | −45 / 15 | −45 / −1 | 1 / 15 |
| 589 | −22 / 17 | −22 / −1 | 1 / 17 |
| 590 | −23 / 28 | −23 / −1 | 1 / 28 |
| 591 | −48 / 47 | −48 / −1 | 1 / 47 |
| 592 | −32 / 28 | −32 / −1 | 1 / 28 |
| 593 | −79 / 91 | −79 / −1 | 1 / 91 |
| 594 | −82 / 108 | −82 / −1 | 1 / 108 |
| 595 | −60 / 54 | −60 / −1 | 1 / 54 |
| 596 | −108 / 53 | −108 / −1 | 1 / 53 |
| 597 | −21 / 46 | −21 / −1 | 1 / 46 |
| 598 | −32 / 300 | −32 / −1 | 1 / 300 |
| 599 | −19 / 46 | −19 / −1 | 1 / 46 |
| 600 | −30 / 27 | −30 / −1 | 1 / 27 |
| 601 | −17 / 68 | −17 / −1 | 1 / 68 |
| 602 | −17 / 68 | −17 / −1 | 1 / 68 |
| 603 | −29 / 40 | −29 / −1 | 1 / 40 |
| 604 | −56 / 66 | −56 / −1 | 1 / 66 |
| 605 | −30 / 11 | −30 / −1 | 1 / 11 |
| 606 | −36 / 14 | −36 / −1 | 1 / 14 |
| 607 | −18 / 118 | −18 / −1 | 1 / 118 |
| 608 | −65 / 129 | −65 / −1 | 1 / 129 |
| 609 | −69 / 72 | −69 / −1 | 1 / 72 |
| 610 | −69 / 179 | −69 / −1 | 1 / 179 |
| 611 | −36 / 13 | −36 / −1 | 1 / 13 |
| 612 | −14 / 72 | −14 / −1 | 1 / 72 |
| 613 | −58 / 86 | −58 / −1 | 1 / 86 |
| 614 | −16 / 105 | −16 / −1 | 1 / 105 |
| 615 | −16 / 146 | −16 / −1 | 1 / 146 |
| 616 | −20 / 90 | −20 / −1 | 1 / 90 |
| 617 | −15 / 56 | −15 / −1 | 1 / 56 |
| 618 | −24 / 75 | −24 / −1 | 1 / 75 |
| 619 | −25 / 144 | −25 / −1 | 1 / 144 |
| 620 | −76 / 91 | −76 / −1 | 1 / 91 |
| 621 | −15 / 55 | −15 / −1 | 1 / 55 |
| 622 | −33 / 348 | −33 / −1 | 1 / 348 |
| 623 | −14 / 25 | −14 / −1 | 1 / 25 |
| 624 | −37 / 13 | −37 / −1 | 1 / 13 |
| 625 | −26 / 25 | −26 / −1 | 1 / 25 |
| 626 | −30 / 212 | −30 / −1 | 1 / 212 |
| 627 | −60 / 94 | −60 / −1 | 1 / 94 |
| 628 | −61 / 28 | −61 / −1 | 1 / 28 |
| 629 | −26 / 47 | −26 / −1 | 1 / 47 |
| 630 | −34 / 20 | −34 / −1 | 1 / 20 |
| 631 | −38 / 83 | −38 / −1 | 1 / 83 |
| 632 | −37 / 129 | −37 / −1 | 1 / 129 |
| 633 | −26 / 154 | −26 / −1 | 1 / 154 |
| 634 | −64 / 27 | −64 / −1 | 1 / 27 |
| 635 | −23 / 234 | −23 / −1 | 1 / 234 |
| 636 | −60 / 133 | −60 / −1 | 1 / 133 |
| 637 | −28 / 79 | −28 / −1 | 1 / 79 |
| 638 | −13 / 108 | −13 / −1 | 1 / 108 |
| 639 | −17 / 27 | −17 / −1 | 1 / 27 |
| 640 | −13 / 96 | −13 / −1 | 1 / 96 |
| 641 | −41 / 102 | −41 / −1 | 1 / 102 |
| 642 | −30 / 202 | −30 / −1 | 1 / 202 |
| 643 | −21 / 40 | −21 / −1 | 1 / 40 |
| 644 | −19 / 15 | −19 / −1 | 1 / 15 |
| 645 | −54 / 161 | −54 / −1 | 1 / 161 |
| 646 | −17 / 10 | −17 / −1 | 1 / 10 |
| 647 | −24 / 61 | −24 / −1 | 1 / 61 |
| 648 | −16 / 35 | −16 / −1 | 1 / 35 |
| 649 | −43 / 24 | −43 / −1 | 1 / 24 |
| 650 | −15 / 48 | −15 / −1 | 1 / 48 |
| 651 | −58 / 121 | −58 / −1 | 1 / 121 |
| 652 | −71 / 167 | −71 / −1 | 1 / 167 |
| 653 | −37 / 141 | −37 / −1 | 1 / 141 |
| 654 | −21 / 75 | −21 / −1 | 1 / 75 |
| 655 | −24 / 17 | −24 / −1 | 1 / 17 |
| 656 | −27 / 86 | −27 / −1 | 1 / 86 |
| 657 | −18 / 232 | −18 / −1 | 1 / 232 |
| 658 | −21 / 130 | −21 / −1 | 1 / 130 |
| 659 | −25 / 214 | −25 / −1 | 1 / 214 |
| 660 | −92 / 116 | −92 / −1 | 1 / 116 |
| 661 | −39 / 47 | −39 / −1 | 1 / 47 |
| 662 | −27 / 13 | −27 / −1 | 1 / 13 |
| 663 | −16 / 49 | −16 / −1 | 1 / 49 |
| 664 | −55 / 75 | −55 / −1 | 1 / 75 |
| 665 | −84 / 125 | −84 / −1 | 1 / 125 |
| 666 | −17 / 19 | −17 / −1 | 1 / 19 |
| 667 | −29 / 15 | −29 / −1 | 1 / 15 |
| 668 | −52 / 111 | −52 / −1 | 1 / 111 |
| 669 | −47 / 17 | −47 / −1 | 1 / 17 |
| 670 | −50 / 168 | −50 / −1 | 1 / 168 |
| 671 | −15 / 201 | −15 / −1 | 1 / 201 |
| 672 | −19 / 115 | −19 / −1 | 1 / 115 |
| 673 | −16 / 69 | −16 / −1 | 1 / 69 |
| 674 | −29 / 263 | −29 / −1 | 1 / 263 |
| 675 | −56 / 66 | −56 / −1 | 1 / 66 |
| 676 | −28 / 31 | −28 / −1 | 1 / 31 |
| 677 | −13 / 86 | −13 / −1 | 1 / 86 |
| 678 | −13 / 86 | −13 / −1 | 1 / 86 |
| 679 | −25 / 83 | −25 / −1 | 1 / 83 |
| 680 | −15 / 168 | −15 / −1 | 1 / 168 |
| 681 | −15 / 83 | −15 / −1 | 1 / 83 |
| 682 | −57 / 126 | −57 / −1 | 1 / 126 |
| 683 | −14 / 126 | −14 / −1 | 1 / 126 |
| 684 | −14 / 45 | −14 / −1 | 1 / 45 |
| 685 | −36 / 65 | −36 / −1 | 1 / 65 |
| 686 | −55 / 286 | −55 / −1 | 1 / 286 |
| 687 | −42 / 66 | −42 / −1 | 1 / 66 |
| 688 | −26 / 54 | −26 / −1 | 1 / 54 |
| 689 | −44 / 114 | −44 / −1 | 1 / 114 |
| 690 | −28 / 102 | −28 / −1 | 1 / 102 |
| 691 | −62 / 137 | −62 / −1 | 1 / 137 |
| 692 | −25 / 155 | −25 / −1 | 1 / 155 |
| 741 | −22 / 156 | −22 / −1 | 1 / 156 |
| 742 | −19 / 71 | −19 / −1 | 1 / 71 |
| 743 | −15 / 110 | −15 / −1 | 1 / 110 |
| 744 | −34 / 102 | −34 / −1 | 1 / 102 |
| 745 | −53 / 185 | −53 / −1 | 1 / 185 |
| 746 | −71 / 35 | −71 / −1 | 1 / 35 |
| 747 | −84 / 39 | −84 / −1 | 1 / 39 |
| 748 | −49 / 26 | −49 / −1 | 1 / 26 |
| 749 | −40 / 40 | −40 / −1 | 1 / 40 |
| 750 | −49 / 278 | −49 / −1 | 1 / 278 |
| 751 | −20 / 215 | −20 / −1 | 1 / 215 |
| 752 | −31 / 21 | −31 / −1 | 1 / 21 |
| 753 | −47 / 182 | −47 / −1 | 1 / 182 |
| 754 | −24 / 68 | −24 / −1 | 1 / 68 |
| 755 | −23 / 402 | −23 / −1 | 1 / 402 |
| 756 | −62 / 25 | −62 / −1 | 1 / 25 |
| 757 | −100 / 49 | −100 / −1 | 1 / 49 |
| 758 | −35 / 152 | −35 / −1 | 1 / 152 |
| 759 | −26 / 97 | −26 / −1 | 1 / 97 |
| 760 | −102 / 51 | −102 / −1 | 1 / 51 |
| 761 | 1 / 72 | — | 1 / 72 |
| 762 | −20 / 155 | −20 / −1 | 1 / 155 |
| 763 | −20 / 283 | −20 / −1 | 1 / 283 |
| 764 | −39 / 26 | −39 / −1 | 1 / 26 |
| 765 | −17 / 120 | −17 / −1 | 1 / 120 |
| 766 | −13 / 141 | −13 / −1 | 1 / 141 |
| 767 | −36 / 67 | −36 / −1 | 1 / 67 |
| 768 | −74 / 12 | −74 / −1 | 1 / 12 |
| 769 | −310 / 85 | −310 / −1 | 1 / 85 |
| 770 | −18 / 75 | −18 / −1 | 1 / 75 |
| 771 | −21 / 40 | −21 / −1 | 1 / 40 |
| 772 | −31 / 300 | −31 / −1 | 1 / 300 |
| 773 | −99 / 111 | −99 / −1 | 1 / 111 |
| 774 | −67 / 12 | −67 / −1 | 1 / 12 |
| 775 | 1 / 84 | — | 1 / 84 |
| 776 | −20 / 72 | −20 / −1 | 1 / 72 |
| 777 | −14 / 196 | −14 / −1 | 1 / 196 |
| 778 | −139 / 66 | −139 / −1 | 1 / 66 |
| 779 | −17 / 68 | −17 / −1 | 1 / 68 |
| 780 | −51 / 64 | −51 / −1 | 1 / 64 |
| 781 | −31 / 50 | −31 / −1 | 1 / 50 |
| 782 | −39 / 196 | −39 / −1 | 1 / 196 |
| 783 | −21 / 41 | −21 / −1 | 1 / 41 |
| 784 | 1 / 11 | — | 1 / 11 |
| 785 | −34 / 22 | −34 / −1 | 1 / 22 |
| 786 | −32 / 67 | −32 / −1 | 1 / 67 |
| 787 | −27 / 133 | −27 / −1 | 1 / 133 |
| 788 | −22 / 37 | −22 / −1 | 1 / 37 |
| 789 | −48 / 64 | −48 / −1 | 1 / 64 |
| 790 | −56 / 55 | −56 / −1 | 1 / 55 |
| 791 | −77 / 67 | −77 / −1 | 1 / 67 |
| 792 | −18 / 92 | −18 / −1 | 1 / 92 |
| 793 | −36 / 43 | −36 / −1 | 1 / 43 |
| 794 | −34 / 162 | −34 / −1 | 1 / 162 |
| 795 | −18 / 159 | −18 / −1 | 1 / 159 |
| 796 | −22 / 83 | −22 / −1 | 1 / 83 |
| 797 | −48 / 100 | −48 / −1 | 1 / 100 |
| 798 | −23 / 236 | −23 / −1 | 1 / 236 |
| 799 | −62 / 49 | −62 / −1 | 1 / 49 |
| 800 | −23 / 288 | −23 / −1 | 1 / 288 |
| 801 | −60 / 31 | −60 / −1 | 1 / 31 |
| 802 | −17 / 270 | −17 / −1 | 1 / 270 |
| 803 | −49 / 55 | −49 / −1 | 1 / 55 |
| 804 | −36 / 48 | −36 / −1 | 1 / 48 |
| 805 | −20 / 111 | −20 / −1 | 1 / 111 |
| 806 | −23 / 108 | −23 / −1 | 1 / 108 |
| 807 | −16 / 319 | −16 / −1 | 1 / 319 |
| 808 | −55 / 29 | −55 / −1 | 1 / 29 |
| 809 | −18 / 262 | −18 / −1 | 1 / 262 |
| 810 | −51 / 89 | −51 / −1 | 1 / 89 |

TABLE III

| Id | Positions of preferred fragments |
|---|---|
| 1 | 1-126, 164-259, 420-432, 1404-1450 |
| 2 | 32-44, 4199-1556 |
| 3 | 1-19, 1011-1058 |
| 4 | 1-16, 108-159, 595-648 |
| 5 | 1-119, 486-665, 1968-2009, 2055-2104 |
| 6 | 424-435, 500-515 |
| 7 | 1-122, 242-661 |
| 8 | 1-16, 649-694 |
| 9 | 1-663, 1070-110 |
| 10 | 1-129, 541-623 |
| 11 | 1-200, 614-657 |
| 12 | 1-419, 1094-1137 |
| 13 | 1-127, 323-331, 595-636 |
| 14 | 804-818 |
| 15 | 1-47, 438-611, 1005-1133, 1846-1888 |
| 16 | 1-430, 527-1894 |
| 17 | 1-119, 1743-1792, 1866-1913 |
| 18 | 1-70, 133-1235, 1729-1744 |
| 19 | 575-615, 896-946 |
| 20 | 513-526, 950-960, 1577-1622 |
| 21 | 1-2, 210-265, 674-715 |
| 22 | 1400-1441, 1508-1549 |
| 23 | 1-4, 1284, 1328 |

TABLE IVa

| Seq Id N° | Preferred fragments |
|---|---|
| 1 | 1-58: 343-1359: 1434-1450 |
| 2 | 455-1556 |
| 3 | 553-634: 1042-1058 |
| 4 | 608-648 |
| 5 | 452-481: 620-2104 |
| 6 | 424-515 |
| 7 | 497-661 |
| 8 | 529-694 |
| 9 | 639-1110 |
| 10 | 505-623 |
| 11 | 536-657 |
| 12 | 444-1137 |
| 13 | 593-636 |
| 14 | 448-818 |
| 15 | 643-1346: 1809-1888 |
| 16 | 276-1894 |
| 17 | 332-1913 |
| 18 | 392-1744 |
| 19 | 578-946 |
| 20 | 1-240: 645-1224: 1341-1622 |
| 21 | 695-715 |
| 22 | 472-706: 924-1549 |
| 23 | 495-1328 |
| 24 | 440-1193: 1494-1515 |
| 25 | 532-1024: 1065-1622 |
| 26 | 495-582: 1412-1448 |
| 27 | 427-894 |
| 28 | 500-1321: 1424-1447 |
| 29 | 487-1540 |
| 30 | 441-1272: 1330-1643 |
| 31 | 915-1314 |
| 32 | 453-2356 |
| 33 | 519-1701 |
| 34 | 550-772 |
| 35 | 340-987 |
| 36 | 467-1324 |
| 37 | 442-1918 |
| 38 | 521-852 |
| 39 | 452-726 |
| 40 | 128-143: 481-1039 |
| 41 | 492-1355 |
| 42 | 527-572 |
| 43 | 521-535 |
| 44 | 526-572 |
| 45 | 512-804 |
| 46 | 552-629 |
| 47 | 655-669 |
| 48 | 423-973 |
| 49 | 529-791 |
| 50 | 642-1110 |

TABLE IVb

| Seq Id N° | Excluded fragments |
|---|---|
| 1 | 59-342: 1360-1433 |
| 2 | 1-454 |
| 3 | 1-552: 635-1041 |
| 4 | 1-607 |
| 5 | 1-451: 482-619 |
| 6 | 1-423 |
| 7 | 1-496 |
| 8 | 1-528 |
| 9 | 1-638 |
| 10 | 1-504 |
| 11 | 1-535 |
| 12 | 1-443 |
| 13 | 1-592 |
| 14 | 1-447 |
| 15 | 1-642: 1347-1808 |
| 16 | 1-275 |
| 17 | 1-331 |
| 18 | 1-391 |
| 19 | 1-577 |
| 20 | 241-644: 1225-1340 |
| 21 | 1-694 |
| 22 | 1-471: 707-923 |
| 23 | 1-494 |
| 24 | 1-439: 1194-1493 |
| 25 | 1-531: 1025-1064 |
| 26 | 1-494: 583-1411 |
| 27 | 1-426 |
| 28 | 1-499: 1322-1423 |
| 29 | 1-486 |
| 30 | 1-440: 1273-1329 |
| 31 | 1-914 |
| 32 | 1-452 |
| 33 | 1-518 |
| 34 | 1-549 |
| 35 | 1-339 |
| 36 | 1-466 |
| 37 | 1-441 |
| 38 | 1-520 |
| 39 | 1-451 |
| 40 | 1-127: 144-480 |
| 41 | 1-491 |
| 42 | 1-526 |
| 43 | 1-520 |
| 44 | 1-525 |
| 45 | 1-511 |
| 46 | 1-551 |
| 47 | 1-654 |
| 48 | 1-422 |
| 49 | 1-528 |
| 50 | 1-641 |

TABLE V

| | Nucleotide | Protein |
|---|---|---|
| Internal designation | SEQ ID NO | SEQ ID NO |
| 105-016-3-0-E3-FL | 1 | 406 |
| 105-031-3-0-D6-FL | 2 | 407 |
| 105-095-1-0-D10-FL | 3 | 408 |
| 105-118-4-0-E6-FL | 4 | 409 |
| 114-025-2-0-F11-FL | 5 | 410 |
| 116-005-4-0-G11-FL | 6 | 411 |
| 116-032-2-0-F9-FL | 7 | 412 |
| 116-047-3-0-B1-FL | 8 | 413 |
| 116-048-4-0-A6-FL | 9 | 414 |
| 116-049-1-0-F2-FL | 10 | 415 |
| 116-050-2-0-A11-FL | 11 | 416 |
| 116-054-3-0-E6-FL | 12 | 417 |
| 116-054-3-0-G12-FL | 13 | 418 |
| 116-073-4-0-C8-FL | 14 | 419 |
| 117-002-3-0-G3-FL | 15 | 420 |
| 117-005-2-0-E10-FL | 16 | 421 |
| 117-005-3-0-F2-FL | 17 | 422 |
| 117-005-4-0-E5-FL | 18 | 423 |
| 117-007-2-0-B5-FL | 19 | 424 |
| 117-007-2-0-C4-FL | 20 | 425 |
| 121-004-3-0-F6-FL | 21 | 426 |
| 122-005-2-0-F11-FL | 22 | 427 |
| 122-007-3-0-D10-FL | 23 | 428 |
| 108-004-5-0-B12-FL | 24 | 429 |
| 108-004-5-0-C10-FL | 25 | 430 |
| 108-004-5-0-G10-FL | 26 | 431 |
| 108-005-5-0-D4-FL | 27 | 432 |
| 108-005-5-0-F9-FL | 28 | 433 |
| 108-006-5-0-C7-FL | 29 | 434 |
| 108-006-5-0-E1-FL | 30 | 435 |
| 108-008-5-0-C5-FL | 31 | 436 |
| 108-008-5-0-G5-FL | 32 | 437 |
| 108-011-5-0-B12-FL | 33 | 438 |
| 108-011-5-0-C7-FL | 34 | 439 |
| 108-011-5-0-G8-FL | 35 | 440 |
| 108-011-5-0-H2-FL | 36 | 441 |
| 108-013-5-0-G5-FL | 37 | 442 |
| 108-013-5-0-H9-FL | 38 | 443 |
| 108-014-5-0-A10-FL | 39 | 444 |
| 108-014-5-0-C7-FL | 40 | 445 |
| 108-014-5-0-D12-FL | 41 | 446 |
| 108-014-5-0-H8-FL | 42 | 447 |
| 108-015-5-0-E2-FL | 43 | 448 |
| 108-016-5-0-C12-FL | 44 | 449 |
| 108-016-5-0-D4-FL | 45 | 450 |
| 108-019-5-0-F10-FL | 46 | 451 |
| 108-019-5-0-F5-FL | 47 | 452 |
| 108-019-5-0-H3-FL | 48 | 453 |
| 108-020-5-0-D4-FL | 49 | 454 |
| 108-020-5-0-E3-FL | 50 | 455 |
| 20-5-2-C3-CL0_4 | 99 | 456 |
| 20-8-4-A11-CL2_6 | 100 | 457 |
| 21-1-4-F2-CL11_1 | 101 | 458 |
| 22-11-2-H9-CL1_1 | 102 | 459 |
| 25-7-3-D4-CL0_2 | 103 | 460 |
| 26-27-3-D7-CL0_1 | 104 | 461 |
| 26-35-4-H9-CL1_1 | 105 | 462 |
| 26-45-2-C4-CL2_6 | 106 | 463 |
| 27-1-2-B3-CL0_1 | 107 | 464 |
| 27-1-2-B3-CL0_2 | 108 | 465 |
| 27-19-3-G7-CL11_2 | 109 | 466 |
| 33-10-4-E2-CL13_4 | 110 | 467 |
| 33-10-4-H2-CL2_2 | 111 | 468 |
| 33-110-4-A5-CL1_1 | 112 | 469 |
| 33-13-1-C1-CL1_1 | 113 | 470 |
| 33-30-2-A6-CL0_1 | 114 | 471 |
| 33-35-4-F4-CL1_2 | 115 | 472 |
| 33-35-4-G1-CL1_2 | 116 | 473 |
| 33-36-3-E2-CL1_1 | 117 | 474 |
| 33-36-3-E2-CL1_2 | 118 | 475 |
| 33-36-3-F2-CL2_2 | 119 | 476 |
| 33-4-2-G5-CL2_1 | 120 | 477 |
| 33-49-1-H4-CL1_1 | 121 | 478 |
| 33-66-2-B10-CL4_1 | 422 | 479 |
| 33-97-4-G8-CL2_2 | 123 | 480 |
| 33-98-4-C1-CL1_3 | 124 | 481 |
| 47-14-1-C3-CL0_5 | 125 | 482 |
| 47-15-1-E11-CL0_1 | 126 | 483 |
| 47-15-1-H8-CL0_2 | 127 | 484 |
| 48-1-1-H7-CL0_1 | 128 | 485 |
| 48-1-1-H7-CL0_4 | 129 | 486 |
| 48-1-1-H7-CL0_5 | 130 | 487 |
| 48-3-1-H9-CL0_6 | 131 | 488 |
| 48-54-1-G9-CL2_1 | 132 | 489 |
| 48-54-1-G9-CL3_1 | 133 | 490 |
| 48-7-4-H2-CL2_2 | 134 | 491 |
| 51-11-3-D5-CL1_3 | 135 | 492 |
| 51-11-3-G9-CL0_1 | 136 | 493 |
| 51-15-4-A12-CL11_3 | 137 | 494 |
| 51-17-4-A4-CL3_1 | 138 | 495 |
| 51-2-3-F10-CL1_5 | 139 | 496 |
| 51-2-4-F5-CL11_2 | 140 | 497 |
| 51-27-4-F2-CL0_2 | 141 | 498 |
| 51-34-3-F8-CL0_2 | 142 | 499 |
| 57-1-4-E2-CL1_2 | 143 | 500 |
| 57-19-2-G8-CL2_1 | 144 | 501 |
| 57-27-3-G10-CL2_2 | 145 | 502 |
| 58-33-3-B4-CL1_2 | 146 | 503 |
| 58-34-3-C9-CL1_2 | 147 | 504 |
| 58-4-4-G2-CL2_1 | 148 | 505 |
| 58-48-1-G3-CL2_4 | 149 | 506 |
| 58-6-1-H4-CL1_1 | 150 | 507 |
| 60-12-1-E11-CL1_2 | 151 | 508 |
| 65-4-4-H3-CL1_1 | 152 | 509 |
| 74-5-1-E4-CL1_2 | 153 | 510 |
| 76-13-3-A9-CL1_2 | 154 | 511 |
| 76-16-1-D6-CL1_1 | 155 | 512 |
| 76-28-3-A12-CL1_5 | 156 | 513 |
| 76-42-2-F3-CL0_1 | 157 | 514 |
| 77-16-4-G3-CL1_3 | 158 | 515 |
| 77-39-4-H4-CL11_4 | 159 | 516 |
| 78-24-3-H4-CL2_1 | 160 | 517 |
| 78-27-3-D1-CL1_6 | 161 | 518 |
| 78-28-3-D2-CL0_2 | 162 | 519 |
| 78-7-1-G5-CL2_6 | 163 | 520 |
| 84-3-1-G10-CL11_6 | 164 | 521 |
| 58-48-4-E2-CL0_1 | 165 | 522 |
| 23-12-2-G6-CL1_2 | 166 | 523 |
| 25-8-4-B12-CL0_5 | 167 | 524 |
| 26-44-3-C5-CL2_1 | 168 | 525 |
| 27-1-2-B3-CL0_3 | 169 | 526 |
| 30-12-3-G5-CL0_1 | 170 | 527 |
| 33-106-2-F10-CL1_3 | 171 | 528 |
| 33-28-4-D1-CL0_1 | 172 | 529 |
| 33-31-3-C8-CL2_1 | 173 | 530 |
| 48-24-1-D2-CL3_2 | 174 | 531 |
| 48-46-4-A11-CL1_4 | 175 | 532 |
| 51-1-4-C1-CL0_2 | 176 | 533 |
| 51-39-3-H2-CL1_2 | 177 | 534 |
| 51-42-3-F9-CL1_1 | 178 | 535 |
| 51-5-3-G2-CL0_4 | 179 | 536 |
| 57-18-4-H5-CL2_1 | 180 | 537 |
| 76-23-3-G8-CL1_1 | 181 | 538 |
| 76-23-3-G8-CL1_3 | 182 | 539 |
| 78-8-3-E6-CL0_1 | 183 | 540 |
| 19-10-1-C2-CL1_3 | 184 | 541 |
| 33-11-1-B11-CL1_2 | 185 | 542 |
| 33-113-2-B8-CL1_2 | 186 | 543 |
| 33-19-1-C11-CL1_1 | 187 | 544 |
| 33-61-2-F6-CL0_2 | 188 | 545 |
| 47-4-4-C6-CL2_2 | 189 | 546 |
| 48-54-1-G9-CL1_1 | 190 | 547 |
| 51-43-3-G3-CL0_1 | 191 | 548 |
| 55-1-3-D11-CL0_1 | 192 | 549 |
| 58-14-2-D3-CL1_2 | 193 | 550 |
| 58-35-2-B6-CL2_3 | 194 | 551 |
| 76-18-1-F6-CL1_1 | 195 | 552 |
| 76-23-3-G8-CL2_2 | 196 | 553 |
| 76-30-3-B7-CL1_1 | 197 | 554 |
| 78-21-3-G7-CL2_1 | 198 | 555 |
| 58-45-4-B11-CL13_2 | 199 | 556 |
| 20-6-1-D11-FL2 | 200 | 557 |
| 20-8-4-A11-FL2 | 201 | 558 |
| 22-6-2-C1-FL2 | 202 | 559 |
| 22-11-2-H9-FL1 | 203 | 560 |
| 23-8-3-B1-FL1 | 204 | 561 |
| 24-3-3-C6-FL1 | 205 | 562 |
| 24-4-1-H3-FL1 | 206 | 563 |
| 26-45-2-C4-FL2 | 207 | 564 |
| 26-48-1-H10-FL1 | 208 | 565 |
| 26-49-1-A5-FL2 | 209 | 566 |
| 30-6-4-E3-FL3 | 210 | 567 |
| 33-6-1-G11-FL1 | 211 | 568 |
| 33-8-1-A3-FL2 | 212 | 569 |
| 33-11-3-C6-FL1 | 213 | 570 |
| 33-14-4-E1-FL1 | 214 | 571 |
| 33-21-2-D5-FL1 | 215 | 572 |
| 33-26-4-E10-FL1 | 216 | 573 |
| 33-27-1-E11-FL1 | 217 | 574 |
| 33-28-4-D1-FL1 | 218 | 575 |
| 33-28-4-E2-FL2 | 219 | 576 |
| 33-30-4-C4-FL1 | 220 | 577 |
| 33-35-4-F4-FL1 | 221 | 578 |
| 33-36-3-F2-FL2 | 222 | 579 |
| 33-52-4-F9-FL2 | 223 | 580 |
| 33-52-4-H3-FL1 | 224 | 581 |
| 33-59-1-B7-FL1 | 225 | 582 |
| 33-71-1-A8-FL1 | 226 | 583 |
| 33-72-2-B2-FL1 | 227 | 584 |
| 33-105-2-C3-FL1 | 228 | 585 |
| 33-107-4-C3-FL1 | 229 | 586 |
| 33-110-2-G4-FL1 | 230 | 587 |
| 47-7-4-D2-FL2 | 231 | 588 |
| 47-10-2-G12-FL1 | 232 | 589 |
| 47-14-3-D8-FL1 | 233 | 590 |
| 47-18-3-C2-FL1 | 234 | 591 |
| 47-18-3-G5-FL2 | 235 | 592 |
| 47-18-4-E3-FL2 | 236 | 593 |
| 48-3-1-H9-FL3 | 237 | 594 |
| 48-4-2-H3-FL1 | 238 | 595 |
| 48-6-1-C9-FL1 | 239 | 596 |
| 48-7-4-H2-FL2 | 240 | 597 |
| 48-8-1-D8-FL3 | 241 | 598 |
| 48-13-3-H8-FL1 | 242 | 599 |
| 48-19-3-A7-FL1 | 243 | 600 |
| 48-19-3-G1-FL1 | 244 | 601 |
| 48-25-4-D8-FL1 | 245 | 602 |
| 48-21-4-H4-FL1 | 246 | 603 |
| 48-26-3-B8-FL2 | 247 | 604 |
| 48-29-1-E2-FL1 | 248 | 605 |
| 48-31-3-F7-FL1 | 249 | 606 |
| 48-47-3-A5-FL1 | 250 | 607 |
| 51-1-1-G12-FL1 | 251 | 608 |
| 51-1-4-E9-FL3 | 252 | 609 |
| 51-1-4-E9-FL2 | 253 | 610 |
| 51-2-1-E10-FL1 | 254 | 611 |
| 51-2-3-F10-FL1 | 255 | 612 |
| 51-2-4-F5-FL1 | 256 | 613 |
| 51-3-3-B10-FL2 | 257 | 614 |
| 51-3-3-B10-FL3 | 258 | 615 |
| 51-7-3-G3-FL1 | 259 | 616 |
| 51-10-3-D11-FL1 | 260 | 617 |
| 51-11-3-D5-FL1 | 261 | 618 |
| 51-13-1-F7-FL3 | 262 | 619 |
| 51-15-4-H10-FL1 | 263 | 620 |
| 51-17-4-A4-FL1 | 264 | 621 |
| 51-18-1-C3-FL1 | 265 | 622 |
| 51-25-3-F3-FL1 | 266 | 623 |
| 51-27-1-E8-FL1 | 267 | 624 |
| 51-28-2-G1-FL2 | 268 | 625 |
| 51-39-3-H2-FL1 | 269 | 626 |
| 51-42-3-F9-FL1 | 270 | 627 |
| 51-44-4-H4-FL1 | 271 | 628 |
| 55-1-3-H10-FL1 | 272 | 629 |
| 55-5-4-A6-FL1 | 273 | 630 |
| 58-26-3-D1-FL1 | 274 | 631 |
| 57-18-1-D5-FL1 | 275 | 632 |
| 57-27-3-A11-FL1 | 276 | 633 |
| 57-27-3-G10-FL2 | 277 | 634 |
| 58-10-3-D12-FL1 | 278 | 635 |
| 58-26-3-D1-FL1 | 274 | 631 |
| 58-11-1-G10-FL1 | 279 | 636 |
| 58-11-2-G8-FL2 | 280 | 637 |
| 58-36-3-A9-FL2 | 281 | 638 |
| 58-38-1-A2-FL2 | 282 | 639 |
| 58-38-1-E5-FL1 | 283 | 640 |
| 58-44-2-B3-FL3 | 284 | 641 |
| 58-45-3-H11-FL1 | 285 | 642 |
| 58-53-2-B12-FL2 | 286 | 643 |
| 59-9-4-A10-FL1 | 287 | 644 |
| 60-16-3-A6-FL1 | 288 | 645 |
| 60-17-3-G8-FL2 | 289 | 646 |
| 62-5-4-B10-FL1 | 290 | 647 |
| 65-4-4-H3-FL1 | 291 | 648 |
| 74-3-1-B9-FL1 | 292 | 649 |
| 76-4-1-G5-FL1 | 293 | 650 |
| 76-7-3-A12-FL1 | 294 | 651 |
| 76-16-4-C9-FL3 | 295 | 652 |
| 76-30-3-B7-FL1 | 296 | 653 |
| 77-5-1-C2-FL1 | 297 | 654 |
| 77-5-4-E7-FL1 | 298 | 655 |
| 77-11-1-A3-FL1 | 299 | 656 |
| 77-16-3-D7-FL1 | 300 | 657 |
| 77-16-4-G3-FL1 | 301 | 658 |
| 77-25-1-A6-FL1 | 302 | 659 |
| 77-26-2-F2-FL3 | 303 | 660 |
| 78-6-2-E3-FL2 | 304 | 661 |
| 78-7-1-G5-FL2 | 305 | 662 |
| 78-16-2-C2-FL1 | 306 | 663 |
| 78-18-3-B4-FL3 | 307 | 664 |
| 78-20-1-G11-FL1 | 308 | 665 |
| 78-22-3-E10-FL1 | 309 | 666 |
| 78-24-2-B8-FL1 | 310 | 667 |
| 78-24-3-A8-FL1 | 311 | 668 |
| 78-24-3-H4-FL2 | 312 | 669 |
| 78-25-1-F11-FL1 | 313 | 670 |
| 78-26-1-B5-FL1 | 314 | 671 |
| 78-27-3-D1-FL1 | 315 | 672 |
| 78-29-1-B2-FL1 | 316 | 673 |
| 78-29-4-B6-FL1 | 317 | 674 |
| 14-1-3-E6-FL1 | 318 | 675 |
| 30-9-1-G8-FL2 | 319 | 676 |
| 33-10-4-H2-FL2 | 320 | 677 |
| 33-10-4-H2-FL1 | 321 | 678 |
| 74-10-3-C9-FL2 | 322 | 679 |
| 33-97-4-G8-FL3 | 323 | 680 |
| 33-97-4-G8-FL2 | 324 | 681 |
| 33-104-4-H4-FL1 | 325 | 682 |
| 47-2-3-B3-FL1 | 326 | 683 |
| 47-37-4-G11-FL1 | 327 | 684 |
| 57-25-1-F10-FL2 | 328 | 685 |
| 58-19-3-D3-FL1 | 329 | 686 |
| 58-34-3-C9-FL2 | 330 | 687 |
| 58-48-4-E2-FL2 | 331 | 688 |
| 76-21-1-C4-FL1 | 332 | 689 |
| 78-26-2-H7-FL1 | 333 | 690 |
| 77-20-2-E11-FL1 | 334 | 691 |
| 47-1-3-F7-FL2 | 335 | 692 |
| 108-002-5-0-B1-FL | 336 | 741 |
| 108-002-5-0-F3-FL | 337 | 742 |
| 108-002-5-0-F4-FL | 338 | 743 |
| 108-003-5-0-A8-FL | 339 | 744 |
| 108-003-5-0-D2-FL | 340 | 745 |
| 108-003-5-0-E5-FL | 341 | 746 |
| 108-003-5-0-H2-FL | 342 | 747 |
| 108-004-5-0-B7-FL | 343 | 748 |
| 108-004-5-0-C8-FL | 344 | 749 |
| 108-004-5-0-D10-FL | 345 | 750 |
| 108-004-5-0-E8-FL | 346 | 751 |
| 108-004-5-0-F5-FL | 347 | 752 |
| 108-004-5-0-G6-FL | 348 | 753 |
| 108-005-5-0-B11-FL | 349 | 754 |
| 108-005-5-0-C1-FL | 350 | 755 |
| 108-005-5-0-F11-FL | 351 | 756 |
| 108-005-5-0-F6-FL | 352 | 757 |
| 108-006-5-0-C2-FL | 353 | 758 |
| 108-006-5-0-E6-FL | 354 | 759 |
| 108-006-5-0-G2-FL | 355 | 760 |
| 108-006-5-0-G4-FL | 356 | 761 |
| 108-008-5-0-A6-FL | 357 | 762 |
| 108-008-5-0-A8-FL | 358 | 763 |
| 108-008-5-0-C10-FL | 359 | 764 |
| 108-008-5-0-E6-FL | 360 | 765 |
| 108-008-5-0-F6-FL | 361 | 766 |
| 108-008-5-0-G12-FL | 362 | 767 |
| 108-008-5-0-G4-FL | 363 | 768 |
| 108-009-5-0-A2-FL | 364 | 769 |
| 108-013-5-0-C12-FL | 365 | 770 |
| 108-013-5-0-G11-FL | 366 | 771 |
| 108-003-5-0-E4-FL | 367 | 772 |
| 108-005-5-0-D6-FL | 368 | 773 |
| 108-008-5-0-G3-FL | 369 | 774 |
| 108-013-5-0-B5-FL | 370 | 775 |
| 26-44-1-B5-CL3_1 | 371 | 776 |
| 47-4-4-C6-CL2_3 | 372 | 777 |
| 47-40-4-G9-CL1_1 | 373 | 778 |
| 48-25-4-D8-CL1_7 | 374 | 779 |
| 48-28-3-A9-CL0_1 | 375 | 780 |
| 51-25-1-A2-CL3_1 | 376 | 781 |
| 55-10-3-F5-CL0_3 | 377 | 782 |
| 57-19-2-G8-CL1_3 | 378 | 783 |
| 58-34-2-H8-CL1_3 | 379 | 784 |
| 76-13-3-A9-CL1_1 | 380 | 785 |
| 78-7-2-B8-FL1 | 381 | 786 |
| 77-8-4-F9-FL1 | 382 | 787 |
| 58-8-1-F2-FL2 | 383 | 788 |
| 77-13-1-A7-FL2 | 384 | 789 |
| 47-2-3-G9-FL1 | 385 | 790 |
| 33-75-4-H7-FL1 | 386 | 791 |
| 51-41-1-F10-FL1 | 387 | 792 |
| 48-51-4-C11-FL1 | 388 | 793 |
| 33-58-3-C8-FL1 | 389 | 794 |
| 76-20-4-C11-FL1 | 390 | 795 |
| 76-28-3-A12-FL1 | 391 | 796 |
| 76-25-4-F11-FL1 | 392 | 797 |
| 58-20-4-G7-FL1 | 393 | 798 |
| 33-54-1-B9-FL1 | 394 | 799 |
| 76-20-3-H1-FL1 | 395 | 800 |
| 47-20-2-G3-FL1 | 396 | 801 |
| 78-25-1-H11-FL1 | 397 | 802 |
| 78-6-2-B10-FL1 | 398 | 803 |
| 58-49-3-G10-FL1 | 399 | 804 |
| 78-21-1-B7-FL1 | 400 | 805 |
| 57-28-4-B12-FL1 | 401 | 806 |
| 33-77-4-E2-FL1 | 402 | 807 |
| 58-19-3-D3-FL2 | 403 | 808 |
| 37-7-4-E7-FL1 | 404 | 809 |
| 60-14-2-H10-FL1 | 405 | 810 |

TABLE VI

| Seq Id No | Tissue expression |
|---|---|
| 1 | prostate: 2 |
| 2 | fetal kidney: 1 prostate: 3 |
| 4 | prostate: 1 |
| 5 | liver: 1 |
| 6 | testis: 1 |
| 7 | testis: 3 |
| 8 | testis: 1 |
| 9 | testis: 1 |
| 10 | testis: 1 |
| 11 | liver: 1 testis: 3 |
| 12 | liver: 1 testis: 3 |
| 13 | testis: 1 |
| 14 | testis: 1 |
| 15 | liver: 2 |
| 16 | liver: 3 |
| 17 | liver: 1 |
| 18 | liver: 1 |
| 19 | brain: 2 liver: 1 placenta: 6 |
| | salivary gland: 1 |
| 20 | fetal brain: 6 |
| 21 | fetal brain: 6 placenta: 2 |
| 22 | fetal brain: 9 |
| 23 | prostate: 2 |
| 24 | prostate: 3 |
| 25 | prostate: 1 |
| 26 | prostate: 1 |
| 27 | prostate: 3 |
| 28 | prostate: 3 |
| 29 | prostate: 2 |
| 30 | prostate: 1 |
| 31 | prostate: 1 |
| 32 | liver: 15 testis: 3 |
| 33 | liver: 1 testis: 8 |
| 34 | brain: 1 |
| 35 | prostate: 1 |
| 36 | liver: 15 |
| 37 | prostate: 2 |
| 38 | testis: 1 |
| 39 | testis: 3 |
| 40 | liver: 2 |
| 41 | liver: 1 testis: 2 |
| 42 | liver: 5 testis: 20 |
| 43 | brain: 4 fetal brain: 10 |
| | fetal kidney: 1 fetal livery: 1 |
| | placenta: 1 prostate: 1 |
| 44 | brain: 3 fetal brain: 4 |
| | fetal kidney: 7 prostate: 1 |
| | salivary gland: 1 testis: 2 |
| 45 | liver: 1 testis: 1 |
| 46 | fetal livery: 1 prostate: 1 |
| | salivary gland: 3 |
| | stomach/intestine: 2 testis: 1 |
| 47 | testis: 1 |
| 48 | fetal brain: 4 |
| 49 | brain: 85 |

TABLE VII

| Seq Id No | Preferential expression |
|---|---|
| 1 | Prostate |
| 2 | Prostate |
| 4 | Prostate |
| 5 | None |
| 6 | None |
| 7 | Testis |
| 8 | None |
| 9 | None |
| 10 | None |
| 11 | Testis |
| 12 | Testis |
| 13 | None |
| 14 | None |
| 15 | Liver |
| 16 | Liver |
| 17 | None |
| 18 | None |
| 19 | Placenta |
| 20 | Fetal brain |
| 21 | None |
| 22 | Fetal brain |
| 23 | Prostate |
| 24 | Prostate |
| 25 | Prostate |
| 26 | Prostate |
| 27 | Prostate |
| 28 | Prostate |
| 29 | Prostate |
| 30 | Prostate |
| 31 | Prostate |
| 32 | Liver |
| 33 | Testis |
| 34 | None |
| 35 | Prostate |
| 36 | Liver |
| 37 | Prostate |
| 38 | None |
| 39 | Testis |
| 40 | Liver |
| 41 | None |
| 42 | Testis |
| 43 | None |
| 44 | Fetal kidney |
| 45 | None |
| 46 | Salivary gland, Stomach/Intestine |
| 47 | None |
| 48 | Fetal brain |
| 49 | Brain |

TABLE VIII

| Seq Id No | Public expression |
|---|---|
| 1 | frontal lobe(2) |
| 2 | B-cell, chronic lymphotic leukemia(2), |
| | “adenocarcinoma”(2), “germinal |
| | center B cell”(2), “liver”(1), |
| | “lung”(1), “tumor”(1) |
| 4 | 2 pooled tumors (clear cell type)(5), |
| | “adenocarcinoma”(1), “anaplastic |
| | oligodendroglioma”(4), “brain”(3), |
| | “breast”(4), “breast tumor”(1), |
| | “carcinoid”(5), “cerebellum”(1), |
| | “colon”(4), “colon tumor RER+”(2), |
| | “frontal lobe”(5), “germinal center B |
| | cell”(4), “glioblastoma (pooled)”(2), |
| | “moderately-differentiated adenocarcinoma”(1), |
| | “normal prostate”(3), “ovary”(2), “parathyroid |
| | tumor”(4), “pectoral muscle (after mastectomy)”(1), |
| | “pooled germ cell tumors”(5), “senescent |
| | fibroblast”(4), “tumor”(1), “tumor, 5 |
| | pooled (see description)”(1) |
| 5 | colon(1), “neuroepithelial cells”(1) |
| 6 | 2 pooled tumors (clear cell type)(2), |
| | “anaplastic oligodendroglioma”(2), |
| | “borderline ovarian carcinoma”(1), |
| | “carcinoid”(3), “colon”(1), “epithelium |
| | (cell line)”(1), “glioblastoma (pooled)”(1), |
| | “ovarian tumor”(1), “pooled germ |
| | cell tumors”(2) |
| 7 | NONE |
| 8 | 2 pooled tumors (clear cell type)(5), |
| | “breast”(1), “carcinoid”(1), “colon tumor, |
| | RER+”(1), “kidney tumor”(1), “pooled |
| | germ cell tumors”(1) |
| 9 | NONE |
| 10 | 2 pooled tumors (clear cell type)(2) |
| 11 | NONE |
| 12 | NONE |
| 13 | 2 pooled tumors (clear cell type)(4), “breast”(1), |
| | “prostate”(1) |
| 14 | pooled germ cell tumors(1) |
| 15 | NONE |
| 16 | liver(2) |
| 17 | B-cell, chronic lymphotic leukemia(2), “brain”(1), |
| | “carcinoid”(1), “colon”(1) |
| 18 | NONE |
| 19 | anaplastic oligodendroglioma(2), “cerebellum”(1), |
| | “colon”(1), “glioblastoma (pooled)”(5), |
| | “metastatic prostate bone lesion”(1), “normal |
| | epithelium”(1), “parathyroid tumor”(1), |
| | “pooled germ cell tumors”(1), “renal cell |
| | tumor”(1), “retina”(2), “squamous cell |
| | carcinoma”(1), “squamous cell carcinoma from |
| | base of tongue”(1), “three pooled meningiomas”(1) |
| 20 | anaplastic oligodendroglioma(1), “brain”(1), |
| | “frontal lobe”(6), “total brain”(2) |
| 21 | Lung(1), “muscle”(1), “parathyroid |
| | tumor”(1), “synovial membrane”(1) |
| 22 | neuroepithelial cells(1), “total brain”(1) |
| 23 | Bone(1), “bone marrow stroma”(1), “brain”(1), |
| | “testis”(1) |
| 24 | NONE |
| 25 | parathyroid tumor(1), “retina”(1), |
| | “total brain”(2) |
| 26 | NONE |
| 27 | ovarian tumor(3), “retina”(1), “senescent |
| | fibroblast”(1) |
| 28 | normal prostate(1) |
| 29 | NONE |
| 30 | foreskin(1) |
| 31 | NONE |
| 32 | NONE |
| 33 | NONE |
| 34 | NONE |
| 35 | adenocarcinoma(1), “pectoral muscle (after |
| | mastectomy)”(1) |
| 36 | juvenile granulosa rumor(1), “liver”(1), |
| | “senescent fibroblast”(1) |
| 37 | 2 pooled tumors (clear cell type)(2), “germinal |
| | center B cell”(6) |
| 38 | NONE |
| 39 | NONE |
| 40 | NONE |
| 41 | NONE |
| 42 | NONE |
| 43 | B-cell, chronic lymphotic leukemia(1), |
| | “adenocarcinoma”(1), “anaplastic |
| | oligodendroglioma”(3), “carcinoid”(3), |
| | “frontal lobe”(2), “glioblastoma |
| | (pooled)”(4), “normal epithelium”(1), |
| | “pooled germ cell tumors”(1) |
| 44 | 2 pooled tumors (clear cell type)(5), |
| | “Lung”(1), “adenocarcinoma”(4), |
| | “adipose tissue, white”(1), “adrenal |
| | adenoma”(1), “anaplastic |
| | oligodendroglioma”(2), “breast tumor”(1), |
| | “carcinoid”(1), “colon”(4), |
| | “epithelium (cell line)”(1), “liver”(1), |
| | “melanocyte”(1), “ovarian tumor”(1), |
| | “parathyroid tumor”(6), “pectoral muscle |
| | (after mastectomy)”(4), “squamous |
| | cell carcinoma”(1), “synovial membrane”(3) |
| 45 | NONE |
| 46 | 2 pooled tumors (clear cell type)(1), “anaplastic |
| | oligodendroglioma”(2), “carcinoid”(3), |
| | “colon”(4), “epithelium (cell line)”(1), |
| | “glioblastoma (pooled)”(1), “normal |
| | prostate”(2), “ovarian tumor”(2), “pooled |
| | germ cell tumors”(3), “senescent |
| | fibroblast”(2), “testis”(1) |
| 47 | NONE |
| 48 | anaplastic oligodendroglioma(2), |
| | “astrocytoma”(1), “glioblastoma |
| | (pooled)”(1), “total brain”(1) |
| 49 | NONE |

TABLE IX

| Seq | | | |
|---|---|---|---|
| Id | | | |
| No | Positions | Motif designation | Database |
| 406 | none | none | none |
| 407 | none | none | none |
| 408 | none | none | none |
| 409 | 33-79 | PHD | Pfam |
| 410 | none | none | none |
| 411 | none | none | none |
| 412 | none | none | none |
| 413 | 28-94 | pfkB | Pfam |
| 414 | none | none | none |
| 415 | none | none | none |
| 416 | none | none | none |
| 417 | none | none | none |
| 418 | none | none | none |
| 419 | 88-213 | lys | Pfam |
| 419 | 183-202 | BL00128C Alpha-lactalbumin / | BLOCKSPLUS |
| | | lysozyme C signature | |
| 419 | 111-120 | PR00135B lysozyme/alpha- | BLOCKSPLUS |
| | | lactalbumin superfamily | |
| | | signature | |
| 419 | 162-180 | Alpha-lactalbumin / | PROSITE |
| | | lysozyme C signature | |
| 420 | 246-266 | PSAP | Pfam |
| 421 | 92-207 | NusB | Pfam |
| 421 | 4-251 | Apolipoprotein | Pfam |
| 421 | 110-263 | Nop | Pfam |
| 422 | none | none | none |
| 423 | 2-134 | mito_carr 1/2 | Pfam |
| 423 | 156-303 | mito_carr 2/2 | Pfam |
| 423 | 5-29 | BL00215A Mitochondrial | BLOCKSPLUS |
| | | energy transfer proteins | |
| 423 | 223-247 | BL00215A Mitochondrial | BLOCKSPLUS |
| | | energy transfer proteins | |
| 423 | 102-125 | BL00215A Mitochondrial | BLOCKSPLUS |
| | | energy transfer proteins | |
| 423 | 169-182 | BL00215B Mitochondrial | BLOCKSPLUS |
| | | energy transfer proteins | |
| 424 | none | none | none |
| 425 | 37-104 | cystatin 1/2 | Pfam |
| 425 | 157-254 | cystatin 2/2 | Pfam |
| 426 | 105-154 | GST | Pfam |
| 427 | 27-131 | Cyt_reductase | Pfam |
| 427 | 158-272 | oxidored_fad | Pfam |
| 427 | 256-265 | PR00406F cytochrome b5 | BLOCKSPLUS |
| | | reductase signature | |
| 427 | 123-138 | PR00406C cytochrome b5 | BLOCKSPLUS |
| | | reductase signature | |
| 427 | 256-268 | BL00559L Eukaryotic | BLOCKSPLUS |
| | | molybdopterin oxidoreductases | |
| | | proteins | |
| 427 | 163-180 | PR00406D cytochrome b5 | BLOCKSPLUS |
| | | reductase signature | |
| 427 | 163-179 | PR00371D flavoprotein | BLOCKSPLUS |
| | | pyridine nucleotide | |
| | | cytochrome reductase | |
| | | signature | |
| 427 | 110-120 | PR00371C flavoprotein | BLOCKSPLUS |
| | | pyridine nucleotide | |
| | | cytochrome reductase | |
| | | signature | |
| 428 | 7-27 | PR00953B flagellar | BLOCKSPLUS |
| | | biosynthetic protein | |
| | | flir signature | |
| 429 | none | none | none |
| 430 | none | none | none |
| 431 | none | none | none |
| 432 | none | none | none |
| 433 | 7-214 | Hydrolase | Pfam |
| 434 | 48-53 | Cytochrome c family | PROSITE |
| | | heme-binding site | |
| 434 | 24-26 | Protein kinase C | PROSITE |
| | | phosphorylation site | |
| 435 | none | none | none |
| 436 | none | none | none |
| 437 | 302-339 | zf-C3HC4 | Pfam |
| 438 | none | none | none |
| 439 | 17-67 | rnaseA | Pfam |
| 440 | none | none | none |
| 441 | none | none | none |
| 442 | 17-40 | A2M_N | Pfam |
| 443 | 52-66 | PR00111B alpha/beta | BLOCKSPLUS |
| | | hydrolase fold signature | |
| 444 | none | none | none |
| 445 | 59-61 | Cell attachment sequence | PROSITE |
| 446 | 258-298 | zf-C3HC4 | Pfam |
| 446 | 257-301 | PHD | Pfam |
| 447 | none | none | none |
| 448 | none | none | none |
| 449 | none | none | none |
| 450 | none | none | none |
| 451 | none | none | none |
| 452 | none | none | none |
| 453 | none | none | none |
| 454 | none | none | none |
| 455 | none | none | none |
| 510 | 110-121 | Aldehyde dehydrogenase | PROSITE |
| | | cysteine active site | |
| 536 | 28-37 | ATP synthase alpha and | PROSITE |
| | | beta subunits signature | |
| 538 | 171-181 | Regulator of chromosome | PROSITE |
| | | condensation (RCC1) | |
| | | signature 2 | |
| 540 | 90-112 | Phosphatidylethanolamine- | PROSITE |
| | | binding protein family | |
| | | signature | |
| 541 | 10-34 | Protein kinases | PROSITE |
| | | ATP-binding region | |
| | | signature | |

TABLE X

| Seq | |
|---|---|
| Id No | Antigenic epitopes |
| 406 | 58, 86-88, 148-149, 175-177, 238-239, 319 |
| 407 | 43-45, 58, 63-64, 72-74, 202, 204-205, 207, 237-238, |
| | 298 |
| 408 | 119, 121 |
| 409 | 21, 40-43 |
| 410 | 41, 43-44, 83, 103-104, 184-185, 187-188, 210-212, |
| | 366-367, 372-373, 396-397, 421, 475-477 |
| 411 | 84, 86-87 |
| 412 | 17, 37-38, 40-41, 43-44 |
| 413 | 97-98 |
| 414 | 34 |
| 415 | 20, 26-30, 83-86, 103, 111-112, 131 |
| 416 | 9-10, 96-97 |
| 417 | 220-222, 230-231 |
| 418 | 36, 44-47, 50-51, 67-68, 81-83 |
| 419 | 44-45, 105-106, 108-109, 147-149, 173, 202-203 |
| 420 | 129-130, 178, 311-312, 333-335, 368-369 |
| 421 | 34, 36-37, 319-320, 331-333 |
| 422 | 60 |
| 423 | 31-32, 157-158, 180, 215-216, 250 |
| 424 | 60-61 |
| 425 | 35, 37-38, 54-55, 57-58, 75-76, 160-161, 183-184, 215- |
| | 216, 230, 291-292, 296, 302, 309 |
| 426 | 5, 9, 11, 99, 184 |
| 427 | 61-62, 87-88, 109-110, 147-148, 216-217, 229-231, 252, 273 |
| 428 | 83, 89, 249-250 |
| 429 | 34-35, 209-211 |
| 430 | 104-106, 199-200, 228-229, 245-246, 292, 326-327, |
| | 342-343 |
| 431 | 25-28, 105-106, 108-109 |
| 432 | 59-60, 97-98, 101-102, 106-107, 159-160, 193-194, |
| | 207-208 |
| 433 | 61 |
| 434 | 56-57, 61-63, 83-84 |
| 435 | 47-48, 77-80, 100, 107 |
| 436 | 92-93 |
| 437 | 3-5, 59, 112-113, 213-214 |
| 438 | 31-32, 66, 108-109, 148-149, 165-167, 170-172, 290- |
| | 291, 339-340 |
| 439 | 32-34, 37-38, 57 |
| 440 | 6-7, 9, 11-12, 56-57 |
| 441 | 47-49, 91-92 |
| 442 | 38-39, 74, 92-93, 108-109, 116 |
| 443 | 17, 96 |
| 444 | 41-43 |
| 445 | 34-34, 84-85 |
| 446 | 83-84, 135-136, 264-265 |
| 447 | 19-23, 41 |
| 448 | 44-44, 109-109 |
| 449 | 4-5, 7-8, 55-56, 94-95 |
| 450 | 31-32, 38-40, 59-60 |
| 451 | 54-55, 59 |
| 452 | 137-137, 139-140 |
| 453 | 56, 86 |
| 454 | 4-5, 58-58, 67-68, 70-72, 74-77, 82-83 |
| 455 | 34 |

TABLE XI

| Seq Id No | Chromosomal location |
|---|---|
| 1 | none |
| 2 | 9 |
| 3 | 20 |
| 4 | 17 |
| 5 | 8 |
| 6 | 16 |
| 7 | 1 |
| 8 | none |
| 9 | none |
| 10 | none |
| 11 | none |
| 12 | none |
| 13 | none |
| 14 | 17 |
| 15 | 12q |
| 16 | 11 |
| 17 | 18 |
| 18 | 14 |
| 19 | 6p23-25.1 |
| 20 | none |
| 21 | 20q12 |
| 22 | none |
| 23 | 3 |
| 24 | none |
| 25 | 1 |
| 26 | 20 |
| 27 | none |
| 28 | 9 |
| 29 | 11q24 |
| 30 | 17 |
| 31 | none |
| 32 | 1 |
| 33 | 3 |
| 34 | 14 |
| 35 | 16 |
| 36 | 11 |
| 37 | 10 |
| 38 | none |
| 39 | none |
| 40 | 19 |
| 41 | none |
| 42 | 6 |
| 43 | X |
| 44 | 6p12.3-21.2 |
| 45 | 5 |
| 46 | none |
| 47 | 16 |
| 48 | 9 |
| 49 | 20 |
| 50 | none |

What is claimed is:

1. An isolated polynucleotide, comprising a nucleic acid sequence selected from the group consisting of:
a) a polynucleotide of any one of SEQ ID NOs: 1-405, or of a human cDNA of a deposited clone, encoding at least any single integer from 6 to 500 amino acids of any one of SEQ ID NOs: 406-810,
b) a polynucleotide of any one of SEQ ID NOs: 1-405, or of a human cDNA of a deposited clone, encoding the signal peptide sequence of any one of SEQ ID NOs: 406-810,
c) a polynucleotide of any one of SEQ ID NOs: 1-405, or of a human cDNA of a deposited clone, encoding a mature polypeptide sequence of any one of SEQ ID NOs: 406-810,
d) a polynucleotide of any one of SEQ ID NOs: 1-405, or of a human cDNA of a deposited clone, encoding a full length polypeptide sequence of any one of SEQ ID NOs: 406-810,
e) a polynucleotide of any one of SEQ ID NOs: 1-405, or of a human cDNA of a deposited clone, encoding a polypeptide sequence of a biologically active fragment of any one of SEQ ID NOs: 406-810,
f) a polynucleotide encoding a polypeptide sequence of at least any single integer from 6 to 500 amino acids of any one of SEQ ID NOs: 406-810 or of a polypeptide encoded by a human cDNA of a deposited clone,
g) a polynucleotide encoding a polypeptide sequence of a signal peptide of any one of SEQ ID NOs: 406-810 or of a signal peptide encoded by a human cDNA of a deposited clone,
h) a polynucleotide encoding a polypeptide sequence of a mature polypeptide of any one of SEQ ID NOs: 406-810 or of a mature polypeptide encoded by a human cDNA of a deposited clone,
i) a polynucleotide encoding a polypeptide sequence of a full length polypeptide of any one of SEQ ID NOs: 406-810 or of a mature polypeptide encoded by a human cDNA of a deposited clone,
j) a polynucleotide encoding a polypeptide sequence of a biologically polypeptide of any one of SEQ ID NOs: 406-810, or of a biologically polypeptide encoded by a human cDNA of a deposited clone,
k) a polynucleotide of any one of a) through j) further comprising an expression vector,
l) a host cell recombinant for a polynucleotide of a) through k) above,
m) a non-human transgenic animal comprising the host cell of k),
n) a polynucleotide of a) through j) further comprising a physiologically acceptable carrier.

2. A polypeptide comprising an amino acid sequence selected from the group consisting of:
a) any single integer from 6 to 500 amino acids of any one of SEQ ID NOs: 406-810 or of a polypeptide encoded by a human cDNA of a deposited clone;
b) a signal peptide sequence of any one of SEQ ID NOs: 406-810 or encoded by a human cDNA of a deposited clone;
c) a mature polypeptide sequence of any one of SEQ ID NOs: 406-810 or encoded by a human cDNA of a deposited clone;
d) a full length polypeptide sequence of any one of SEQ ID NOs: 406-810 or encoded by a human cDNA of a deposited clone;
e) a polypeptide of a) through d) further comprising a physiologically acceptable carrier.

3. A method of making a polypeptide, said method comprising
a) providing a population of host cells comprising the polynucleotide of claim 1;
b) culturing said population of host cells under conditions conducive to the production of a polypeptide of claim 2 within said host cells; and
c) purifying said polypeptide from said population of host cells.

4. A method of making a polypeptide, said method comprising:
a) providing a population of cells comprising a polynucleotide encoding the polypeptide of claim 2, operably linked to a promoter;
b) culturing said population of cells under conditions conducive to the production of said polypeptide within said cells; and
c) purifying said polypeptide from said population of cells.

5. An antibody that specifically binds to the polypeptide of claim 2.

6. A method of binding a polypeptide of claim 2 to an antibody of claim 5, comprising contacting said antibody with said polypeptide under conditions in which antibody can specifically bind to said polypeptide.

7. A method of determining whether a GENSET gene is expressed within a mammal, said method comprising the steps of:
a) providing a biological sample from said mammal
b) contacting said biological sample with either of:
i) a polynucleotide that hybridizes under stringent conditions to the polynucleotide of claim 1; or
ii) a polypeptide that specifically binds to the polypeptide of claim 2; and
c) detecting the presence or absence of hybridization between said polynucleotide and an RNA species within said sample, or the presence or absence of binding of said polypeptide to a protein within said sample;
wherein a detection of said hybridization or of said binding indicates that said GENSET gene is expressed within said mammal.

8. The method of claim 7, wherein said polynucleotide is a primer, and wherein said hybridization is detected by detecting the presence of an amplification product comprising the sequence of said primer.

9. The method of claim 7, wherein said polypeptide is an antibody.

10. A method of determining whether a mammal has an elevated or reduced level of GENSET gene expression, said method comprising the steps of:
a) providing a biological sample from said mammal; and
b) comparing the amount of the polypeptide of claim 2, or of an RNA species encoding said polypeptide, within said biological sample with a level detected in or expected from a control sample;
wherein an increased amount of said polypeptide or said RNA species within said biological sample compared to said level detected in or expected from said control sample indicates that said mammal has an elevated level of said GENSET gene expression, and wherein a decreased amount of said polypeptide or said RNA species within said biological sample compared to said level detected in or expected from said control sample indicates that said mammal has a reduced level of said GENSET gene expression.

11. A method of identifying a candidate modulator of a GENSET polypeptide, said method comprising:
a) contacting the polypeptide of claim 2 with a test compound; and
b) determining whether said compound specifically binds to said polypeptide;
wherein a detection that said compound specifically binds to said polypeptide indicates that said compound is a candidate modulator of said GENSET polypeptide.

12. The method of claim 11, further comprising testing the biological activity of said GENSET polypeptide in the presence of said candidate modulator, wherein an alteration in the biological activity of said GENSET polypeptide in the presence of said compound in comparison to the activity in the absence of said compound indicates that the compound is a modulator of said GENSET polypeptide.

13. A method for the production of a pharmaceutical composition comprising
a) identifying a modulator of a GENSET polypeptide using the method of claim 11; and
b) combining said modulator with a physiologically acceptable carrier.
